SEQUENCE LISTING (164 sequences)

SEQ ID NO: 1 - protein, 344 aa, Homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 2 - DNA, 1821 bp, Homo sapiens
gacaaattgggaatgtttaagtctctgaaactctgcactgaaaagaaaataagattgataacttaagcttaacattctgaggcataaagaaacattaactttggagtattcatcttgactactgaaatacaagtttagaagacaagtggtttcattctggtcacagatcacagcttttctttaaatttataatcctatgggttggactcgttgactgtattttttaaaggttgctcgtcagttaactgagccttggaattcatggattttctaaagactaacaaatgaaaatattttcctgttgaagaacccagcggaaattttacagcaacaaatttcatgtttcttttgggtatttctgagaaaaaggaaatatttataaaaccatccaaagatccagataatttgcaaataaattggaggttatagaggttataatctgaatcccaaaggagactgcagctgatgaaagtgcttccaaactgaaaattggacgtgcctttacgatggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagcaaaataaacaaaactaaggttttaaaaatgatttttgtacatttgatcatattctgtttctgttttgttccttacaatatcaatcttattttatattctcttgtgagaacacaaacatttgttaattgctcagtagtggcagcagtaaggacaatgtacccaatcactctctgtattgctgtttccaactgttgttttgaccctatagtttactactttacatcggacacaattcagaattcaataaaaatgaaaaactggtctgtcaggagaagtgacttcagattctctgaagttcatggtgcagagaattttattcagcataacctacagaccttaaaaagtaagatatttgacaatgaatctgctgcctgaaataaaaccattaggactcactgggacagaactttcaagttccttcaactgtgaaaagtgtctttttggacaaactatttttccacctccaaaagaaattaacacatggacattttaaagtctttagtataaagaaaatttgtattcaatgtgttaagcattaacatgtattttatttgtgtatccactccatctgatttttctgagccattttgatttgttccttcattaaaaaaaatctcttaaagctaaaaaaaaaaaaaaaaaaaaaaaaaaaaaa

SEQ ID NO: 3 - protein, 344 aa, Homo sapiens
MVSVNSSHCFYNDSFKYTLYGCMFSMVFVLGLISNCVAIYIFICVLKVRNETTTYMINLAMSVLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 4 - protein, 344 aa, Homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFFEIVGFFIPLILNVTCSSMVLKTLTKPVTLSRSKINKTKVLKMIFVHLIIFCFCFVPYNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 5 - protein, 344 aa, Homo sapiens [sequence omitted]

SEQ ID NO: 6 - protein, 344 aa, Homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVRTQTFVNCSVVAAVRTMYPITLYIAVSNCCFDPIVYYFTSDTIQNSIKMKNWSVRRSDFRFSEVHGAENFIQHNLQTLKSKIFDNESAA

SEQ ID NO: 7 - DNA, 1039 bp, Homo sapiens
atggtaagcgttaacagctcccactgcttctataatgactcctttaagtacactttgtatgggtgcatgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagcaaaataaacaaaactaaggttttaaaaatgatttttgtacatttgatcatattctgtttctgttttgttccttacaatatcaatcttattttatattctcttgtgagaacacaaacatttgttaattgctcagtagtggcagcagtaaggacaatgtacccaatcactctctgtattgctgtttccaactgttgttttgaccctatagtttactactttacatcggacacaattcagaattcaataaaaatgaaaaactggtctgtcaggagaagtgacttcagattctctgaagttcatggtgcagagaattttattcagcataacctacagaccttaaaaagtaagatatttgacaatgaatctgctgcctga

SEQ ID NO: 8 - DNA, 1030 bp, Homo sapiens
atggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgatttgcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagcaaaataaacaaaactaaggttttaaaaatgatttttgtacatttgatcatattctgtttctgttttgttccttacaatatcaatcttattttatattctcttgtgagaacacaaacatttgttaattgctcagtagtggcagcagtaaggacaatgtacccaatcactctctgtattgctgtttccaactgttgttttgaccctatagtttactactttacatcggacacaattcagaattcaataaaaatgaaaaactggtctgtcaggagaagtgacttcagattctctgaagttcatggtgcagagaattttattcagcataacctacagaccttaaaaagtaagatatttgacaatgaatctgctgcctga

SEQ ID NO: 9 - protein, 344 aa, Mus musculus
MVSSNGSQCPYDDSFKYTLYGCMFSMVFVLGLISNCVAIYIFICALKVRNETTTYMINLAMSDLLFVFTLPFRIFYFATRNWPFGDLLCKISVMLFYTNMYGSILFLTCISVDRFLAIVYPFKSKTLRTKRNAKIVCIAVWFTVMGGSAPAVFFQSTHSQGNNTSEACFENFPAATWKTYLSRIVIFIEIVGFFIPLILNVTCSSMVLRTLNKPVTLSRSKMNKTKVLKMIFVHLVIFCFCFVPYNINLILYSLMRTQTFVNCSVVAAVRTMYPITLCIAVSNCCFDPIVYYFTSDTIQNSIKMKNWSVRRSDSRFSEVQGTENFIQHNLQTLKNKIFDNESAI

SEQ ID NO: 10 - DNA, 2447 bp, Mus musculus
aaaggaactgcaaacaactgggttgaagccttcctttgtccaaacccagcctcttcttcctgtgatgtcatattacaaatctgggaaggctttcttgctcacttcagagacagcccatctcacaatacagctggcaacctccgaaaggcctctccattcagcaagcgcgaacatgcttaggaatttatctgggatcccttaaacgactgcctatcgccgtccggaatcaatgtagaaatacaaagtttgagaataaaaagaaggaagaagtacccgaggacgacgggcggacggacgcacggcgagtgtttgtgactgaagtaaagctggtttggaccctggcggctgaagcacaagtttccacgcggactggtctggtccgacttggaacagtttttccttacactttcagctttatgggttggcttccttgactgcattttctgtcagttaactaaactccagactcatggattttctcgaccagaaaatcagactattttcctgaataatctactagaaacttttacggaacacatttcatgtttcctttgaagagttaagagaagaaagtatttgtaagaacaggaaaagaaacaaatactttgcaaataaactggctgctgctgtgaccacatctgaatagcaaaggcgatcgatcaagcgctgcggacaaaaggcctcctgtaagctgcactgcctgacaatggtaagctccaatggctcccagtgcccttatgacgactcctttaagtacactctgtacgggtgcatgttcagcatggtcttcgtgcttgggctgatatccaactgtgttgcgatatacattttcatctgtgccctcaaagtgagaaatgaaactacaacgtacatgattaacctggcaatgtcagatttacttttcgtctttactttgccatttcggattttttactttgcaacacggaattggccatttggagatctactctgtaagatttcagtaatgctgttttacaccaatatgtatggaagcattctgttcttaacctgtatcagtgtagatcgatttctggcaattgtctacccatttaagtcaaagactttaagaacgaaacgaaatgcaaagatcgtttgcattgctgtgtggttcacagtgatgggaggaagtgcgcctgcagttttctttcagtcgacccactctcaggggaacaatacctcagaagcctgctttgagaactttccagcggccacatggaaaacttatctctccaggattgtgattttcattgaaatagtgggcttttttatccctctcattttgaacgtaacttgttctagtatggtgctaagaactttaaataaacctgttacattaagtagaagcaaaatgaacaaaactaaggttttaaaaatgatttttgtccacttggtcatcttctgtttctgttttgtgccctacaacatcaacctcattttgtactcgctcatgaggacacagacctttgttaactgctctgtggtggcggcagtgaggaccatgtacccgatcactctctgcatcgctgtttccaactgctgctttgaccctattgtttactacttcacctcagacacaattcagaactcaataaaaatgaaaaactggtcggttagaagaagtgactccaggttctctgaagttcagggcactgagaattttatccaacacaacctacagaccttaaaaaataagatatttgataatgaatctgcaatataagctgcctgactaagccactgggactgctccgtgttcaactgtgaaaactgtgttcttgggaactatctctccggctccaacaaaaaatatttttaaaggaagtttgtgtctgatgtgttaaacattaaaatatattctattcttgtatgcacgccattttactttcttgaaccactttaacgtgttttttcctcattaaaaaaaaaaactccataaagttaaggtctaaaagcaattatgatttaaataatgtgatatatctgtacgtttaaaattttgtatatcatgaaatgatttaatcagaaatctgttaaatggttttatactgaaccgagatgttgtttacggaatagtttactaagtagatttcacttatattttattttagattaaaagtacttatcatgccttaaactgtaaacagtgaagaaaaccagtttgaactagtcacacagttttagagcatttcctccaagagactactgcagttttctgcatgacattttatctttttaggcaactcataaaaaagaaaaggtatctaactatatagtatattttgaaattactattttgatttatattctggctgcaacacccaatttcaaaatgatatatagtataaaaacttagcataaagaaactaatgaaatgttatcttgctaggtaaattattttgaaagtacaataaaaaccttgtattttc

SEQ ID NO: 11 - DNA, 27 bp, Artificial Sequence
  Description of Artificial Sequence Synthetic primer
catctacaaagaaccaagaattgtgag

SEQ ID NO: 12 - DNA, 20 bp, Artificial Sequence
  Description of Artificial Sequence Synthetic primer
tccaaatggccaattccgtg

SEQ ID NO: 13 - DNA, 21 bp, Artificial Sequence
  Description of Combined DNA/RNA Molecule Synthetic oligonucleotide
gguguuugugcuuggguuatt

SEQ ID NO: 14 - DNA, 21 bp, Artificial Sequence
  Description of Combined DNA/RNA Molecule Synthetic oligonucleotide
ugcacuggcgugugguuaatt

SEQ ID NO: 15 - DNA, 21 bp, Artificial Sequence
  Description of Combined DNA/RNA Molecule Synthetic oligonucleotide
ggguaacaaugccucagaatt

SEQ ID NO: 16 - DNA, 21 bp, Artificial Sequence
  Description of Combined DNA/RNA Molecule Synthetic oligonucleotide
guggcagcaguaaggacaatt

SEQ ID NO: 17 - DNA, 22 bp, Artificial Sequence
  Description of Artificial Sequence Synthetic primer
gttcccttggaggtgaaaatga

SEQ ID NO: 18 - DNA, 26 bp, Artificial Sequence
  Description of Artificial Sequence Synthetic primer
catcttgataagtgtgaagtcttttg

SEQ ID NO: 19 - DNA, 22 bp, Artificial Sequence
  Description of Artificial Sequence Synthetic primer
tttattcctgggcacttgggtt

SEQ ID NO: 20 - DNA, 25 bp, Artificial Sequence
  Description of Artificial Sequence Synthetic primer
ctttggttccttaattagaggccaa

SEQ ID NO: 21 - DNA, 20 bp, Artificial Sequence
  Description of Artificial Sequence Synthetic primer
gtcaggagttcaagaccagc

SEQ ID NO: 22 - DNA, 22 bp, Artificial Sequence
  Description of Artificial Sequence Synthetic primer
cctaggtatattcccagcagac

SEQ ID NO: 23 - DNA, 22 bp, Artificial Sequence
  Description of Artificial Sequence Synthetic primer
gacaagtggtttcattctggtc

SEQ ID NO: 24 - DNA, 20 bp, Artificial Sequence
  Description of Artificial Sequence Synthetic primer
tcccaaaggagactgcagct

SEQ ID NO: 25 - DNA, 20 bp, Artificial Sequence
  Description of Artificial Sequence Synthetic primer
cacggaattggccatttgga

SEQ ID NO: 26 - DNA, 20 bp, Artificial Sequence
  Description of Artificial Sequence Synthetic primer
ccagaagccacatggaaaac

SEQ ID NO: 27 - DNA, 20 bp, Artificial Sequence
  Description of Artificial Sequence Synthetic primer
tagtgagcgcgtcacttcct

SEQ ID NO: 28 - DNA, 20 bp, Artificial Sequence
  Description of Artificial Sequence Synthetic primer
agccacatgcccagctgatt

SEQ ID NO: 29 - DNA, 24 bp, Artificial Sequence
  Description of Artificial Sequence Synthetic primer
ggttagcttacccaaataaacacc

SEQ ID NO: 30 - DNA, 19 bp, Artificial Sequence
  Description of Artificial Sequence Synthetic primer
acctgcgtcagctcgctct

SEQ ID NO: 31 - DNA, 22 bp, Artificial Sequence
  Description of Artificial Sequence Synthetic primer
agttcacagtcaatagcggact

SEQ ID NO: 32 - DNA, 23 bp, Artificial Sequence
  Description of Artificial Sequence Synthetic primer
atagcctagccaattcagttacg

SEQ ID NO: 33 - DNA, 22 bp, Artificial Sequence
  Description of Artificial Sequence Synthetic primer
atagtctgggtgtcttctgcat

SEQ ID NO: 34 - DNA, 20 bp, Artificial Sequence
  Description of Artificial Sequence Synthetic primer
aaggaaggcaggtgactgag

SEQ ID NO: 35 - DNA, 23 bp, Artificial Sequence
  Description of Artificial Sequence Synthetic primer
gaaccagcgaaaatgagtatgtc

SEQ ID NO: 36 - DNA, 25 bp, Artificial Sequence
  Description of Artificial Sequence Synthetic primer
gcataaagggaaaagtaagaactag

SEQ ID NO: 37 - DNA, 20 bp, Artificial Sequence
  Description of Artificial Sequence Synthetic primer
gcgagaacataaccctggag

SEQ ID NO: 38 - DNA, 26 bp, Artificial Sequence
  Description of Artificial Sequence Synthetic primer
caagacagtatattcttttccttctg

SEQ ID NO: 39 - DNA, 20 bp, Artificial Sequence
  Description of Artificial Sequence Synthetic primer
gtaggtgatgtgggcatggt

SEQ ID NO: 40 - DNA, 20 bp, Artificial Sequence
  Description of Artificial Sequence Synthetic primer
ctgcctggttccatcgaaca

SEQ ID NO: 41 - DNA, 21 bp, Artificial Sequence
  Description of Artificial Sequence Synthetic primer
cagaactgaaggggcagagaa

SEQ ID NO: 42 - DNA, 23 bp, Artificial Sequence
  Description of Artificial Sequence Synthetic primer
ggaagatgattcctgtttggatg

SEQ ID NO: 43 - DNA, 23 bp, Artificial Sequence
  Description of Artificial Sequence Synthetic primer
tcatttgtggaagctgtgctaca

SEQ ID NO: 44 - DNA, 21 bp, Artificial Sequence
  Description of Artificial Sequence Synthetic primer
gtcagtgtccagttgagacag

SEQ ID NO: 45 - DNA, 20 bp, Artificial Sequence
  Description of Artificial Sequence Synthetic primer
ccacatcagtctcatgccag

SEQ ID NO: 46 - DNA, 23 bp, Artificial Sequence
  Description of Artificial Sequence Synthetic primer
cctactttgtggaagcagtgaaa

SEQ ID NO: 47 - DNA, 20 bp, Artificial Sequence
  Description of Artificial Sequence Synthetic primer
acttgtttctgccccggtca

SEQ ID NO: 48 - DNA, 23 bp, Artificial Sequence
  Description of Artificial Sequence Synthetic primer
agtaccagtagagtagtgccata

SEQ ID NO: 49 - DNA, 20 bp, Artificial Sequence
  Description of Artificial Sequence Synthetic primer
tcacagaggccatctgtcct

SEQ ID NO: 50 - DNA, 20 bp, Artificial Sequence
  Description of Artificial Sequence Synthetic primer
agtggctgagtgagcaactg

SEQ ID NO: 51 - DNA, 22 bp, Artificial Sequence
  Description of Artificial Sequence Synthetic primer
atgccatcatttcctccacttg

SEQ ID NO: 52 - DNA, 24 bp, Artificial Sequence
  Description of Artificial Sequence Synthetic primer
cctagagagatgtaatcagtaagc

SEQ ID NO: 53 - DNA, 21 bp, Artificial Sequence
  Description of Artificial Sequence Synthetic primer
cacacactaggccagttttag

SEQ ID NO: 54 - DNA, 20 bp, Artificial Sequence
  Description of Artificial Sequence Synthetic primer
agtccctcttctgctcgcaa

SEQ ID NO: 55 - DNA, 20 bp, Artificial Sequence
  Description of Artificial Sequence Synthetic primer
ctgttggggttgtttcctgg

SEQ ID NO: 56 - DNA, 20 bp, Artificial Sequence
  Description of Artificial Sequence Synthetic primer
ttgatcctgcgtggctgctt

SEQ ID NO: 57 - DNA, 27 bp, Artificial Sequence
  Description of Artificial Sequence Synthetic primer
cctttgacaagaatatctctttaggtt

SEQ ID NO: 58 - DNA, 24 bp, Artificial Sequence
  Description of Artificial Sequence Synthetic primer
taacttggatttatccaggctttc

SEQ ID NO: 59 - DNA, 23 bp, Artificial Sequence
  Description of Artificial Sequence Synthetic primer
gtgtgagctcattctggtaaact

SEQ ID NO: 60 - DNA, 22 bp, Artificial Sequence
  Description of Artificial Sequence Synthetic primer
agagtatagcaaggttgctgga

SEQ ID NO: 61 - DNA, 20 bp, Artificial Sequence
  Description of Artificial Sequence Synthetic primer
ccaagtgctgggattccatg

SEQ ID NO: 62 - DNA, 25 bp, Artificial Sequence
  Description of Artificial Sequence Synthetic primer
gttgtatatgctgatctcatcctac

SEQ ID NO: 63 - DNA, 23 bp, Artificial Sequence
  Description of Artificial Sequence Synthetic primer
caacgagtccaacccataggatt

SEQ ID NO: 64 - DNA, 20 bp, Artificial Sequence
  Description of Artificial Sequence Synthetic primer
tccaaatggccaattccgtg

SEQ ID NO: 65 - DNA, 20 bp, Artificial Sequence
  Description of Artificial Sequence Synthetic primer
gttttccatgtggcttctgg

SEQ ID NO: 66 - DNA, 22 bp, Artificial Sequence
  Description of Artificial Sequence Synthetic primer
cagcaatacagagagtgattgg

SEQ ID NO: 67 - DNA, 25 bp, Artificial Sequence
  Description of Artificial Sequence Synthetic primer
gacacttttcacagttgaaggaact

SEQ ID NO: 68 - DNA, 20 bp, Artificial Sequence
  Description of Artificial Sequence Synthetic primer
aagcaagcacggcgtgagtt

SEQ ID NO: 69 - DNA, 20 bp, Artificial Sequence
  Description of Artificial Sequence Synthetic primer
cagaccacttgctctcctga

SEQ ID NO: 70 - DNA, 24 bp, Artificial Sequence
  Description of Artificial Sequence Synthetic primer
ttccctaaccagaccttattcttg

SEQ ID NO: 71 - DNA, 20 bp, Artificial Sequence
  Description of Artificial Sequence Synthetic primer
cgagaacgagcacaaacgca

SEQ ID NO: 72 - DNA, 23 bp, Artificial Sequence
  Description of Artificial Sequence Synthetic primer
cattcctgttcccacacaaagta

SEQ ID NO: 73 - DNA, 21 bp, Artificial Sequence
  Description of Artificial Sequence Synthetic primer
ctggcaatggtgagtgacaag

SEQ ID NO: 74 - DNA, 23 bp, Artificial Sequence
  Description of Artificial Sequence Synthetic primer
ccagaaccttaacttttcaggtc

SEQ ID NO: 75 - DNA, 22 bp, Artificial Sequence
  Description of Artificial Sequence Synthetic primer
tatgtggcagaggtttctaagg

SEQ ID NO: 76 - DNA, 22 bp, Artificial Sequence
  Description of Artificial Sequence Synthetic primer
attccaaccttgtcatctctgg

SEQ ID NO: 77 - DNA, 23 bp, Artificial Sequence
  Description of Artificial Sequence Synthetic primer
cctgtagtttacatttccctgct

SEQ ID NO: 78 - DNA, 20 bp, Artificial Sequence
  Description of Artificial Sequence Synthetic primer
ccagtggtacctgacagcat

SEQ ID NO: 79 - DNA, 20 bp, Artificial Sequence
  Description of Artificial Sequence Synthetic primer
tactgactggctgggagcat

SEQ ID NO: 80 - DNA, 23 bp, Artificial Sequence
  Description of Artificial Sequence Synthetic primer
catttcagagttgacttcctgtg

SEQ ID NO: 81 - DNA, 21 bp, Artificial Sequence
  Description of Artificial Sequence Synthetic primer
cctatcggtgaccaataggtg

SEQ ID NO: 82 - DNA, 23 bp, Artificial Sequence
  Description of Artificial Sequence Synthetic primer
ccccaagatcttcaatttggcaa

SEQ ID NO: 83 - DNA, 20 bp, Artificial Sequence
  Description of Artificial Sequence Synthetic primer
ttctcaagggcagctgcgaa

SEQ ID NO: 84 - DNA, 23 bp, Artificial Sequence
  Description of Artificial Sequence Synthetic primer
ccaggactactatagaagtctac

SEQ ID NO: 85 - DNA, 20 bp, Artificial Sequence
  Description of Artificial Sequence Synthetic primer
gtgggacataccatactggc

SEQ ID NO: 86 - DNA, 23 bp, Artificial Sequence
  Description of Artificial Sequence Synthetic primer
tgatatctttgccagcgaagact

SEQ ID NO: 87 - DNA, 23 bp, Artificial Sequence
  Description of Artificial Sequence Synthetic primer
atcacaatcaccaaagaccagtg

SEQ ID NO: 88 - DNA, 20 bp, Artificial Sequence
  Description of Artificial Sequence Synthetic primer
ccctgagctctttcccagat

SEQ ID NO: 89 - DNA, 20 bp, Artificial Sequence
  Description of Artificial Sequence Synthetic primer
ccgctccactgtatgcttgt

SEQ ID NO: 90 - DNA, 23 bp, Artificial Sequence
  Description of Artificial Sequence Synthetic primer
tagaaaccaccttgtcagttcct

SEQ ID NO: 91 - DNA, 20 bp, Artificial Sequence
  Description of Artificial Sequence Synthetic primer
gcagacacctgggttccatt

SEQ ID NO: 92 - DNA, 20 bp, Artificial Sequence
  Description of Artificial Sequence Synthetic primer
ctctggcttttgcaggggaa

SEQ ID NO: 93 - DNA, 24 bp, Artificial Sequence
  Description of Artificial Sequence Synthetic primer
gactatgagtgaatgaagatggac

SEQ ID NO: 94 - DNA, 23 bp, Artificial Sequence
  Description of Artificial Sequence Synthetic primer
cagcaactacttttgttgagcca

SEQ ID NO: 95 - DNA, 23 bp, Artificial Sequence
  Description of Artificial Sequence Synthetic primer
ccgaacttgaacgcttttctgaa

SEQ ID NO: 96 - DNA, 20 bp, Artificial Sequence
  Description of Artificial Sequence Synthetic primer
gttcgcgtgctttcggaaag

SEQ ID NO: 97 - DNA, 20 bp, Artificial Sequence
  Description of Artificial Sequence Synthetic primer
agtccctcttctgctcgcaa

SEQ ID NO: 98 - DNA, 26 bp, Artificial Sequence
  Description of Artificial Sequence Synthetic primer
cacgaattaacaggaactcactattg

SEQ ID NO: 99 - DNA, 20 bp, Artificial Sequence
  Description of Artificial Sequence Synthetic primer
tcccaaaggagactgcagct

SEQ ID NO: 100 - DNA, 20 bp, Artificial Sequence
  Description of Artificial Sequence Synthetic primer
ctgcgtcctcaaagtccgaa

SEQ ID NO: 101 - DNA, 30 bp, Artificial Sequence
  Description of Artificial Sequence Synthetic primer
caacttacatgattaacttggcaatgtgag

SEQ ID NO: 102 - DNA, 20 bp, Artificial Sequence
  Description of Artificial Sequence Synthetic primer
cacggaattggccatttgga

SEQ ID NO: 103 - DNA, 20 bp, Artificial Sequence
  Description of Artificial Sequence Synthetic primer
cacggaattggccatttgga

SEQ ID NO: 104 - DNA, 20 bp, Artificial Sequence
  Description of Artificial Sequence Synthetic primer
tccaaatggccaattccgtg

SEQ ID NO: 105 - DNA, 23 bp, Artificial Sequence
  Description of Artificial Sequence Synthetic primer
ggtagacaattgccagaaatcga

SEQ ID NO: 106 - DNA, 23 bp, Artificial Sequence
  Description of Artificial Sequence Synthetic primer
ggtagacaattgccagaaatcga

SEQ ID NO: 107 - DNA, 22 bp, Artificial Sequence
  Description of Artificial Sequence Synthetic primer
cagcaatacagagagtgattgg

SEQ ID NO: 108 - DNA, 22 bp, Artificial Sequence
  Description of Artificial Sequence Synthetic primer
cagcaatacagagagtgattgg

SEQ ID NO: 109 - protein, 344 aa, Homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPCNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 110 - protein, 344 aa, Homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIRGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 111 - DNA, 1018 bp, Homo sapiens
atggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgttcactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagcaaaataaacaaaactaaggttttaaaaatgatttttgtacatttgatcatattctgtttctgttttgttccttacaatatcaatcttattttatattctcttgtgagaacacaaacatttgttaattgctcagtagtggcagcagtaaggacaatgtacccaatcactctctgtattgctgtttccaactgttgttttgaccctatagtttactactttacatcggacacaattcagaattcaataaaaatgaaaaactggtctgtcaggagaagtgacttcagattctctgaagttcatggtgcagagaattttattcagcataacctacagaccttaaaaagtaagatatttgacaatgaatctgctgcctga

SEQ ID NO: 112 - protein, 235 aa, Artificial Sequence
  Description of Artificial Sequence Synthetic polypeptide
MNGTEGPNFYVPFSNKTGVVRSPFEAPQYYLAEPWQFSMLAAYMFLLIMLGFPINFLTLYVTVQHKKLRTPLNYILLNLAVADLFMVFGGFTTTLYTSLHGYFVFGPTGCNLEGFFATLGGEIALWSLVVLAIERYVVVCKPMSNFRFGENHAIMGVAFTWVMALACAAPPLVGWSRYIPEGMQCSCGIDYYTPHEETNNESFVIYMFVVHFIIPLIVIFFCYGQLVFTVKEAAA

SEQ ID NO: 113 - protein, 90 aa, Artificial Sequence
  Description of Artificial Sequence Synthetic polypeptide
ATTQKAEKEVTRMVIIMVIAFLICWLPYAGVAFYIFTHQGSDFGPIFMTIPAFFAKTSAVYNPVIYIMMNKQFRNCMVTTLCCGKNPLGD

SEQ ID NO: 114 - protein, 15 aa, Artificial Sequence
  Description of Artificial Sequence Synthetic peptide
STTVSKTETSQVAPA

SEQ ID NO: 115 - protein, 140 aa, Artificial Sequence
  Description of Artificial Sequence Synthetic polypeptide
MNGTEGPNFYVPFSNKTGVVRSPFEAPQYYLAEPWQFSMLAAYMFLLIMLGFPINFLTLYVTVQHKKLRTPLNYILLNLAVADLFMVFGGFTTTLYTSLHGYFVFGPTGCNLEGFFATLGGEIALWSLVVLAIERYVVVC

SEQ ID NO: 116 - protein, 78 aa, Artificial Sequence
  Description of Artificial Sequence Synthetic polypeptide
GENHAIMGVAFTWVMALACAAPPLVGWSRYIPEGMQCSCGIDYYTPHEETNNESFVIYMFVVHFIIPLIVIFFCYGQL

SEQ ID NO: 117 - protein, 83 aa, Artificial Sequence
  Description of Artificial Sequence Synthetic polypeptide
QKAEKEVTRMVIIMVIAFLICWLPYAGVAFYIFTHQGSDFGPIFMTIPAFFAKTSAVYNPVIYIMMNKQFRNCMVTTLCCGKN

SEQ ID NO: 118 - DNA, 19 bp, Homo sapiens
tgcatgcatgttcagcatg

SEQ ID NO: 119 - DNA, 15 bp, Homo sapiens
tgcatgttcagcatg

SEQ ID NO: 120 - DNA, 13 bp, Homo sapiens
atgatttgcaatg

SEQ ID NO: 121 - DNA, 18 bp, Homo sapiens
atgattaacttggcaatg

SEQ ID NO: 122 - DNA, 15 bp, Homo sapiens
  CDS (1)..(15)
atgtcagtcttgctt

SEQ ID NO: 123 - DNA, 15 bp, Homo sapiens
  CDS (1)..(15)
atgtcagacttgctt

SEQ ID NO: 124 - DNA, 15 bp, Homo sapiens
  CDS (1)..(15)
attttcttcgaaata

SEQ ID NO: 125 - DNA, 15 bp, Homo sapiens
  modified_base (7)..(7): a, c, g, t, unknown or other
attttcntcgaaata

SEQ ID NO: 126 - DNA, 15 bp, Homo sapiens
  CDS (1)..(15)
attttcatcgaaata

SEQ ID NO: 127 - DNA, 14 bp, Homo sapiens
ttctcaaaatagtg

SEQ ID NO: 128 - DNA, 15 bp, Homo sapiens
  CDS (1)..(15)
ttcatcaaaatagtg

SEQ ID NO: 129 - DNA, 13 bp, Homo sapiens
ttctcgaaaagtg

SEQ ID NO: 130 - DNA, 15 bp, Homo sapiens
actctctatattgct

SEQ ID NO: 131 - DNA, 15 bp, Homo sapiens
  modified_base (8)..(8): a, c, g, t, unknown or other
actctctntattgct

SEQ ID NO: 132 - DNA, 15 bp, Homo sapiens
actctctgtattgct

SEQ ID NO: 133 - protein, 98 aa, Homo sapiens
FKYTLYGCMFSMVFVLGLISNCVAIYIFICVLKVRNETTTYMINLAMSDLLFVFTLPFRIFYFTTRNWPFGDLLCKISVMLFYTNMYGSILFLTCISV

SEQ ID NO: 134 - protein, 34 aa, Homo sapiens
PFKSKTLRTKRNAKIVCTGVWLTVIGGSAPAVFV

SEQ ID NO: 135 - protein, 41 aa, Homo sapiens
FENFPEATWKTYLSRIVIFIEIVGFFIPLILNVTCSSMVLK

SEQ ID NO: 136 - protein, 39 aa, Homo sapiens
VTLSRSKINKTKVLKMIFVHLIIFCFCFVPYNINLILYS

SEQ ID NO: 137 - protein, 32 aa, Homo sapiens
FVNCSVVAAVRTMYPITLCIAVSNCCFDPIVY

SEQ ID NO: 138 - protein, 5 aa, Homo sapiens
NESAA

SEQ ID NO: 139 - DNA, 19 bp, Homo sapiens
  modified_base (7)..(19): a, c, g, t, unknown or other
tgcatgnnnnnnnnnnnnn

SEQ ID NO: 140 - DNA, 18 bp, Homo sapiens
  modified_base (7)..(8): a, c, g, t, unknown or other
atgattnncnnngnnann

SEQ ID NO: 141 - protein, 344 aa, Pan troglodytes; identical to SEQ ID NO: 1

SEQ ID NO: 142 - protein, 344 aa, Macaca mulatta
MVSVNSSHCFYNDSFKYTLYGCMFSTVFVLGLISNCVAIYIFICVLKVRNETTTYMINLAMSDLLFVFTLPFRIFYFTTQNWPFGDLLCKISVMLFYTNMYGSILFLTCISVDRFLAIVYPFKSKTLRTKRNAKIVCIGVWLTVIGGSAPAVLVQSTHSQGNNASEACFENFPEATWKTYLSRIVIFIEIVGFFIPLILNVTCSSMVLKTLNKPVTLSRSKINKTKVLKMIFVHLIIFCFCFVPYNINLILYSLVRTQTFVNCSVVAAVRTMYPITLCIAVSNCCFDPVVYYFTSDTIQNSIKMKNWSTRRSDFRFSEVHGTENFIQHNLQTLKRKIFDNESAA

SEQ ID NO: 143 - protein, 341 aa, Bos taurus
MVSNNSSDCIYNDSFKYTLYGCMFSMVFVLGLISNCVAIYIFICTLKVRNETTTYMINLAMSDLLFVFTLPFRIFYFATRNWPFGDLLCKISVMSFYTNMYGSILFLTCISADRFLAIVYPFKSKTLRTKRNAKIVCIAVWLTVMGGSAPAVFVPSPGSNTSATCFENFPAATWKTFLSRIVIFIEIVGFFIPLILNVTCSSMVLRTLNKPVTLSRSKINKTKVLRMIFVHLVIFCFCFVPYNINLILYSLMRTQTFVNCSAAIAVRTTYPITLCIAVLNCCFDPIIYYFTSDTIQNSIKMKNWSTRRSDSRFSEVQGTENFIQHNLQTLKNKIPDNESTI

SEQ ID NO: 144 - protein, 344 aa, Rattus norvegicus
MVSANGSHCPYDDSFKYTLYGCMFSMVFVLGLISNCVAIYIFICALKVRNETTTYMINLAMSDLLFVFTLPFRIFYFATRNWPFGDLLCKISVMLFYTNMYGSILFLTCISVDRFLAIVYPFKSKTLRTKRNAKIVCIAVWFTVMGGSAPAVFFQSTHSQGNNTSEACFENFPAATWKTYLSRIVIFIEIVGFFIPLILNVTCSSMVLRTLNKPVTLSRSKMNKTKVLKMIFVHLVIFCFCFVPYNINLILYSLVRTQTFVNCSVGAAVRTMYPITLCIAVSNCCFDPIVYYFTSDTIQNSIKMKSWSVRRSDSRFSEVQGTENFIQHNLQTLKNKIFDNESAI

SEQ ID NO: 145 - protein, 308 aa, Gallus gallus
MVSSNCSTEDSFKYTLYGCVFSMVFVLGLIANCVAIYIFTFTLKVRNETTTYMLNLAISDLLFVFTLPFRIYYFVVRNWPFGDVLCKISVTLFYTNMYGSILFLTCISVDRFLAIVHPFRSKTLRTKRNARIVCVAVWITVLAGSTPASFFQSTNRQNNTEQRTCFENFPESTWKTYLSRIVIFIEIVGFFIPLILNVTCSTMVLRTLNKPLTLSRNKLSKKKVLKMIFVHLVIFCFCFVPYNITLILYSLMRTQTWINCSVVTAVRTMYPVTLCIAVSNCCFDPIVYYFTSDTNSELDKKQQVHQNT

SEQ ID NO: 146 - protein, 328 aa, Danio rerio
MTSNNTTVTDCGTSHFRYPLFTSTYTVVLLFALPLNCVSLWILVCRNGLKKSVPVIYMANLALSDLLFTLSLPFRIIYFATGKWTLGNTLCMIPGTLFAVNIYSSSFFIMLISVDRMLAVVYPLRSRSLRTAPVAWMFCALVWLLIAGLAVPIAQNHPEINDTACNITRCFEKYSTDSWNNGFKLICLATFFGMLVPFSIILGCTVAVVRQLRGYSMATSSFNAELSKSKIVKLFLSNLLIYTICFIPFHVIYILFTLVKLEMLHNGSHLEVYFNLHNITLCLASTNSCLDPLIYYFSAKNIQSRSRCGSASKTVGLGLVQSTSWTGQ

SEQ ID NO: 147 - protein, 5 aa, Homo sapiens
MSVLL

SEQ ID NO: 148 - protein, 5 aa, Homo sapiens
  MOD_RES (3)..(3): Asp or Val
MSXLL

SEQ ID NO: 149 - DNA, 15 bp, Homo sapiens
  CDS (1)..(15)
  misc_feature (7)..(9): The codon “gwc” may code for “Asp” or “Val”
atgtcagwcttgctt

SEQ ID NO: 150 - protein, 5 aa, Homo sapiens
MSDLL

SEQ ID NO: 151 - protein, 5 aa, Homo sapiens
IFFEI

SEQ ID NO: 152 - protein, 5 aa, Homo sapiens
  MOD_RES (3)..(3): Ile or Phe
IFXEI

SEQ ID NO: 153 - protein, 5 aa, Homo sapiens
IFIEI

SEQ ID NO: 154 - protein, 5 aa, Homo sapiens
FIKIV

SEQ ID NO: 155 - protein, 5 aa, Homo sapiens
  MOD_RES (3)..(3): Glu or Lys
FIXIV

SEQ ID NO: 156 - DNA, 15 bp, Homo sapiens
  CDS (1)..(15)
  misc_feature (7)..(9): The codon “naa” may code for “Glu” or “Lys”
ttcatcnaaatagtg

SEQ ID NO: 157 - protein, 5 aa, Homo sapiens
FIEIV

SEQ ID NO: 158 - DNA, 15 bp, Homo sapiens
  CDS (1)..(15)
ttcatcgaaatagtg

SEQ ID NO: 159 - DNA, 18 bp, Homo sapiens
agattgttcactgtgatc

SEQ ID NO: 160 - DNA, 35 bp, Homo sapiens
agattgtttgcactggcgtgtggttaactgtgatc

SEQ ID NO: 161 - DNA, 15 bp, Homo sapiens
  CDS (1)..(15)
  misc_feature (7)..(9): The codon “wtc” may code for “Ile” or “Phe”
attttcwtcgaaata

SEQ ID NO: 162 - protein, 586 aa, Artificial Sequence
  Description of Artificial Sequence Synthetic polypeptide
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVTTFVNCSVVAAVRTMYPITLCIAVSNCCFDPIVYYFTSDTIQNSIKMKNWSVRRSDFRFSEVHGAENFIQHNLQTLKSKIFDNESAATQLSCTKWSTRENLYFQMRKLLSSIEQACDICRLKKLKCKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISASSSEESSNKGQRQLTVSPEPGIWAPPTDVSLGDELHLDGEDVAMAHADALDDFDLDMLGDGDSPGPGFTPHDSAPYGALDMADFEFEQMFTDALGIDEYG

SEQ ID NO: 163 - DNA, 7481 bp, Artificial Sequence
  Description of Artificial Sequence Synthetic polynucleotide
atgcatgtcgttacataacttacggtaaatggcccgcctggctgaccgcccaacgacccccgcccattgacgtcaataatgacgtatgttcccatagtaacgccaatagggactttccattgacgtctgggtggagtatttacggtaaactgcccacttggcagtacatcaagtgtatcatatgccaagtacgccccctattgacgtcaatgacggtaaatggcccgcctggcattatgcccagtacatgaccttgggactttcctacttggcagtacatctacgtattagtcatcgctattaccatggtgatgcggttttggcagtacatcaatgggcgtggatagcggtttgactcacggggatttccaagtctccaccattgacgtcaatgggagtttgttttggcaccaaaatcaacgggactttccaaaatgtcgtaacaactccgccccattgacgcaaatgggcggtaggcgtgtacggtgggaggtctatataagcaagcctccctatcagtgatagagatctccctatcagtgatagagatcgtcgacgagctcgtttagtgaaccgtcagatcgcctggagacgccatccacgctgttttgacctccatagaagacaccggaccgtccagcctccggactctagtaccggtgatatcctcgagcccatcaacaagtttgtacaaaaaagcaggcaccatggtaagcgttaacagctcccactgcttctataatgactcctttaagtaactttgtatgggtgcatgttcagcatggtgtttgtgcttgggttaatatccaattgtgttgccatatacattttcattgcgtcctcaaagtccgaaatgaaactacaacttacatgattaacttggaatgtcagacttgctttttgtttttactttacccttcaggattttttacttcacaacacggaattggccatttggagattactttgtaagatttctgtgatgctgttttataccaacatgtacggagcattctgttcttaacctgtattagtgtagatcgatttctggcaattgtctacccatttaagtcaaagactctaagaacaaaagaaatgcaaagattgtttgcactggcgtgtggttaactgtgatggaggaagtgcacccgccgtttttgttcagtctacccactctcagggtaacaatgcctcagaagcctgctttgaaaatttccagaagccacatggaaaacatatctctcaaggattgtaattttcacgaaatagtgggattttttattcctctaattttaaatgtaacttgttctagtatggtgctaaaaactttaaccaaacctgtacattaagtagaagcaaaataaacaaaactaaggttttaaaaatgtttttgtacatttgatcatattctgtttctgttttgttccttacaatatcaatcttattttatattctcttgtgagaacacaacatttgttaattgctcagtagtggcagcagtaaggacaatgtaccaatcactctctgtattgctgtttccaactgttgttttgaccctatagtttactactttacatcggacacaattcagaattcataaaaatgaaaaactggtctgtcaggagaagtgacttcagatctctgaagttcatggtgcagagaattttattcagcataacctacagaccttaaaaagtaagatatttgacaatgaatctgctgccacccagctttcttgtacaaagtggtccacgcgtgagaacctgacttccagatgagaaagctactgtcttctatcgaacaagcatgcgatatttgccgacttaaaaagctcaagtgctccaaagaaaaaccgaagtgcgccaagtgtctgaagaacaactgggagtgtcgtactctcccaaaacaaaaggtctccgctgactagggcacatctgacagaagtggaatcaaggctagaaagactggaacagctatttctactgatttttcctcgagaagaccttgacatgattttgaaatggattctttacagatataaaagcattgttaacaggattatttgtacaagataatgtgaataaagatgccgtcacagatagattggcttcagtggagactgatatgcctctaacattgagacagatagaataagtgcgactcatcatcggaagagagtagtaacaaaggtcaaagacagttgactgtatcgccggaattcccggggatctgggcccccccgaccgatgtcagcctgggggacgagctccattagacggcgaggacgtgcgatggcgcatgccgacgcgctagacgatttcgatctggacatgttgggggacggggattccccgggtccgggatttaccccccacgactccgccccctacggcgctcggatatggccgacttcgatttgagcagatgtttaccgatgcccttggaattgacgagtacggttagtaatgagctagcttgggatctttgtgaaggaaccttacttctgtggtgtgacataattggcaaactacctacagagatttaaagctctaaggtaaatataaaatttttaagtgtataatgtgttaaatagctgcatatgcttgctgcttgagagttttgcttactgagtatgatttatgaaaatatatacacaggagctagtgattctaattgtttgtgtattttagattcacagtcccaaggctcatttcaggccctcagtcctcacagtctgttcatgatcataatcagccataccacatttgtagaggtttacttgctttaaaaaacctcccacacctccccctgaacctgaaacataaaatgaatgcaattgttgttttaacttgtttattgcagcttataatggttacaaataaagcaatagcatcacaaattcacaaataaagcatttttttcactgcattctagttgtggtttgtccaaactcatcaatgtatcttatcattctggatcgatcctgcattaatgaatcggccaacgcgcggggagaggcggtttgcgattggctggcgtaatagcgaagaggcccgcaccgatcgcccttcccaacagttgcgcagcctgaatggcgatgggacgcgccctgtagcggcgcattaagcgcggcgggtgtggtggttacgcgcacgtgaccgctacacttgccagcgccctagcgcccgctcctttcgctttcttcccttcctttctcgccacgttgccggctttccccgtcaagctctaaatcgggggctccctttagggttccgatttgtgctttacggcacctcgaccccaaaaaacttgattagggtgatggttcacgtagtgggccatcgccctgataacggtttttcgccctttgacgttggagtccacgttctttaatagtggactcttttccaaactggaacaacactcaaccctatctcggtctattcttttgatttataagggattttgccgatttcggctattggttaaaaaatgagctgatttaacaaatatttaacgcgaattttaacaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcattagggtgtggaaagtccccaggctccccagaggcagaagtatgcaaagcatgcatctcaattagtcagcaaccaggtgtggaaagtccccaggctccccagcaggcagaagtatgcaaagcatgctctcaattagtcagcaaccatagtcccgccccaactccgcccatcccgcccctaactccgcccagttccgcccattctccgccccatggctgactaattttttttatttatgcagaggccgaggcccctcggcctctgagctattccagaagtagtgagaggcttttttggaggcctaggcttttgcaaaaagcttgattcttctgacacaacagtctcgaacttaaggctagagccaccatgattgaacaaatggattgcacgcaggttctccggccgcttgggtgagaggctattcggctatgactgggcacaacagacaatcggctgctctgatgccgccgtgttccggctgtcagcgcaggggcgcccggttcttttgtcaagaccgacctgtccggtgccctgaatgactgcaggacgaggcagcgcggctatcgtggctggccacgacgggcgttccttgcgcagctgtgctcgacgttgtcactgaagcgggaagggctggctgctattgggcgaagtgccggggcaggatctctgtcatctcaccttgctcctgccgagaaagtatccatcatggctgatgcaatgcggcggctgcatacgcttgatccggctacctgcccatcgaccaccaagcgaaacatcgcatcgagcgagcacgactcggatggaagccggtcttgtcgatcaggatgatctggacgaagagcatcaggggctcgcgccagccgaactgttcgccaggctcaagcgcgcatgcccgacggcgaggatctcgtcgtgacccaggcgatgcctgcttgccgaatatcatggtggaaaatggccgcttttctggattcatcgactgtggccggctgggtgtggcggaccgcttcaggacatagcgttggctacccgtgatattgctgaagacttggcggcgaatgggctgaccgcttcctcgtgctttacggtatcgccgctcccgattcgcagcgcatcgccttctatcgccttcttacgagttcttctgagcgggactctggggttcgaaatgaccaccaagcgacgcccaacctgccatcacgatggccgcaataaaatatctttattttcattacatctgtgtgttggttttttgtgtgatcgatacattaattgcgttgcgctcactgcccgctttccagcgggaaacctgtcgtgccagctgcattaatgaatcggccaacgcgcggggagaggcggtttgcgtattgggcgctcttccgcttctcgctcactgactcgctgcgctcggtcgttcggctgcggcgacggtatcagctcactcaaaggcggtaatacggttatccacagaatcaggggataacgcaggaaagaacatgtgagcaaaaggccgcaaaaggccaggaaccgtaaaaaggccgcgttgctggcgttttccataggctccgcccccctgacgagcatcacaaaaatcgacgctcaagtcagaggtggcgaaacccgacaggactataaagaaccaggcgtttccccctggaagctccctcgtgcgctctcctgttcgaccctgccgcttaccggatacctgtccgcctttctcccttcgggaagcgtggcgctttctcatagctcacgctgtaggtactcagttcggtgtaggtcgttcgctccaagctgggctgtgtgcacaaccccccgttcagcccgaccgctgcgccttatccggtaactatcgtcttgagtccaacccggtaagacacgacttatcgcactggcagcagccactggtaacaggattagcagagcgaggtatgtagcggtgctacagagttcttgaagtggtggcctaactacggctacactagaaggacagtatttggtatctgcgctctgctgagccagttaccttcggaaaaagagttggtagctcttgatccggcaaaaaaccaccgctggtagcggtggtttttttgtttgcaagcagcagattacgcgcagaaaaaaaggatctcaagaagatccttgatcttttctacggggtctgacgctcagtggaacgaaaactcacgtaagggattttggtcatgagattatcaaaaaggatcttcacctagatccttttaaattaaaaatgaagttttaaatcaactaaagtatatatgagtaaacttggtctgacagttaccaatgcttaatcgtgaggcacctatctcagcgatctgtctatttcgttcatccatagttgcctgactccccgtcgtgtagataactacgtacgggagggcttaccatctggccccagtgctgcaatgataccgcgagaccacgctcaccggctccagatttatcagcaataaaccagccagccggaagggccgagcgcagaagtggtcctgcaacttatccgcctccatccagtctattaattgttgccgggaagctagagtaagtgttcgccagttaatagtttgcgcaacgttgttgccattgctacaggcatcgtggtgtcacgctcgtcgtttggtaggcttcattcagctccggttcccaacgatcaaggcgagttacatgatccccctgttgtgcaaaaaagcggttagctccttcggtcctccgatcgttgtcagaagtaagttggccgcagtgttatcatcatggttatggcagcactgcataattctcttactgtcatgccatccgtaagagcttttctgtgactggtgagtactcaaccaagtcattctgagaatagtgtatgcggcgaccgagttgctcttgccggcgtcaatacgggataataccgcgccacatagcagaactttaaaagtgctctcattggaaaacgttcttcggggcgaaaactctcaaggatcttaccgctgttgagatccagttcgatgtaaccactcgtgcacccaactgatcttcagcatcttttactttcaccagcgtttctggggagcaaaaacaggaaggcaaaatgccgcaaaaaagggaataagggcgacacggaaatgttgaatactcatatcttcctttttcaatattattgaagcatttatcagggttattgtctcatgagcggaacatatttgaatgtatttagaaaaataaacaaataggggttccgcgcacatttccccgaaaagtgccaccgacgtcgacggatcgggagatctcccgatcccctatggtgcactctcagtacaatctctctgatgccgcatagttaagccagtatctgctccctgcttgtgtgttggaggccatgttctttcctgcttatcccctgattctgtggataaccgtattaccgcctttgagtgagctgataccgctcccgcagccgaacgaccgagcgcagcgagtcagtgagcgaggaagcggaagagcgcccaatacgcaaacgcctctccccgcgcgttggccgattcattaatgcagagcttgcaattcgcgcgtttttcatattattgaagcatttatcagggttattgtctcatgagcggatacatatttgaatgtatttagaaaataaacaaataggggttccgcgcacatttccccgaaaagtgccacctgacgtctaagaaacattattatcatgacattaacctataaaaataggcgtagtacgaggccctttcactcattag

SEQ ID NO: 164 - protein, 7 aa, Artificial Sequence
  Description of Artificial Sequence Synthetic peptide
ENLYFQM

(19) United States
(12) Patent Application Publication
(10) Pub. No.: US 2014/0134188 A1
(43) Pub. Date: May 15, 2014

(54) Methods for P2RY5 Mediated Regulation of Hair Growth and Mutants Thereof

(71) Applicant: The Trustees of Columbia University in the City of New York, New York, NY (US)

(72) Inventor: Angela M. Christiano, Saddle River, NJ (US)

(73) Assignee: The Trustees of Columbia University in the City of New York, New York, NY (US)

(21) Appl. No.: 14/042,476

(22) Filed: Sep. 30, 2013

Related U.S. Application Data
(62) Division of application No. 12/831,879, filed on Jul. 7, 2010, now abandoned.
Application No. 12/831,879 is a continuation-in-part of application No. PCT/US09/30480, filed on Jan. 8, 2009.
(60) Provisional application No. 61/019,733, filed on Jan. 8, 2008.
(60) Provisional application No. 61/044,309, filed on Apr. 11, 2008.

(51) Int. Cl. A61K 39/395; A61K 38/17; A61K 38/10; A61K 38/08; A61K 31/713

(52) U.S. Cl. CPC A61K 39/3955; A61K 38/08; A61K 31/713; A61K 38/10; A61K 38/1709
USPC 424/172.1; 514/21.6; 514/44 A; 514/21.5; 514/21.4; 514/21.3; 514/21.2

(57) ABSTRACT

The invention provides for a method for screening compounds that bind to and modulate the hair-specific G-protein coupled receptor, P2RY5. The invention farther provides for methods for controlling hair growth by administering a P2RY5 modulating compound to a subject.

This application is a continuation-in-part of International Application No. PCT/US09/30480 filed on Jan. 8, 2009, which claims the benefit of priority of U.S. Ser. No. 61/019,733, filed on Jan. 8, 2008, and U.S. Ser. No. 61/044,309, filed on Apr. 11, 2008, the contents of each which are hereby incorporated by reference in their entireties.

### GOVERNMENT INTERESTS

The work described herein was supported in whole, or in part, by National Institute of Health Grant No. NIAMS ROI AR44924, Thus, the United States Government has certain rights to the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### LENGTHY TABLE

A lengthy table (for example, Table 6) is referenced in this application and has been filed as an Appendix to this invention. The specification of the application contains reference to the single table, Table 6, which consists of more than 51 pages, and is hereby incorporated by reference in its entirety. Table 6 contains information encompassing the atomic coordinates for residues of a Rhodopsin crystal.

### BACKGROUND OF THE INVENTION

P2Y receptors belong to a class of G-protein coupled receptors (GPCRs) that activate various intracellular signaling pathways. Generally, a change in the conformation of the intracellular region of the receptor occurs when an endogenous ligand binds to its receptor. Activation of the receptor thus leads to coupling of the intracellular region of the protein to an intracellular G-protein. GPCRs can interact with more than one G protein, and are deemed to be promiscuous with respect these G proteins (such as Gq, Gs, Gi, Gz, Gt, and Go). Endogenous ligand-activated GPCR coupling with the G-protein begins a signaling cascade process (referred to as signal transduction). Under normal conditions, signal transduction subsequently leads to cellular activation or cellular inhibition. The cloning of several ATP receptors has been reported, for example the P2 purinergic receptors. These receptors can be subdivided into two classes: the G protein-coupled receptors, P2Y receptors, and the ion channel-coupled receptors, P2X receptors. In addition, the 6H1 orphan receptor (P2RY5), cloned from activated chicken T lymphocytes, exhibits a significant degree of homology to the P2Y1 and P2Y2 receptors, indicating that it also belongs to the P2Y family.

### BRIEF DESCRIPTION OF THE FIGURES

To conform to the requirements for U.S. patent applications, many of the figures presented herein are black and white representations of images originally created in color, such as many of those figures based on immunofluorescence microscopy, e.g., DAPI (blue) staining and P2RY5 staining in green. In the below descriptions and the examples, this colored staining is described in terms of its appearance in black and white. For example, DAPI staining which appeared blue in the original appears as a dark grey stain when presented in black and white. The original color versions can be viewed in Shimomura et al., Nat Genet. 2008 March; 40(3):335-9 (including the accompanying Supplementary Information available in the on-line version of the manuscript available on the Nature Genetics web site). For the purposes of the U.S., the contents of Shimomura et al., Nat Genet. 2008 March; 40(3):335-9, including the accompanying “Supplementary Information,” are herein incorporated by reference.

FIG. 1 shows light microscopy images depicting the dystrophic appearance in the proximal portion of hairs taken from a member of the HYP7 family (FIGS. 1A-B), the HYP15 family (FIGS. 1C-D), or the HYP31 family (FIG. 1E). FIG. 1F is a candidate member of a HYP family.

FIG. 2 represents light microscopy images that depict the wavy appearance of hairs taken from a member of the HYP7 family (FIG. 2A), the HYP24 family (FIG. 2B), the HYP31 family (FIG. 2C), or the HYP15 family (FIG. 2D), as compared to hairs obtained from RCO3 mice having a mutation in the K6irs1 gene (FIG. 2E).

FIG. 3 shows light microscopy images depicting the thin and tapered end of the distal portion of hairs taken from a member of the HYP31 family (FIG. 3A), the HYP15 family (FIGS. 3B-C), or the HYP7 family (FIG. 3D).

FIG. 4 shows DNA chromatogram traces of a patient having a CATG insertion mutation starting at nucleotide position 69 of SEQ ID NO: 2 [see SEQ ID NO: 7] in the P2RY5 gene of a HYP2 or HYP7 family member (FIG. 4A). DNA chromatogram traces are also shown for a carrier of the CATG insertion mutation (FIG. 4B) and a wild type patient (FIG. 4C). FIG. 4 discloses SEQ ID NOS: 118 and 119, respectively in order of appearance.

FIG. 5 shows DNA chromatogram traces of a patient having an AACT_G deletion mutation starting at nucleotide position 172 of SEQ ID NO: 2 [see SEQ ID NO: 8] in the P2RY5 gene of a HYP 18 family member (FIG. 5A), and a wild type patient (FIG. 5B). FIG. 5 discloses SEQ ID NOS: 120 and 121, respectively in order of appearance.

FIG. 6 shows DNA chromatogram traces of a patient having an A>T substitution mutation at nucleotide position 188 of SEQ ID NO: 2 resulting in a D>V amino acid substitution at amino acid position 63 of SEQ ID NO: 1 [see SEQ ID NO: 3] in the P2RY5 gene of a HYP15 or HYP31 family member (FIG. 6A). DNA chromatogram traces are also shown for a carrier of the A>T substitution mutation (FIG. 6B) and a wild type patient (FIG. 6C). FIG. 6 discloses SEQ ID NOS: 122 and 123, respectively in order of appearance.

FIG. 7 shows DNA chromatogram traces of a patient having an A>T substitution mutation at nucleotide position 562 of SEQ ID NO: 2, resulting in an I>F amino acid substitution at amino acid position 188 of SEQ ID NO: 1 [see SEQ ID NO: 4] in the P2RY5 gene of a HYP24 or HY5 family member (FIG. 7A). DNA chromatogram traces are also shown for a carrier of the A>T substitution mutation (FIG. 78) and a wild type patient (FIG. 7C). FIG. 7 discloses SEQ ID NOS: 124-126, respectively in order of appearance.

FIG. 8 shows DNA chromatogram traces of a patient having an G>A substitution mutation at nucleotide position 565 of SEQ ID NO: 2 resulting in an E>K amino acid substitution at amino acid position 189 of SEQ ID NO: 1 [see SEQ ID NO: 5] in the P2RY5 gene of a HYP16 family member (FIG. 8A). DNA chromatogram traces are also shown for a carrier of the G>A substitution mutation (FIG. 8B), and a wild type patient (FIG. 8C). FIG. 8 discloses SEQ ID NOS: 127-129, respectively in order of appearance.

FIG. 9 shows DNA chromatogram traces of a patient having an G>A substitution mutation at nucleotide position 833 of SEQ ID NO: 2 resulting in a C>Y amino acid substitution at amino acid position 278 of SEQ ID NO: 1 [see SEQ ID NO: 6] in the P2RY5 gene of a Brazilian family member (FIG. 9A). DNA chromatogram traces are also shown for a carrier of the G>A substitution mutation (FIG. 9B) and a wild type patient (FIG. 9C). FIG. 9 discloses SEQ ID NOS: 130-132, respectively in order of appearance.

FIG. 10 is a DNA gel stained with Ethidium Bromide depicting P2RY5 expression in a human adult hair follicle. Lane 1—molecular weight markers (MWM); Lane 2—P2RY5 in the presence of RT; Lane 3—P2RY5 in the absence of RT; Lane 4—GAPDH in the presence of RT; and Lane 5—GAPDH in the absence of RT.

FIG. 11 is a western blot using an anti-P2RY5 antibody that depicts P2RY5 protein expression in the back skin (lane 1) or foot pad (lane 2) of a mouse. Lane 3 shows P2RY5 expression of primary mouse keratinocytes cultured in high calcium media. The bottom panel represents β-actin as a loading control.

FIG. 12 is a western blot using an anti-P2RY5 antibody that depicts P2RY5 protein expression of primary mouse keratinocytes cultured in high calcium media at Days 1, 3, 6, and 10. The bottom panel represents β-actin as a loading control

FIG. 13 are immunofluorescent micrographs that depict P2RY5 expression in the suprabasal layers of the human epidermis in the presence (light grey, FIG. 13A) or absence (FIG. 13B; rabbit serum alone) of the anti-P2RY5 antibody. The DAPI staining represents the nuclei of cells (dark grey) (FIGS. 13A-B).

FIG. 14 are immunofluorescent micrographs that depict P2RY5 expression in the foot pad epidermis of mouse in the presence (FIG. 14A) or absence (FIG. 14B; rabbit serum alone) of the anti-P2RY5 antibody. The DAPI staining represents the nuclei of cells (dark grey) (FIGS. 14A-B).

FIG. 15 are immunofluorescent micrographs depicting P2RY5 expression in the Henle's layer of the human hair follicle (light grey, FIGS. 15A-B). K6 staining (grey) depicts the inner root sheath (FIG. 15A) or the hair follicle (FIG. 1513). The DAPI staining represents the nuclei of cells (dark grey) (FIGS. 15A-B).

FIG. 16 are immunofluorescent micrographs depicting P2RY5 expression in the Henle's layer (tight grey, FIG. 16A) and Huxley's layer (light grey, FIG. 16B) of the human hair follicle. K6 staining (grey) depicts the hair follicle (FIGS. 16 A-B). The DAPI staining represents the nuclei of cells (dark grey) (FIGS. 16A-B).

FIG. 17 are immunofluorescent micrographs depicting P2RY5 expression in the Henle's layer of the human hair follicle (light grey, FIGS. 17A-B). K6 staining (grey) depicts the inner root sheath (FIGS. 17A-B). The DAPI staining represents the nuclei of cells (dark grey) (FIGS. 17A-B).

FIG. 18 are immunofluorescent micrographs depicting P2RY5 expression in the Henle's layer (light grey, FIGS. 18A-B) and Huxley's layer (light grey, FIG. 18C) of the human hair follicle. The DAPI staining represents the nuclei of cells (dark grey) (FIGS. 18A-C).

FIG. 19 are immunofluorescent micrographs depicting P2RY5 expression in the rat whisker hair follicles (light grey, FIGS. 19A-B). K6 staining (grey) depicts the hair follicle (FIGS. 19A-B). The DAPI staining represents the nuclei of cells (dark grey) (FIGS. 19A-B).

FIG. 20 are immunofluorescent micrographs depicting P2RY5 expression in the human hair follicle (red, FIGS. 20A-B). K6 staining (light grey) depicts the inner root sheath (FIG. 20A). E-Cadherin staining (light grey) localizes to the inner root sheath (FIG. 20B). The DAPI staining represents the nuclei of cells (dark grey) (FIGS. 20A-B).

FIG. 21 are immunofluorescent micrographs depicting P2RY5 expression in the Henle's layer of the human hair follicle (light grey, FIGS. 21A-B). K6 staining (grey) depicts the inner root sheath (FIG. 21A) or the hair follicle (FIG. 21B). The DAPI staining represents the nuclei of cells (dark grey) (FIGS. 21A-B).

FIG. 22 are immunofluorescent micrographs that depict P2RY5 expression in the human hair follicle in the presence (light grey, FIG. 22A) or absence (FIG. 22B; rabbit serum alone) of the anti-P2RY5 antibody. The DAPI staining represents the nuclei of cells (dark grey) (FIGS. 22A-B). The white arrow points to the position where the Henle's Layer is keratinized.

FIG. 23 is a ribbon model of the membrane-spanning region of the rhodopsin crystal structure.

FIG. 24 is a serpentine amino acid representation (SEQ ID NOS: 133-138, respectively, in order of appearance) of the human P2RY5 receptor.

FIGS. 25A-B are photographs depicting the clinical appearance of autosomal recessive woolly hair (ARWH), which is mapped to chromosome 13q14.2-14.3.

FIG. 25C-D are micrograph images of plucked hairs of affected ARWH individuals under light microscopy. Scale bars: 100 μm.

FIG. 25E is a graph showing the results of ARWH autozygosity mapping. The maximum LOD score was obtained for a region on chromosome 13.

FIG. 25F is a schematic representation of haplotype analysis of ARWH24. The linked haplotype is indicated in red, and critical recombination events in each family are indicated by an arrow.

FIG. 25G is a schematic representation of haplotype analysis of ARWH5. The linked haplotype is indicated in red, and critical recombination events in each family are indicated by an arrow.

FIG. 26A is a schematic representation of the candidate region harboring the ARWH gene. Arrows indicate the position and the direction of transcription of genes in the region.

FIG. 26B depicts a haplotype analysis diagram (Left), a DNA chromatogram (Top Right), and a photograph of a DNA gel of ARWH2. Haplotypes and homozygous 69insCATG mutation in the P2RY5 of ARWH2 are represented (Left diagram). The box in the patient's sequence indicates the 4 bp insertion (Top Right, DNA Chromatogram). Results of restriction enzyme analysis are shown in the DNA gel below the DNA chromatograms. Affected individuals are colored in red. C, control individuals. FIG. 26B discloses SEQ ID NOS: 118, 139, and 119, respectively in order of appearance.

FIG. 26C depicts a haplotype analysis diagram (Left), a DNA chromatogram (Top Right), and a photograph of a DNA gel of ARWH18. Haplotype analysis for haplotypes and homozygous 172-175delAACT; and the 177delG mutation in P2RY5 in the ARWH18 family is shown (Left). The boxes in the control sequence indicate the deleted nucleotides (Top DNA Chromatogram). Results of restriction enzyme analysis are shown below the DNA chromatograms. Affected individuals are colored in red. C, control individuals. FIG. 26C discloses SEQ ID NOS: 120, 140 and 121, respectively in order of appearance.

FIG. 27A. depicts a multiple amino acid sequence alignment (SEQ ID NOS: 1, 141-144, 9 and 145-146, respectively, in order of appearance) of P2RY5 between different species. The position of each mutation is indicated by an arrow. Residues, 63D, 188I, and 189E, are indicated in red. Residues that are conserved among at least 6 species are colored light yellow. Transmembrane domains (TMs) are boxed.

FIG. 27B are ribbon diagrams that depict structural positions of P2RY5 point mutations. A ribbon diagram is shown for a homology model of P2RY5 based on the crystal structure of rhodopsin. For each mutant position, a space filling representation of the native amino acid is shown in red. The transmembrane helices are numbered. Two orthogonal views are shown.

FIG. 28A represents P2RY5 expression in the human hair follicle. The DNA gel represents RT-PCR amplification of the P2RY5 mRNA from plucked human hair follicles. RT(+)/RT(−) denote controls with or without reverse transcription. MWM, molecular weight marker.

FIG. 28B shows haematoxylin and eosin staining of human anagen hair follicle. ORS, outer root sheath; IRS, inner root sheath; DP, dermal papilla.

FIGS. 28C-E are fluorescence microscopy images of P2RY5 expression. Expression is predominantly expressed in both Henle's and Huxley's layers of IRS in human hair follicles (FIG. 28C), which is further confirmed by double immunostaining of P2RY5 with K6irs3 (IRS cuticle-specific keratin) (FIG. 28D) or K6hf (a companion layer-specific keratin) (FIG. 28E), Asterisks indicate the position where Henle's layer is completely keratinized (FIGS. 28B-C). Scale bars: 100 μm.

FIG. 28F represents global frequencies of SNP rs12430215. Allele frequencies of SNP rs12430215 are shown in European (CEU), African (YRI), Chinese (CHB), and Japanese (JPT) populations. C- and G-alleles are indicated in blue and red, respectively. The position of rs12430215 within intron 2 of the P2RY5 gene is, shown at the bottom.

FIG. 29 is a graph showing the results of parametric linkage analysis on chromosome 13. In addition to autozygosity mapping (in grey), parametric linkage analysis is performed twice, once using snps (in light grey) and once using haplotypes that are inferred from the data (in dark grey).

FIG. 30 is a graph depicting the results of multipoint, TDTAE, and two-point linkage analysis for four ARWH pedigrees. The values for the multipoint LOD scores are either computed using the method in SIMWALK2 or are interpolated linearly based on the map distance among markers. The LOD scores refer to two-point LOD scores maximized over recombination fraction values between 0.0 and 0.50, in increments of 0.02. The TDTAE values are computed by dividing the results of the TDTAE score (which is has a central chi-square distribution with 1 degree of freedom under the null hypothesis) by 2 ln(10).

FIG. 31A shows the identification of a missense mutation in the P2RY5 gene. A haplotype analysis (Top), DNA Chromatogram (Middle), and DNA gel image (Bottom) is shown for haplotypes and a homozygous 188A>T (D63V) mutation in ARWH15. Results of restriction enzyme analyses are shown below DNA chromatograms. Affected individuals are colored in red. C, control individuals. FIG. 31A discloses the amino acid sequences as SEQ ID NOS: 147-148 and 150, and the nucleotide sequences as SEQ ID NOS: 122, 149 and 123, respectively, in order of appearance.

FIG. 31B shows the identification of a missense mutation in the P2RY5 gene. A haplotype analysis (Top), DNA Chromatogram (Middle), and DNA gel image (Bottom) is shown for a homozygous 562A>T (I188F) mutation in both ARWH24 and ARWH5. Results of restriction enzyme analyses are shown below DNA chromatograms. Affected individuals are colored in red. C, control individuals. FIG. 31B discloses the amino acid sequences as SEQ ID NOS: 151-153 and the nucleotide sequences as SEQ ID NOS: 124, 161 and 126, respectively, in order of appearance.

FIG. 31C shows the identification of a missense mutation in the P2RY5 gene. A haplotype analysis (Top), DNA Chromatogram (Middle), and DNA gel image (Bottom) is shown for haplotypes and a homozygous 5650>A (E189K) mutation in ARWH 16. Results of restriction enzyme analyses are shown below DNA chromatograms. Affected individuals are colored in red. C, control individuals. FIG. 31C discloses the amino acid sequences as SEQ ID NOS: 154-155 and 157 and the nucleotide sequences as SEQ ID NOS: 128, 156 and 158, respectively, in order of appearance.

FIG. 32 represents that P2RY5 is expressed in suprabasal layers of the human epidermis. The junction between epidermis and dermis is indicated by dashes in the fluorescence micrograph of FIG. 32A. Scale bar: 100 μm, FIG. 32B is an image of a Western blot analysis of cell lysates obtained from normal human keratinocytes (NHK) grown on feeder layers in serum containing media. P2RY5 expression increases in a differentiation dependent manner. Cytokeratin 1 (K1), which is a differentiation marker of the epidermal keratinocytes, shows a similar expression pattern.

FIG. 33 are fluorescence micrographs showing the expression of mouse and rat P2RY5 in the hair follicles and skin. FIG. 33A is an image of a mouse whisker follicle (C57BL/6; post-natal day 30). FIG. 33B is an image of mouse back skin (C57BL/6; post-natal day 7). FIG. 33C is an image of mouse foot pad skin (C57BL/6; post-natal day 30). Note that the signal in the cornified layer is non-specific, since it is also detected in a control section incubated with normal rabbit serum. FIG. 33D is an image of a rat whisker follicle. Scale bars: (A, C and D), 100 μm, (B), 20 μm.

FIG. 34 is a schematic representation of the location of mouse P2RY5 gene and two mouse mutations on chromosome 14. Both wal and spc mutations map closely to the P2RY5 on chromosome 14.

FIG. 35 is diagram showing the location of sops within P2RY5 reported in HapMap rel21A/phaseII, January 2007, There is little reported variation within the gene. SNP rs12430215 has the most significant variation between ethnic groups (frequencies are reported below pie charts).

FIG. 36 is a schematic representation of the location of P2RY5 gene and other nested genes. FIG. 36A shows P2RY5 is a nested gene embedded within intron 17 of the RB1 gene. FIG. 36B demonstrates examples of other nested genes. Arrows indicate the direction of transcription. Exons are boxed, and coding and non-coding regions are colored in red and blue, respectively.

FIG. 37 is a schematic representation of P2RY5 gene and mRNA, in addition to the positions of target sequences for P2RY5 siRNAs.

FIG. 38 is a graph depicting siRNA knockdown of P2RY5 in HaCat Keratinocytes and analysis using real-time PCR.

FIG. 39 is, a schematic representation showing that lipase H produces 2-acyl-lysophosphatidic acid (2-acyl-LPA) from phosphatidic acid.

FIG. 40 is a photograph of a patient having a. mutation in the P2RY5 gene.

FIG. 41 is a photograph of a patient having a mutation in the P2RY5 gene.

FIG. 42 is a photograph of patients having a mutation in the P2RY5 gene.

FIG. 43 is a photograph of a patient having a mutation in the P2RY5 gene.

FIG. 44 is a DNA chromatogram depicting the identification of a 409T>C; 410-426del17 mutation in the P2RY5 gene in HYP51. FIG. 44 discloses SEQ ID NOS: 159-160, respectively, in order of appearance.

FIG. 45 is a schematic of the vector map for pEXP P2RY5 Gal4VP16M.

FIG. 46 is an amino acid sequence (SEQ ID NO: 162) where the amino sequence corresponding to P2RY5 is in upper case letters, the TEV recognition sequence is in upper case letters and underlined, and the Gal4VP16 region is in bolded and italicized upper case letters.

FIG. 47 is a nucleic acid sequence (SEQ ID NO: 163) where the nucleic sequence corresponding to P2RY5 is in upper case letters, the TEV recognition sequence is in upper case letters and underlined, and the Gal4VP16 region is in bolded and italicized upper case letters. The vector sequence is depicted in lower case letters.

FIG. 48 shows graphs where FBS was able to induce a slight beta-lactamase response through the P2RY5 receptor. Responsive cells were sorted into single cell clones and a pooled population.

FIG. 49 is a graph showing that FBS was able to induce a beta-lactamase response in the P2RY5 sorted pool that was greater than the response demonstrated by the original antibiotic selected pool.

FIG. 50 shows fluorescent photomicrographs of cells from the Tango™ P2RY5 antibiotic selected pool (FIG. 50A) and cells from the Tango™ GPCR U2OS parental cell line (FIG. 50B).

FIG. 51 is graph showing that TEV protease transfection can stimulate beta-lactamase reporter activity.

FIG. 52 shows photomicrographs of Tang™ P2RY5 cells transfected with a TEV protease expression plasmid (FIG. 52B) and cells not transfected (FIG. 52A). TEV protease transfection stimulates beta-lactamase reporter activity.

FIG. 53 shows graphs indicating that RS was able to induce a beta-lactamase response through the P2RY5 receptor. Responsive cells were sorted into single cell clones and green un-stimulated and blue stimulated pooled cell populations were collected.

FIG. 54 is a graph that shows both the blue and green sorted pools demonstrated an inducible beta-lactamase response to FBS.

FIG. 55 are fluorescent photomicrographs of the stimulated (+FBS) (TOP) and un-stimulated (−FBS) (BOTTOM) green sort pooled cells.

FIG. 56 is a graph showing that the Green sort pool cell demonstrated a slight inducible beta-lactamase response through the P2RY5 receptor to PMA.

FIG. 57 is a graph showing that FACS sorted clones demonstrated an inducible beta-lactamase response to dFBS.

FIG. 58 is a graph showing that all 5 clones demonstrated a concentration dependant beta-lactamase response to dFBS.

FIG. 59 is a graph that shows that PMA induced a concentration dependant beta-lactamase response through the P2RY5 receptor in the sorted clones.

FIG. 60 is a graph of an Assay for DMSO Tolerance.

FIG. 61 is a graph showing the effect of PMA stimulation time.

FIG. 62 shows graphs of cryopreserved cells stimulated with PMA for 18 h (FIG. 62A) or 5 hr (FIG. 62B).

FIG. 63 is a graph showing day to day performance of the assay. The assay performance is reproducible when run on different days under optimized conditions.

FIG. 64 is a graph showing the dose-response of Tango™ P2RY5-bla U2OS cells to PMA.

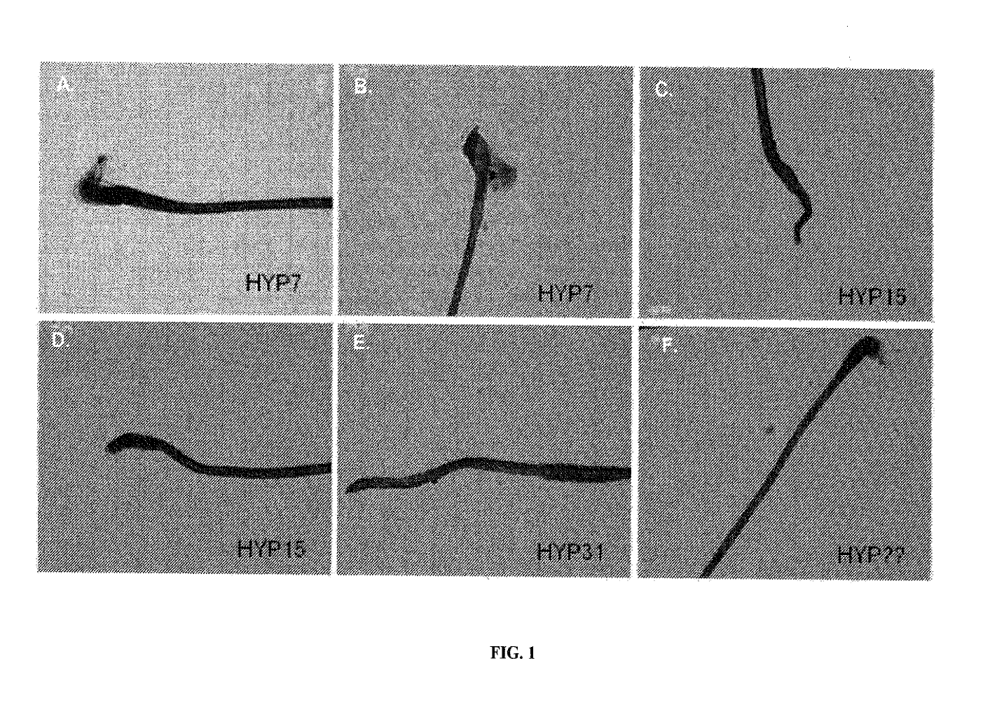

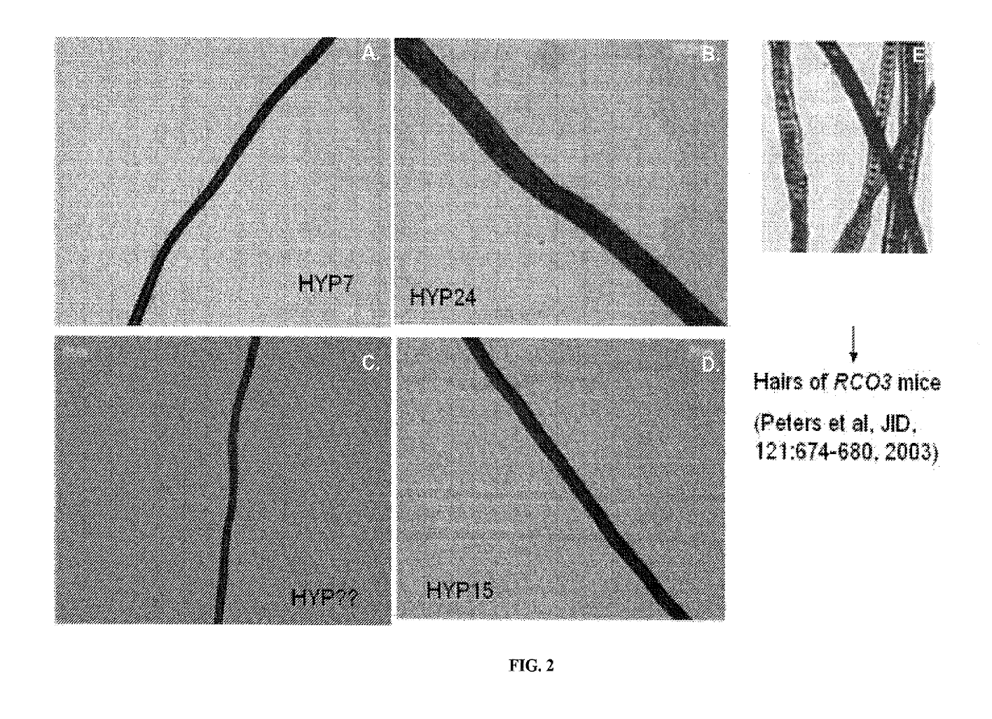

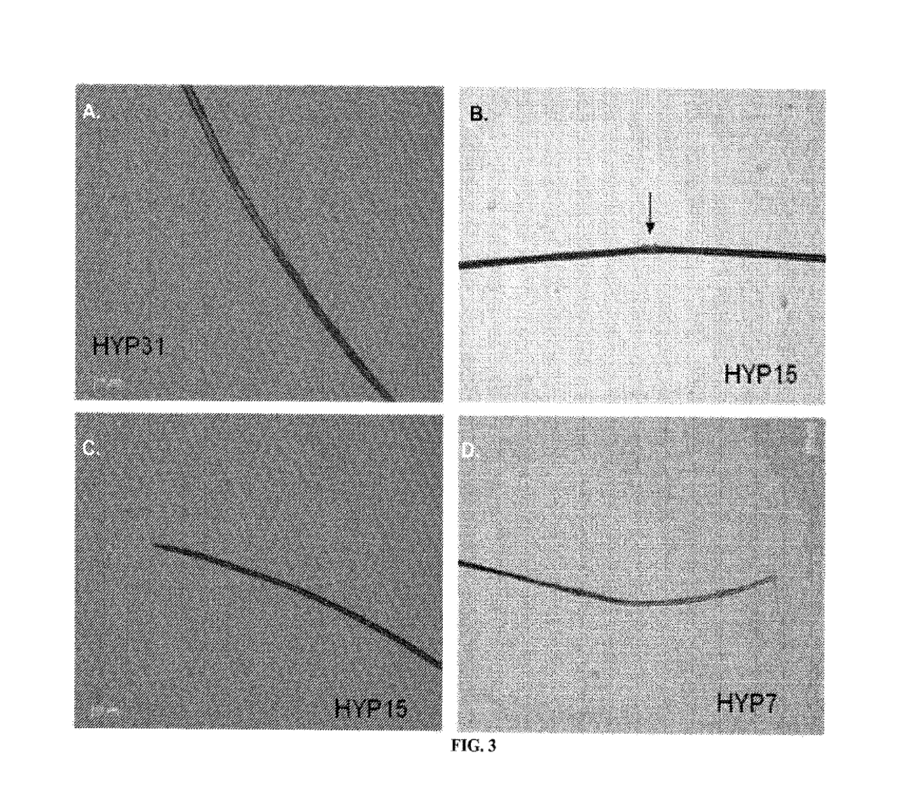

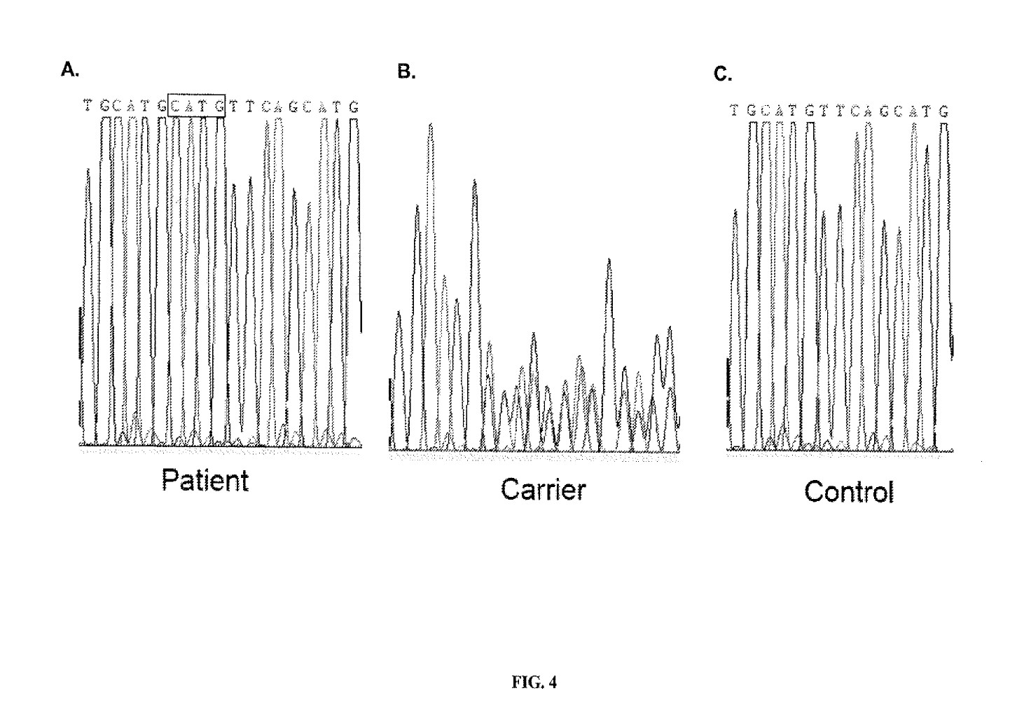

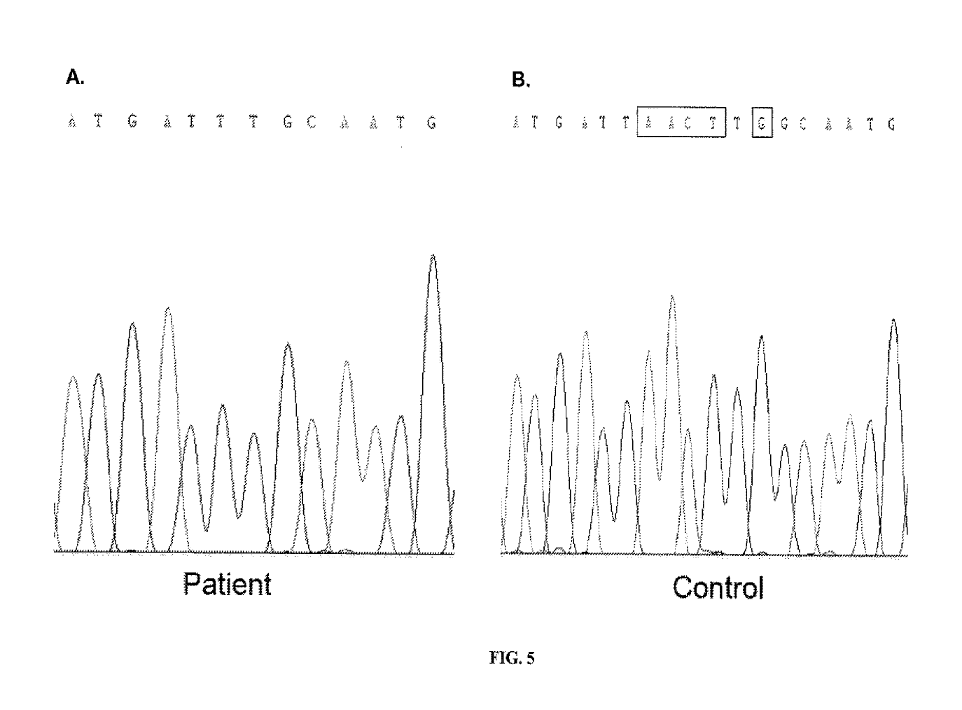

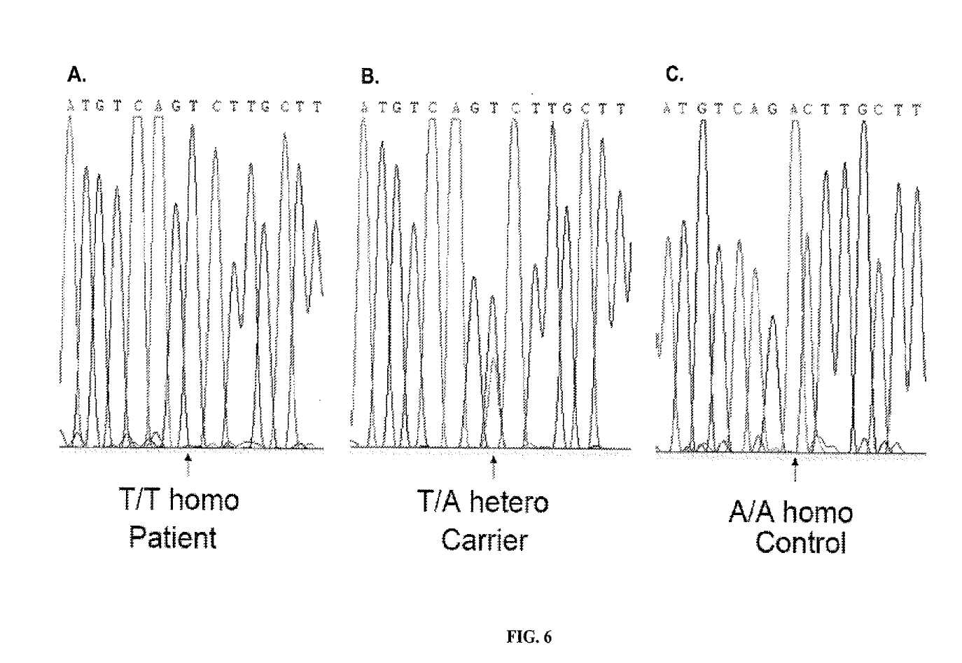

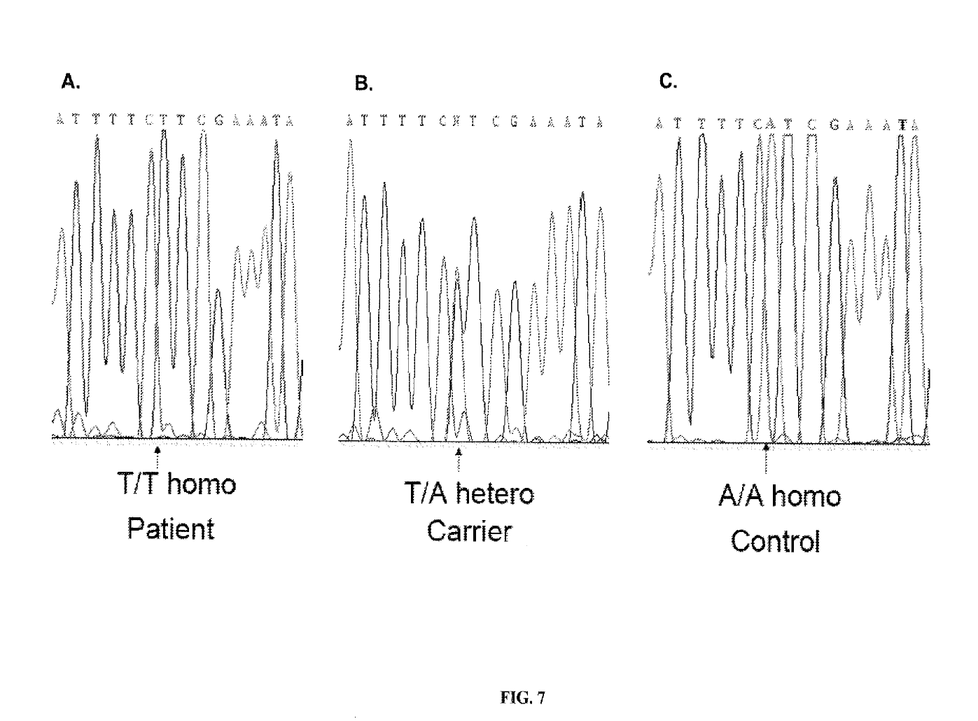

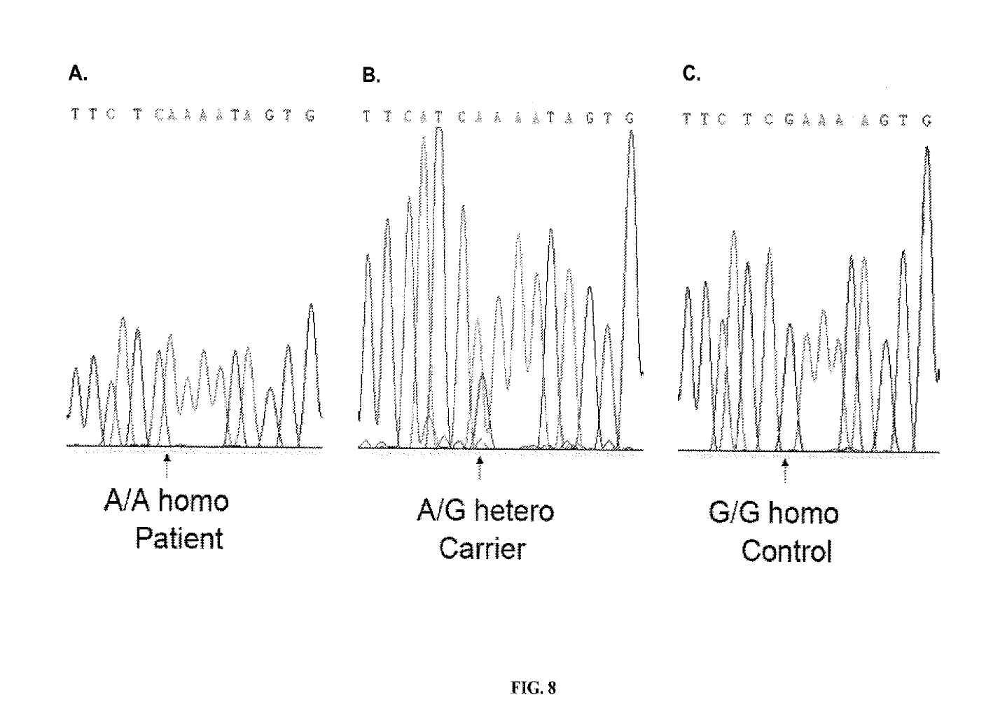

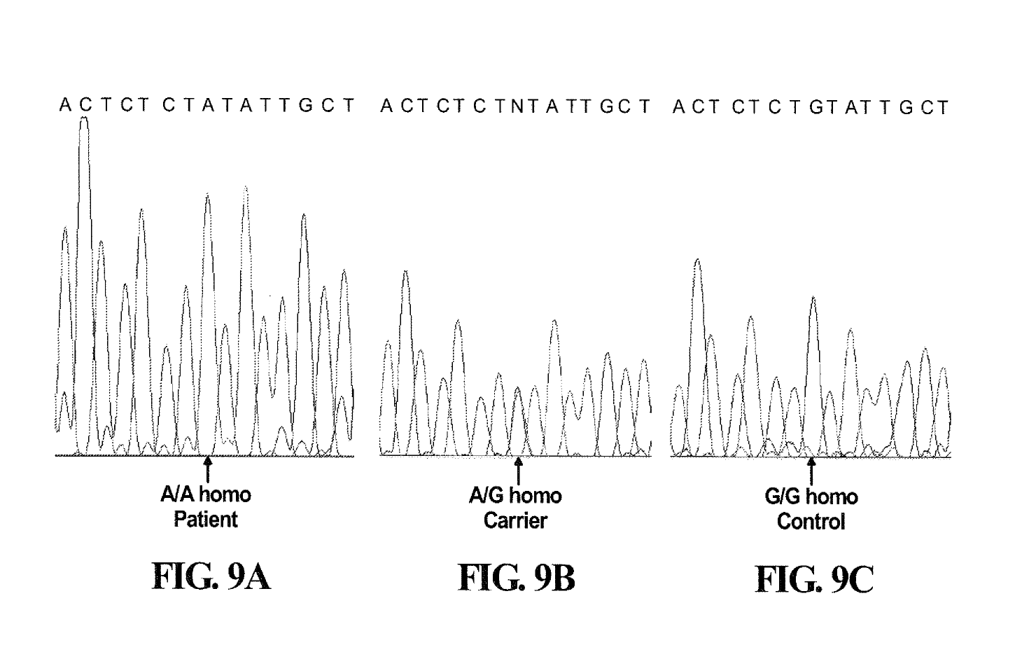

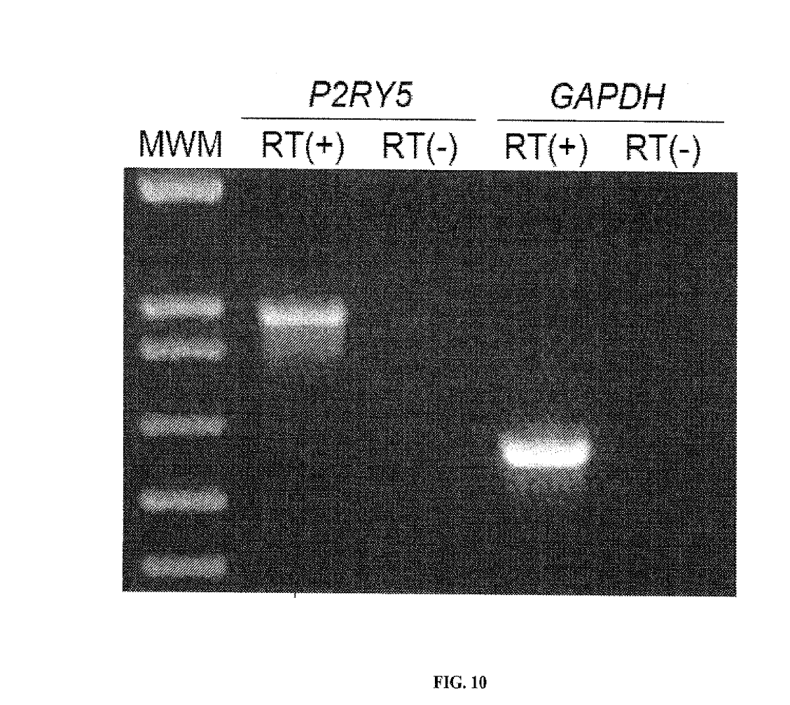

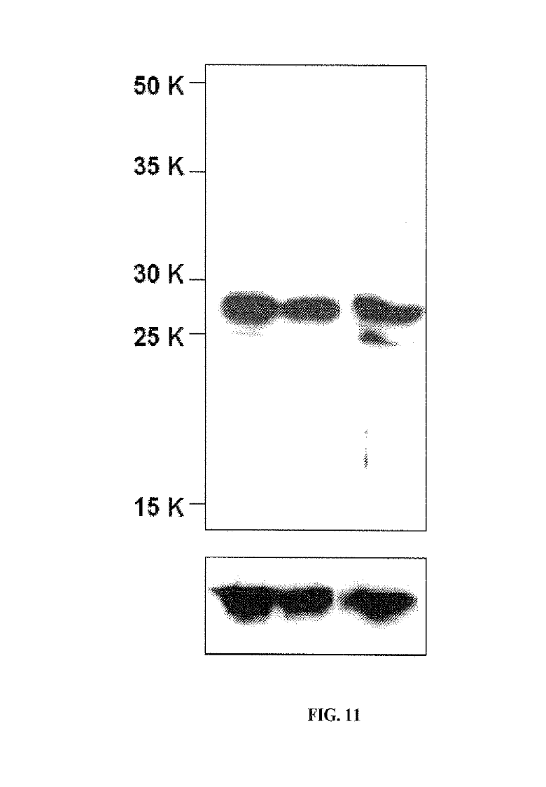

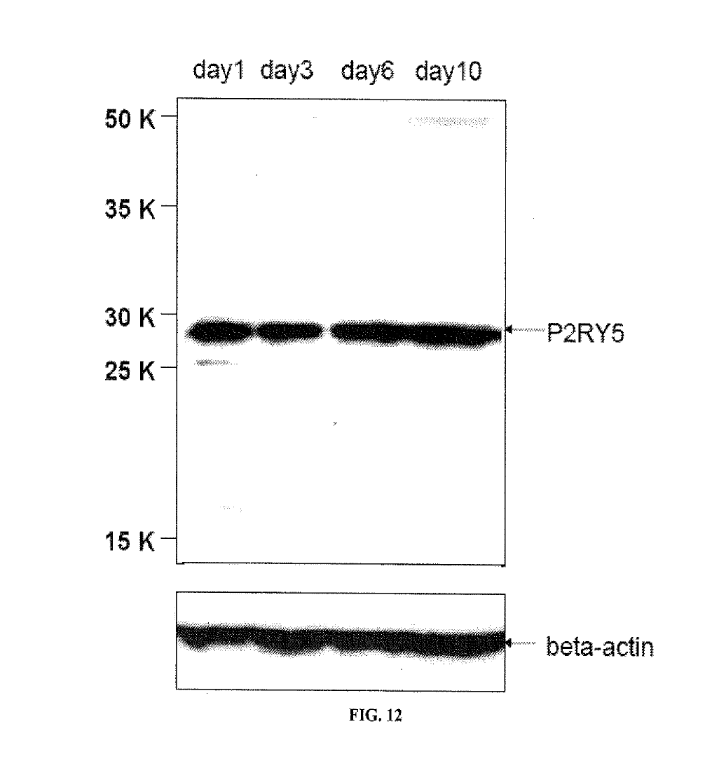

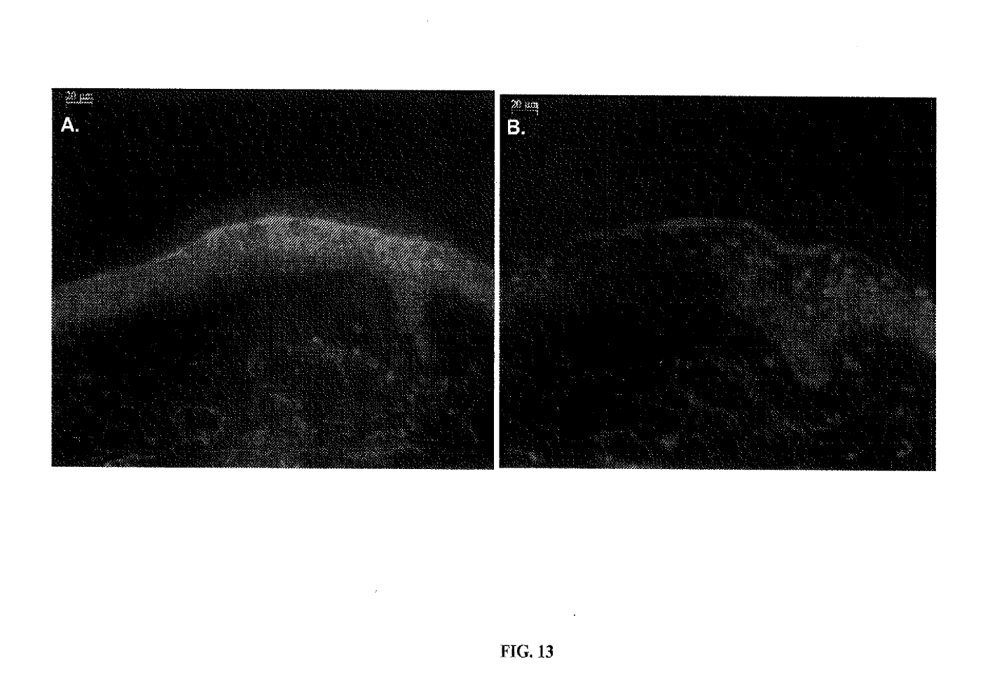

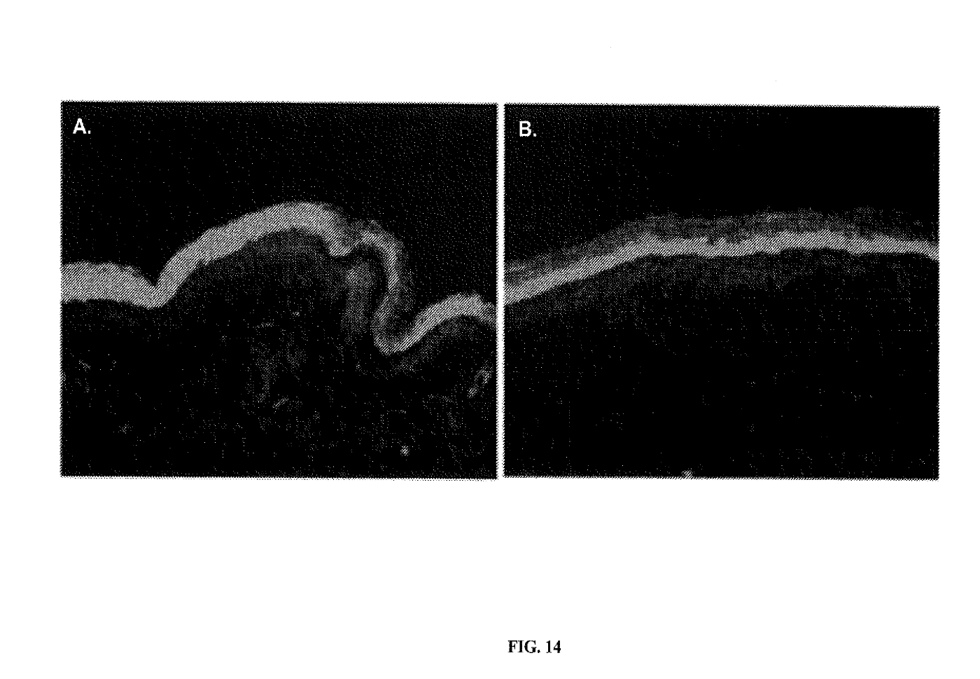

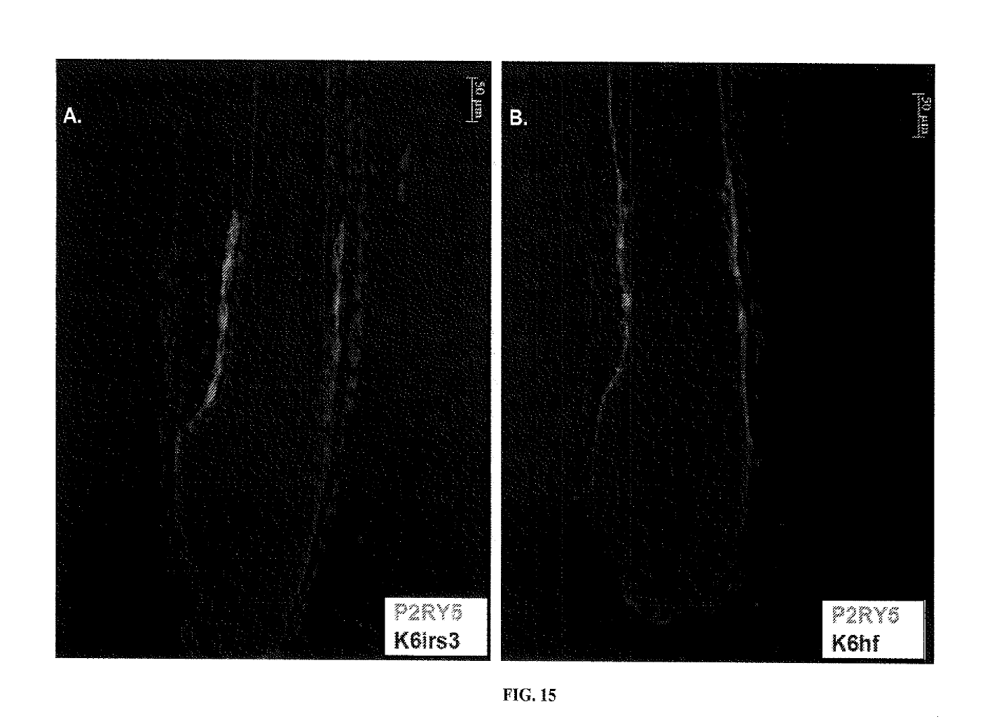

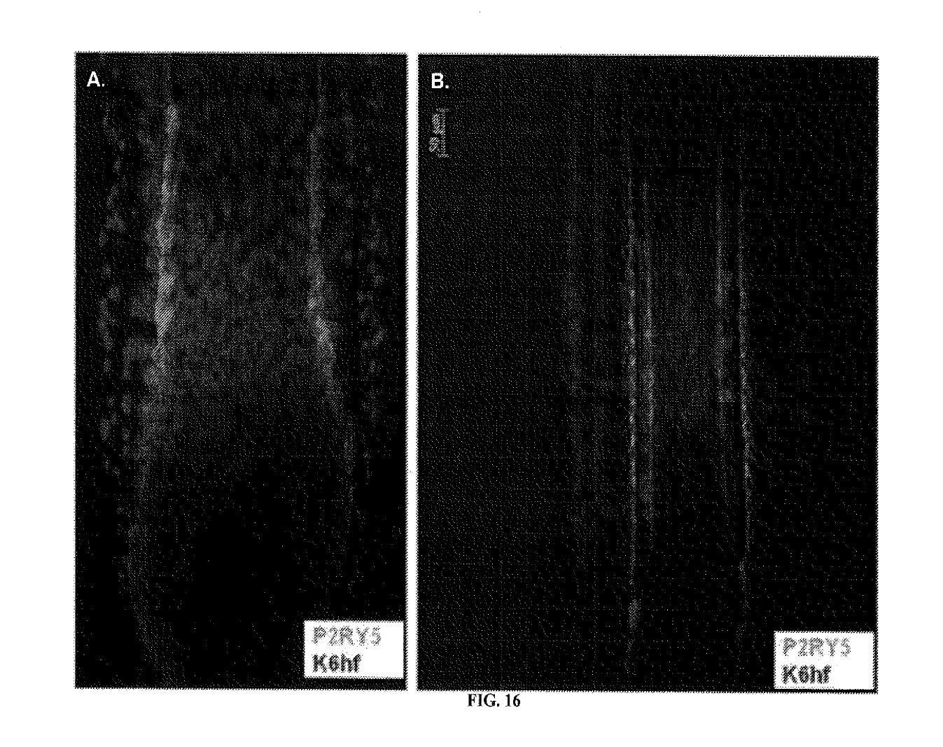

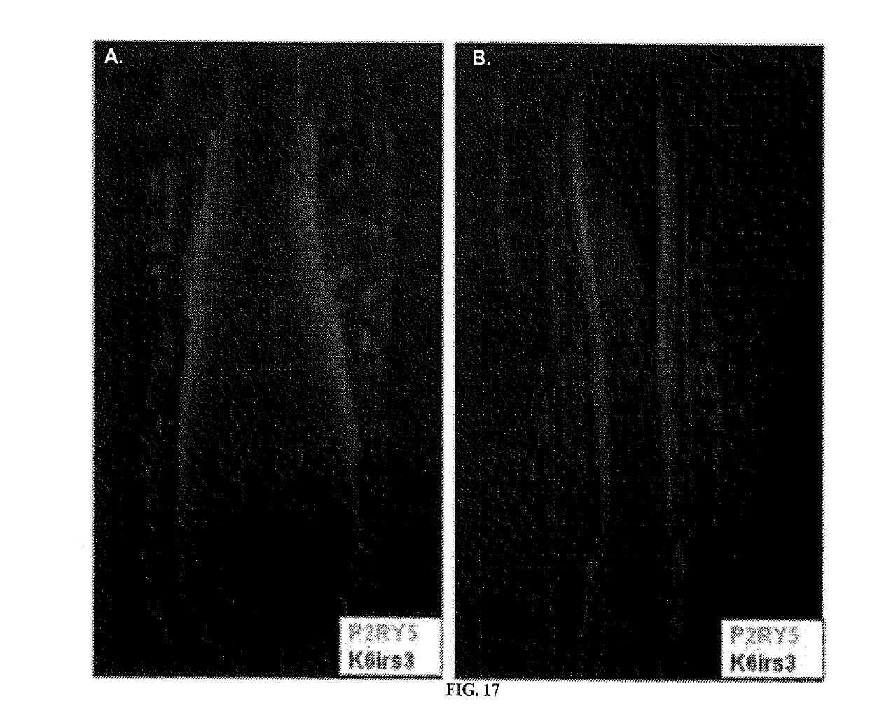

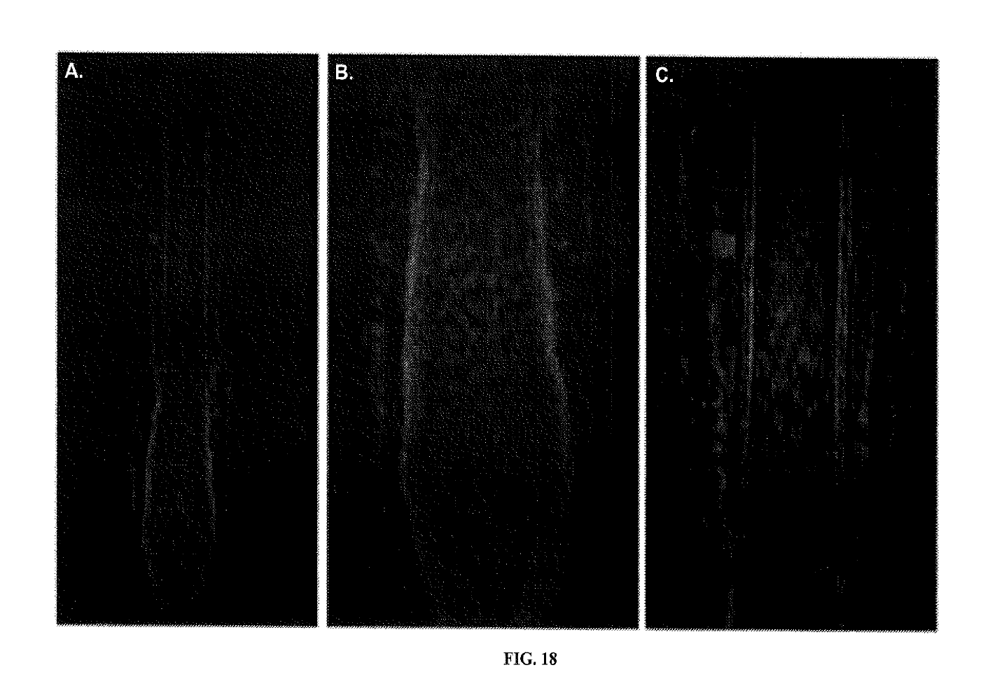

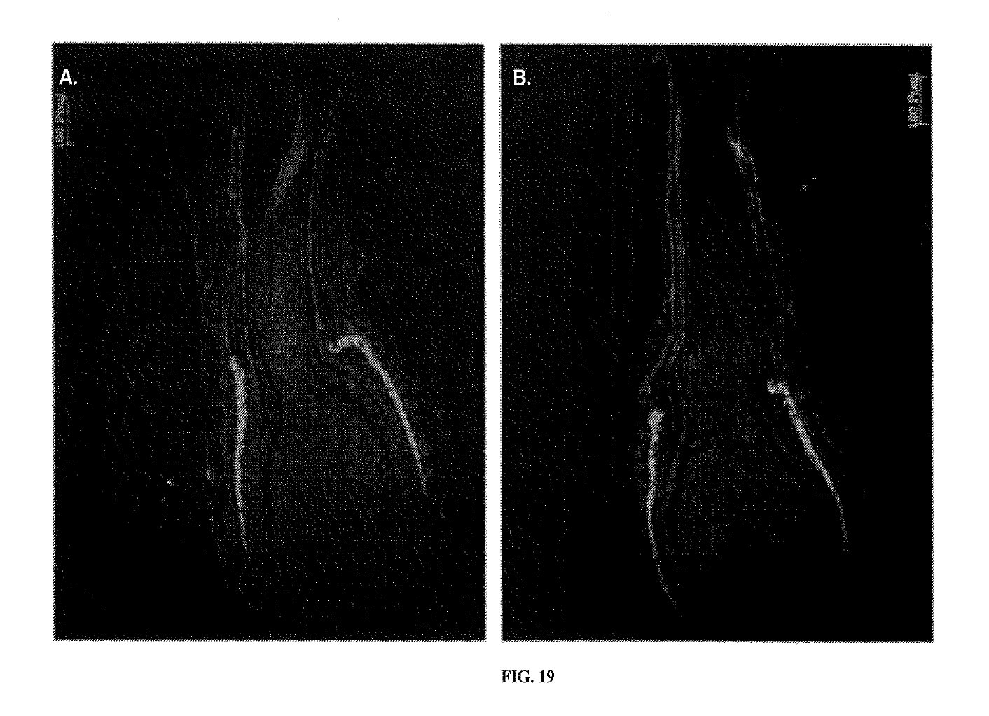

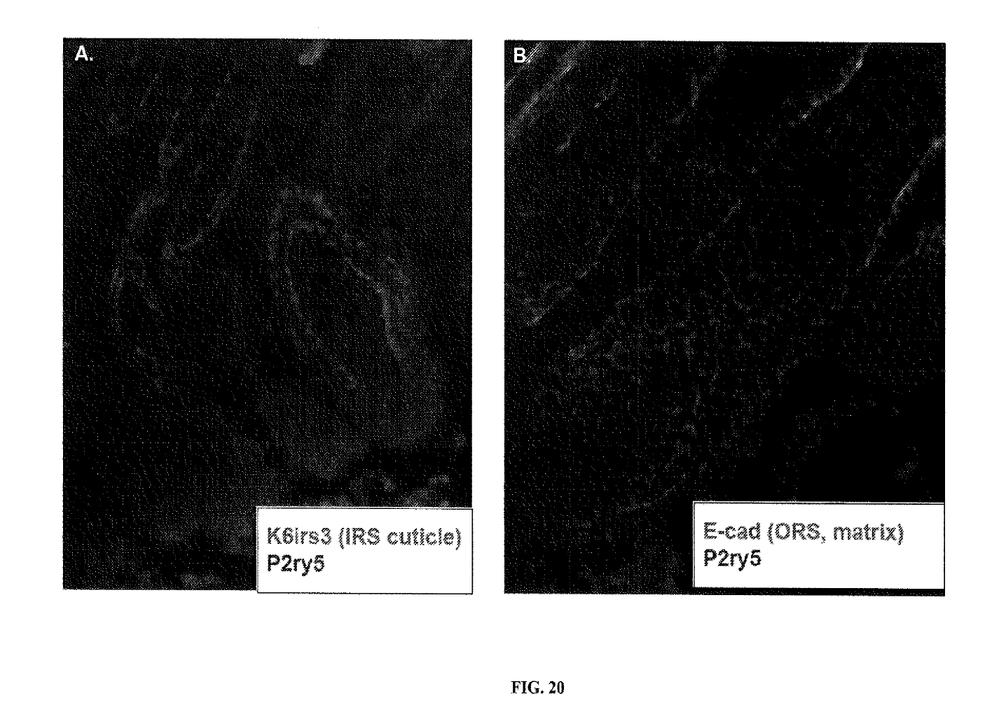

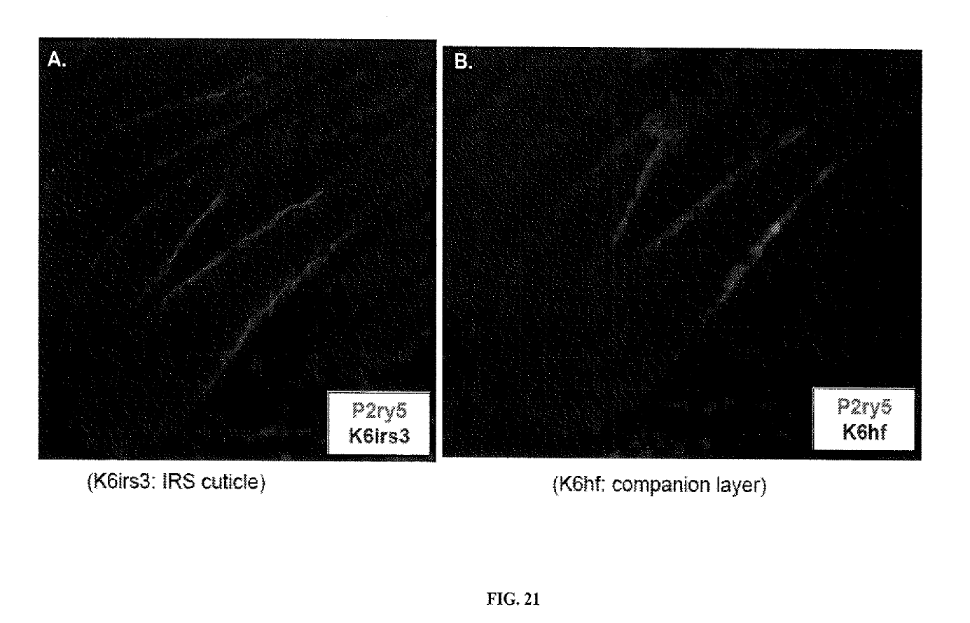

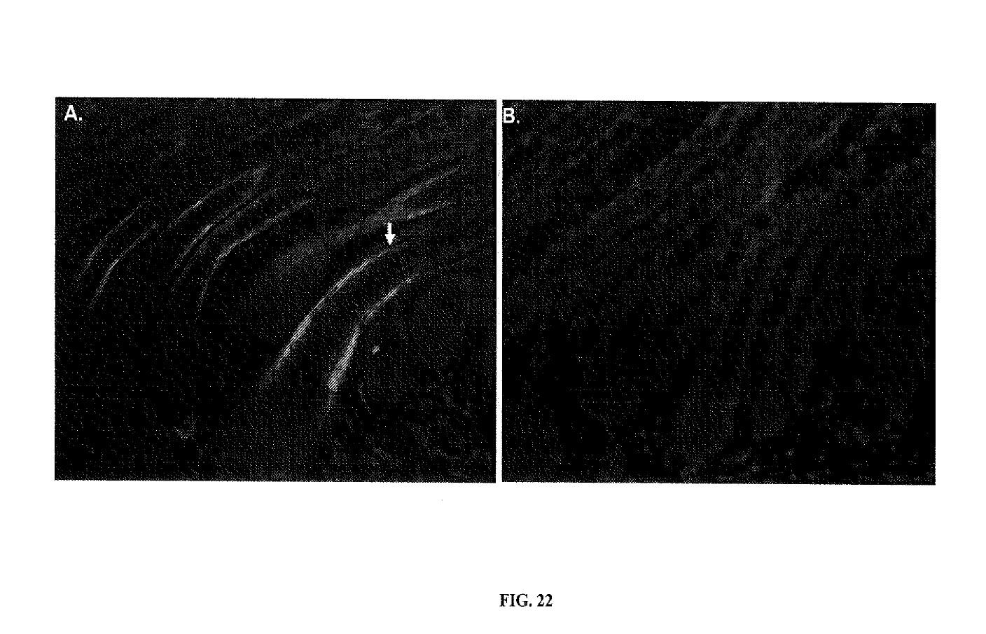

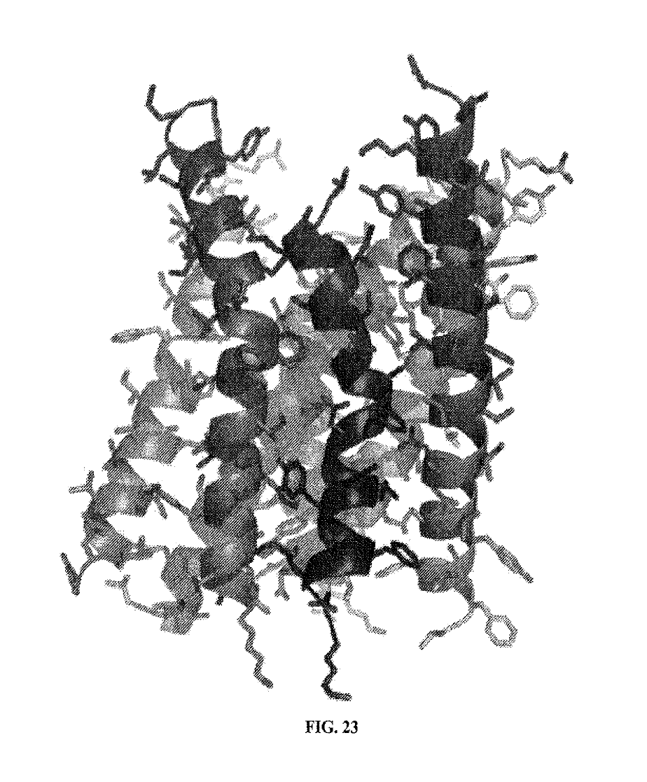

### SUMMARY OF THE INVENTION

The invention provides for an isolated mutant human P2RY5 polypeptide comprising at least 1 amino acid mutation in transmembrane domain (TMD) I, wherein TMD I comprises amino acids at positions of about 20 to about 42 of SEQ ID NO:1; TMD II, wherein TMD II comprises amino acids at positions of about 55 to about 77 of SEQ ID NO:1; TMD III, wherein TMD III comprises amino acids at positions of about 100 to about 122 of SEQ ID NO:1; TMD IV, wherein TMD IV comprises amino acids at positions of about 135 to about 154 of SEQ ID NO:1; TMD V, wherein TMD V comprises amino acids at positions of about 179 to about 201 of SEQ ID NO:1; TMD VI, wherein TMD VI comprises amino acids at positions of about 230 to about 252 of SEQ ID NO:1; TMD VII, wherein TMD VII comprises amino acids at positions of about 272 to about 294 of SEQ ID NO:1; or a combination thereof, wherein the P2RY5 polypeptide comprises an amino acid sequence of SEQ ID NO: 1.

In one embodiment, the mutation is a D>V mutation at amino acid position 63 of SEQ ID NO: 1, comprising the amino acid sequence of SEQ ID NO: 3. In one embodiment, the mutation is an I>F mutation at amino acid position 188 of SEQ ID NO: 1, comprising the amino acid sequence of SEQ ID NO: 4. In one embodiment, the mutation is an E>K mutation at amino acid position 189 of SEQ ID NO: 1, comprising the amino acid sequence of SEQ ID NO: 5. In one embodiment, the mutation is a C>Y mutation at amino acid position 278 of SEQ ID NO: 1, comprising the amino acid sequence of SEQ ID NO: 6. In one embodiment, the mutation is a Y>C mutation at amino acid position 245 of SEQ ID NO: 1, comprising the amino acid sequence of SEQ ID NO: 109. In one embodiment, the mutation is a G>R mutation at amino acid position 146 of SEQ ID NO: 1, comprising the amino acid sequence of SEQ ID NO: 110.

The invention provides an isolated mutant human P2RY5 polypeptide encoded by a nucleic acid sequence comprising at least about 50%, at least about 60%, at least about 70%, at least about 75%, at least about 80%, at least about 90%, at least about 95%, or at least about 99% identity of SEQ ID NO: 2. In one embodiment, the nucleic acid sequence comprises the nucleic acid sequence of SEQ ID NO: 7 or SEQ ID NO: 8.

The invention provides for a nucleic acid encoding any of the polypeptides described herein. The invention provides for a nucleic acid consisting essentially of a nucleic acid encoding any of the polypeptides described herein. The invention provides for a nucleic acid consisting of a nucleic acid encoding any of the polypeptides described herein. The invention provides a vector containing one or more of the nucleic acids described herein. The invention provides a vector comprising one or more of the nucleic acids described herein. The invention provides a vector consisting essentially of one or more of the nucleic acids described herein. The invention provides a vector consisting of one or more of the nucleic acids described herein.

The invention provides for a method for identifying a compound that binds to a P2RY5 protein, the method comprising: providing an electronic library of test compounds; providing atomic coordinates listed herein for at least 20 amino acid residues for the binding pocket of the P2RY5 protein, wherein the coordinates have a root mean square deviation therefrom, with respect to at least 50% of Cα atoms, of not greater than about 5 Å, in a computer readable format; converting the atomic coordinates into electrical signals readable by a computer processor to generate a three dimensional model of the P2RY5 protein; performing a data processing method, wherein electronic test compounds from the library are superimposed upon the three dimensional model of the P2RY5 protein; and determining which test compound fits into the binding pocket of the three dimensional model of the P2RY5 protein, thereby identifying which compound would bind to P2RY5.

The invention provides for a method for identifying a compound that modulates P2RY5 protein activity, the method comprising; (1) expressing P2RY5 protein in a cell; (2) contacting a cell with a ligand source for an effective period of time; (3) measuring a secondary messenger response, wherein the response is indicative of a ligand binding to P2RY5 protein; (4) isolating the ligand from the ligand source; and (5) identifying the structure of the ligand that binds P2RY5 protein, thereby identifying which compound would modulate the activity of P2RY5 protein. The method can further comprise: obtaining or synthesizing the compound determined to bind to P2RY5 protein or to modulate P2RY5 protein activity; contacting P2RY5 protein with the compound under a condition suitable for binding; and determining whether the compound modulates P2RY5 protein activity using a diagnostic assay. In one embodiment, the compound is a P2RY5 agonist or a P2RY5 antagonist. In another embodiment, the antagonist decreases P2RY5 protein or RNA expression or P2RY5 activity by at least about 10%, at least about 20%, at least about 30%, at least about 40%, at least about 50%, at least about 60%, at least about 70%, at least about 75%, at least about 80%, at least about 90%, at least about 95%, at least about 99%, or 100%. In a further embodiment, the agonist increases P2RY5 protein or RNA expression or P2RY5 activity by at least about 10%, at least about 20%, at least about 30%, at least about 40%, at least about 50%, at least about 60%, at least about 70%, at least about 75%, at least about 80%, at least about 90%, at least about 95%, at least about 99%, or 100%. In some embodiments, the compound comprises an antibody that specifically binds to a P2RY5 protein or a fragment thereof; an antisense RNA or antisense DNA that inhibits expression of P2RY5 polypeptide; a siRNA that specifically targets a P2RY5 gene; a peptide comprising at least 10 amino acids of SEQ ID NO:1 wherein the peptide competes with endogenous P2RY5 receptor for ligand binding; or a combination of the compounds mentioned herein. In further embodiments, the compound comprises Formula I, Formula II, Formula III, or Formula IV. In one embodiment, the cell is a bacterium, a yeast, an insect cell, or a mammalian cell. In another embodiment, the ligand source is a compound library, a tissue extract, or a neurotransmitter collection. In a further embodiment, the measuring comprises detecting an increase or decease in a secondary messenger concentration. In some embodiments, the assay determines the concentration of the secondary messenger within the cell. In other embodiments, the secondary messenger comprises adenylyl cyclase, cyclic AMP, phospholipase C, Ca²⁺, inositol 1,4,5-triphosphate (IP₃), or a combination thereof. In one embodiment, the contacting comprises administering the compound to a mammal in vivo or a cell in vitro. In some embodiments, the mammal is a mouse. In another embodiment, the assay is a cell-based assay or a cell-free assay. In some embodiments, the compound increases or decreases downstream receptor signaling of the P2RY5 protein. In other embodiments, the assay measures an intracellular concentration of ATP, adenylyl cyclase, cyclic AMP, phospholipase C, Ca²⁺, or inositol 1,4,5-triphosphate (IP₃).

The invention provides a method for controlling hair growth in a subject, the method comprising: administering to the subject an effective amount of a P2RY5 receptor modulating compound, thereby controlling hair growth in the subject. In one embodiment, the subject is a human, a primate, a feline, a canine, or an equine. In another embodiment, the compound comprises an antibody that specifically binds to a P2RY5 protein or a fragment thereof; an antisense RNA or antisense DNA that inhibits expression of P2RY5 polypeptide; a siRNA that specifically targets a P2RY5 gene, a peptide comprising at least 10 amino acids of SEQ ID NO:1 wherein the peptide competes with endogenous P2RY5 receptor for ligand binding; or a combination of the compounds described herein. In further embodiments, the compound comprises Formula I, Formula II, Formula III, or Formula IV. In a further embodiment, the subject is afflicted with a hair-loss disorder. In some embodiments, the hair-loss disorder comprises androgenetic alopecia, Telogen effluvium, Alopecia areata, telogen effluvium, Alopecia areata, Tinea capitis, alopecia totalis, or alopecia universalis. In one embodiment, the subject is treated with a P2RY5 agonist. In another embodiment, administering comprises dispersing the P2RY5 modulating compound to a subject via subcutaneous, intra-muscular, intra-peritoneal, or intravenous injection; infusion; oral, nasal, or topical delivery; or a combination thereof. In a further embodiment, the P2RY5 agonist comprises a nucleic acid encoding human P2RY5 protein. In some embodiments, controlling hair growth comprises a promotion of hair growth in the subject; a promotion of hair loss in the subject; or a straightening of hair in the subject. In other embodiments, straightening comprises relaxing a hair shaft. In further embodiments, the hair shaft is an Afraid shaft or Caucasoid shaft.

The invention provides a composition for controlling hair growth in a subject, the composition in an admixture of a pharmaceutically acceptable carrier comprising a P2RY5 modulating compound. In one embodiment, controlling hair growth comprises a promotion of hair growth in the subject; a promotion of hair loss in the subject; or a straightening of hair in the subject. In another embodiment, the pharmaceutically acceptable carrier comprises water, a glycol, an ester, an alcohol, a lipid, or a combination of the carriers listed herein. In a further embodiment, straightening comprises relaxing a hair shaft. In some embodiments, the hair shaft is an Afroid shaft or Caucasoid shaft. In further embodiments, the compound comprises Formula I, Formula II, Formula III, or Formula IV.

The invention provides a kit for controlling hair growth, wherein the kit comprises a container having a composition of the invention disposed therein and instructions for use, wherein the composition is in an admixture of a pharmaceutically acceptable carrier comprising a P2RY5 modulating compound. In some embodiments, the compound comprises Formula I, Formula II, Formula III, or Formula IV.

The invention provides a composition for modulating P2RY5 protein expression or activity, wherein the composition comprises an siRNA that specifically targets a P2RY5 gene. In one embodiment, the siRNA comprises a nucleic acid sequence comprising SEQ ID NO: 13, 14, 15, or 16. In another embodiment, P2RY5 expression is decreased by at least about 40%, at least about 45%, at least about 50%, at least about 55%, at least about 60%, at least about 65%, at least about 70%, at least about 75%, at least about 80%, at least about 90%, at least about 95%, at least about 99%, or 100%.

### DETAILED DESCRIPTION OF THE INVENTION

Although features of P2Y receptor signaling in certain cell types are well known, the physiological roles of P2Y receptors in the hair, such as P2YR5, however, are not well-characterized. The invention provides for isolated mutants of P2RY5 as well as compounds that modulate P2RY5 protein expression or activity. The invention provides for methods of using P2RY5 protein, or agonists or antagonists thereof to control and regulate hair growth and texture in a subject. For example, damaging and harsh hair treatments available in the current market in order to control and regulate hair growth and texture (such as through hair relaxers and home perms) cannot be used continuously. As such, the invention meeis a long-felt need in the hair care industry, one that generates about $1.3 Billion in sales, since the methods and compositions disclosed herein can circumvent the damaging and harsh treatments used today to control hair growth and texture.

The invention provides for the discovery that P2RY5 is a gene involved in regulating/controlling hair growth. P2RY5 is a 7 transmembrane (TM), G-protein coupled receptor (GPCR). G-protein mediated signal transduction pathways mediate numerous medically significant biological processes. The family of G-protein coupled receptors (GPCRs) includes receptors for hormones, neurotransmitters, growth factors, and viruses. Various examples of GPCRs include receptors for agents as dopamine, calcitonin, adrenergic hormones, endotheline, cAMP, adenosine, acetylcholine, serotonine, histamine, thrombin, kinine, follicle stimulating hormone, opsins, endothelial differentiation gene-1, rhodopsins, odorants, cytomegalovirus, G-proteins themselves, effector proteins such as phospholipase C, adenyl cyclase, and phosphodiesterase, and actuator proteins such as protein kinase A and protein kinase C. Thus, P2RY5 is a desirable drug target to identify small molecules to either inhibit or enhance hair growth.

Overview of the Integument

The integument (or skin) is the largest organ of the body and is a highly complex organ covering the external surface of the body. It merges, at various body openings, with the mucous membranes of the alimentary and other canals. The integument performs a number of essential functions such as maintaining a constant internal environment via regulating body temperature and water loss; excretion by the sweat glands; but predominantly acts as a protective barrier against the action of physical, chemical and biologic agents on deeper tissues. Skin is elastic and except for a few areas such as the soles, palms, and ears, it is loosely attached to the underlying tissue. It also varies in thickness from 0.5 mm (0.02 inches) on the eyelids (“thin skin”) to 4 mm (0.17 inches) or more on the palms and soles (“thick skin”) (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The skin is composed of two layers: a) the epidermis and b) the dermis. The epidermis, or cuticle, is the outer layer, which is comparatively thin (0.1 mm). It is several cells thick and is composed of 5 layers: the stratum germinativum, stratum spinosum, stratum granulosum, stratum lucidum (which is limited to thick skin), and the stratum comeum. The outermost epidermal layer (the stratum corneum) consists of dead cells that are constantly shed from the surface and replaced from below by a single, basal layer of cells, called the stratum germinativum. The epidermis is composed predominantly of keratinocytes, which make up over 95% of the cell population. Keratinocytes of the basal layer (stratum germinativum) are constantly dividing, and daughter cells subsequently move upwards and outwards, where they undergo a period of differentiation, and are eventually sloughed off from the surface. The remaining cell population of the epidermis includes dendritic cells such as Langerhans cells and melanocytes. The epidermis is essentially cellular and non-vascular, containing little extracellular matrix except for the layer of collagen and other proteins beneath the basal layer of keratinocytes (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The dermis is the inner layer of the skin and is composed of a network of collagenous extracellular material, blood vessels, nerves, and elastic fibers. Within the dermis are hair follicles with their associated sebaceous glands (collectively known as the pilosebaceous unit) and sweat glands. The interface between the epidermis and the dermis is extremely irregular and uneven, except in thin skin. The junction between the two layers consists of a succession of finger like connective tissue protrusions, called dermal papillae (DP). Beneath the basal epidermal cells along the epidermal-dermal interface, the specialized extracellular matrix is organized into a distinct structure called the basement membrane (Ross MH, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The mammalian hair fiber is composed of keratinized cells and develops from the hair follicle. The hair follicle is a peg of tissue derived from a downgrowth of the epidermis, which lies immediately underneath the skin's surface. The distal part of the hair follicle is in direct continuation with the external, cutaneous epidermis. Although a small structure, the hair follicle comprises a highly organized system of recognizably different layers arranged in concentric series. Active hair follicles extend down through the dermis, the hypodermis (which is a loose layer of connective tissue), and into the fat or adipose layer (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

At the base of an active hair follicle lies the hair bulb. The bulb consists of a body of dermal cells, known as the dermal papilla, contained in an inverted cup of epidermal cells known as the epidermal matrix. Irrespective of follicle type, the germinative epidermal cells at the very base of this epidermal matrix produce the hair fiber, together with several supportive epidermal layers. The lowermost dermal sheath is contiguous with the papilla basal stalk, from where the sheath curves externally around all of the hair matrix epidermal layers as a thin covering of tissue. The lowermost portion of the dermal sheath then continues as a sleeve or tube for the length of the follicle (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

Developing skin appendages, such as hair and feather follicles, rely on the interaction between the epidermis and the dermis, the two layers of the skin. In embryonic development, a sequential exchange of information between these two layers supports a complex series of morphogenetic processes, which results in the formation of adult follicle structures. However, in contrast to general skin dermal and epidermal cells, certain hair follicle cell populations, following maturity, retain their embryonic-type interactive, inductive, and biosynthetic behaviors. These properties can be derived from the very dynamic nature of the cyclical productive follicle, wherein repeated tissue remodeling necessitates a high level of dermal-epidermal interactive communication, which is vital for embryonic development and would be desirable in other forms of tissue reconstruction.

The hair fiber is produced at the base of an active follicle at a very rapid rate. For example, follicles produce hair fibers at a rate 0.4 mm per day in the human scalp and up to 1.5 mm per day in the rat vibrissa or whiskers, which means that cell proliferation in the follicle epidermis ranks amongst the fastest in adult tissues (Malkinson F D and JT Kearn, Int J Dermatol 1978, 17:536-551). Hair grows in cycles. The anagen phase is the growth phase, wherein up to 90% of the hair follicles said to be in anagen; catagen is the involuting or regressing phase which accounts for about 1-2% of the hair follicles; and telogen is the resting or quiescent phase of the cycle, which accounts for about 10-14% of the hair follicles. The cycle's length varies on different parts of the body.

Hair follicle formation and cycling is controlled by a balance of inhibitory and stimulatory signals. The signaling cues are potentiated by growth factors that are members of the TGFβ-BMP family. A prominent antagonist of the members of the TGFβ-BMP family is follistatin. Follistatin is a secreted protein that inhibits the action of various BMPs (such as BMP-2, -4, -7, and -11) and activins by binding to said proteins, and may play a role in the development of the hair follicle (Nakamura M, et al., FASEB J, 2003, 17(3):497-9; Patel K Intl J Biochem Cell Bio, 1998, 30:1087-93; Ueno N, et al., PNAS, 1987, 84:8282-86; Nakamura T, et al., Nature, 1990, 247:836-8; Iemura S, et al., PNAS, 1998, 77:649-52; Fainsod A, et al., Mech Dev, 1997, 63:39-50; Gamer L W, et al., Dev Bial, 1999, 208:222-32).

The deeply embedded end bulb, where local dermal-epidermal interactions drive active fiber growth, is the most dynamic region of the hair follicle. This same region is also central to the tissue remodeling and developmental changes involved in the hair fiber's or appendage's precise alternation between growth and regression phases. The dermal papilla, a key player in these activities, appears to orchestrate the complex program of differentiation that characterizes hair fiber formation from the primitive germinative epidermal cell source (Oliver R F, J Soc Cosmet Chem, 1971, 22:741-755; Oliver R F and C A Jahoda, Biology of Wool and Hair (eds Roger et al.), 1971, Cambridge University Press: 51-67; Reynolds A J and CA Jahoda, Development, 1992, 115:587-593; Reynolds A J, et. al., J Invest Dermatol, 1993, 101:634-38).

The lowermost dermal sheath arises below the basal stalk of the papilla, from where it curves outwards and upwards. This dermal sheath then externally encloses the layers of the epidermal hair matrix as a thin cup of tissue and continues as a tubular arrangement for the length of the follicle. The epidermal outer root sheath (ORS) also continues for the length of the follicle, which lies immediately internal to the dermal sheath in between the two layers, and forms a specialized basement membrane termed the glassy membrane. The outer root sheath constitutes little more than an epidermal monolayer in the lower follicle, but becomes increasingly thickened as it approaches the surface. The inner root sheath (IRS) forms a mold for the developing hair shaft. It comprises three parts: the Henley layer, the Huxley layer, and the cuticle, with the cuticle being the innermost portion that touches the hair shaft. The IRS cuticle layer is a single cell thick and is located adjacent to the hair fiber. It closely interdigitates with the hair fiber cuticle layer. The Huxley layer can comprise up to four cell layers. The IRS Henley layer is the single cell layer that runs adjacent to the ORS layer (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

G-Protein Coupled Receptors and Signaling

G-Protein Coupled Receptors (GPCRs) consist of seven conserved membrane-spanning domains connecting at least eight divergent hydrophilic loops. The seven hydrophobic stretches, designated as TM1, TM2, TM3, TM4, TM5, TM6, and TM7, comprise about 20 to 30 amino acids each. GPCRs are also known as seven transmembrane, 7TM, receptors. Most GPCRs have single conserved cysteine residues in each of the first two extracellular loops, which form disulfide bonds that are believed to stabilize functional protein structure. Phosphorylation and lipidation (for example, palmitylation or farnesylation) of cysteine residues can affect signal transduction of some GPCRs. A GPCR can contain potential phosphorylation sites within the third cytoplasmic loop and/or its carboxy terminus. For example, phosphorylation induces receptor desensitization in various GPCRs, such as the beta-adrenergic receptor, the phosphorylation event is mediated by protein kinase A and/or specific receptor kinases (Oh et al (2006) Int Rev Cytol. 252:163-218; Kristiansen (2004) Pharmacol Ther. 103(1):21-80)).

The ligand binding sites of some GPCRs are believed to consist of hydrophilic pockets that are formed by several GPCR transmembrane domains. The hydrophilic pockets are surrounded by hydrophobic residues, wherein the hydrophilic side of each GPCR TM helix may face inward and form a polar ligand binding site. In several GPCRs, TM3, which is implicated in signal transduction, contains an aspartate residue that may act as a ligand binding site. TM5 serines, a TM6 asparagine, and TM6 or TM7 phenylalanines or tyrosines also are implicated in ligand binding (Oh et al (2006) Int Rev Cytol. 252:163-218; Kristiansen (2004) Pharmacol Ther. 103(1):21-80).

GPCRs are coupled by intracellular heterotrimeric G-proteins to various ion channels, intracellular enzymes, and transporters. Different G-protein alpha-subunits (for example, Gs, Gi, Gq, G_{12/13}) preferentially stimulate effectors to regulate various cellular biological functions via linking them to a secondary messenger pathway. The heterotrimeric G-protein consists of three subunits, an alpha-subunit that binds and hydrolyses GTP, and a βγ-subunit, which forms a dimer. When GDP is bound to the heterotrimer, the alpha-subunit (Gα) is associated with the βγ-subunit, forming an inactive heterotrimer that binds to the GPCR. When a ligand binds to the receptor and is subsequently activated, a signal is transduced due to a change in protein conformation, resulting in activation of the heterotrimeric G-protein, wherein GDP is exchanged for GTP on the alpha-subunit. Gα then dissociates from the GPCR and the γγ-dimer, and activates downstream secondary messengers (such as adenylyl cyclase, phospholipase C, etc.). Hydrolysis of GTP to GDP, catalyzed by Gα itself, returns the G-protein to its GDP-bound, inactive state. See Luttrell ((2006) Methods Mol Biol. 332:3-49) for further G-protein signaling cascades, which is hereby incorporated by reference.

Purinergic Receptors

Purinergic receptors comprise a family of receptors that are activated by purine-containing compounds such as the nucleotides ATP and UTP and adenosine. The members of the family include the P1 receptors and the P2 receptors. The P2 family is further broken down into 2 subgroups: the P2X receptors (a family of cation-permeable ligand gated ion channels that open in response to extracellular ATP) and the P2Y receptors (which are GPCRs). The P1 receptors bind adenosine while the P2 receptors recognize primarily ATP, ADP, UTP, and UDP. P1 receptors couple to G proteins and have been further subdivided into four subtypes: A1, A2A, A2B, and A3. There are 7 subtypes of the P2X receptor (P2X₁₋₇) and eleven mammalian P2Y receptors that have been identified to date (P2Y1-11).). Very low concentrations of ATP activate the two subtypes (P2X and P2Y) of purinergic receptors (0.1-10 .mu.M) (Ralevic and Burnstock, 1998; Schwiebert and Kishore, 2001). The following reviews further discuss the purinergic receptors: Volonte et al., (2006) Pharma Therap. 112:264-80; Erb et al., (2006) Eur J Physiol. 452:552-62; Burnstock, G., (2004) Curr Top Med Chem. 4:793-803; Adrian et al., (2000) Biochem Biophys Acta 1492: 127-138; Schweibert and Zsembery, (2003) Biochem Biophys Acta 1615: 7-32; Bucheimer and Linden (2003), J Physiol 552(2): 311-321; Burnstock, G., (2006) Pharma Rev. 58(1):58-86, all of which are hereby incorporated by reference in their entirety.

P2X receptors are ATP-gated ion channels that are made up of three protein subunits. These receptors mediate the rapid (within 10 ms) and selective permeability to cations (for example, Na⁺, K⁺ and Ca²+) (Bean, 1992; Dubyak and el-Moatassim, 1993; North, 1996). They can be found on excitable cells (such as smooth muscle cells, neurons, and glial cells). P2X receptors mediate fast excitatory neurotransmission to ATP in both the central and peripheral nervous systems, which contrasts with the slower response (less than 100 ms) to ATP acting at P2Y receptors due to triggering G proteins and their associated secondary messenger systems (Volonte et al., (2006) Pharma Therap. 112:264-80; Erb et al., (2006) Eur J Physiol. 452:552-62).

P2Y receptors are purine and pyrimidine nucleotide receptors that are coupled to G proteins, and thus have broad natural ligand specificity, recognizing ATP, ADP, UTP, UDP, and the diadenosine polyphosphates. ATP is the ligand for P2Y2 and P2Y11; P2Y2 and P2Y4 bind ADP and UTP, P2Y6 binds UDP. P2Y receptors are about 308 to 377 amino acid proteins with a molecular weight of about 41 to 53 kDa, once glycosylated. The tertiary structure of P2Y receptors resembles that of other 7TD GPCRs. In the human P2Y receptor, the most important residues for ATP binding are found to TMDs 3 and 7 on the exofacial side of the receptor (Jiang et al., 1997).

Most P2Y receptors act by coupling to its G protein and subsequently activating PLC. This leads to the formation of IP₃ and intracellular Ca²⁺ mobilization. It has been reported that some P2Y receptors can activate adenylate cyclase. The response time of P2Y receptors is longer than those rapid responses mediated by P2X receptors due to secondary messenger systems and/or ionic conductances that are mediated by G protein coupling to the P2Y receptor. For example, the P2Y1, P2Y2, P2Y6, and P2Y14 receptors are coupled to Gq/11, while P2Y4 can associate with Gi and Gq/11. P2Y11 couples to the G-proteins Gs and Gq/11, and the receptors P2Y12 and P2Y13 associate with Gi. Some members of the P2Y family activate phospholipase C (for example, P2Y1 and P2Y2 receptors). (Volonte et al., (2006) Pharma Therap. 112:264-80; Erb et al., (2006) Eur J Physiol. 452:552-62; Burnstock, G., (2004) Curr Top Med Chem. 4:793-803; Adrian et al., (2000) Biochem Biophys Acta 1492: 127-138; Schweibert and Zsembery, (2003) Biochem Biophys Acta 1615: 7-32; Bucheimer and Linden (2003), J Physiol 552(2): 311-321; and Burnstock, G., (2006) Pharma Rev. 58(1):58-86).

P2RY5

P2RY5 is a purinergic receptor of the GPCR type, characterized by an extracellular N terminus, 7 transmembrane regions, and an intracellular C terminus. The P2RY5 gene resides within intron 17 of the retinoblastoma susceptibility gene (Herzog et al., (1996) Gen Res 6: 858-61). It is also referred to as the P2Y5 receptor or the 6H1 orphan receptor in the literature (Webb et al., (1996) Biochem Biophys Res Com 219: 105-110; Li et al., (1997) Biochem Biophys Res Com 236: 455-460). P2RY5 is an orphan receptor without a known ligand, comprising approximately 344 amino acids and having a molecular weight of about 29 kDa (See Example 2). Responsiveness of P2RY5 to specific nucleotides has not yet been conclusively demonstrated (Webb et al., (1996) Biochem Biophys Res Com 219: 105-110; Li et al., (1997) Biochem Biophys Res Com 236: 455-460).

As used herein, a “P2RY5 molecule” refers to a P2RY5 protein that includes a polypeptide that exhibits a 7 transmembrane (TM) GPCR topology. For example, a P2RY5 molecule can be the human P2RY5 protein (e.g., having the amino acid sequence shown in SEQ ID NO: 1). The P2RY5 molecule can be encoded by a nucleic acid (including genomic DNA, complementary DNA (cDNA), synthetic DNA, as well as any form of corresponding RNA). For example, a P2RY5 molecule can be encoded by a recombinant nucleic acid encoding human P2RY5 protein. The P2RY5 molecules of the invention can be obtained from various sources and can be produced according to various techniques known in the art. For example, a nucleic acid that encodes a P2RY5 molecule can be obtained by screening DNA libraries, or by amplification from a natural source. The P2RY5 molecules of the invention can be produced via recombinant DNA technology and such recombinant nucleic acids can be prepared by conventional techniques, including chemical synthesis, genetic engineering, enzymatic techniques, or a combination thereof. A non-limiting example of a P2RY5 molecule is the polypeptide encoded by the nucleic acid having the nucleotide sequence shown in SEQ ID NO: 2.

According to this invention, a P2RY5 molecule encompasses orthologs of human P2RY5 protein. For example, a P2RY5 molecule encompasses the ortholog in mouse, rat, non-human primates, canines, goat, rabbit, porcine, bovine, chickens, feline, and horses. In other words, a P2RY5 molecule can comprise a protein encoded by a nucleic acid sequence homologous to the human nucleic acid, wherein the nucleic acid is found in a different species and wherein that homolog encodes a protein with a GPCR function similar to a P2RY5 protein.

A P2RY5 molecule of this invention also encompasses variants of the human P2RY5 protein. The variants can comprise naturally-occurring variants due to allelic variations between individuals (e.g., polymorphisms), mutated alleles, related to hair growth or texture, or alternative splicing forms. In one embodiment, a P2RY5 molecule is encoded by a nucleic acid variant of the nucleic acid having the sequence shown in SEQ ID NO: 2, wherein the variant has a nucleotide sequence identity to SEQ ID NO:2 of at least about 50%, at least about 60%, at least about 65%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% with SEQ ID NO: 2.

In one embodiment, a P2RY5 molecule comprises a protein or polypeptide encoded by a P2RY5 nucleic acid sequence, such as the sequence shown in SEQ ID NO: 1. In another embodiment, the polypeptide can be modified, such as by glycosylations and/or acetylations and/or chemical reaction or coupling, and can contain one or several non-natural or synthetic amino acids. An example of a P2RY5 molecule is the polypeptide having the amino acid sequence shown in SEQ ID NO: 1. In certain embodiments, the P2RY5 molecule of the invention includes variants of the human P2RY5 protein (having the amino acid sequence shown in SEQ ID NO: 1). Such variants can include those having at least from about 46% to about 50% identity to SEQ ID NO: 1, or having at least from about 50.1% to about 55% identity to SEQ ID NO: 1, or having at least from about 55.1% to about 60% identity to SEQ ID NO: 1, or having from at least about 60.1% to about 65% identity to SEQ ID NO: 1, or having from about 65.1% to about 70% identity to SEQ ID NO: 1, or having at least from about 70.1% to about 75% identity to SEQ ID NO: 1, or having at least from about 75.1% to about 80% identity to SEQ ID NO: 1, or having at least from about 80.1% to about 85% identity to SEQ ID NO: 1, or having at least from about 85.1% to about 90% identity to SEQ ID NO: 1, or having at least from about 90.1% to about 95% identity to SEQ ID NO: 1, or having at least from about 95.1% to about 97% identity to SEQ ID NO: 1, or having at least from about 97.1% to about 99% identity to SEQ ID NO: 1.

The polypeptide sequence of human P2RY5 is depicted in SEQ ID NO:1. The nucleotide sequence of the human P2RY5 receptor is shown in SEQ ID NO: 2. Sequence information related to P2RY5 is accessible in public databases by GenBank Accession number BC070295.

SEQ ID NO: 1 is the human wild type amino acid sequence corresponding to the P2RY5 receptor (residues 1-344):

MVSVNSSHCFYNDSFKYTLYGCMFSMVFVLGLISNCVAIYIFICVLKVRNETTTYMINLAM
SDLLFVFTLPFRIFYFTTRNWPFGDLLCKISVMLFYTNMYGSILFLTCISVDRFLAIVYPFKS
KTLRTKRNAKIVCTGVWLTVIGGSAPAVFVQSTHSQGNNASEACFENFPEATWKTYLSRIV
IFIEIVGFFIPLILNVTCSSMVLKTLTKPVTLSRSKINKTKVLKMIFVHLIIFCFCFVPYNINLI
LYSLVRTQTFVNCSVVAAVRTMYPITLCIAVSNCCFDPIVYYFTSDTIQNSIKMKNWSVRRS
DFRFSEVHGAENFIQHNLQTLKSKIFDNESAA

The underlined amino acid sequences above in SEQ ID NO: 1 refer to predicted TMD regions of the P2RY5 polypeptide molecule.

SEQ ID NO: 2 is the human wild type nucleotide sequence corresponding to the P2RY5 receptor (nucleotides 1-1821), wherein the underscored ATG denotes the beginning of the open reading frame (ORF):

gacaaattgg gaatgtttaa gtctctgaaa ctctgcactg aaaagaaaat aagattgata
acttaagctt aacattctga ggcataaaga aacattaact ttggagtatt catcttgact
actgaaatac aagtttagaa gacaagtggt ttcattctgg tcacagatca cagcttttct
ttaaatttat aatcctatgg gttggactcg ttgactgtat tttttaaagg ttgctcgtca
gttaactgag ccttggaatt catggatttt ctaaagacta acaaatgaaa atattttcct
gttgaagaac ccagcggaaa ttttacagca acaaatttca tgtttctttt gggtatttct
gagaaaaagg aaatatttat aaaaccatcc aaagatccag ataatttgca aataaattgg
aggttataga ggttataatc tgaatcccaa aggagactgc agctgatgaa agtgcttcca
aactgaaaat tggacgtgcc tttacgatgg taagcgttaa cagctcccac tgcttctata
atgactcctt taagtacact ttgtatgggt gcatgttcag catggtgttt gtgcttgggt
taatatccaa ttgtgttgcc atatacattt tcatctgcgt cctcaaagtc cgaaatgaaa
ctacaactta catgattaac ttggcaatgt cagacttgct ttttgttttt actttaccct
tcaggatttt ttacttcaca acacggaatt ggccatttgg agatttactt tgtaagattt
ctgtgatgct gttttatacc aacatgtacg gaagcattct gttcttaacc tgtattagtg
tagatcgatt tctggcaatt gtctacccat ttaagtcaaa gactctaaga accaaaagaa
atgcaaagat tgtttgcact ggcgtgtggt taactgtgat cggaggaagt gcacccgccg
tttttgttca gtctacccac tctcagggta acaatgcctc agaagcctgc tttgaaaatt
ttccagaagc cacatggaaa acatatctct caaggattgt aattttcatc gaaatagtgg
gattttttat tcctctaatt ttaaatgtaa cttgttctag tatggtgcta aaaactttaa
ccaaacctgt tacattaagt agaagcaaaa taaacaaaac taaggtttta aaaatgattt
ttgtacattt gatcatattc tgtttctgtt ttgttcctta caatatcaat cttattttat
attctcttgt gagaacacaa acatttgtta attgctcagt agtggcagca.gtaaggataa
tgtacccaat cactctctgt attgctgttt ccaactgttg ttttgaccct atagtttact
actttacatc ggacacaatt cagaattcaa taaaaatgaa aaactggtct gtcaggagaa
gtgacttcag attctctgaa gttcatggtg cagagaattt tattcagcat aacctacaga
ccttaaaaag taagatattt gacaatgaat ctgctgcctg aaataaaacc attaggactc
actgggacag aactttcaag ttccttcaac tgtgaaaagt gtctttttgg acaaactatt
tttccacctc caaaagaaat taacacatgg acattttaaa gtctttagta taaagaaaat
ttgtattcaa tgtgttaagc attaacatgt attttatttg tgtatccact ccatctgatt
tttctgagcc attttgattt gttccttcat taaaaaaaat ctcttaaagc taaaaaaaaa
aaaaaaaaaa aaaaaaaaaa a

The mouse polypeptide sequence of P2RY5 is depicted in SEQ ID NO: 9 The mouse nucleotide sequence of the P2RY5 receptor is shown in SEQ ID NO: 10. (accessible in public databases by GenBank accession number NM_{—}175116)

SEQ ID NO: 9 is the mouse wild type amino acid sequence corresponding to the P2RY5 receptor (residues 1-344):

MVSSNGSQCPYDDSFKYTLYGCMFSMVFVLGLISNCVAIYIFICALKVRNETTTYMIN
LAMSDLLFVFTLPFRIFYFATRNWPFGDLLCKISVMLFYTNMYGSILFLTCISVDRFLA
IVYPFKSKTLRTKRNAKIVCIAVWFTVMGGSAPAVFFQSTHSQGNNTSEACFENFPA
ATWKTYLSRIVIFIEIVGFFIPLILNVICSSMVLRTLNKPVTLSRSKMNKTKVLKMIFV
HLVIFCFCFVPYNINLILYSLMRTQTFVNCSVVAAVRTMYPITLCIAVSNCCFDPIVYY
FTSDTIQNSIKMKNWSVRRSDSRFSEVQGTENFIQHNLQTLKNKIFDNESAI

SEQ ID NO: 10 is the mouse wild type nucleotide sequence corresponding to the P2RY5 receptor (nucleotides 1-2447):

aaaggaactg caaacaactg ggttgaagcc ttcctttgtc caaacccagc ctcttcttcc
tgtgatgtca tattacaaat ctgggaaggc tttcttgctc acttcagaga cagcccatct
cacaatacag ctggcaacct ccgaaaggcc tctccattca gcaagcgcga acatgcttag
gaatttatct gggatccctt aaacgactgc ctatcgccgt ccggaatcaa tgtagaaata
caaagtttga gaataaaaag aaggaagaag tacccgagga cgacgggcgg acggacgcac
ggcgagtgtt tgtgactgaa gtaaagctgg tttggaccct ggcggctgaa gcacaagttt
ccacgcggac tggtctggtc cgacttggaa cagtttttcc ttacactttc agctttatgg
gttggcttcc ttgactgcat tttctgtcag ttaactaaac tccagactca tggattttct
cgaccagaaa atcagactat tttcctgaat aatctactag aaacttttac ggaacacatt
tcatgtttcc tttgaagagt taagagaaga aagtatttgt aagaacagga aaagaaacaa
atactttgca aataaactgg ctgctgctgt gaccacatct gaatagcaaa ggcgatcgat
caagcgctgc ggacaaaagg cctcctgtaa gctgcactgc ctgacaatgg taagctccaa
tggctcccag tgcccttatg acgactcctt taagtacact ctgtacgggt gcatgttcag
catggtcttc gtgcttgggc tgatatccaa ctgtgttgcg atatacattt tcatctgtgc
cctcaaagtg agaaatgaaa ctacaacgta catgattaac ctggcaatgt cagatttact
tttcgtcttt acttatctct ttcggatttt ttactttgca acacggaatt ggccatttgg
agatctactc tgtaagattt cagtaatgct gttttacacc aatatgtatg gaagcattct
gttcttaacc tgtatcagtg tagatcgatt tctggcaatt gtctacccat ttaagtcaaa
gactttaaga acgaaacgaa atgcaaagat cgtttgcatt gctgtgtggt tcacagtgat
gggaggaagt gcgcctgcag ttttctttca gtcgacccac tctcagggga acaatacctc
agaagcctgc tttgagaact ttccagcggc cacatggaaa acttatctct ccaggattgt
gattttcatt gaaatagtgg gcttttttat ccctctcatt ttgaacgtaa cttgttctag
tatggtgcta agaactttaa ataaacctgt tacattaagt agaagcaaaa tgaacaaaac
taaggtttta aaaatgattt ttgtccactt ggtcatcttc tgtttctgtt ttgtgcccta
caacatcaac ctcattttgt actcgctcat gaggacacag acctttgtta actgctctgt
ggtggcggca gtgaggacca tgtacccgat cactctctgc atcgctgttt ccaactgctg
ctttgaccct attgtttact acttcacctc agacacaatt cagaactcaa taaaaatgaa
aaactggtcg gttagaagaa gtgactccag gttctctgaa gttcagggca ctgagaattt
tatccaacac aacctacaga ccttaaaaaa taagatattt gataatgaat ctgcaatata
agctgcctga ctaagccact gggactgctc cgtgttcaac tgtgaaaact gtgttcttgg
gaactatctc tccggctcca acaaaaaata tttttaaagg aagtatttgt ctgatgtgtt
aaacattaaa atatattcta ttcttgtatg cacgccattt tactttcttg aaccacttta
acgtgttttt tcctcattaa aaaaaaaaac tccataaagt taaggtctaa aagcaattat
gatttaaata atgtgatata tctgtacgtt taaaattttg tatatcatga aatgatttaa
tcagaaatct gttaaatggt tttatactga accgagatgt tgtttacgga attgtttact
aagtagattt cacttatatt ttattttaga ttaaaagtac ttatcatgcc ttaaactgta
aacagtgaag aaaaccagtt tgaactagtc acacagtttt agagcatttc ctccaagaga
ctactgcagt tttctgcatg acattttatc tttttaggca actcataaaa aagaaaaggt
atctaactat atagtatatt ttgaaattac tattttgatt tatattctgg ctgcaacacc
caatttcaaa atgatatata gtataaaaac ttagcataaa gaaactaatg aaatgttatc
ttgctaggta aattattttg aaagtacaat aaaaaccttg tattttc

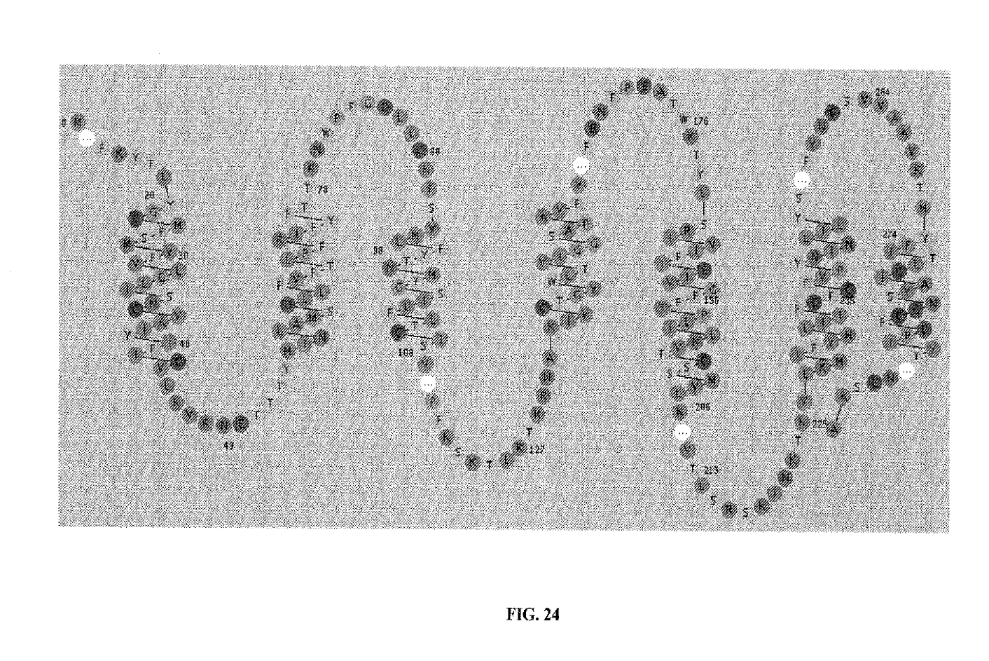

Transmembrane domain (TMD) I of the P2RY5 receptor comprises amino acid residues from about position 20 to about position 42 of SEQ ID NO: 1, whereas TMD II comprises amino acid residues from about position 55 to about position 77 of SEQ ID NO: 1. TMD III of P2RY5 comprises amino acid residues from about position 100 to about position 122 of SEQ ID NO: 1 and TMD IV comprises amino acid residues from about position 135 to about position 154 of SEQ ID NO: 1. TMD V of P2RY5 comprises amino acid residues from about position 179 to about position 201 of SEQ ID NO: 1; TMD VI comprises amino acid residues from about position 230 to about position 252 of SEQ ID NO: 1; and TMD VII comprises amino acid residues from about position 272 to about position 294 of SEQ ID NO 1. (see FIG. 24).

Mutations that affect hair growth regulation and texture have been localized to the TMDs of P2RY5 (see EXAMPLE 2), which is described in Table 1 below.

TABLE 1
Summary of TMD mutations in P2RY5 gene and associated phenotypes.

| | | | | Phenotype | | | |
|---|---|---|---|---|---|---|---|
| | | | | Scalp hair | Facial hair | Body hair | |
| | Mutation | | | (woolly hair, | (eyebrow, | (extremities, | |
| | localized | | Mutation in | sparse hair, | eyelash, | trunk, axilla, | |
| family | to TMD | Origin | the P2RY5 | fragile hair, etc.) | beard hair) | genital) | FIG. |
| HYP2 | I | Pakistan | 69insCATG | woolly | | | 40 |
| HYP7 | I | Pakistan | (homo) | sparse, but | | | 41 |
| | | | | not so | | | |
| | | | | woolly | | | |
| HYP38 | I | Pakistan | | | | | |
| HYP18 | II | Pakistan | 172delAACT_G | | | | |
| | | | (homo) | | | | |
| HYP15 | II | Pakistan | D63V | sparse, | Beard hair | | |
| | | | (homo) | woolly, | is thin, but | | |
| | | | | and fragile | improving. | | |
| HYP31 | II | Pakistan | | woolly | Eyebrow | | 42 |
| | | | | | and eyelash | | |
| | | | | | look sparse | | |
| HYP5 | V | Pakistan | I188F | woolly | | | |
| HYP24 | V | Pakistan | (homo) | sparse, | sparse | No hair | 43 |
| | | | | woolly, | | | |
| | | | | and fragile | | | |
| HYP 42 | V | Pakistan | | | | | |
| HYP 44 | V | Pakistan | | | | | |
| HYP 45 | V | Pakistan | | | | | |
| HYP16 | V | Pakistan | E189K | woolly | | | |
| | | | (homo) | | | | |
| HYP51 | IV | Pakistan | 409T > C; | | | | |
| | | | 410-426del17 | | | | |
| | | | (homo) | | | | |
| HYP60 | VI | Pakistan | Y245C | | | | |
| | | | (homo) | | | | |
| Fam 241 | VII | Brazil | C278Y | No hair | | | |
| | | | (homo) | | | | |
| Fam 333 | IV | Iran | G146R | | | | |
| | | | (homo) | | | | |

For example, the HYP18, HYP38, HYP42, HYP44, HYP45, HYP51, and HYP60 family (from Table 1 above), as well as Family 133 from Iran all showed woolly hair at birth. The hair in the individuals of these families gradually fell out to a greater or lesser degree so that as adults some individuals had no hair, some had sparse hair, and some had nearly normal density woolly hair.

The invention provides for isolated mutants of the human P2RY5 receptor. In one embodiment, the P2RY5 molecule can comprise at least 1 amino acid mutation in transmembrane domain (TMD) I, TMD II, TMD III, TMD IV, TMD V, TMD VI, TMD VII, or a combination of the various P2RY5 TMDs, wherein the P2RY5 molecule comprises a polypeptide having an amino acid sequence of SEQ ID NO: 1. In another embodiment at least 1 amino acid mutation is in TMD I of the P2RY5 receptor, wherein TMD I comprises amino acid residues from about position 20 to about position 42 of SEQ ID NO: 1. In other embodiments, at least 1 amino acid mutation is in TMD II of the P2RY5 receptor, wherein TMD II comprises amino acid residues from about position 55 to about position 77 of SEQ ID NO: 1. In some embodiments, at least 1 amino acid mutation is in TMD III of the P2RY5 receptor, wherein TMD III of P2RY5 comprises amino acid residues from about position 100 to about position 122 of SEQ ID NO: 1. In further embodiments, at least 1 amino acid mutation is in TMD IV of the P2RY5 receptor, wherein TMD IV comprises amino acid residues from about position 135 to about position 154 of SEQ ID NO: 1. In some embodiments, at least 1 amino acid mutation is in TMD V of the P2RY5 receptor, wherein TMD V of P2RY5 comprises amino acid residues from about position 179 to about position 201 of SEQ ID NO: 1. In other embodiments of the invention, at least 1 amino acid mutation is in TMD VI of the P2RY5 receptor, wherein TMD VI comprises amino acid residues from about position 230 to about position 252 of SEQ ID NO: 1. In further embodiments, at least 1 amino acid mutation is in TMD VII of the P2RY5 receptor, wherein TMD VII comprises amino acid residues from about position 272 to about position 294 of SEQ ID NO: 1.

In one embodiment, the amino acid mutation in the P2RY5 receptor can comprise a D>V mutation at amino acid position 63 of SEQ ID NO: 1. This mutation can comprise the amino acid sequence of SEQ ID NO: 3.

SEQ ID NO: 3 is a human P2RY5 receptor amino acid sequence (residue at amino acid position 1 to residue at amino acid position 344) having a D>V substitution mutation at amino acid position 63, which is depicted in BOLD and underlined:

MVSVNSSHCFYNDSFKYTLYGCMFSMVFVLGLISNCVAIYIFICVLKVRNETTTYMIN
LAMSVLLFVFTLPFRIFYFTTRNWPFGDLLCKISVMLFYTNMYGSILFLTCISVDRFLA
IVYPFKSKTLRTKRNAKIVCTGVWLTVIGGSAPAVFVQSTHSQGNNASEACFENFPEA
TWKTYLSRIVIFIEIVGFFIPLILNVTCSSMVLKTLTKPVTLSRSKINKTKVLKMIFVHLI
IFCFCFVPYNINLILYSLVRTQTFVNCSVVAAVRTMYPITLCIAVSNCCFDPIVYYFTSD
TIQNSIKMKNWSVRRSDFRFSEVHGAENFIQHNLQTLKSKIFDNESAA

In a further embodiment, the amino acid mutation in the P2RY5 receptor can comprise an I>F mutation at amino acid position 188 of SEQ ID NO: 1. This mutation can comprise the amino acid sequence of SEQ ID NO: 4.

SEQ ID NO: 4 is a human P2RY5 receptor amino acid sequence (residue at amino acid position 1 to residue at amino acid position 344) having an I>F substitution mutation at amino acid position 188, which is depicted in BOLD and underlined:

MVSVNSSHCFYNDSFKYTLYGCMFSMVFVLGLISNCVAIYIFICVLKVRNETTTYMIN
LAMSDLLFVFTLPFRIFYFTTRNWPFGDLLCKISVMLFYTNMYGSILFLTCISVDRFLA
IVYPFKSKTLRTKRNAKIVCTGVWLTVIGGSAPAVFVQSTHSQGNNASEACFENFPEA
TWKTYLSRIVIFFEIVGFFIPLILNVTCSSMVLKTLTKPVTLSRSKINKTKVLKMIFVHLI
IFCFCFVPYNINLILYSLVRTQTFVNCSVVAAVRTMYPITLCIAVSNCCFDPIVYYFTSD
TIQNSIKMKNWSVRRSDFRFSEVHGAENFIQHNLQTLKSKIFDNESAA

In other embodiments, the amino acid mutation in the human P2RY5 receptor can comprise an E>K mutation at amino acid position 189 of SEQ ID NO: 1. This mutation can comprise the amino acid sequence of SEQ ID NO: 5.

SEQ ID NO: 5 is a human P2RY5 receptor amino acid sequence (residue at amino acid position 1 to residue at amino acid position 344) having an E>K mutation at amino acid position 189, which is depicted in BOLD and underlined:

MVSVNSSHCFYNDSFKYTLYGCMFSMVFVLGLISNCVAIYIFICVLKVRNE
TTTYMINLAMSDLLFVFTLPFRIFYFTTRNWPFGDLLCKISVMLFYTNMYGSILFLTCI
SVDRFLAIVYPFKSKTLRTKRNAKIVCTGVWLTVIGGSAPAVFVQSTHSQGNNASEA
CFENFPEATWKTYLSRIVIFIKIVGFFIPLILNVTCSSMVLKTLTKPVTLSRSKINKTKVL
KMIFVHLIIFCFCFVPYNINLILYSLVRTQTFVNCSVVAAVRTMYPITLCIAVSNCCFDP
IVYYFTSDTIQNSIKMKNWSVRRSDFRFSEVHGAENFIQHNLQTLKSKIFDNESAA

In yet a further embodiment, the amino acid mutation in the human P2RY5 receptor can comprise a C>Y mutation at amino acid position 278 of SEQ ID NO: 1. This mutation can comprise the amino acid sequence of SEQ ID NO: 6.

SEQ ID NO: 6 is a human P2RY5 receptor amino acid sequence (residue at amino acid position 1 to residue at amino acid position 344) having a C>Y mutation at amino acid position 278, which is depicted in BOLD and underlined:

MVSVNSSHCFYNDSFKYTLYGCMFSMVFVLGLISNCVAIYIFICVLKVRNETTTYMIN
LAMSDLLFVFTLPFRIFYFTTRNWPFGDLLCKISVMLFYTNMYGSILFLTCISVDRFLA
IVYPFKSKTLRTKRNAKIVCTGVWLTVIGGSAPAVFVQSTHSQGNNASEACFENFPEA
TWKTYLSRIVIFIEIVGFFIPLILNVTCSSMVLKTLTKPVTLSRSKINKTKVLKMIFVHLI
IFCFCFVPYNINLILYSLVRTQTFVNCSVVAAVRTMYPITLYIAVSNCCFDPIVYYFTSD
TIQNSIKMKNWSVRRSDFRFSEVHGAENFIQHNLQTLKSKIFDNESAA

In another embodiment, the amino acid mutation in the P2RY5 receptor can comprise a Y>C mutation at amino acid position 245 of SEQ ID NO: 1. This mutation can comprise the amino acid sequence of SEQ ID NO: 109.

SEQ ID NO: 109 is a human P2RY5 receptor amino acid sequence (residue at amino acid position 1 to residue at amino acid position 344) having a Y>C mutation at amino acid position 245, which is depicted in BOLD and underlined:

MVSVNSSHCFYNDSFKYTLYGCMFSMVFVLGLISNCVAIYIFICVLKVRNETTTYMIN
LAMSDLLFVFTLPFRIFYFTTRNWPFGDLLCKISVMLFYTNMYGSILFLTCISVDRFLA
IVYPFKSKTLRTKRNAKIVCTGVWLTVIGGSAPAVFVQSTHSQGNNASEACFENFPEA
TWKTYLSRIVIFIEIVGFFIPLILNVTCSSMVLKTLTKPVTLSRSKINKTKVLKMIFVHLI
IFCFCFVPCNINLILYSLVRTQTFVNCSVVAAVRTMYPITLCIAVSNCCFDPIVYYFTSD
TIQNSIKMKNWSVRRSDFRFSEVHGAENFIQHNLQTLKSKIFDNESAA

In another embodiment, the amino acid mutation in the P2RY5 receptor can comprise a G>R mutation at amino acid position 146 of SEQ ID NO: 1: This mutation can comprise the amino acid sequence of SEQ ID NO: 110.

SEQ ID NO: 110 is a human P2RY5 receptor amino acid sequence (residue at amino acid position 1 to residue at amino acid position 344) having a G>R mutation at amino acid position 146, which is depicted in BOLD and underlined:

MVSVNSSHCFYNDSFKYTLYGCMFSMVFVLGLISNCVAIYIFICVLKVRNETTTYMIN
LAMSDLLFVFTLPFRIFYFTTRNWPFGDLLCKISVMLFYTNMYGSILFLTCISVDRFLA
IVYPFKSKTLRTKRNAKIVCTGVWLTVIRGSAPAVFVQSTHSQGNNASEACFENFPEA
TWKTYLSRIVIFIEIVGFFIPLILNVTCSSMVLKTLTKPVTLSRSKINKTKVLKMIFVHLI
IFCFCFVPYNINLILYSLVRTQTFVNCSVVAAVRTMYPITLCIAVSNCCFDPIVYYFTSD
TIQNSIKMKNWSVRRSDFRFSEVHGAENFIQHNLQTLKSKIFDNESAA

The invention also provides for isolated mutants of the human P2RY5 receptor, wherein the isolated mutant human P2RY5 receptor is encoded by a nucleic acid sequence comprising at least about 50%, at least about 60%, at least about 65%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identify with SEQ ID NO: 2.

In one embodiment, the nucleic acid sequence encoding a mutant human P2RY5 receptor can comprise the nucleic acid sequence of SEQ ID NO: 7.

SEQ ID NO: 7 is a human P2RY5 nucleic acid sequence (nucleotide base at position 1 to nucleotide base at position 1042) having a CATG insertion mutation starting at nucleotide base at position 70, which is depicted in BOLD and underlined:

ATGGTAAGCGTTAACAGCTCCCACTGCTTCTATAATGACTCCTTTAAGT
ACACTTTGTATGGGTGCATGCATGTTCAGCATGGTGTTTGTGCTTGGGT
TAATATCCAATTGTGTTGCCATATACATTTTCATCTGCGTCCTCAAAGT
CCGAAATGAAACTACAACTTACATGATTAACTTGGCAATGTCAGACTTG
CTTTTTGTTTTTACTTTACCCTTCAGGATTTTTTACTTCACAACACGGA
ATTGGCCATTTGGAGATTTACTTTGTAAGATTTCTGTGATGCTGTTTTA
TACCAACATGTACGGAAGCATTCTGTTCTTAACCTGTATTAGTGTAGAT
CGATTTCTGGCAATTGTCTACCCATTTAAGTCAAAGACTCTAAGAACCA
AAAGAAATGCAAAGATTGTTTGCACTGGCGTGTGGTTAACTGTGATCGG
AGGAAGTGCACCCGCCGTTTTTGTTCAGTCTACCCACTCTCAGGGTAAC
AATGCCTCAGAAGCCTGCTTTGAAAATTTTCCAGAAGCCACATGGAAAA
CATATCTCTCAAGGATTGTAATTTTTCATCGAAATAGTGGGATTTTTTA
TTCCTCTAATTTTAAATGTAACTTGTTCTAGTATGGTGCTAAAAACTTT
AACCAAACCTGTTACATTAAGTAGAAGCAAAATAAACAAAACTAAGGTT
TTAAAAATGATTTTTGTACATTTGATCATATTCTGTTTCTGTTTTGTTC
CTTACAATATCAATCTTATTTTATATTCTCTTGTGAGAACACAAACATT
TGTTAATTGCTCAGTAGTGGCAGCAGTAAGGACAATGTACCCAATCACT
CTCTGTATTGCTGTTTCCAACTGTTGTTTTGACCCTATAGTTTACTACT
TTACATCGGACACAATTCAGAATTCAATAAAAATGAAAAACTGGTCTGT
CAGGAGAAGTGACTTCAGATTCTCTGAAGTTCATGGTGCAGAGAATTTT
ATTCAGCATAACCTACAGACCTTAAAAAGTAAGATATTTGACAATGAAT
CTGCTGCCTGA

In another embodiment, the nucleic acid sequence encoding a mutant human P2RY5 receptor can comprise the nucleic acid sequence of SEQ ID NO: 8.

SEQ ID NO: 8 is a human P2RY5 nucleic acid sequence (nucleotide base at position 1 to nucleotide base at position 1042) having an AACT_G deletion mutation starting at nucleotide base at position 172 from the ATG start sequence of SEQ ID NO:2:

ATGGTAAGCGTTAACAGCTCCCACTGCTTCTATAATGACTCCTTTAAGT
ACACTTTGTATGGGTGCATGTTCAGCATGGTGTTTGTGCTTGGGTTAAT
ATCCAATTGTGTTGCCATATACATTTTCATCTGCGTCCTCAAAGTCCGA
AATGAAACTACAACTTACATGATTTGCAATGTCAGACTTGCTTTTTGTT
TTTACTTTACCCTTCAGGATTTTTTACTTCACAACACGGAATTGGCCAT
TTGGAGATTTACMGTAAGATTTCTGTGATGCTGTTTTATACCAACATGT
ACGGAAGCATTCTGTTCTTAACCTGTATTAGTGTAGATCGATTTCTGGC
AATTGTCTACCCATTTAAGTCAAAGACTCTAAGAACCAAAAGAAATGCA
AAGATTGTTTGCACTGGCGTGTGGTTAACTGTGATCGGAGGAAGTGCAC
CCGCCGTTTTTGTTCAGTCTACCCACTCTCAGGGTAACAATGCCTCAGA
AGCCTGCTTTGAAAATTTTCCAGAAGCCACATGGAAAACATATCTCTCA
AGGATTGTAATTTTCATCGAAATAGTGGGATTTTTTATTCCTCTAATTT
TAAATGTAACTTGTTCTAGTATGGTGCTAAAAACTTTAACCAAACCTGT
TACATTAAGTAGAAGCAAAATAAACAAAACTAAGGTTTTAAAAATGATT
TTTGTACATTTGATCATATTCTGTTTCTGTTTTGTTCCTTACAATATCA
ATCTTATTTTATATTCTCTTGTGAGAACACAAACATTTGTTAATTGCTC
AGTAGTGGCAGCAGTAAGGACAATGTACCCAATCACTCTCTGTATTGCT
GTTTCCAACTGTTGTTTTGACCCTATAGTTTACTACTTTACATCGGACA
CAATTCAGAATTCAATAAAAATGAAAAACTGGTCTGTCAGGAGAAGTGA
CTTCAGATTCTCTGAAGTTCATGGTGCAGAGAATTTTATTCAGCATAAC
CTACAGACCTTAAAAAGTAAGATATTTGACAATGAATCTGCTGCCTGA

In a further embodiment, the nucleic acid sequence encoding a mutant human P2RY5 receptor can comprise the nucleic acid sequence of SEQ ID NO: 111.

SEQ ID NO: 111 is a human P2RY5 nucleic acid sequence (nucleotide base at position 1 to nucleotide base at position 1018) having a T>C substitution at nucleotide base 409 (highlighted, underlined, and bold), and a 17 nucleic acid base deletion mutation starting at nucleotide base at position 410 to nucleotide base at position 426 from the ATG start sequence of SEQ ID NO:2:

ATGGTAAGCGTTAACAGCTCCCACTGCTTCTATAATGACTCCTTTAAGT
ACACTTTGTATGGGTGCATGTTCAGCATGGTGTTTGTGCTTGGGTTAAT
ATCCAATTGTGTTGCCATATACATTTTCATCTGCGTCCTCAAAGTCCGA
AATGAAACTACAACTTACATGATTAACTTGGCAATGTCAGACTTGCTTT
TTGTTTTTACTTTACCCTTCAGGATTTTTTACTTCACAACACGGAATTG
GCCATTTGGAGATTTACTTTGTAAGATTTCTGTGATGCTGTTTTATACC
AACATGTACGGAAGCATTCTGTTCTTAACCTGTATTAGTGTAGATCGAT
TTCTGGCAATTGTCTACCCATTTAAGTCAAAGACTCTAAGAACCAAAAG
AAATGCAAAGATTGTT [symbol] ACTGTGATCGGAGGAAGTGCACCCGCCGTT
TTTGTTCAGTCTACCCACTCTCAGGGTAACAATGCCTCAGAAGCCTGCT
TTGAAAATTTTCCAGAAGCCACATGGAAAACATATCTCTCAAGGATTGT
AATTTTCATCGAAATAGTGGGATTTTTTATTCCTCTAATTTTAAATGTA
ACTTGTTCTAGTATGGTGCTAAAAACTTTAACCAAACCTGTTACATTAA
GTAGAAGCAAAATAAACAAAACTAAGGTTTTAAAAATGATTTTTGTACA
TTTGATCATATTCTGTTTCTGTTTTGTTCCTTACAATATCAATCTTATT
TTATATTCTCTTGTGAGAACACAAACATTTGTTAATTGCTCAGTAGTGG
CAGCAGTAAGGACAATGTACCCAATCACTCTCTGTATTGCTGTTTCCAA
CTGTTGTTTTGACCCTATAGTTTACTACTTTACATCGGACACAATTCAG
AATTCAATAAAAATGAAAAACTGGTCTGTCAGGAGAAGTGACTTCAGAT
TCTCTGAAGTTCATGGTGCAGAGAATTTTATTCAGCATAACCTACAGAC
CTTAAAAAGTAAGATATTTGACAATGAATCTGCTGCCTGA

Substitution, insertion, and deletion mutants of the P2RY5 nucleic acid sequence or amino acid sequence can be generated as discussed below.

DNA and Amino Acid Manipulation Methods and Purification Thereof

The present invention utilizes conventional molecular biology, microbiology, and recombinant DNA techniques available to one of ordinary skill in the art. Such techniques are well known to the skilled worker and are explained fully in the literature. See, e.g., Maniatis, Fritsch & Sambrook, “Molecular Cloning: A Laboratory Manual” (1982): “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRI. Press, 1986): B. Perbal, “A Practical Guide to Molecular Cloning” (1984), and Sambrook, et al., “Molecular Cloning: a Laboratory Manual” (1989).

One skilled in the art can obtain a P2RY5 protein or a variant thereof, in several ways, which include, but are not limited to, isolating the protein via biochemical means or expressing a nucleotide sequence encoding the protein of interest by genetic engineering methods.

The invention provides for a nucleic acid encoding a P2RY5 molecule or variants thereof. In one embodiment, the nucleic acid is expressed in an expression cassette, for example, to achieve overexpression in a cell. The nucleic acids of the invention can be an RNA, cDNA, cDNA-like, or a DNA of interest in an expressible format, such as an expression cassette, which can be expressed from the natural promoter or an entirely heterologous promoter. The nucleic acid of interest can encode a protein, and may or may not include introns.

Protein variants can involve amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture.

Techniques for making substitution mutations at predetermined sites in DNA having a known sequence are well known, for example M13 primer mutagenesis and PCR mutagenesis. Amino acid substitutions can be single residues, but can occur at a number of different locations at once. In one non-limiting embodiment, insertions can be on the order of about from 1 to about 10 amino acid residues, while deletions can range from about 1 to about 30 residues. Deletions or insertions can be made in adjacent pairs (for example, a deletion of about 2 residues or insertion of about 2 residues). Substitutions, deletions, insertions, or any combination thereof can be combined to arrive at a final construct. The mutations cannot place the sequence out of reading frame and should not create complementary regions that can produce secondary mRNA structure. Substitutional variants are those in which at least one residue has been removed and a different residue inserted in its place.

In one embodiment, an isolated mutant human P2RY5 polypeptide can contain a D>V mutation at amino acid position 63 of SEQ ID NO: 1. The P2RY5 D>V mutant can comprise the amino acid sequence of SEQ ID NO: 3. In another embodiment, an isolated mutant human P2RY5 polypeptide can contain an I>F mutation at amino acid position 188 of SEQ ID NO: 1. The P2RY5 I>F mutant can comprise the amino acid sequence of SEQ ID NO: 4. In a further embodiment, an isolated mutant human P2RY5 polypeptide can contain an E>K mutation at amino acid position 189 of SEQ ID NO: 1. The P2RY5 E>K mutant can comprise the amino acid sequence of SEQ ID NO: 5. In yet another embodiment of the invention, an isolated mutant human P2RY5 polypeptide can contain a C>Y mutation at amino acid position 278 of SEQ ID NO: 1, The P2RY5 C>Y mutant can comprise the amino acid sequence of SEQ ID NO: 6.

The invention also provides for isolated human P2RY5 polypeptides that contain an insertional or deletional mutations at the nucleic acid level. In one embodiment, an isolated mutant human P2RY5 polypeptide can be encoded by a nucleic acid sequence comprising at least about 50%, at least about 60%, at least about 70%, at least about 75%, at least about 80%, at least about 90%, at least about 95%, or at least about 99% identify to SEQ ID NO: 2. In another embodiment, the isolated human P2RY5 polypeptide is encoded by a nucleotide sequence that comprises the nucleic acid sequence of SEQ ID NO: 7. For example, the nucleic acid sequence of this mutant contains an insertion mutation of 4 nucleotides, CATG, starting at position 70 of SEQ ID NO:2, and comprises SEQ ID NO: 7. In a further embodiment, the isolated human P2RY5 polypeptide is encoded by a nucleotide sequence that comprises the nucleic acid sequence of SEQ ID NO: 8. For example, the nucleic acid sequence of this mutant contains a deletion mutation of 5 nucleotides, AACT_G (wherein “_” designates an unchanged nucleotide), starting at position 172 of SEQ ID NO:2, and comprises SEQ ID NO: 8.

Substantial changes in function or immunological identity are made by selecting selecting residues that differ more significantly in their effect on maintaining (a) the structure of the polypeptide backbone in the area of the substitution, for example as a sheet or helical conformation, (b) the charge or hydrophobicity of the molecule at the target site or (e) the bulk of the side chain. The substitutions that can produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Minor variations in the amino acid sequences of P2RY5 molecules can be encompassed by the present invention, providing that the variations in the amino acid sequence maintain at least about 30%, at least about 40%, at least about 50%, at least about 60%, at least about 70%, at least about 75%, at least about 80%, at least about 90%, at least about 95%, or at least about 99% identify to SEQ ID NO:1. For example, conservative amino acid replacements can be utilized. Conservative replacements are those that take place within a family of amino acids that are related in their side chains, wherein the interchangeability of residues have similar side chains.

Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) a group of amino acids having aliphatic-hydroxyl side chains, such as serine and threonine; (ii) a group of amino acids having amide-containing side chains, such as asparagine and glutamine; (iii) a group of amino acids having aliphatic side chains such as glycine, alanine, valine, leucine, and isoleucine; (iv) a group of amino acids having aromatic side chains, such as phenylalanine, tyrosine, and tryptophan; and (v) a group of amino acids having sulfur-containing side chains, such as cysteine and methionine. Useful conservative amino acids substitution groups are: valine-leucine-isoleucine, phenylalanine-tyrosine, lysine-arginine, alanine valine, glutamic-aspartic, and asparagine-glutamine.

For example, the replacement of one amino acid residue with another that is biologically and/or chemically similar is known to those skilled in the art as a conservative substitution, For example, a conservative substitution would be replacing one hydrophobic residue for another, or one polar residue for another. The substitutions include combinations such as, for example, Gly, Ala; Val, Ile, Leu; Asp, Glu; Asn, Gin; Ser, Thr; Lys, Arg; and Phe, Tyr. Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Bacterial and Yeast Expression Systems

In bacterial systems, a number of expression vectors can be selected. For example, when a large quantity of P2RY5 protein is needed for the induction of antibodies, vectors which direct high level expression of fusion proteins that are readily purified can be used. Non-limiting examples of such vectors include multifunctional E. coli cloning and expression vectors such as BLUESCRIPT (Stratagene). pIN vectors or pGEX vectors (Promega, Madison, Wis.) also can be used to express foreign polypeptide molecules as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

Plant and Insect Expression Systems

If plant expression vectors are used, the expression of sequences encoding a P2RY5 molecule can be driven by any of a number of promoters. For example, viral promoters such as the 35S and 19S promoters of CaMV can be used alone or in combination with the omega leader sequence from TMV. Alternatively, plant promoters such as the small subunit of RUBISCO or heat shock promoters, can be used. These constructs can be introduced into plant cells by direct DNA transformation or by pathogen-mediated transfection.

An insect system also can be used to express P2RY5 molecules. For example, in one such system Autographa californica nuclear polyhedrosis virus (AcNPV) is used as a vector to express foreign genes in Spodoptera frugiperda cells or in Trichoplusia larvae. Sequences encoding a P2RY5 molecule can be cloned into a non-essential region of the virus, such as the polyhedrin gene, and placed under control of the polyhedrin promoter. Successful insertion of P2RY5 nucleic acid sequences will render the polyhedrin gene inactive and produce recombinant virus lacking coat protein. The recombinant viruses can then be used to infect S. frugiperda cells or Trichoplusia larvae in which P2RY5 or a variant thereof can be expressed.

Mammalian Expression Systems

An expression vector can include a nucleotide sequence that encodes a P2RY5 molecule linked to at least one regulatory sequence in a manner allowing expression of the nucleotide sequence in a host cell. A number of viral-based expression systems can be used to express a P2RY5 molecule or a variant thereof in mammalian host cells. For example, if an adenovirus is used as an expression vector, sequences encoding a P2RY5 molecule can be ligated into an adenovirus transcription/translation complex comprising the late promoter and tripartite leader sequence. Insertion into a non-essential E1 or E3 region of the viral genome can be used to obtain a viable virus which is capable of expressing a P2RY5 molecule in infected host cells. Transcription enhancers, such as the Rous sarcoma virus (RSV) enhancer, can also be used to increase expression in mammalian host cells.

Regulatory sequences are well known in the art, and can be selected to direct the expression of a protein or polypeptide of interest (such as a P2RY5 molecule) in an appropriate host cell as described in Goeddel, Gene Expression Technology: Methods in Enzymology 185, Academic Press, San Diego, Calif. (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L, et al., Curr Opin Genet Dev, 2002, 12(2):137-41) enhancers, and other expression control elements.

Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (for example, a P2RY5 molecule) is stably integrated into the genome of eukaryotic cells (for example mammalian cells, such as cells from the end bulb of the hair follicle), resulting in the stable expression of transfected genes. An exogenous nucleic acid sequence can be introduced into a cell (such as a mammalian cell, either a primary or secondary cell) by homologous recombination as disclosed in U.S. Pat. No. 5,641,670, the contents of which are herein incorporated by reference.

A gene that encodes a selectable marker (for example, resistance to antibiotics or drugs, such as ampicillin, neomycin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule (for example, P2RY5).

Cell Transfection

A eukaryotic expression vector can be used to transfect cells in order to produce proteins (for example, a P2RY5 molecule) encoded by nucleotide sequences of the vector. Mammalian cells (such as isolated cells from the hair bulb; for example dermal sheath cells and dermal papilla cells) can contain an expression vector (for example, one that contains a gene encoding P2RY5 molecule) via introducing the expression vector into an appropriate host cell via methods known in the art.

A host cell strain can be chosen for its ability to modulate the expression of the inserted sequences or to process the expressed P2RY5 polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and W138), are available from the American Type Culture Collection (ATCC; 10801 University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextrin-mediated transfection, or electroporation. Electroporation is carried out at approximate voltage and capacitance to result in entry of the DNA construct(s) into cells of interest (such as cells of the end bulb of a hair follicle, for example dermal papilla cells or dermal sheath cells). Other methods used to transfect cells can also include modified calcium phosphate precipitation, polybrene precipitation, liposome fusion, and receptor-mediated gene delivery.

Cells to be genetically engineered can be primary and secondary cells obtained from various tissues, and include cell types which can be maintained and propagated in. culture. Non-limiting examples of primary and secondary cells include epithelial cells (for example, dermal papilla cells), neural cells, endothelial cells, glial cells, fibroblasts, muscle cells (such as myoblasts) keratinocytes, formed elements of the blood (e.g., lymphocytes, bone marrow cells), and precursors of these somatic cell types.

Vertebrate tissue can be obtained by methods known to one skilled in the art, such a punch biopsy or other surgical methods of obtaining a tissue source of the primary cell type of interest. In one embodiment, a punch biopsy or removal can be used to obtain a source of keratinocytes, fibroblasts, endothelial cells, or mesenchymal cells (for example, hair follicle cells or dermal papilla cells). In another embodiment, removal of a hair follicle can be used to obtain a source of fibroblasts, keratinocytes, endothelial cells, or mesenchymal cells (for example, hair follicle cells or dermal papilla cells). A mixture of primary cells can be obtained from the tissue, using methods readily practiced in the art, such as explanting or enzymatic digestion (for examples using enzymes such as pronase, trypsin, collagenase, elastase dispase, and chymotrypsin). Biopsy methods have also been described in United States Patent Application Publication 2004/0057937 and ACT application publication WO 2001/32840, and are hereby incorporated by reference.

Primary cells can be acquired from the individual to whom the genetically engineered primary or secondary cells are administered. However, primary cells can also be obtained from a donor, other than the recipient, of the same species. The cells can also be obtained from another species (for example, rabbit, cat, mouse, rat, sheep, goat, dog, horse, cow, bird, or pig). Primary cells can also include cells from an isolated vertebrate tissue source grown attached to a tissue culture substrate (for example, flask or dish) or grown in a suspension; cells present in an explant derived from tissue; both of the aforementioned cell types plated for the first time; and cell culture suspensions derived from these plated cells. Secondary cells can be plated primary cells that are removed from the culture substrate and replated, or passaged, in addition to cells from the subsequent passages. Secondary cells can be passaged one or more times. These primary or secondary cells can contain expression vectors having a gene that encodes a protein of interest (for example, a P2RY5 molecule).

Cell Culturing

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized. Non-limiting examples of medium include, for example, Minimal Essential Medium (MEM, Sigma, St. Louis, Mo.); Dulbecco's Modified Eagles Medium (DMEM, Sigma); Ham's F10 Medium (Sigma); HyClone cell culture medium (HyClone, Logan, Utah); RPMI-1640 Medium (Sigma); and chemically-defined (CD) media, which are formulated for various cell types, e.g., CD-CHO Medium (Invitrogen, Carlsbad, Calif.).

The cell culture media can be supplemented as necessary with supplementary components or ingredients, including optional components, in appropriate concentrations or amounts, as necessary or desired. Cell culture medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that can be required at very low concentrations, usually in the micromolar range.

The medium also can be supplemented selectively with one or more components from any of the following categories: (1) salts, for example, magnesium, calcium, and phosphate; (2) hormones and other growth factors such as, serum, insulin, transferrin, and epidermal growth factor; (3) protein and tissue hydrolysates, for example peptone or peptone mixtures which can be obtained from purified gelatin, plant material, or animal by products; (4) nucleosides and bases such as, adenosine, thymidine, and hypoxanthine; (5) buffers, such as HEPES; (6) antibiotics, such as gentamycin or ampicillin; (7) cell protective agents, for example pluronic polyol; and (8) galactose. In one embodiment, soluble factors can be added to the culturing medium.

The mammalian cell culture that can be used with the present invention is prepared in a medium suitable for the type of cell being cultured. In one embodiment, the cell culture medium can be any one of those previously discussed (for example, MEM) that is supplemented with serum from a mammalian source (for example, fetal bovine serum (FBS)). In another embodiment, the medium can be a conditioned medium to sustain the growth of epithelial cells or cells obtained from the hair bulb of a hair follicle (such as dermal papilla cells or dermal sheath cells). For example, epithelial cells can be cultured according to Barnes and Mather in Animal Cell Culture Methods (Academic Press, 1998), which is hereby incorporated by reference in its entirety. In a further embodiment, epithelial cells or hair follicle cells can be transfected with DNA vectors containing genes that encode a polypeptide or protein of interest (for example, a P2RY5 molecule). In other embodiments of the invention, cells are grown in a suspension culture (for example, a three-dimensional culture such as a hanging drop culture) in the presence of an effective amount of enzyme, wherein the enzyme substrate is an extracellular matrix molecule in the suspension culture. For example, the enzyme can be a hyaluronidase. Epithelial cells or hair follicle cells can be cultivated according to methods practiced in the art, for example, as those described in PCT application publication WO 2004/044188 and in U.S. Patent Application Publication No. 2005/0272150, or as described by Harris in Handbook in Practical Animal Cell Biology: Epithelial Cell Culture (Cambridge Univ. Press, Great Britain; 1996; see Chapter 8), which are hereby incorporated by reference.

A suspension culture is a type of culture wherein cells, or aggregates of cells (such as aggregates of DP cells), multiply while suspended in liquid medium. A suspension culture comprising mammalian cells can be used for the maintenance of cell types that do not adhere or to enable cells to manifest specific cellular characteristics that are not seen in the adherent form. Some types of suspension cultures can include three-dimensional cultures or a hanging drop culture. A hanging-drop culture is a culture in which the material to be cultivated is inoculated into a drop of fluid attached to a flat surface (such as a coverglass, glass slide, Petri dish, flask, and the like), and can be inverted over a hollow surface. Cells in a hanging drop can aggregate toward the hanging center of a drop as a result of gravity. However, according to the methods of the invention, cells cultured in the presence of a protein that degrades the extracellular matrix (such as collagenase, chondroitinase, hyaluronidase, and the like) will become more compact and aggregated within the hanging drop culture, for degradation of the ECM will allow cells to become closer in proximity to one another since less of the ECM will be present.

Cells obtained from the hair bulb of a hair follicle (such as dermal papilla cells or dermal sheath cells) can be cultured as a single, homogenous population (for example, comprising DP cells) in a hanging drop culture so as to generate an aggregate of DP cells. Cells can also be cultured as a heterogeneous population (for example, comprising DP and DS cells) in a hanging drop culture so as to generate a chimeric aggregate of DP and DS cells. Epithelial cells can be cultured as a monolayer to confluency as practiced in the art. Such culturing methods can be carried out essentially according to methods described in Chapter 8 of the Handbook in Practical Animal Cell Biology: Epithelial Cell Culture (Cambridge Univ. Press, Great Britain; 1996); Underhill CB, J Invest Dermatol, 1993, 101(6):820-6); in Armstrong and Armstrong, (1990) J Cell Biol 110:1439-55; or in Animal Cell Culture Methods (Academic Press, 1998), which are all hereby incorporated by reference in their entireties.

Three-dimensional cultures can be formed from agar (such as Gey's Agar), hydrogels (such as matrigel, agarose, and the like; Lee et al., (2004) Biomaterials 25: 2461-2466) or polymers that are cross-linked. These polymers can comprise natural polymers and their derivatives, synthetic polymers and their derivatives, or a combination thereof. Natural polymers can be anionic polymers, cationic polymers, amphipathic polymers, or neutral polymers. Non-limiting examples of anionic polymers can include hyaluronic acid, alginic acid (alginate), carageenan, chondroitin sulfate, dextran sulfate, and pectin. Some examples of cationic polymers, include but are not limited to, chitosan or polylysine. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73). Examples of amphipathic polymers can include, but are not limited to collagen, gelatin, fibrin, and carboxymethyl chitin. Non-limiting examples of neutral polymers can include dextran, agarose, or pullulan. (Peppas et al., (2006) Adv Mater, 18: 1345-60; Hoffman, A. S., (2002) Adv Drug Deliv Rev. 43: 3-12; Hoffman, A. S., (2001) Ann NY Aced Sci 944: 62-73).

The cells suitable for culturing according to methods of the invention can have introduced expression vectors, such as plasmids. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

Obtaining and Purifying Polypeptides

A P2RY5 polypeptide molecule or a variant thereof, can be obtained by purification from human cells expressing a P2RY5 molecule by in vitro or in vivo expression of a nucleic acid sequence encoding a P2RY5 molecule; or by direct chemical synthesis.

Detecting Polypeptide Expression

Host cells which contain a nucleic acid encoding a P2RY5 molecule, and which subsequently express P2RY5, can be identified by various procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a nucleic acid encoding a P2RY5 molecule can be detected by DNA-DNA or DNA-RNA hybridization or amplification using probes or fragments of nucleic acids encoding a P2RY5 molecule. In one embodiment, a P2RY5 fragment encompasses any portion of at least about 8 consecutive nucleotides of SEQ ID NO: 2. In one embodiment, the fragment can comprise at least about 15 nucleotides, at least about 20 nucleotides, or at least about 30 nucleotides of SEQ ID NO: 2. Fragments include all possible nucleotide lengths between about 8 and 100 nucleotides, for example, lengths between about 15 and 100, or between about 20 and 100. Nucleic acid amplification-based assays involve the use of oligonucleotides selected from sequences encoding a P2RY5 polypeptide to detect transformants which contain a nucleic acid encoding a P2RY5 molecule.

Protocols for detecting and measuring the expression of a P2RY5 polypeptide using either polyclonal or monoclonal antibodies specific for the polypeptide are well established. Non-limiting examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a P2RY5 polypeptide can be used, or a competitive binding assay can be employed.

Labeling and conjugation techniques are known by those skilled in the art and can be used in various nucleic acid and amino acid assays. Methods for producing labeled hybridization or PCR probes for detecting sequences related to nucleic acid sequences encoding P2RY5 include, but are not limited to, oligolabeling, nick translation, end-labeling, or PCR amplification using a labeled nucleotide. Alternatively, nucleic acid sequences encoding a P2RY5 polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, and/or magnetic particles.

Expression and Purification of Polypeptides

Host cells transformed with a nucleic acid sequence encoding a P2RY5 molecule can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding a P2RY5 molecule can be designed to contain signal sequences which direct secretion of soluble P2RY5 polypeptide molecules or a variant thereof, through a prokaryotic or eukaryotic cell membrane or which direct the membrane insertion of membrane-bound P2RY5 polypeptide molecule or a variant thereof.

Other constructions can also be used to join a sequence encoding a P2RY5 polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Including cleavable linker sequences (i.e., those specific for Factor Xa or enterokinase (Invitrogen, San Diego, Calif.)) between the purification domain and a P2RY5 polypeptide also can be used to facilitate purification. One such expression vector provides for expression of a fusion protein containing P2RY5 and 6 histidine residues preceding a thioredoxin or an enterokinase cleavage site. The histidine residues facilitate purification by immobilized metal ion affinity chromatography, while the enterokinase cleavage site provides a means for purifying the P2RY5 polypeptide.

A P2RY5 polypeptide molecule can be purified from any human or non-human cell which expresses the receptor, including those which have been transfected with expression constructs that express a P2RY5 molecule. A purified P2RY5 molecule can be separated from other compounds which normally associate with P2RY5 in the cell, such as certain proteins, carbohydrates, or lipids, using methods practiced in the art. Non-limiting methods include size exclusion chromatography, ammonium sulfate fractionation, ion exchange chromatography, affinity chromatography, and preparative gel electrophoresis.

Chemical Synthesis

Nucleic acid sequences encoding a P2RY5 polypeptide can be synthesized, in whole or in part, using chemical methods known in the art. Alternatively, a P2RY5 molecule can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431 A Peptide Synthesizer (Perkin Elmer). Optionally, fragments of P2RY5 molecules (such as those comprising P2RY5 nucleic acid or amino acid sequences) can be separately synthesized and combined using chemical methods to produce a full-length molecule. In one embodiment, a P2RY5 fragment encompasses any portion of at least about 8 consecutive nucleotides of SEQ ID NO: 2. In one embodiment, the fragment can comprise at least about 15 nucleotides, at least about 20 nucleotides, or at least about 30 nucleotides of SEQ ID NO: 2. Fragments include all possible nucleotide lengths between about 8 and 100 nucleotides, for example, lengths between about 15 and 100, or between about 20 and 100. In another embodiment.

A P2RY5 fragment can also be a fragment of a P2RY5 protein. For example, the P2RY5 fragment can encompass any portion of at least about 8 consecutive amino acids of SEQ ID NO: 1. The fragment can comprise at least about 10 amino acids, a least about 20 amino acids, at least about 30 amino acids, at least about 40 amino acids, a least about 50 amino acids, at least about 60 amino acids, or at least about 75 amino acids of SEQ ID NO: 1. Fragments include all possible amino acid lengths between about 8 and 100 about amino acids, for example, lengths between about 10 and 100 amino acids, between about 15 and 100 amino acids, between about 20 and 100 amino acids, between about 35 and 100 amino acids, between about 40 and 100 amino acids, between about 50 and 100amino acids, between about 70 and 100 amino acids, between about 75 and 100 amino acids, or between about 80 and 100 amino acids.

The newly synthesized peptide can be substantially purified via high performance liquid chromatography (HPLC). The composition of a synthetic P2RY5 molecule can be confirmed by amino acid analysis or sequencing. Additionally, any portion of the amino acid sequence of P2RY5 can be altered during direct synthesis and/or combined using chemical methods with sequences from other proteins to produce a variant polypeptide or a fusion protein.

Identifying P2RY5 Modulating Compounds

The invention provides methods for identifying compounds which can be used for controlling and/or regulating hair growth in a subject. In addition, the invention provides methods for identifying compounds which can be used for the treatment of a hair loss disorder. Non-limiting examples of hair loss disorders include: androgenetic alopecia, Telogen effluvium, Alopecia areata, telogen effluvium, Alopecia areata, Tinea capitis, alopecia totalis, and alopecia universalis. The methods can comprise the identification of test compounds or agents (e.g., peptides (such as antibodies or fragments thereof), small molecules, nucleic acids (such as siRNA or antisense RNA), or other agents) that can bind to a P2RY5 polypeptide molecule and/or have a stimulatory or inhibitory effect on the biological activity of P2RY5 or its expression, and subsequently determining whether these compounds can regulate hair growth in a subject or can have an effect on symptoms associated with the hair loss disorders in an in vivo assay (i.e., examining an increase or reduction in hair growth).

As used herein, a “P2RY5 modulating compound” refers to a compound that interacts with the P2RY5 receptor and modulates its GPCR signaling activity and/or its expression. The compound can either increase P2RY5's activity or expression. Conversely, the compound can decrease P2RY5's activity or expression. The compound can be a P2RY5 agonist or a P2RY5 antagonist. Some non-limiting examples of P2RY5 modulating compounds include peptides (such as P2RY5 peptide fragments, or antibodies or fragments thereof), small molecules, and nucleic acids (such as P2RY5 siRNA or antisense RNA specific for P2RY5 nucleic acid). A P2RY5 modulating compound can be an agonist or antagonist. Agonists of a P2RY5 molecule can be molecules which, when bound to P2RY5 increase or prolong the activity of a P2RY5 molecule. Agonists of P2RY5 include, but are not limited to, proteins, nucleic acids, small molecules, or any other molecule which activates P2RY5. Antagonists of a P2RY5 molecule can be molecules which, when bound to P2RY5 or a variant thereof, decrease the amount or the duration of the activity of a P2RY5 molecule. Antagonists include proteins, nucleic acids, antibodies, small molecules, or any other molecule which decrease the activity of P2RY5.

The term “modulate”, as it appears herein, refers to a change in the activity or expression of a P2RY5 molecule. For example, modulation can cause an increase or a decrease in protein activity, binding characteristics, or any other biological, functional, or immunological properties of a P2RY5 molecule.

In one embodiment, a P2RY5 modulating compound can be a peptide fragment of a P2RY5 protein that binds to the GPCR. For example, the P2RY5 molecule can encompass any portion of at least about 8 consecutive amino acids of SEQ ID NO: 1. The fragment can comprise at least about 10 amino acids, a least about 20 amino acids, at least about 30 amino acids, at least about 40 amino acids, a least about 50 amino acids, at least about 60 amino acids, or at least about 75 amino acids of SEQ ID NO: 1. Fragments include all possible amino acid lengths between and including about 8 and 100 about amino acids, for example, lengths between about 10 and 100 amino acids, between about 15 and 100 amino acids, between about 20 and 100 amino acids, between about 35 and 100 amino acids, between about 40 and 100 amino acids, between about 50 and 100 amino acids, between about 70 and 100 amino acids, between about 75 and 100 amino acids, or between about 80 and 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England). The P2RY5 peptide fragments can be isolated from a natural source, genetically engineered, or chemically prepared. These methods are well known in the art.

A P2RY5 modulating compound can also be a protein, such as an antibody (monoclonal, polyclonal, humanized, and the like), or a binding fragment thereof, directed against the GPCR, P2RY5. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fe, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

Inhibition of RNA encoding a P2RY5 receptor can effectively modulate the expression of the P2RY5 gene from which the RNA is transcribed. Inhibitors are selected from the group comprising: siRNA, interfering RNA or RNAi; dsRNA; RNA Polymerase III transcribed DNAs; ribozymes; and antisense nucleic acid, which can be RNA, DNA, or artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a P2RY5 polypeptide can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59).

siRNA comprises a double stranded structure containing from about 15 to about 50 base pairs, for example from about 21 to about 25 base pairs, and having a nucleotide sequence identical or nearly identical to an expressed target gene or RNA within the cell. Antisense nucleotide sequences include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The P2RY5 modulating compound can contain ribonucleotides, deoxyribonucleotides, synthetic nucleotides, or any suitable combination such that the target RNA and/or gene is inhibited. In addition, these forms of nucleic acid can be single, double, triple, or quadruple stranded. (see for example Bass (2001) Nature, 411, 428 429; Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914).

A P2RY5 modulating compound can also be a small molecule that binds to the P2RY5 receptors and disrupts its function, or conversely, enhances its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate P2RY5 can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described below (Werner et al., (2006) Brief Funct. Genomic Proteomic 5(1):32-6).

In one embodiment, a modulating compound can be of Formula I:

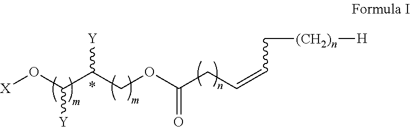

wherein
X is —PO₃R¹ or —SO₃R¹;
R¹ is —H, or a metal cation;
Y is —OR² or —NR²₂,
Y¹ is H, —OR² or —NR²₂;
each R² is independently —H, —C₁-C₆alkyl, —C₃-C₈cycloalkyl, or —C(═O)R³, wherein R³ is —H, —C₁-C₆ alkyl, or —C₃-C₈ cycloalkyl;
each m is independently 0-6;
each n is independently 1-10;
the starred carbon may be chiral, such as of R or S configuration, or may be a mixture of both; and
the double bond may be E or Z, or a mixture of both,
   and pharmaceutically acceptable salts thereof, such as sodium, potassium, lithium, calcium, or magnesium salts.

In one embodiment, X is —PO₃R¹. In another embodiment X is —SO₃R¹.

In one embodiment, R¹ is H. In another embodiment, R¹ is sodium.

In one embodiment, R² is H. In another, embodiment, R² is —C(═O)R³.

In one embodiment, R³ is —C₁-C₆ alkyl.

In one embodiment, m is 1-2. In another embodiment, m is 1.

In one embodiment, n is 1-6. In another embodiment, n is 6-10. In yet another embodiment, n is 6. In still another embodiment, n is 7.

In one embodiment, the starred carbon is in the R configuration. In another embodiment, the starred carbon is in the S configuration.

In one embodiment, the double bond is in the E configuration.

In another embodiment, the double bond is in the Z configuration.

In another embodiment, a modulating compound can be of Formula II:

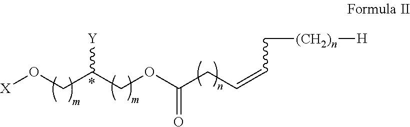

wherein
X is —PO₃R¹ or —SO₃R¹;
R¹ is —H, or a metal cation;
Y is —OR² or —NR²₂, and
each R² is independently —H, —C₁-C₆ alkyl, —C₃-C₈ cycloalkyl, or —C(C═O)R³, wherein R³ is —H, —C₁-C₆ alkyl, or —C₃-C₈ cycloalkyl;
each m is independently 0-6;
each n is independently 1-10;
the starred carbon may be chiral, such as of R or S configuration, or may be a mixture of both; and
the double bond may be E or Z, or a mixture of both,
   and pharmaceutically acceptable salts thereof, such as sodium, potassium, lithium, calcium, or magnesium salts:

In one embodiment, X is —PO₃R¹. In another embodiment X is —SO₃R¹.

In one embodiment, R¹ is H. In another embodiment, R¹ is sodium.

In one embodiment, R² is H. In another embodiment, R² is —C(═O)R³.

In one embodiment, R³ is —C₁-C₆ alkyl.

In one embodiment, m is 1-2. In another embodiment, m is 1.

In one embodiment, n is 1-6. In another embodiment, n is 6-10. In yet another embodiment, n is 6. In still another embodiment, n is 7.

In one embodiment, the starred carbon is in the R configuration. In another embodiment, the starred carbon is in the S configuration.

In one embodiment, the double bond is in the E configuration.

In another embodiment, the double bond is in the Z configuration.

In further embodiments, the molecule can be a compound depicted below:

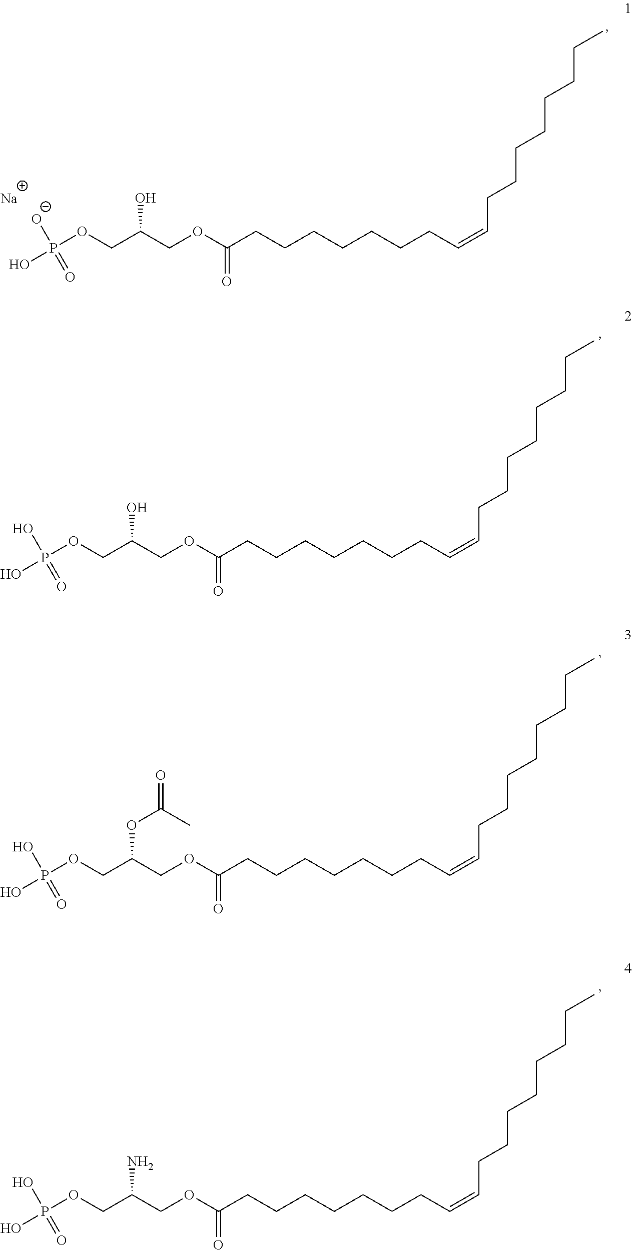 [SMILES: [Na+] ; CCCCCCCC/C=C\CCCCCCCC(=O)OC[C@@H](O)COP(=O)([O-])O ; CCCCCCCC/C=C\CCCCCCCC(=O)OC[C@@H](O)COP(=O)(O)O ; CCCCCCCC/C=C\CCCCCCCC(=O)OC[C@H](COP(=O)(O)O)OC(C)=O ; CCCCCCCC/C=C\CCCCCCCC(=O)OC[C@@H](N)COP(=O)(O)O]

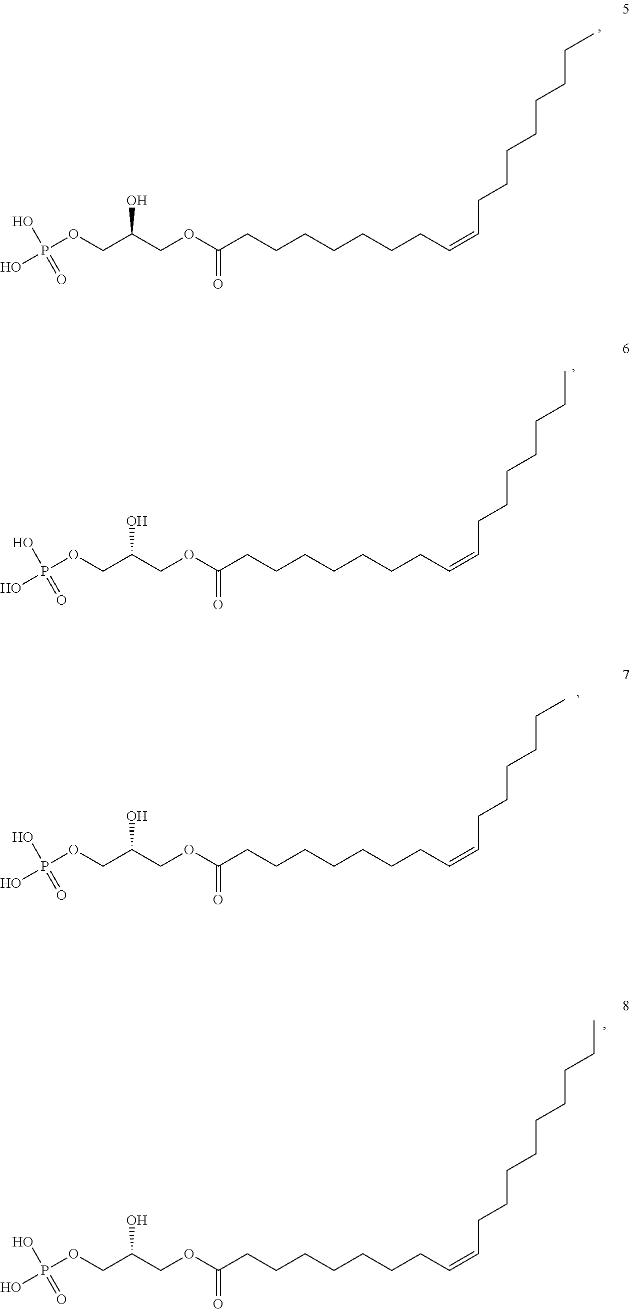 [SMILES: CCCCCCCC/C=C\CCCCCCCC(=O)OC[C@H](O)COP(=O)(O)O ; CCCCCCC/C=C\CCCCCCCC(=O)OC[C@@H](O)COP(=O)(O)O ; CCCCCC/C=C\CCCCCCCC(=O)OC[C@@H](O)COP(=O)(O)O ; CCCCCCCCC/C=C\CCCCCCCC(=O)OC[C@@H](O)COP(=O)(O)O]

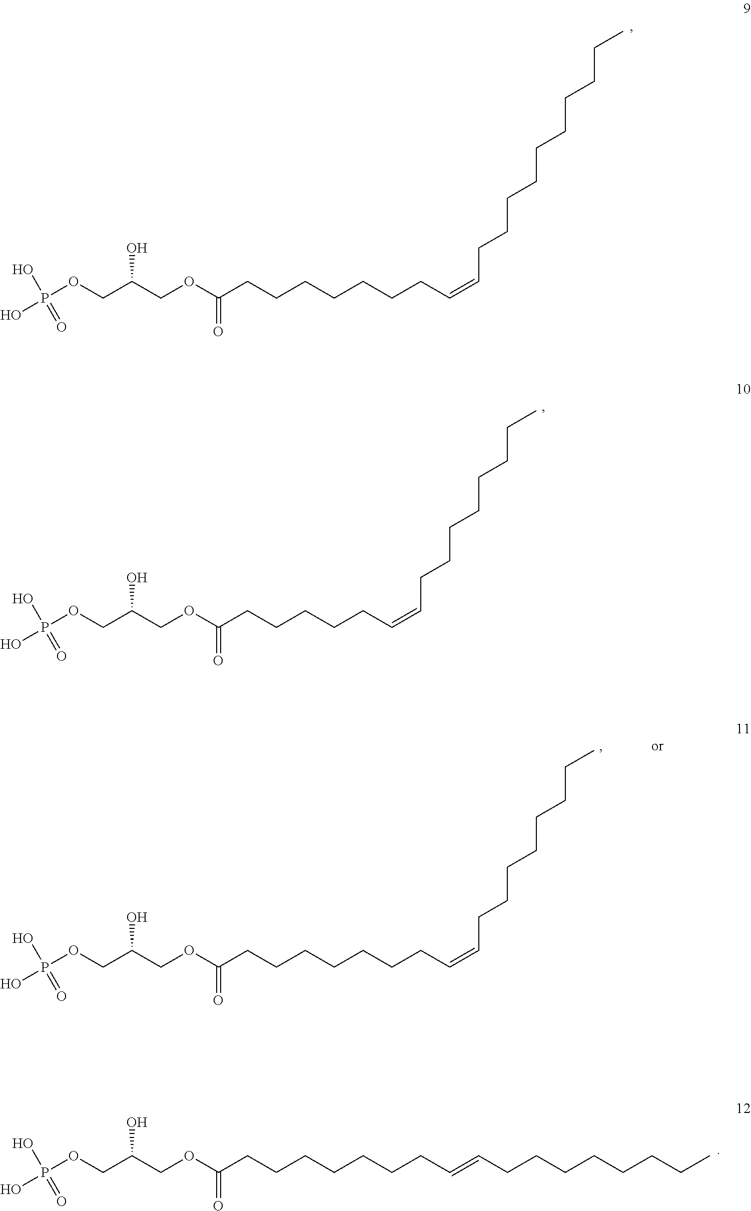 [SMILES: CCCCCCCCCC/C=C\CCCCCCCC(=O)OC[C@@H](O)COP(=O)(O)O ; CCCCCCCC/C=C\CCCCCC(=O)OC[C@@H](O)COP(=O)(O)O ; CCCCCCCC/C=C\CCCCCCCC(=O)OC[C@@H](O)COP(=O)(O)O ; CCCCCCCC/C=C/CCCCCCCC(=O)OC[C@@H](O)COP(=O)(O)O]

In one embodiment, the molecule is not a compound depicted below:

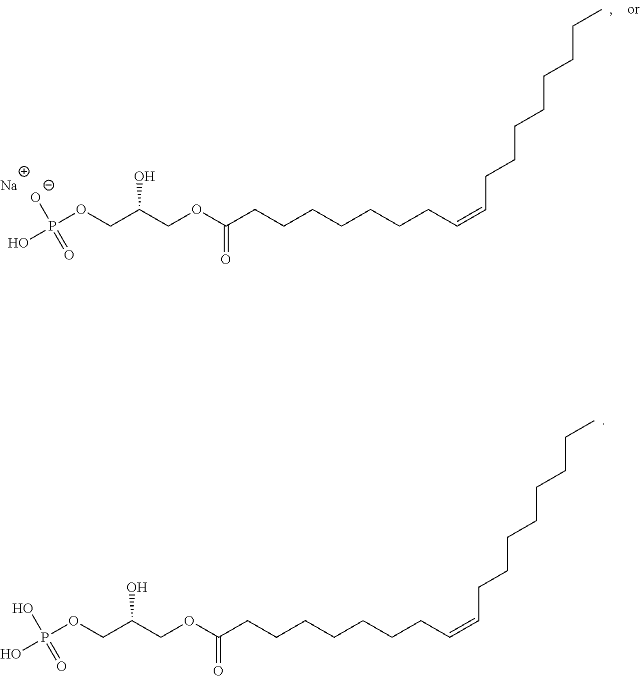 [SMILES: [Na+] ; CCCCCCCC/C=C\CCCCCCCC(=O)OC[C@@H](O)COP(=O)([O-])O ; CCCCCCCC/C=C\CCCCCCCC(=O)OC[C@@H](O)COP(=O)(O)O]

In one embodiment, the molecule can be 2-acyl-lysophosphatidic acid. In a further embodiment, the molecule can be 1-acyl-lysophosphatidic acid.

Knowledge of the primary sequence of a molecule of interest, such as a P2RY5 polypeptide, and the similarity of that sequence with proteins of known function (such as other P2 purinergic receptors), can provide information as to the inhibitors or antagonists of the protein of interest in addition to agonists. Identification and screening of agonists and antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Test compounds, such as P2RY5 modulating compounds, can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76). Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. Libraries are also meant to include for example but are not limited to peptide-on-plasmid libraries, polysome libraries, aptamer libraries, synthetic peptide libraries, synthetic small molecule libraries, neurotransmitter libraries, and chemical libraries. The libraries can also comprise cyclic carbon or heterocyclic structure and/or aromatic or polyaromatic structures substituted with one or more of the functional groups described herein.

As used herein, the term “ligand source” can be any compound library described herein, a library of neurotransmitters, or tissue extract prepared from various organs in an organism's system, that can be used to screen for compounds that would act as an agonist or antagonist of P2RY5. Screening compound libraries listed herein [also see U.S. Patent Application Publication No. 2005/0009163, which is hereby incorporated by reference in its entirety], in combination with in vivo animal studies and functional and signaling assays described below can be used to identify P2RY5 modulating compounds capable of regulating hair growth or treating hair loss disorders.

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci, USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383.

Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/18318.

In vitro translation-based libraries include but are not limited to those described in PCT Publication No, WO 91/05058; and Mattheakis et al., (1994) Proc. Natl. Acad. Sci. USA 91:9022-9026.

In one non-limiting example, non-peptide libraries, such as a benzodiazepine library (see e.g., Bunin et al., (1994) Proc. Natl. Acad. Sci. USA 91:4708-4712), can be screened. Peptoid libraries, such as that described by Simon et al., (1992) Proc. Natl. Acad. Sci. USA 89:9367-9371, can also be used. Another example of a library that can be used, in which the amide functionalities in peptides have been permethylated to generate a chemically transformed combinatorial library, is described by Ostresh et al. (1994), Proc. Natl. Acad. Sci. USA 91:11138-11142.

Computer modeling and searching technologies permit the identification of compounds, or the improvement of already identified compounds, that can modulate P2RY5 expression or activity. Having identified such a compound or composition, the active sites or regions of a P2RY5 molecule can be subsequently identified via examining the sites as to which the compounds bind. These active sites can be ligand binding sites and can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318. Specifically, an orphan GPCR, such as a P2RY5 polypeptide molecule, can be de-orphanized according to methods known in the art, wherein a ligand source can be any compound library described herein, a library of neurotransmitters, or tissue extract prepared from various organs in an organism's system (See Civelli et al., (2001) Trends Neurosci 24(4):230-237; Lin and Civelli (2004) Ann Med 36:204-14; and Civelli et al., (2006) Pharma Therap 110:525-532, which is hereby incorporated by reference).

The three dimensional geometric structure of an active site, for example that of a P2RY5 polypeptide can be determined by known methods in the art, such as X-ray crystallography, which can determine a complete molecular structure. Solid or liquid phase NMR can be used to determine certain intramolecular distances. Any other experimental method of structure determination can be used to obtain partial or complete geometric structures. The geometric structures can be measured with a complexed ligand, natural or artificial, which can increase the accuracy of the active site structure determined. Potential P2RY5 modulating compounds can also be identified using the X-ray coordinates of another GPCR that is similar in structure to P2RY5, for example, using the coordinates of rhodopsin. In one embodiment, a compound that binds to a P2RY5 protein can be identified via: (1) providing an electronic library of test compounds; (2) providing atomic coordinates listed in Table 6 for at least 20 amino acid residues for the binding pocket of the rhodopsin protein (see PDB entry no. 119H; Tokada et al., (2002) Proc. Nat'l. Acad. Sci. USA, 99:5982), wherein the coordinates have a root mean square deviation therefrom, with respect to at least 50% of Cα atoms, of not greater than about 5 Å, in a computer readable format; (3) converting the atomic coordinates into electrical signals readable by a computer processor to generate a three dimensional model of the rhodopsin protein, which is similar to the P2RY5 protein; (4) performing a data processing method, wherein electronic test compounds from the library are superimposed upon the three dimensional model of the protein; and determining which test compound fits into the binding pocket of the three dimensional model, thereby identifying which compound would bind to P2RY5.

Table 6, which contains information encompassing the atomic coordinates for residues of a Rhodopsin crystal (PDB entry no. 119H; Tokada et al., (2002) Proc. Nat'l. Acad. Sci. USA, 99:5982), can be found as an appendix to this application, and is incorporated by reference herein in its entirety.

Potential P2RY5 modulating compounds can also be identified by testing compounds with some structural relationship to compounds that modulate the activity of rhodopsin. For example, one could test the efficacy of a compound such as retinal (U.S. Patent Publication No. 2006/0135460; Proc. Natl. Acad. Sci., 94:13559-64 (1997)). In one embodiment, the invention encompasses to compounds of Formula III:

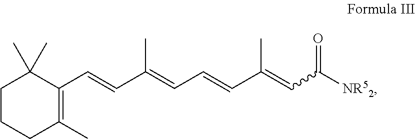

wherein
   each R⁵ is independently —H, —C₁-C₆ alkyl, —C₃-C₈ cycloalkyl, or aryl, wherein aryl may be substituted by substituents such as is —OH, —C₁-C₆ alkyl, or halogen;
   the double bond may be E or Z, or a mixture of both, and pharmaceutically acceptable salts thereof.

In one embodiment, both. R⁵ are H. In another embodiment, one R⁵ is H and the other is aryl.

In one embodiment, aryl is phenyl.

In one embodiment, aryl is phenyl substituted by —OH, such as at the 4 position of the phenyl.

In one embodiment, the double bond is in the E configuration. In another embodiment, the double bond is in the Z configuration.

In one embodiment, methods of the invention comprise administering the compound:

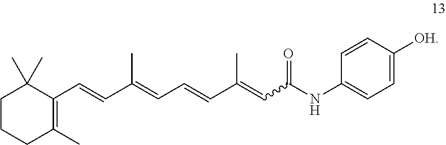 [SMILES: CC(=CC(=O)Nc1ccc(O)cc1)/C=C/C=C(C)/C=C/C1=C(C)CCCC1(C)C]

In one embodiment, a compound of the invention is not:

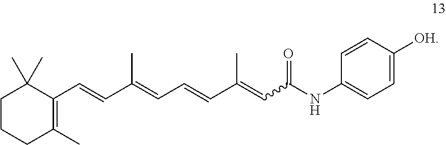 [SMILES: CC(=CC(=O)Nc1ccc(O)cc1)/C=C/C=C(C)/C=C/C1=C(C)CCCC1(C)C]

In one embodiment, the invention encompasses compounds of Formula IV:

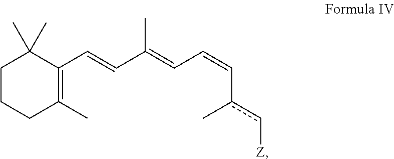

wherein
   Z is —C(═O)R⁶, NR⁵₂, or —(CH₂)ₙ—NR⁵₂;
   each R⁵ is independently —H, —C₁-C₆ alkyl, —C₃-C₈cycloalkyl, or aryl, wherein aryl may be substituted by substituents such as is —OH, —C₁-C₆ alkyl, or halogen; and
   R⁶ is H, —C₁-C₆ alkyl, —C₃-C₈ cycloalkyl, C₃-C₁₀ aryl, or —NH₂; and
   n is 1-6, and pharmaceutically acceptable salts thereof.

In one embodiment, Z is —C(═O)H. In another embodiment, Z is —NH₂. In yet another embodiment, Z is —(CH₂)—NH(C₁-C₆ alkyl). In still another embodiment, Z is —(CH₂)—NH(propyl).

In one embodiment, the double bond is in the E configuration. In another embodiment, the double bond is in the Z configuration.

In one embodiment, a compound of the invention is:

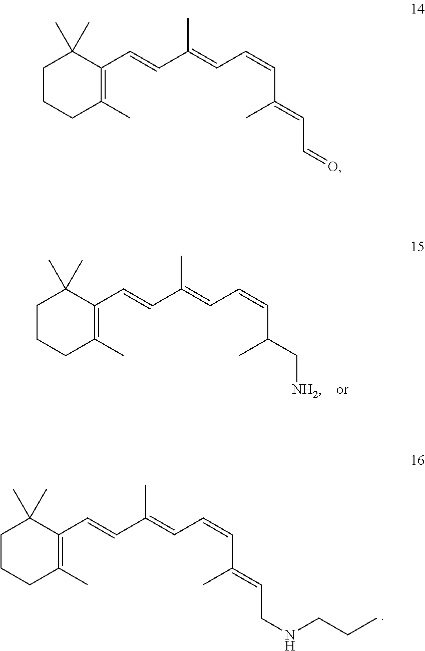

In one embodiment, a compound of the invention is not:

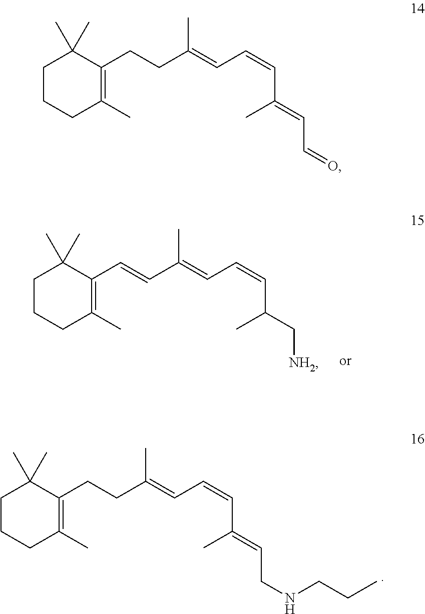 [SMILES: CC1=C(CC/C(C)=C/C=C\C(C)=C\C=O)C(C)(C)CCC1 ; CC1=C(/C=C/C(C)=C/C=C\C(C)CN)C(C)(C)CCC1 ; CCCNC/C=C(C)/C=C\C=C(/C)CCC1=C(C)CCCC1(C)C]

Methods for predicting the effect on protein conformation of a change in protein sequence, are known in the art, and the skilled artisan can thus design a variant which functions as an antagonist according to known methods. One example of such a method is described by Dahiyat and Mayo in Science (1997) 278:82 87, which describes the design of proteins de novo. The method can be applied to a known protein to vary only a portion of the polypeptide sequence. By applying the computational methods of Dahiyat and Mayo, P2RY5 modulating compounds or specific variants thereof confined to regions which bind the active site of a P2 purinergic receptor, such as P2RY5, can be tested to determine whether the compound or the variant retains a desired conformation. Similarly, Blake (U.S. Pat. No. 5,565,325) teaches the use of known ligand structures to predict and synthesize variants with similar or modified function.

Other methods for preparing or identifying peptides that bind to a target are known in the art. Molecular imprinting, for instance, can be used for the de novo construction of macromolecular structures such as peptides that bind to a molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Mosbach, (1994) Trends in Biochem. Sci., 19(9); and Wulff, G., in Polymeric Reagents and Catalysts (Ford, W. T., Ed.) ACS Symposium Series No. 308, pp 186-230, American Chemical Society (1986). One method for preparing mimics of a P2RY5 modulating compound involves the steps of (i) polymerization of functional monomers around a known substrate (the template) that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are prepared by the free radical polymerization of functional monomers, resulting in a compound with a nonbiodegradable backbone. Other methods for designing such molecules include for example drug design based on structure activity relationships, which require the synthesis and evaluation of a number of compounds and molecular modeling.

Screening Assays

P2RY5 Modulating Compounds

A P2RY5 modulating compound can be a compound that affects the activity and/or expression of a P2RY5 molecule in vivo and/or in vitro. P2RY5 modulating compounds can be agonists and antagonists of a P2RY5 molecule, and can be compounds that exert their effect on the activity of P2RY5 via the expression, via post-translational modifications, or by other means.

Test compounds or agents which bind to a P2RY5 molecule, and/or have a stimulatory or inhibitory effect on the activity or the expression of a P2RY5 molecule, can be identified by two types of assays: (a) cell-based assays which utilize cells expressing a P2RY5 molecule or a variant thereof on the cell surface; or (b) cell-free assays, which can make use of isolated P2RY5 molecules or P2RY5 mutants described herein. These assays can employ various P2RY5 molecules (e.g., a biologically active fragment of P2RY5, full-length P2RY5, a fusion protein which includes all or a portion of P2RY5, or a P2RY5 mutant previously presented—having the biochemical variations just described, i.e., a fusion protein or fragments thereof). A P2RY5 molecule can be obtained from any suitable mammalian species (e.g., human P2RY5, rat P2RY5, chick P2RY5, or murine P2RY5). The assay can be a binding assay comprising direct or indirect measurement of the binding of a test compound or a known P2RY5 ligand. The assay can also be an activity assay comprising direct or indirect measurement of the activity of a P2RY5 molecule. The assay can also be an expression assay comprising direct or indirect measurement of the expression of P2RY5 mRNA or protein. The various screening assays can be combined with an in vivo assay comprising measuring the effect of the test compound on the symptoms of a hair loss disorder or disease in a subject (for example, androgenetic alopecia, Telogen effluvium, Alopecia areata, telogen effluvium, Alopecia areata, Tinea capitis, alopecia totalis, or alopecia universalis).

An in vivo assay can also comprise assessing the effect of a test compound on regulating hair growth in known mammalian models that display defective or aberrant hair growth phenotypes (such as mouse models having mutations in the P2RY5 receptor) or mammals that contain a mutation in the P2RY5 open reading frame (ORF) that affects hair growth regulation (Konyukhov et al., (2004) Russian J Gen 40(7): 968-74; Peters et al., (2003) J Invest Dermatol 121(4): 674-680; Green (1974) Mouse News Lett 51:1-23]. In one embodiment, controlling hair growth can comprise a promotion of hair growth in the subject. In another embodiment, controlling hair growth can comprise promoting hair loss in a subject. In a further embodiment, controlling hair growth can comprise a straightening of hair in the subject. For example, the straightening of hair can comprise relaxing an Afroid or Caucasoid hair shaft, wherein the shaft would resemble that of a Mongoloid hair shaft (i.e., straight). Here, the compound's effect in regulating hair growth can be observed either visually via examining the organism's physical hair growth or loss, or by assessing protein or mRNA expression using methods known in the art.

General hair shaft characteristics are described relative to the three true hair classes: Mongoloid, Caucasoid, and Afroid. Mongoloid (i.e., Asian) hair-shafts are the thickest and coarsest, are usually straight and have a circular cross section. Their hair follicles, which number from about 90,000 to about 120,000, are large and straight with a circular cross section. Caucasoid hair-shafts can be straight, wavy or curly. The hair follicle is circular oval or kidney shaped in cross-section. The number of follicles ranges between 86,000-145,000. For example, Titian haired people have about 86,000+follicles while brunettes have about 100,000+follicles. For example, black haired people have about 110,000+follicles and blondes (fine haired) can have about 145,000+follicles. Afroid hair-shafts, however, have tight helices. in cross section, the hair-follicles are elliptical or can be almost ribbon-like flat in cross section. Follicle numbers fall in the range between about 50,000 to about 110,000.

Assays for screening test compounds that bind to or modulate the activity of a P2RY5 molecule can also be carried out. The test compound can be obtained by any suitable means, such as from conventional compound libraries. Determining the ability of the test compound to bind to a membrane-bound form of the P2RY5 molecule can be accomplished via coupling the test compound with a radioisotope or enzymatic label such that binding of the test compound to the P2RY5-expressing cell can be measured by detecting the labeled compound in a complex. For example, the test compound can be labelled with ³H, ¹⁴C, ³⁵S, or ¹²⁵I, either directly or indirectly, and the radioisotope can be subsequently detected by direct counting of radioemmission or by scintillation counting. Alternatively, the test compound can be enzymatically labelled with, for example, horseradish peroxidase, alkaline phosphatase, or luciferase, and the enzymatic label detected by determination of conversion of an appropriate substrate to product.

A cell-based assay can comprise contacting a cell expressing a membrane-bound form of a P2RY5 molecule (for example, a biologically active fragment of P2RY5 or a variant thereof; full-length P2RY5 or a variant thereof; or a fusion protein which includes all or a portion of P2RY5 or a variant thereof) expressed on the cell surface with a test compound and determining the ability of the test compound to modulate (such as increase or decrease) the activity of the membrane-bound form of a P2RY5 molecule. Determining the ability of the test compound to modulate the activity of the membrane-bound P2RY5 molecule can be accomplished by any method suitable for measuring the activity of a G-protein coupled receptor or other seven-transmembrane receptor. The activity of a 7 TMD receptor can be measured in various ways, such as activation of phospholipase C, alteration in intracellular Ca²⁺concentration, alteration in intracellular diacylglycerol (DAG) concentration, alteration in intracellular inositol triphosphate (IP₃) concentration, alteration in intracellular adenosine cyclic 3′,5′-monophosphate (cAMP) concentration, or a combination thereof (see also Jala and Haribabu (2006) Methods Mol Biol. 332:159-165; Thomsen et al., (2005) Curr Opin Biotech 16:655-665; Oh et al., (2006) Intl Rev Cytol 252:163-218; and Kristiansen, K., (2004) Pharma Therap 103:21-80, which are all hereby incorporated by reference in their entireties).

The ability of a test compound to modulate the activity of a P2RY5 molecule or a variant thereof can be accomplished via determining the ability of the molecule to bind to or interact with a target molecule. The target molecule can be a molecule that binds or interacts with P2RY5 or a P2RY5 mutant in nature. Non-limiting examples include: a molecule on the surface of a cell which expresses P2RY5 or a variant thereof, a molecule in the extracellular milieu, a molecule on the surface of a second cell, a cytoplasmic molecule, or a molecule. associated with the internal surface of a cell membrane. The target molecule can be a component of a signal transduction pathway which transduces an extracellular signal (for example, a signal generated by binding of a ligand of P2RY5, through the cell membrane and into the cell).

The cell-free assays of the present invention entail use of either a membrane-bound form of a P2RY5 molecule or a P2RY5 mutant described herein, or a soluble fragment thereof. In the case of cell-free assays comprising the membrane-bound form of the polypeptide, a solubilizing agent can be used in order for the membrane-bound form of the polypeptide to be maintained in solution. Examples of such solubilizing agents include but are not limited to non-ionic detergents such as Triton X-100, Triton X-114, n-octylglucoside, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), n-dodecylglucoside, n-dodecylmaltoside, octanoyl-N-methylglucamide, decanoyl-N-methylglucamide, Thesit, or N-dodecyl=N,N-dimethyl-3-ammonio-1-propane sulfonate.

A P2RY5 molecule or a P2RY5-target molecule can be immobilized to facilitate the separation of complexed from uncomplexed forms of one or both of the proteins. Binding of a test compound to a P2RY5 molecule or a variant thereof, or interaction of P2RY5 with a target molecule in the presence and absence of a test compound, can be accomplished in any vessel suitable for containing the reactants. Examples of such vessels include microtiter plates, test tubes, and micro-centrifuge tubes, in one embodiment, a fusion protein can be provided which adds a domain that allows one or both of the proteins to be bound to a matrix (for example, glutathione-S-transferase (GST) fusion proteins or glutathione-S-transferase fusion proteins can be adsorbed onto glutathione sepharose beads (Sigma Chemical; St. Louis, Mo.) or glutathione derivatized microtiter plates).

A P2RY5 molecule or a variant thereof, can also be immobilized via being bound to a solid support. Non-limiting examples of suitable solid supports include glass or plastic slides, tissue culture plates, microtiter wells, tubes, silicon chips, or particles such as beads (including, but not limited to, latex, polystyrene, or glass beads). Any method known in the art can be used to attach a polypeptide (or polynucleotide) corresponding to P2RY5 or a variant thereof, or test compound to a solid support, including use of covalent and non-covalent linkages, or passive absorption.

The diagnostic assay of the screening methods of the invention can also involve monitoring the expression of a P2RY5 molecule. For example, regulators of the expression of a P2RY5 molecule can be identified via contacting a cell with a test compound and determining the expression of P2RY5 protein or P2RY5 mRNA in the cell. The protein or mRNA expression level of P2RY5 in the presence of the test compound is compared to the protein or mRNA expression level of P2RY5 in the absence of the test compound. The test compound can then be identified as a regulator of P2RY5 expression based on this comparison. For example, when expression of P2RY5 protein or mRNA is statistically or significantly greater in the presence of the test compound than in its absence, the test compound is identified as a stimulator/enhancer of expression of P2RY5 protein or mRNA. In other words, the test compound can be said to be a P2RY5 modulating compound (such as an agonist). Alternatively, when expression of P2RY5 protein or mRNA is statistically or significantly less in the presence of the test compound than in its absence, the compound is identified as an inhibitor of the expression of P2RY5 protein or mRNA. In other words, the test compound can also be said to be a P2RY5 modulating compound (such as an antagonist). The expression level of P2RY5 protein or mRNA in cells can be determined by methods previously described

For binding assays, the test compound can be a small molecule which binds to and occupies the active site of a P2RY5 polypeptide, or a variant thereof. This can make the ligand binding site inaccessible to substrate such that normal biological activity is prevented. Examples of such small molecules include, but are not limited to, small peptides or peptide-like molecules. In binding assays, either the test compound or the P2RY5 polypeptide can comprise a detectable label, such as a fluorescent, radioisotopic, chemiluminescent, or enzymatic label (for example, alkaline phosphatase, horseradish peroxidase, or luciferase). Detection of a test compound which is bound to a polypeptide of P2RY5 or a P2RY5 mutant described herein can then be determined via direct counting of radioemmission, by scintillation counting, or by determining conversion of an appropriate substrate to a detectable product.

Determining the ability of a test compound to bind to a P2RY5 molecule or a variant thereof, such as a P2RY5 mutant described herein, also can be accomplished using real-time Bimolecular Interaction Analysis (BIA) [McConnell, (1992); Sjolander, (1991)]. BIA is a technology for studying biospecific interactions in real time, without labeling any of the interactants (for example, BIA-core™). Changes in the optical phenomenon surface plasmon resonance (SPR) can be used as an indication of real-time reactions between biological molecules.

To identify other proteins which bind to or interact with a P2RY5 molecule and modulate its activity, a P2RY5 polypeptide can be used as a bait protein in a two-hybrid assay or three-hybrid assay [Szabo, (1995); U.S. Pat. No. 5,283,317), according to methods practiced in the art. The two-hybrid system is based on the modular nature of most transcription factors, which consist of separable DNA-binding and activation domains.

Functional Assays

Test compounds can be tested for the ability to increase or decrease the activity of a P2RY5 molecule, or a variant thereof. Activity can be measured after contacting either a purified P2RY5 molecule, a cell membrane preparation, or an intact cell with a test compound. A test compound that decreases the activity of a P2RY5 molecule by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for decreasing the activity of a P2RY5 molecule. A test compound that increases the activity of a P2RY5 molecule by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for increasing the activity of a P2RY5 molecule.

Other screening techniques include the use of cells which express a P2RY5 molecule (for example, transfected CHO cells) in a system which measures extracellular pH changes caused by receptor activation (Iwabuchi et al., (1993) Oncogene 8, 1693-1696). For example, compounds can be contacted with a cell which expresses the P2RY5 receptor and a secondary messenger response (such as signal transduction or pH changes) can be Measured to determine whether the potential compound activates or inhibits the receptor, Another screening technique that can be utilized involves expressing a P2RY5 molecule in cells (such as endothelial cells, smooth muscle cells, embryonic kidney cells, etc.) in which the receptor is linked to a phospholipase C or D. The screening can be accomplished as described previously by quantifying the degree of activation of the receptor from changes in the phospholipase activity.

Pharmaceutical Compositions and Administration for Therapy

P2RY5 molecules and P2RY5 modulating compounds of the invention can be incorporated into pharmaceutical compositions suitable for administration. Such compositions can comprise a P2RY5 molecule or a P2RY5 modulating compound and a pharmaceutically acceptable carrier.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

The invention also provides for a kit that comprises a pharmaceutically acceptable carrier and a P2RY5 modulating compound identified using the screening assays of the invention packaged with instructions for use, For modulators that are antagonists of the activity of a P2RY5 molecule, or which reduce the expression of a P2RY5 molecule, the instructions would specify use of the pharmaceutical composition for promoting the loss of hair on the body surface of a mammal (for example, the arms, legs, bikini area, face, and the like).

For P2RY5 modulating compounds that are agonists of the activity of a P2RY5 molecule or increase the expression of a P2RY5, the instructions would specify use of the pharmaceutical composition for regulating hair growth. In one embodiment, the instructions would specify use of the pharmaceutical composition for the treatment of hair loss disorders. In another embodiment, the instructions would specify use of the pharmaceutical composition for promoting hair growth in a subject. In a further embodiment, the instructions would specify use of the pharmaceutical composition for straightening hair in a subject. For example, administering a P2RY5 agonist can relax the Afroid or Caucasoid hair shaft, wherein the shaft would then resemble that of a Mongoloid hair shaft.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition containing a P2RY5 modulating compound can be administered in conjunction with a pharmaceutically acceptable carrier, for any of the therapeutic effects discussed herein. Such pharmaceutical compositions can comprise, for example antibodies directed to P2RY5 human receptor or a variant thereof, P2RY5 agonists, P2RY5 antagonists, or P2RY5 inhibitors. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the P2RY5 modulating compound (e.g., a polypeptide or antibody) in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art

In some embodiments, the P2RY5 modulating compound can be applied via transdermal delivery systems, which slowly releases the active compound for percutaneous absorption. Permeation enhancers can be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

“Subcutaneous” administration can refer to administration just beneath the skin (i.e., beneath the dermis). Generally, the subcutaneous tissue is a layer of fat and connective tissue that houses larger blood vessels and nerves. The size of this layer varies throughout the body and from person to person. The interface between the subcutaneous and muscle layers can be encompassed by subcutaneous administration.

This mode of administration can be feasible where the subcutaneous layer is sufficiently thin so that the factors present in the compositions can migrate or diffuse from the locus of administration and contact the hair follicle cells responsible for hair formation. Thus, where intradermal administration is utilized, the bolus of composition administered is localized proximate to the subcutaneous layer.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Localization and Expression of P2RY5 in Human and Mouse

A commercially available antibody was used to localize P2RY5 in the human and mouse hair follicle. In human, P2RY5 localized to only one layer of the hair follicle (called Henle's layer) and in the upper layers of epidermis, in addition to the outer root sheath (ORS), cortex, and the cuticle. In mouse, P2RY5 was localized only in the hair follicle and not in epidermis. It is not uncommon to see that P2RY5 localizes in one epidermis and not the other, for the mouse epidermis is very thin and often does not reflect same as human Immunofluorescence methods were carried out as described in Example 2.

### Example 2

### Disruption of the P2RY5 Gene, an Orphan G-Protein Coupled Receptor, Underlies Autosomal Recessive Woolly Hair

Mutations in the P2RY5 gene encoding an orphan G-protein coupled receptor, cause autosomal recessive woolly hair in humans.

The genetic determinants of hair texture in humans are largely unknown. Several human syndromes exist in which woolly hair comprises a part of the phenotype, however, simple autosomal recessive inheritance of isolated woolly hair (OMIM 278150) has only rarely been reported (1, 2). To identify a gene involved in the control of hair texture, genetic linkage analysis was performed in 6 families of Pakistani origin with autosomal recessive woolly hair (ARWH), as well as associated features including sparse, fragile and hypopigmented hair shafts. Using the Affymetrix 10K array and subsequent narrowing of the interval with microsatellite markers, a region of linkage on chromosome 13q14.2-14.3 was identified in all families studied, with a maximum multipoint LOD score of Z=17.97. Fine mapping of critical recombination events delineated a region of 2.1 Mb, containing 10 known genes, 2 pseudogenes and 1 predicted transcript. One of the known genes, P2RY5, encodes a heptaspan transmembrane G-protein coupled receptor (GPCR) and is a nested gene residing within intron 17 of the retinoblastoma 1 (RB1) gene. Resequencing of P2RY5 revealed homozygous pathogenic mutations in all families studied, including insertions, deletions and missense mutations in evolutionarily conserved residues from human to zebrafish. P2RY5 is expressed in both Henle's and Huxley's layers of the hair follicle inner root sheath in human, in addition to the outer root sheath (ORS), cortex, and the cuticle; mouse and rat follicles; as well as in epidermal keratinocytes in a differentiation dependent manner. These findings indicate that disruption of P2RY5 underlies autosomal recessive woolly hair, and more broadly, uncover a novel gene involved in the determination of hair texture in humans.

The hair shaft is a highly keratinized tissue of the hair follicle (HF) that emerges from the surface of the skin. The hair shaft displays wide variability in texture and color among individuals of different populations around the world (3). Variation in hair forms are believed to be programmed by intrinsic asymmetries in differentiation of keratinocytes in the bulb region of the HF, from which hair shaft growth originates (4, 5). A number of neuroectodermal syndromes and metabolic disorders have been known to show abnormalities in hair texture. For example, trichothiodystrophy (OMIM 601675) is characterized by sulfur-deficient brittle hair that shows alternating light and dark bands along the hair shaft under polarized microscopy, thus called a tiger tail hair (6). Patients with Menkes disease (OMIM 309400) show light-colored, lusterless and twisted hair, termed kinky or steely hair (6). Causative genes for both diseases have been previously identified (7, 8), and mutations in these genes directly affect the synthesis of hair keratins and/or their keratinization in the hair shaft (6). Woolly hair refers to a phenotypic variant with fine and tightly curled hair (9). In some cases, it appears as a part of syndromes, such as Naxos disease (OMIM 601214) that is characterized by cardiomyopathy, palmoplantar keratoderma and woolly hair, and is caused by mutations in either the desmoplakin (10) or plakoglobin (11) gene. Woolly hair without any additional systemic manifestations can be inherited as an either autosomal dominant (ADWH; OMIM 194300) (2) or autosomal recessive (ARWH; OMIM 278150) (1, 2) trait. Although tightly curled hair is the major clinical finding, the additional presence of hair shaft fragility and markedly reduced hair density have also been reported in families with ADWH (12). In addition, affected individuals with ARWH showed not only woolly hair, but also sparse and depigmented hair (1, 2). To date, however, the pathogenesis of hereditary woolly hair remains largely unknown. This study identified a gene involved in isolated ARWH, with the goal of dissecting the genetic determinants of hair texture in humans.

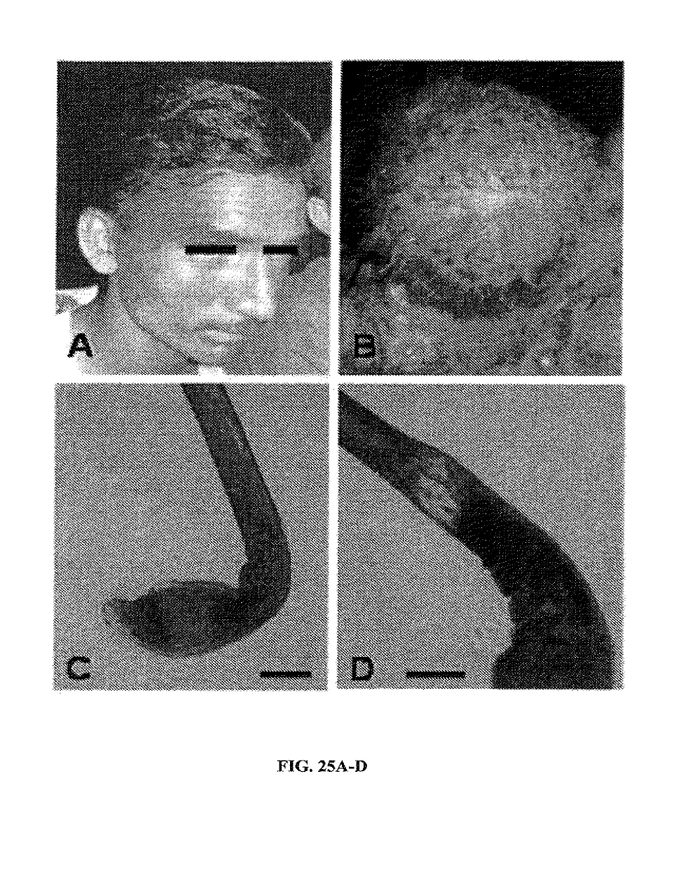

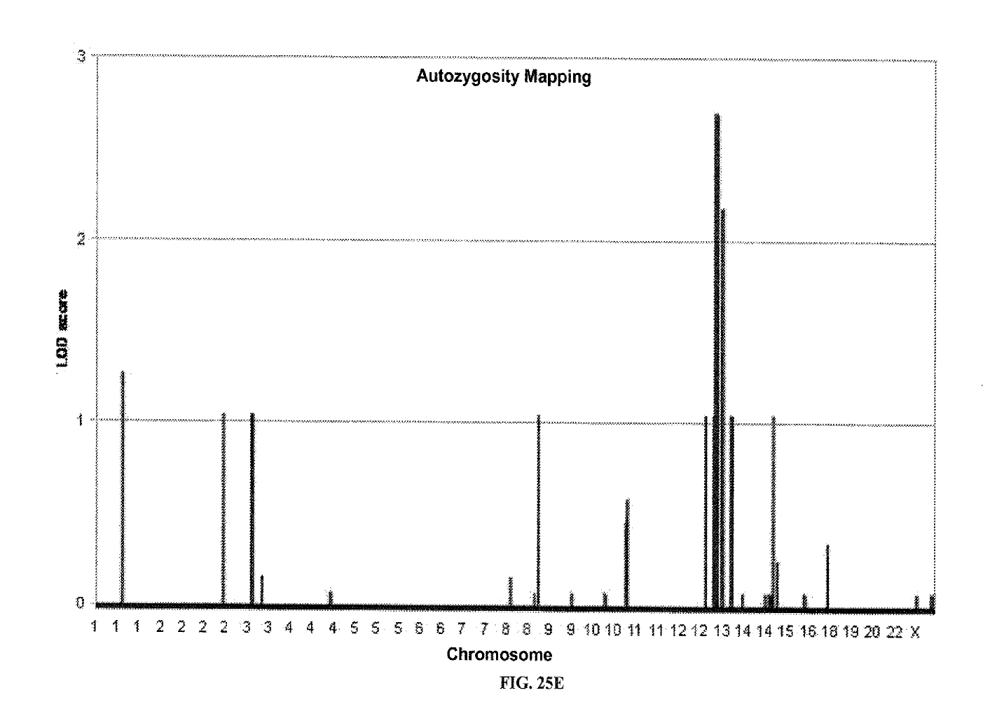

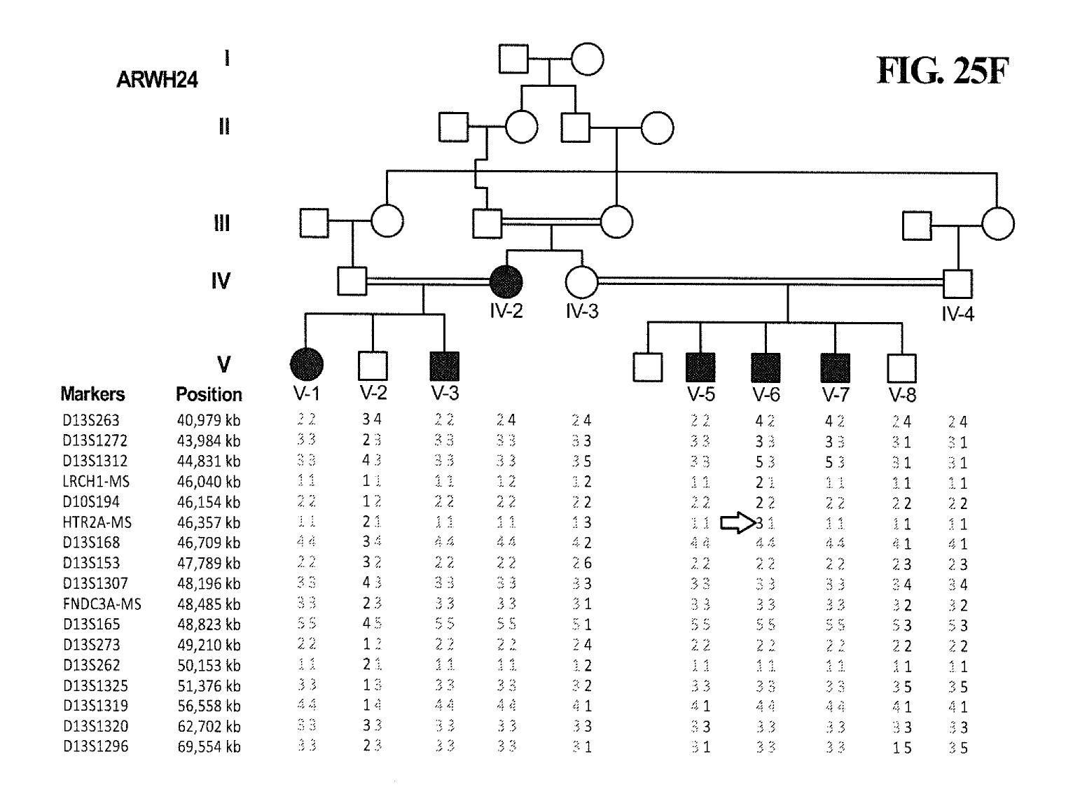

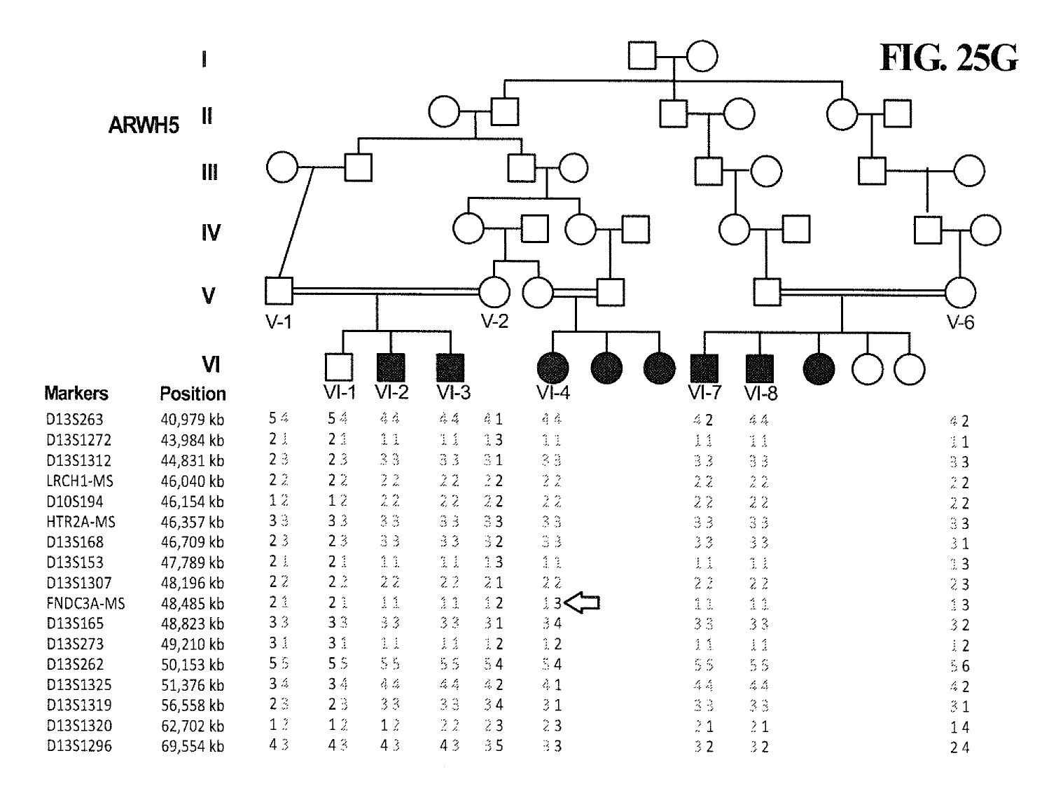

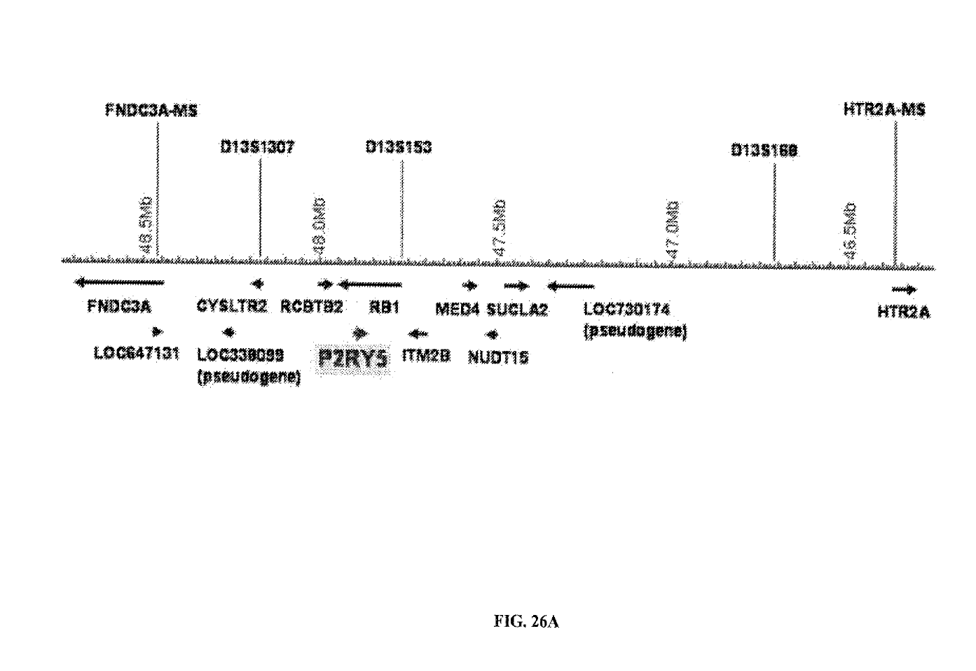

Several consanguineous Pakistani families were recently identified with features consistent with ARWH. Pedigrees of all families were consistent with recessive inheritance, and each family had multiple affected individuals. Clinical features were present at birth. The hair over the entire scalp region is coarse, lusterless, thy, and tightly curled, leading to a diffuse woolly hair phonotype (FIGS. 25A and 25B). Some affected individuals have thin and light-colored hair that can be easily plucked. Hair density is variable from normal to less dense among affected individuals within a family and between families. Hair growth is generally slow and stops after a few inches, and the hair shafts have tapered ends. Most of the plucked hairs show dystrophic features without root sheath components in the bulb portion (FIGS. 25C and 25D). Eyebrow, eyelash, and beard hairs appear normal. Affected individuals in all families show normal teeth, nails, and sweating, and do not show palmoplantar. hyperkeratosis or keratosis pilaris. There was no familial history of either heart disease, early sudden death, neurologic abnormalities or a high prevalence of cancers.

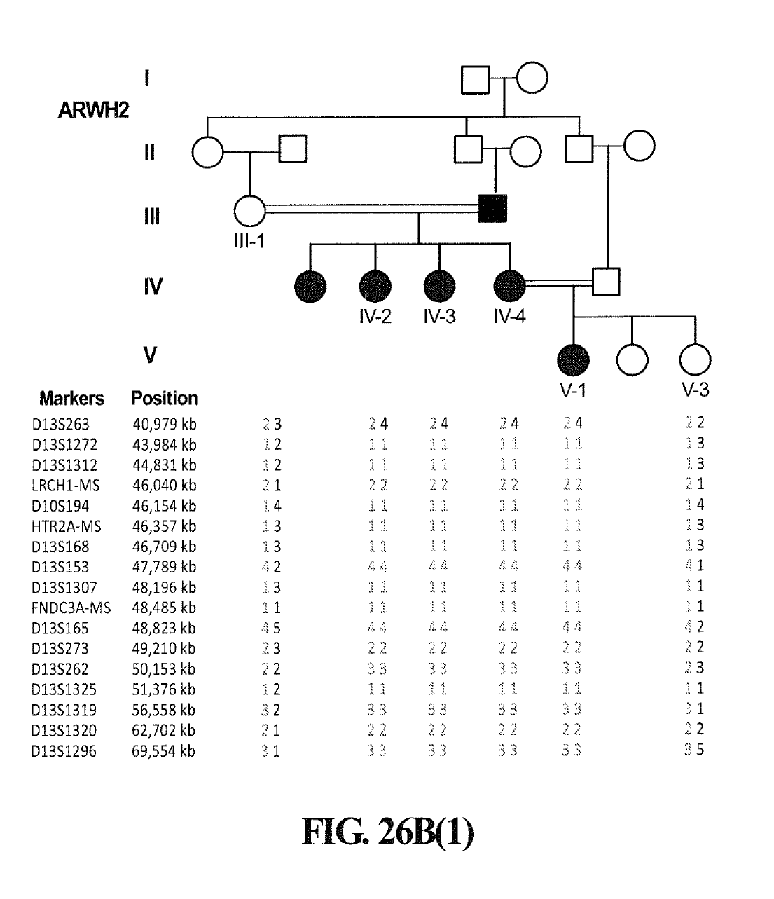

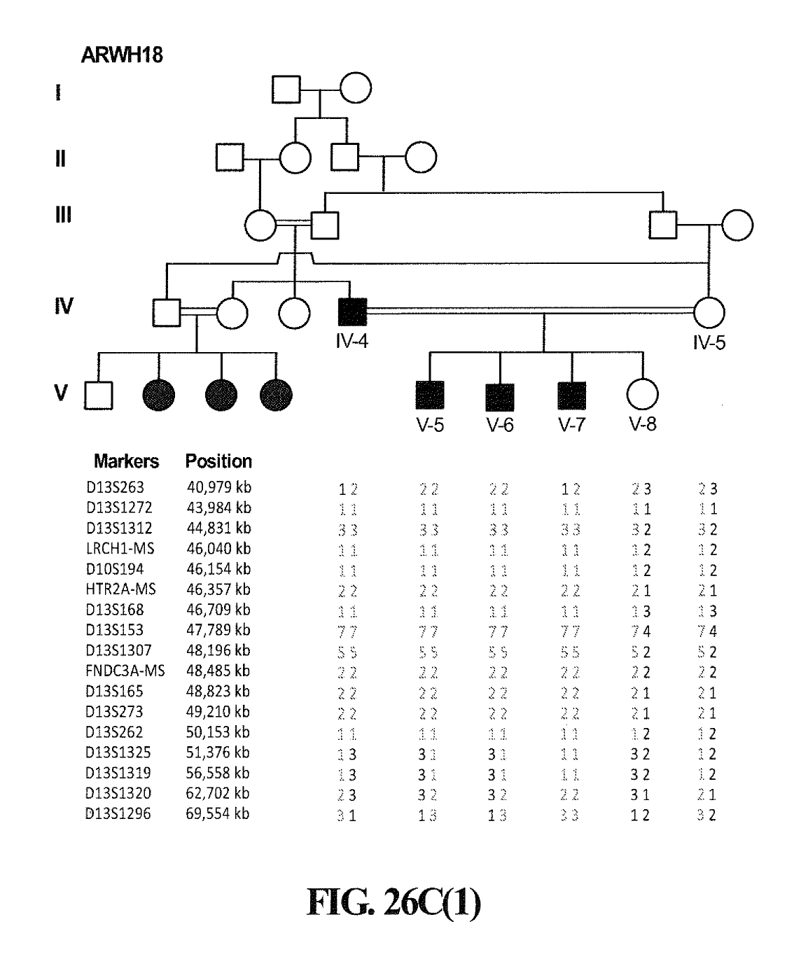

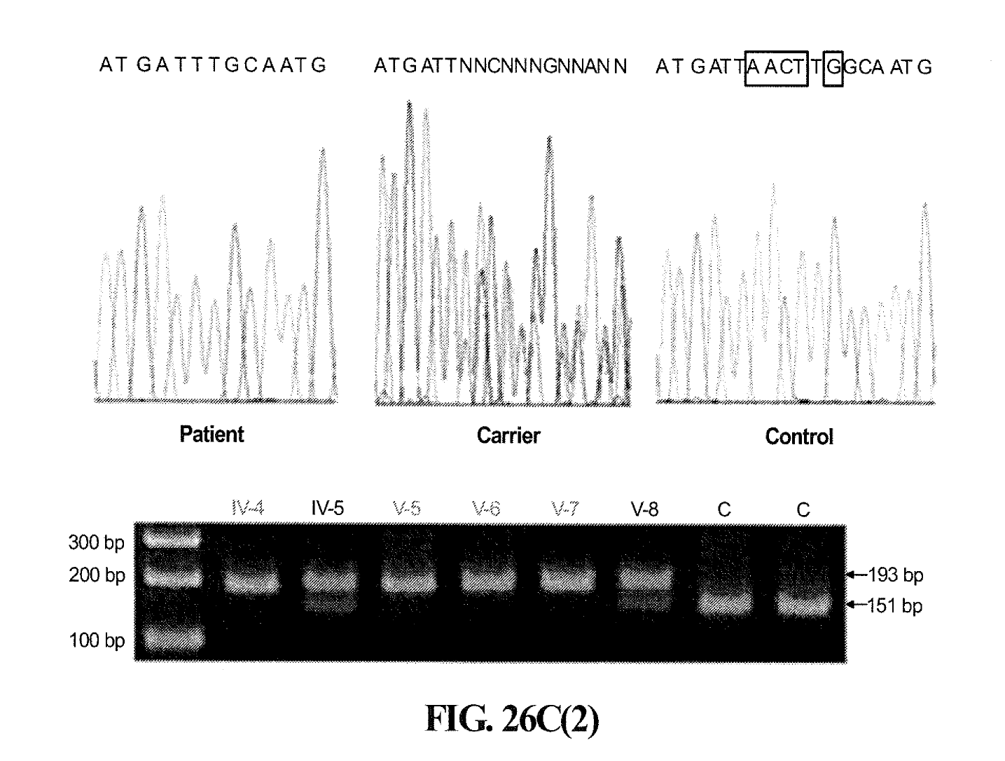

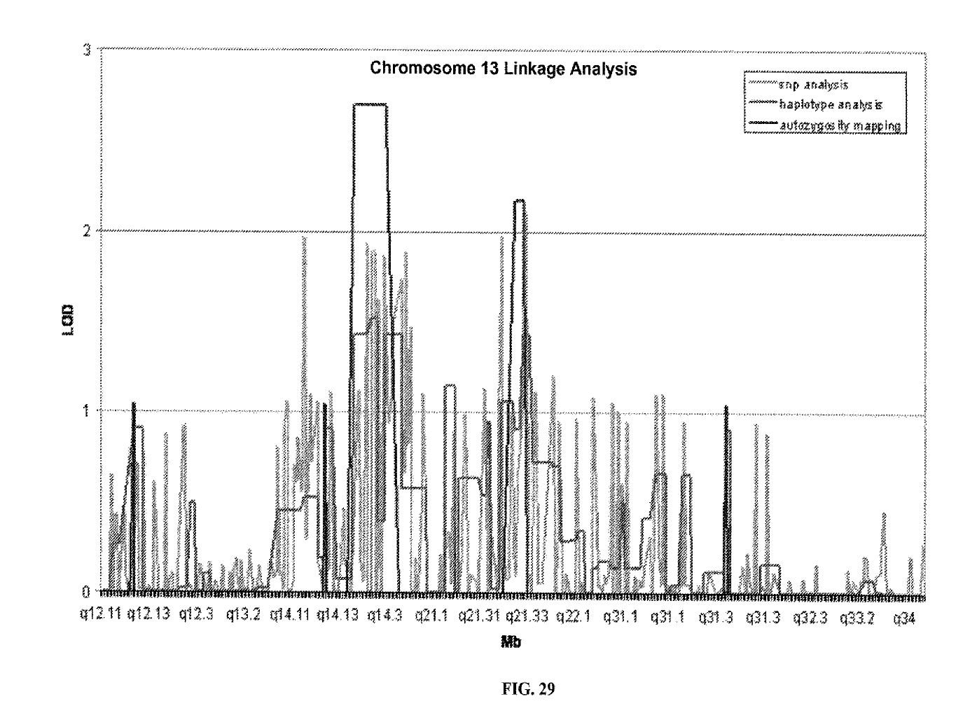

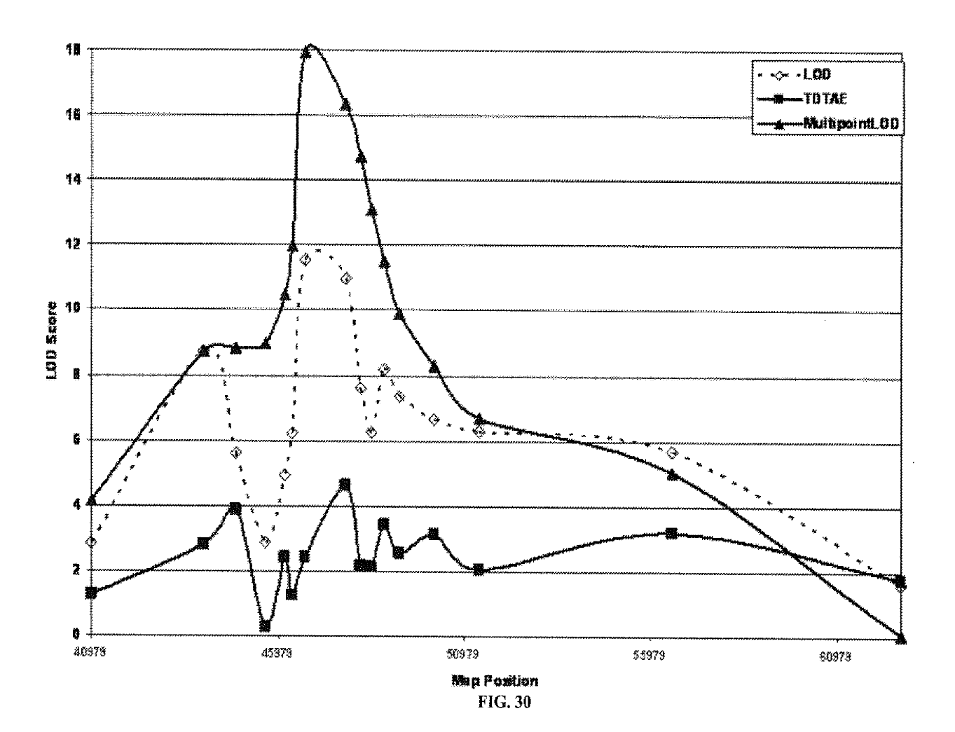

A whole genome scan on one family, ARWH24, was initially performed using the Affymetrix 10K SNP array. Parametric statistical tests identified a region of excess homozygosity shared among affected individuals on chromosome 13q14.2-14.3 (FIG. 25E) with a LOD score of 2.70. Haplotype analysis of this region identified a cluster of 4 snps that spanned an interval from 47.27 Mb to 48.35 Mb (Z=1.52) (FIG. 29). Microsatellite markers were then placed across the region of autozygosity and used to genotype other families, revealing linkage to the same location (Z=17.972) (FIG. 30). Critical recombination events were detected between markers HTR2A-MS and D13S168 in the affected individual V-6 of ARWH24 (FIG. 25F) as well as between markers D13S1307 and FNDC3A-MS in the affected individual VI-4 of ARWH5 (FIG. 25G), which allowed the interval of linkage to be narrowed to 2.1 Mb flanked by markers HTR2A-MS and FNDC3A-MS. The critical region contained 10 known genes, 2 pseudogenes and 1 unknown predicted transcript (FIG. 26A). Four of the known genes were excluded since they were already associated with other disorders, and ranked P2RY5 last on the prioritized list, since it was embedded within intron 17 of the RB1 gene.

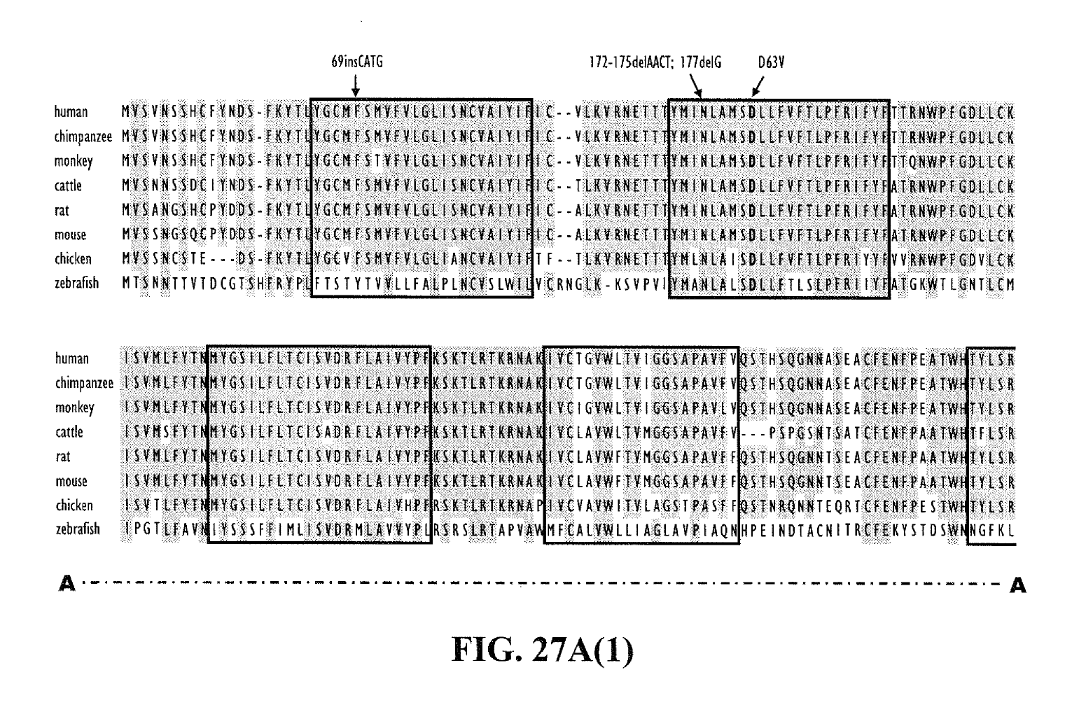

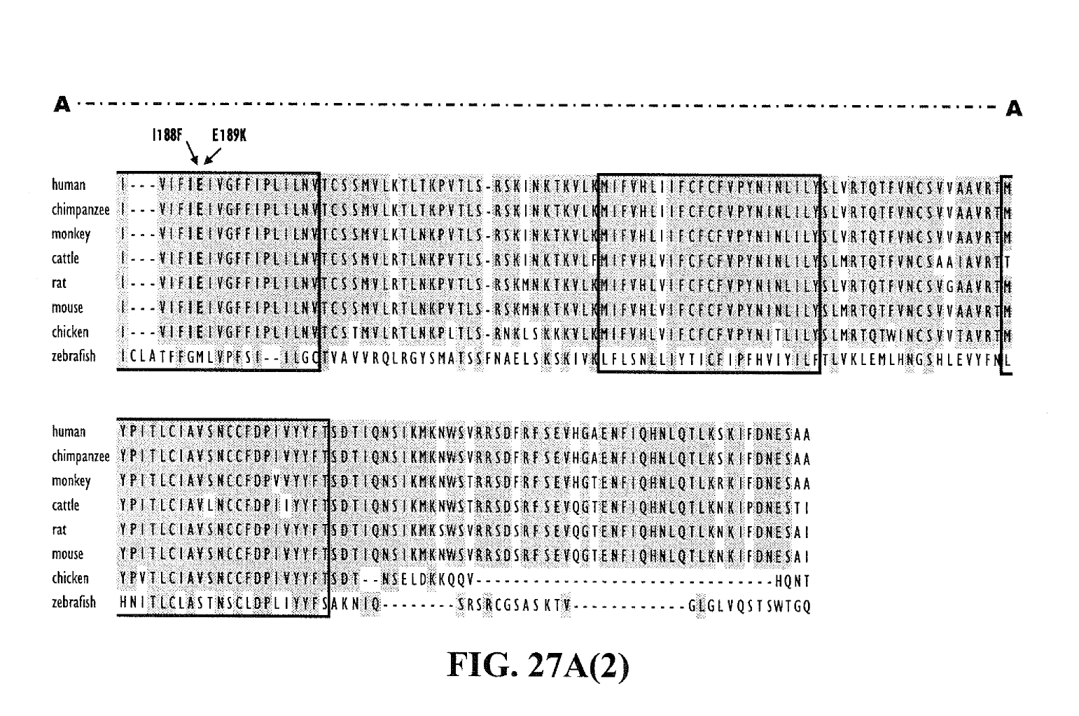

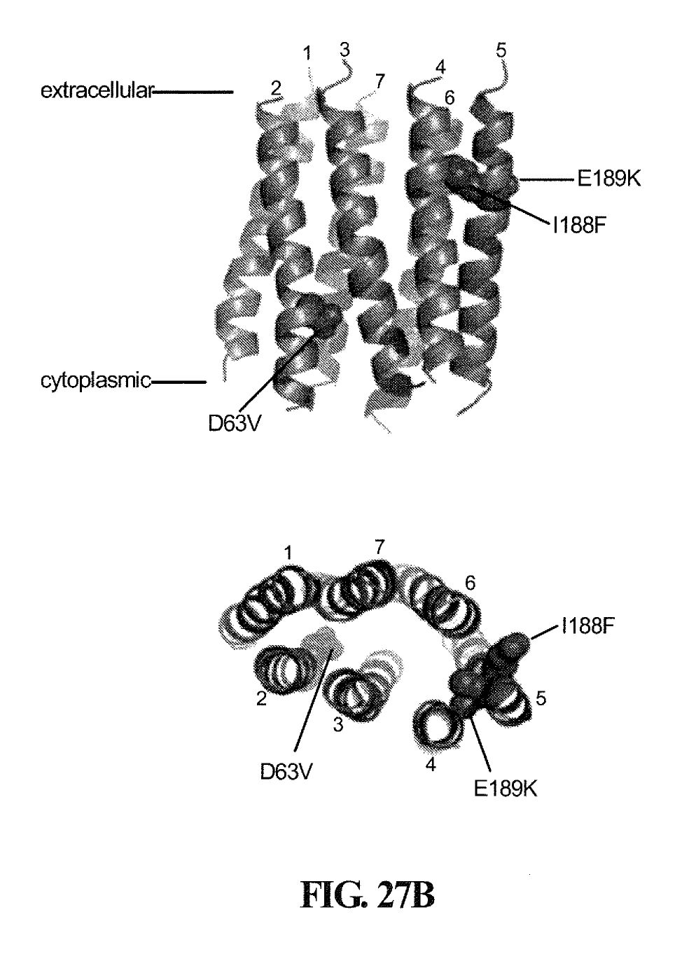

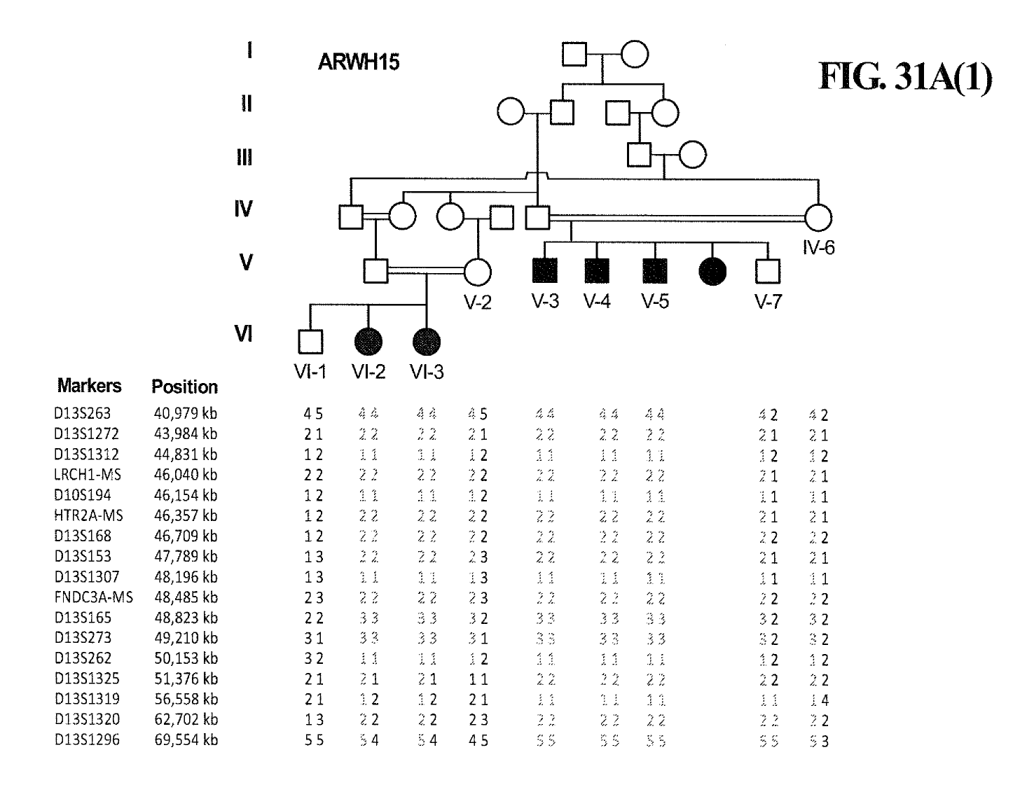

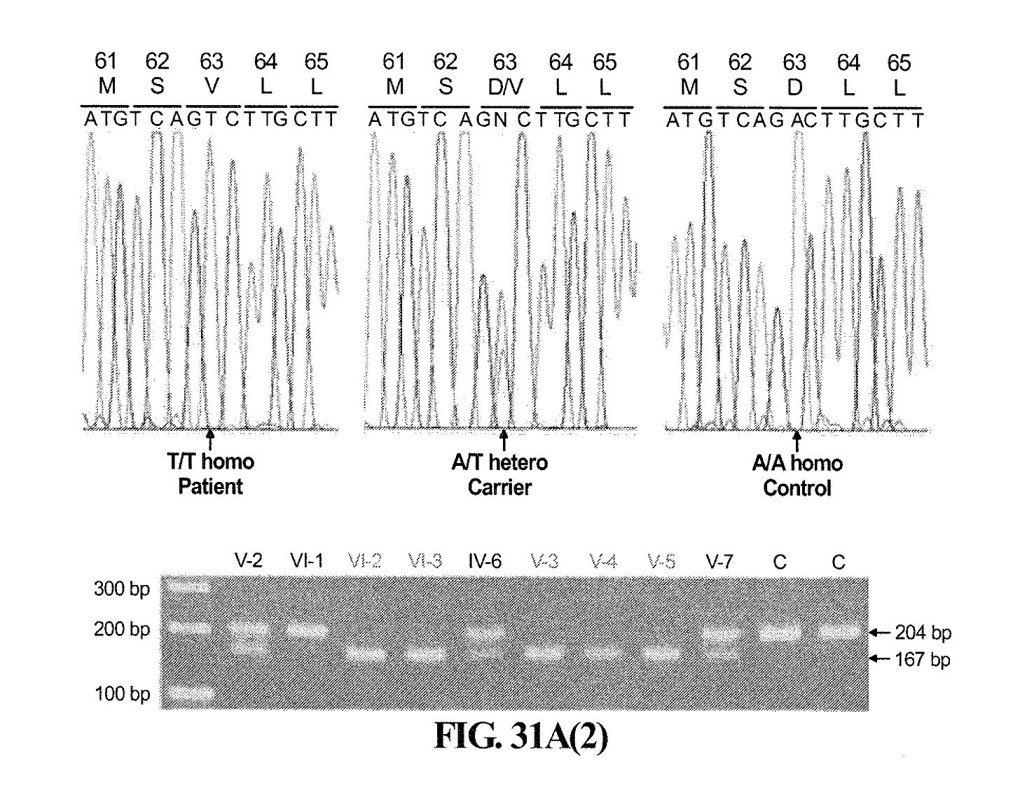

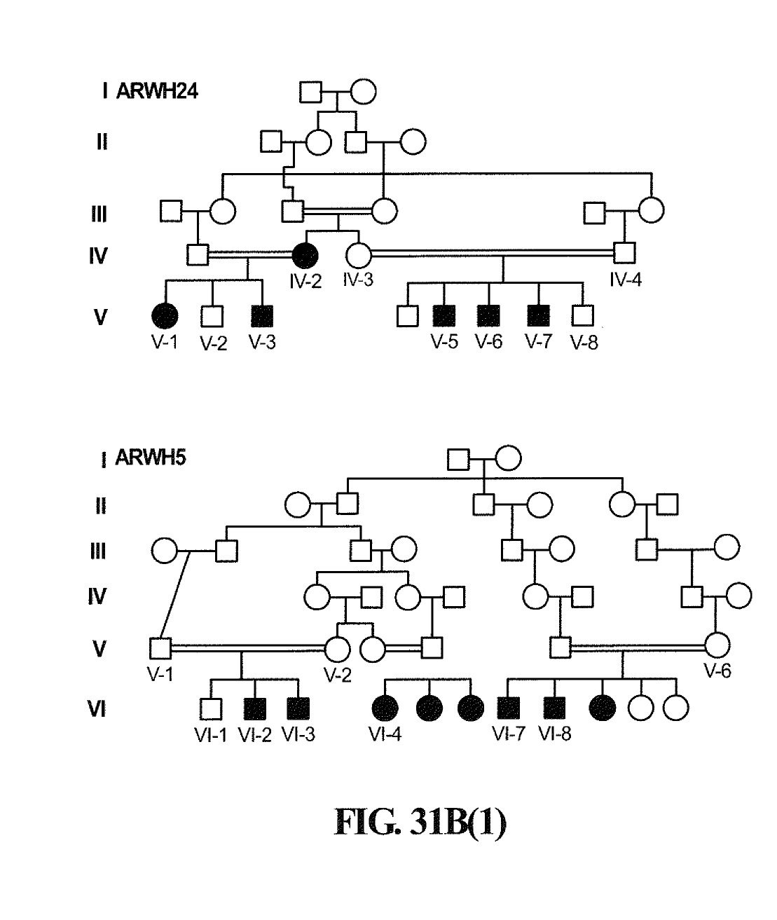

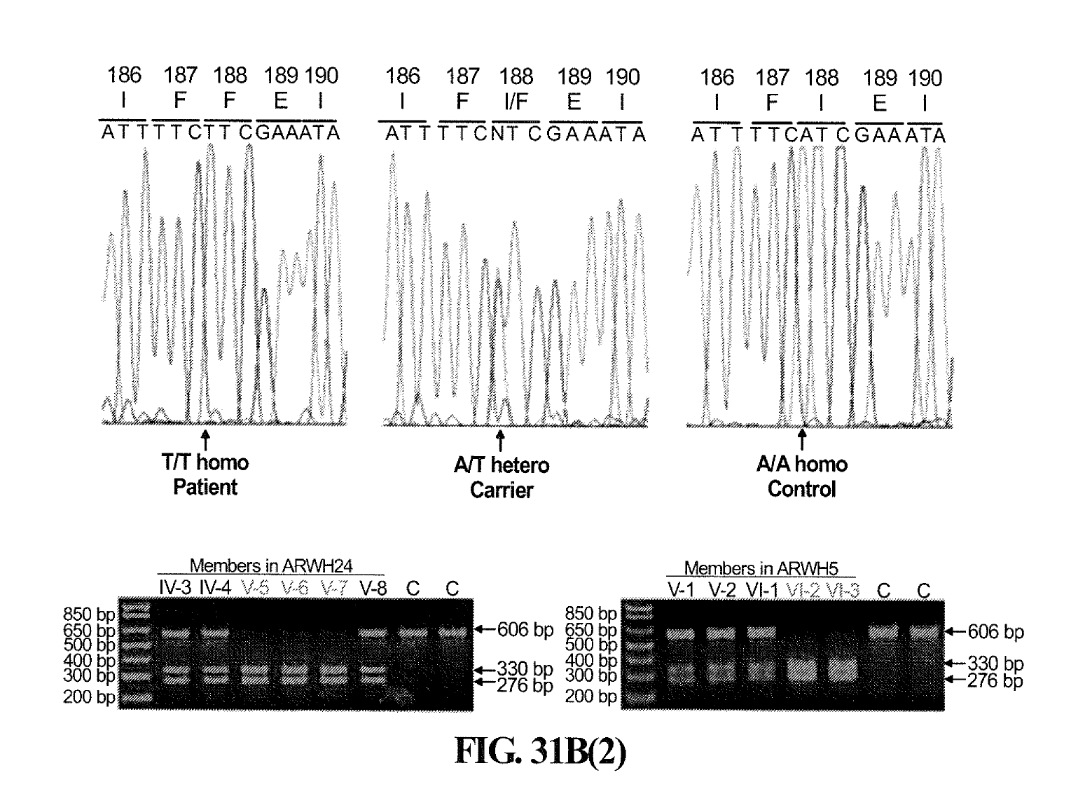

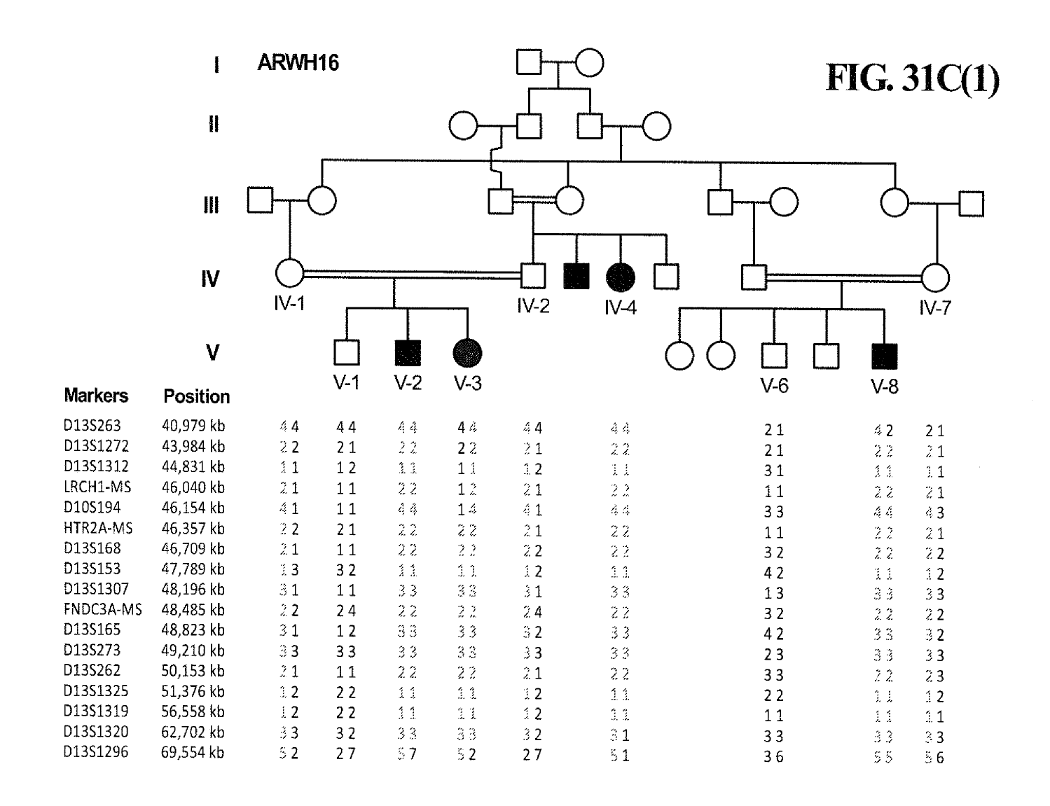

After exclusion of the other known genes, resequencing of affected individuals revealed mutations in P2RY5 in all families. First, a homozygous four nucleotide insertion was identified in the coding sequence of the P2RY5 in affected individuals of ARWH2, designated 69insCATG (FIG. 26B), which results in a frameshift at codon 24 and a premature termination codon (PTC) (Table 2), Second, affected individuals in ARWH18 are homozygous for a complex mutation which consists of a complex four nucleotide deletion at position 172-175 and a one nucleotide deletion at position 177 in P2RY5, designated 172-175delAACT; 177delG (FIG. 26C), which leads to a frameshift and a PTC (Table 2). In addition, a total of three distinct missense mutations, D63V, I188F, and E189K, were found in the other 4 families (Table 2 and FIG. 31A-C). P2RY5 is highly conserved in vertebrate evolution, with homologs present in genomes as distant as zebrafish (FIG. 27A). Each of the three missense mutations disrupt residues conserved from chicken to human (I188F and E189K) in the fifth transmembrane domain, and from zebrafish to human (D63V) in the second transmembrane domain, indicating that they play a critical role in protein function (FIG. 27A). Two homology models of P2RY5 were examined: one based on the crystal structure of rhodopsin and the other based on bacteriorhodopsin, produced by the G Protein-Coupled Receptor Data Base (GPCRDB) (FIG. 27B). In both models, residue D63 is positioned mid-membrane in helix 2, with the side chain buried within the protein core. Similarly, residue E189 is positioned mid-membrane in helix 5. These negatively charged side chains are almost certain to interact with other protein groups to avoid the hydrophobic environment of the lipid membrane. Both the D63V and E189K mutants would result in an alteration of the balance of the buried charge. Such mutations are among those to disrupt protein folding. The I188F mutation is less dramatic, substituting a larger for a smaller hydrophobic side chain. Nevertheless, mutations of this type often result in loss of function or folding integrity in other proteins (13).

TABLE 2
P2RY5 mutations in ARWH families. (PTC + n)
indicates the generation of premature termination
codon at ‘n’ residues downstream of the mutation.

| Family | Mutation | Consequence | Verification |
|---|---|---|---|
| ARWH2 | 69insCATG | frameshift at | NsiI digestion |
| | | codon 24 | |
| | | (PTC + 29) | |
| ARWH18 | 172-175delAACT; | frameshift at | MseI digestion |
| | 177delG | codon 58 | |
| | | (PTC + 31) | |
| ARWH15 | 188A > T | D63V | mismatch PCR |
| | | | followed by |
| | | | MlyI digestion |
| ARWH5 | 562A > T | I188F | BstBI digestion |
| ARWH24 | | | |
| ARWH16 | 565G > A | E189K | TaqI digestion |

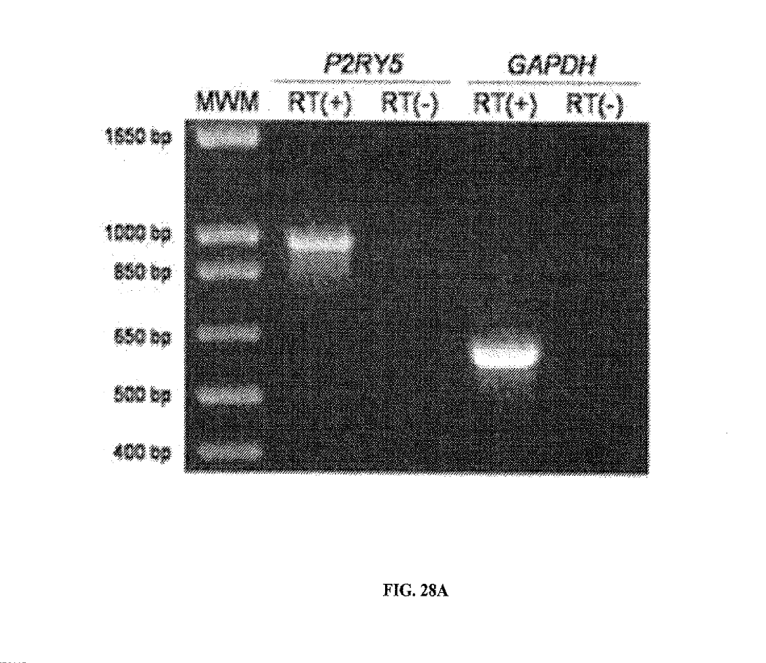

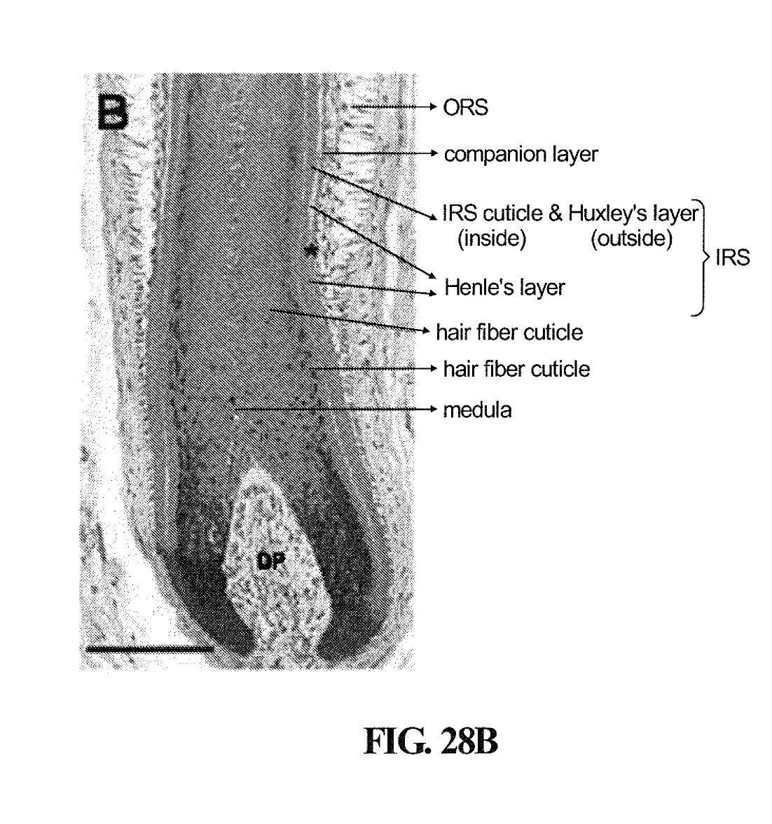

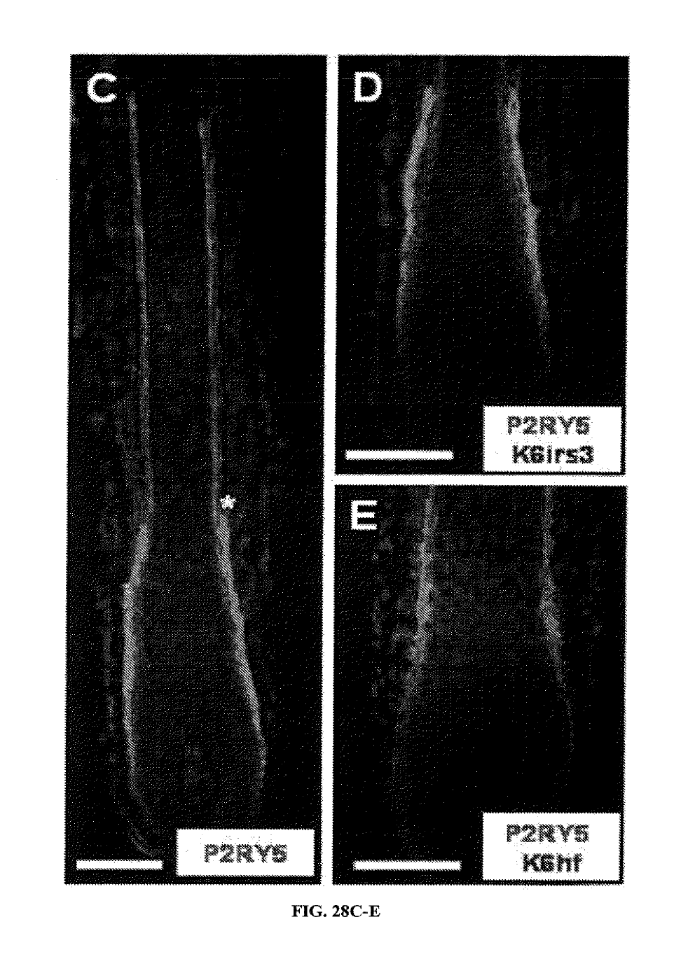

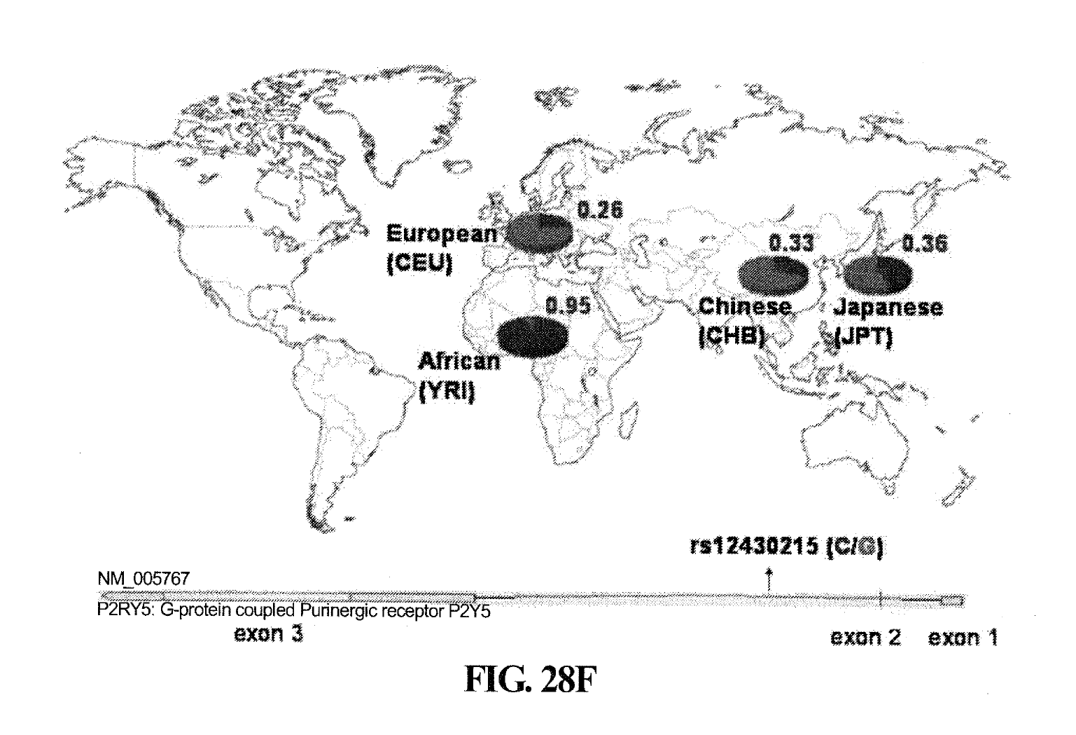

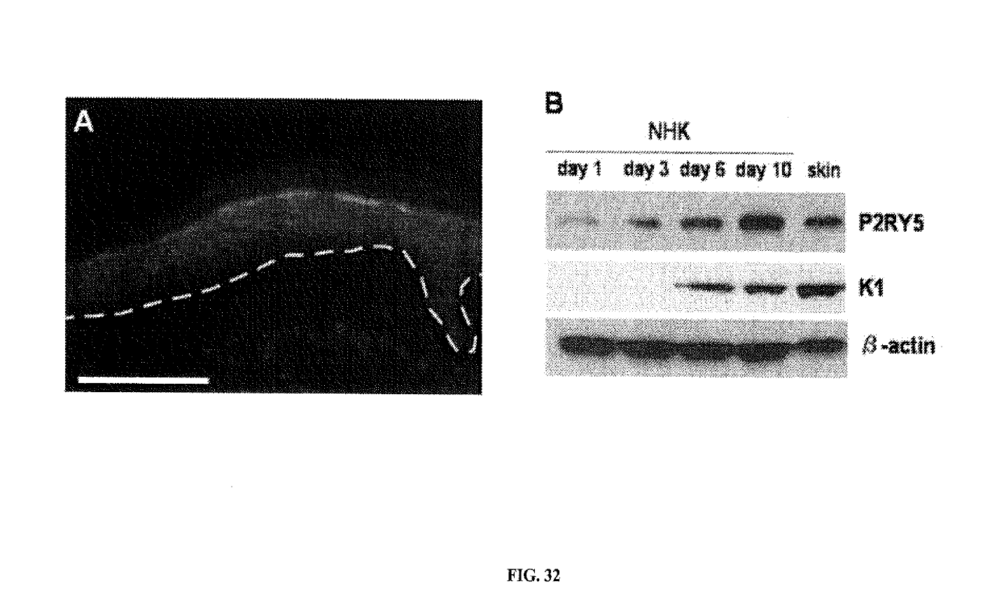

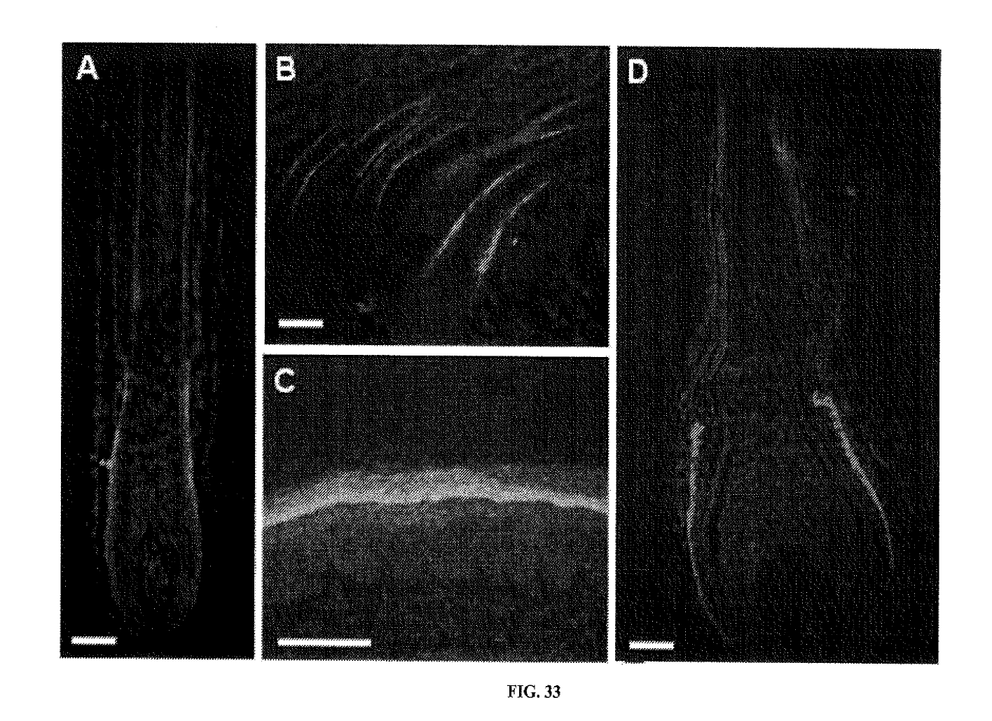

The expression pattern of P2RY5 reported in the literature/databases denotes high expression in placenta, thymus, spleen, and prostate, however, to implicate P2RY5 in the pathogenesis of ARWH, its expression was examined in the HF. RT-PCR was first performed using total RNA extracted from plucked human HFs using gene-specific primers, which confirmed the expression of P2RY5-transcripts in the human HF (FIG. 28A). Immunofluorescence analysis of human HFs with a commercially available anti-P2RY5 antibody (MBL International) revealed prominent expression in both Henle's and Huxley's layers of the inner root sheath (IRS) (FIG. 28B-E). P2RY5 was also found localized to the outer root sheath (ORS), cortex, and the cuticle. In the upper portion of the hair bulb, a strong signal is detected in Henle's layer (FIG. 28C-E). After the complete keratinization of Henle's layer occurs, the expression switches predominantly to Huxley's layer (FIG. 28C). In addition to the HF, P2RY5 is also expressed in suprabasal layers of the epidermis, where expression levels increases with differentiation (FIG. 32A). Normal human keratinocytes in high calcium media were cultured to induce differentiation, and performed western blotting using cell lysates, which showed similar P2RY5 expression kinetics to the epidermis (FIG. 32B). Both mouse and rat P2RY5 show essentially the same expression pattern as human P2RY5 (FIG. 33). Despite the presence of P2RY5 expression in differentiating epidermal cells, no evidence of skin phenotype in any affected family members was found. Perhaps the function of P2RY5 in epidermal differentiation can be compensated by the presence of other P2Y family members such as P2Y1 and P2Y2 (14).

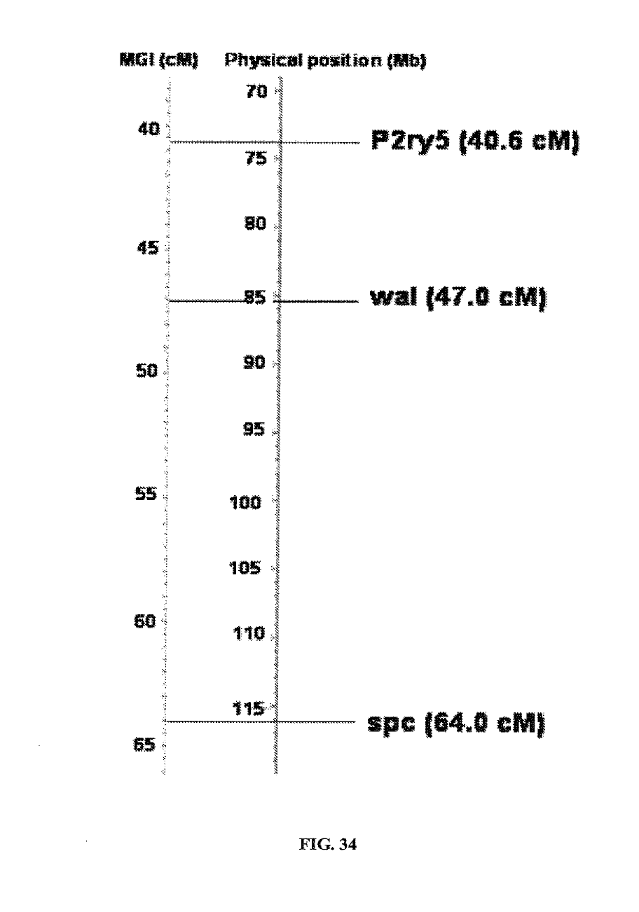

To ask whether mutations in the mouse can similarly result in a woolly hair pheonotype, two spontaneous mutations were investigated that were known to map closely to the Rb1 gene on mouse chromosome 14 (FIG. 34). These genes are known as waved alopecia (wal) (15) and sparse coat (spc) (16). Mutations in the genes are characterized by matted or wavy hair, as well as hair thinning that becomes more pronounced with age (17). Sequence analysis of P2RY5 in both wal and spc revealed no mutations in either mouse model, and correlation with the mouse therefore awaits targeted disruption of P2RY5.

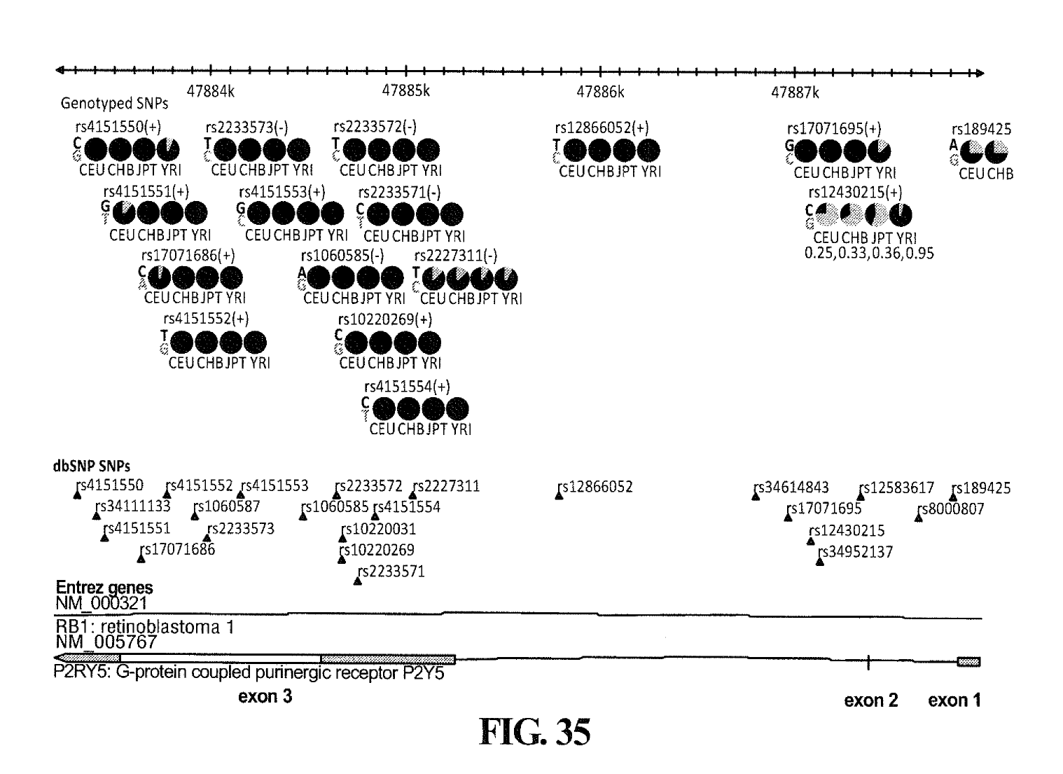

To investigate whether variation in P2RY5 was associated with common ethnic variations in hair texture, whether SNPs in or near P2RY5 occur with differing frequencies among different human populations was examined. The HapMap database identified 16 SNPs in the P2RY5 gene (NCBI build 35). In general, there is surprisingly little variation reported within this gene (FIG. 35 and Table 3). Of the 16 SNPs for which frequency data is available for European and African populations, 4 (rs12430215, rs17071695, rs1894255, and rs4151551) show statistically significant variation between these ethnic groups (Pearson's Chi Square, p<0.05). rs12430215, which is located within intron 2 of the P2RY5, shows the most ethnic variation (FIG. 28F and Table 3). Based on the distribution of allele frequency divergence between African Americans and European Americans reported in the literature, for three of these SNPs, the observed divergence is less than the expected value. However, the probability of observing a difference as extreme as that for rs12430215 is less than 0.001 (18). Interestingly, several groups have hypothesized that extreme differences in allele frequencies between races indicate the presence of a gene that contributes to phenotype differences among human populations, such as hair texture or skin pigmentation. One group conducted a genome wide search for regions that harbor an excess of such SNPs, and one of the regions identified included the P2RY5 gene (19).

The expression studies in the HF show that P2RY5 is expressed in both Henle's and Huxley's layers of the IRS, in addition to the outer root sheath (ORS), cortex, and the cuticle. The IRS directly surrounds the hair fiber and plays an essential role in supporting, anchoring, and molding the growing hair shaft (20). It has been reported that a mouse mutation known as caracul (Cα), has heterozygous mutations in an IRS-specific type II keratin, mK6irs1 (mK71), and shows wavy coat phenotype (21). The HFs of the Cα mutant are twisted and/or curved due to the disturbance of IRS architecture and keratinization (21). Importantly, mK6irs1 shares the same expression pattern in the IRS with P2RY5 (22). In addition, the bulb region of plucked hairs from most affected individuals with P2RY5 mutations demonstrated irregular bending without attachment of root sheath components. Thus, mutations in P2RY5 result in disruption of the IRS structure in the lower HF. Such an abnormal IRS would compromise its function in anchoring the hair shaft, and would cause the abnormal bending of the bulb region, leading to the woolly hair phenotype and related defects.

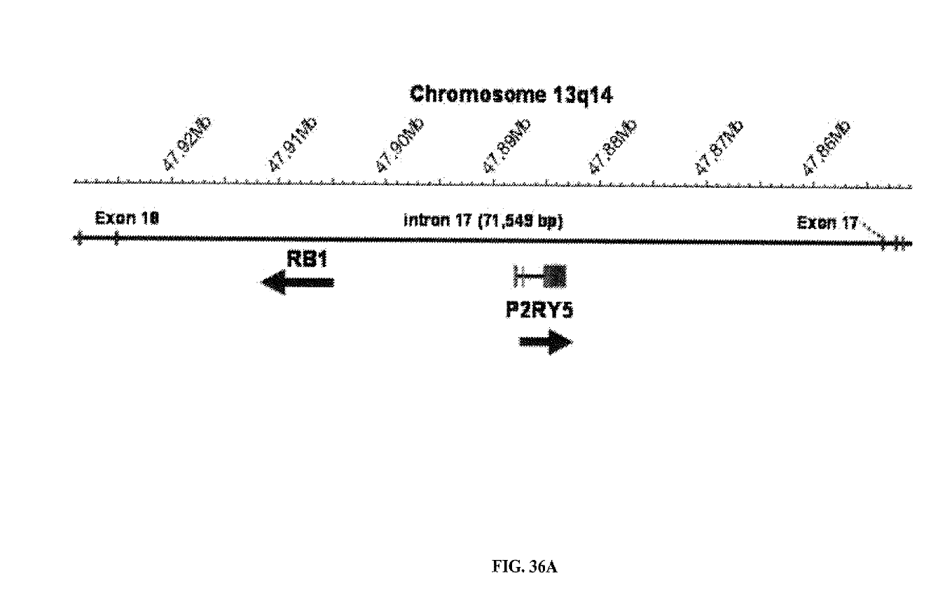

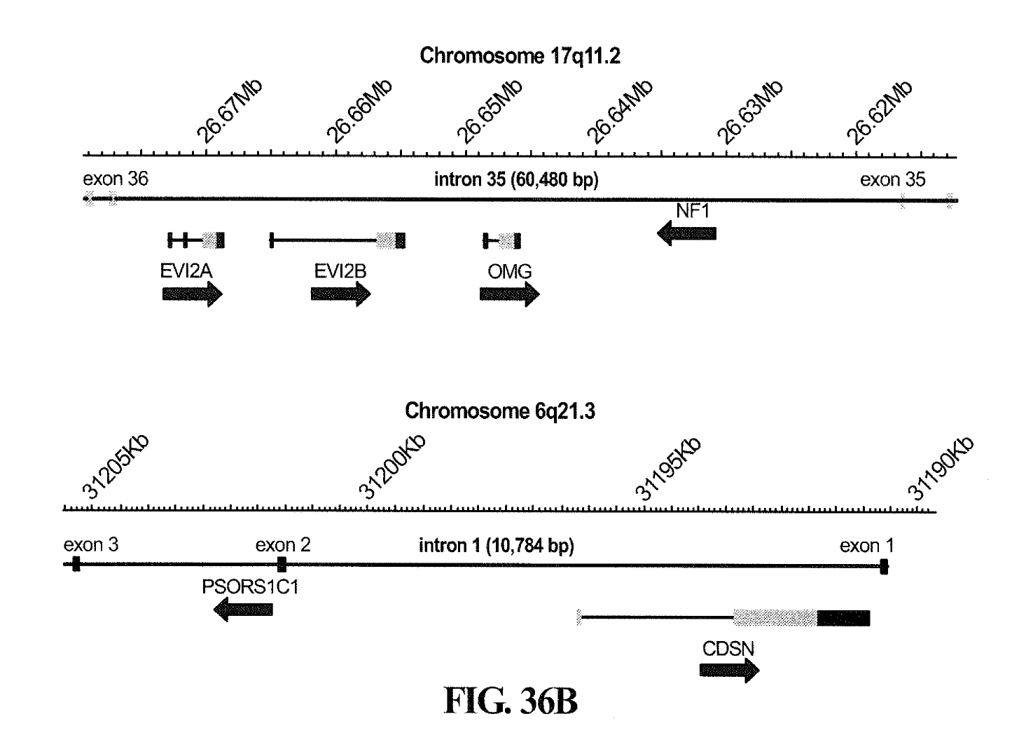

P2RY5 is embedded within the largest intron (˜70 kb) of the RB1 gene, which occupies ˜180 kb of chromosome 13, and is transcribed in the opposite orientation to RB1 (FIG. 36A). To date, many examples of such ‘nested genes’ have been identified in the human genome (23), For example, the EVI2A, EVI2B, and OMG genes are located within intron 35 of the NF1 gene on chromosome 17q, and the CDSN gene exists within intron 1 of the PSORS1C1 gene on chromosome 6q (FIG. 36B) (23). It is noteworthy that the nested gene CDSN has been shown to be the causative gene for another hereditary hair disease, hypotrichosis simplex of the scalp (OMIM 146520) and is expressed in the IRS of the HE (24). The results also provide insight into the functional importance of nested genes. Presumably, P2RY5 is simultaneously deleted when RB1 is lost by interstitial deletion of chromosome 13 in children affected with RB (25). However, since this is a somatic event in the eye, a woolly hair phenotype does not develop, although P2RY5 can be homozygously deleted. Interestingly, P2RY5 has also been found to be associated with cancers (26, 27). Lee et al. recently identified heterozygous germ-line or somatic cell mutations in the P2RY5 of patients with bladder cancer, and showed that homozygous inactivation of P2RY5 caused by deletion of RB1 in the wild type allele contributes to the expansion of in situ neoplasia (27). The ARWH families reported here with P2RY5 mutations do not exhibit a higher prevalence of cancers.

The human P2RY5 gene has 2 introns and is ˜4.7 kb in size, and is not well studied in the literature. P2RY5 was originally reported as the human homolog of a chicken T-cell-specific receptor gene, known as 6H1 (28). P2RY5 is seven-pass transmembrane protein that is an orphan member of the G-protein coupled receptor (GPCR) superfamily, and by sequence homology bears most resemblance to the P2Y family of purinergic GPCRs (28). P2RY5, along with P2RY9 and P2RY10, while bearing homology to the purinergic GPCRs, do not function in this manner. Although P2RY5 was originally reported to exhibit nucleotide-promoted second messenger responses (29), a later report refuted these findings (30). Its true function(s), ligands and the intracellular signaling cascade that it modulates (if any) remain to be determined by deophanization. P2RY5 is a GPCR to be implicated in a human hair disorder, rendering it a druggable target using small molecules. Therapeutic manipulation of P2RY5 can be envisaged for the treatment of excessive or unwanted hair, or as an alternative to current methods of changing the texture of the hair fiber, such as chemical straightening or permanent waving. The findings indicate that disruption of P2RY5 underlies autosomal recessive woolly hair, and more broadly, uncover a novel gene involved in the determination of hair texture in humans.
1. T. Salamon, Hautarzt 14, 540 (1963).
2. P. E. Hutchinson, R. J. Cairns, R. S. Wells, Trans. St. Johns Hosp. Dermatol. Soc. 60, 160 (1974).
3. D. Hrdy, Am. J. Phys. Anthropol. 39, 7 (1973).
4. B. A. Bernard, J. Am. Acad. Dermatol. 48, S120 (2003).
5. S. Thibaut, Br. J. Dermatol. 152, 632 (2005).
6. J. Stratigos, H. P. Baden, Arch. Dermatol. 137, 1465 (2001).
7. G. Weeda et al., Am. J. Hum. Genet. 60, 320 (1997).
8. C. Vulpe, B. Levinson, S. Whitney, S. Packman, J. Gitschier, Nat. Genet. 3, 7 (1993).
9. J. Chien, M. C. Valentine, V. P. Sybert, J. Am. Acad. Dermatol. 54, S35 (2006).
10. E. E. Norgett et al., Hum. Mot Genet. 9, 2761 (2000).
11. G. McKoy et al., Lancet 355, 2119 (2000).
12. C. Chen, P. E. LeBoit, V. H. Price, J. Am. Acad. Dermatol. 54, 165 (2006).
13. J. H. Robben, N. V. Knoers, P. M. Deen, Am. J. Physiol. Renal Physiol. 289, F265 (2005).
14. V. Greig, C. Linge, G. Terenghi, D. A. McGrouther, G. Burnstock, J. Invest. Dermatol. 120, 1007 (2003).
15. J. D. Sorokina, Z. K. Blandova, Mouse news Lett. J. 730, 23 (1985).
16. M. C. Green, Mouse news Lett. J. 51, 23 (1974).
17. H. P. Baden, M. F. Champliaud, J. P. Sundberg, A. Viel, Genesis 42, 219 (2005).
18. R. D. Miller et al., Genomics 86, 117 (2005).
19. S. Myles et al., Ann. Hum. Genet. Online (2007),
20. J. Schweizer, L. Langbein, M. A. Rogers, H. Winter, Exp. Cell Res. 313, 2010 (2007).
21. Y. Kikkawa et al., Genetics 165, 721 (2003),
22. N. Aoki et al., J. Invest. Dermatol. 116, 359 (2001).
23. P. Yu, D. Ma, M. Xu, Genomics 86, 414 (2005).
24, E. Levy-Nissenbaum et al., Nat. Genet. 34, 151 (2003).
25. D. W. Goodrich, W. H. Lee, Biochim. Biophys. Acta 1155, 43 (1993).
26. H. Song et al., Cancer Res. 66, 10220 (2006).
27. S. Lee et al., Proc. Natl. Acad. Sci, U.S.A. 104, 13732 (2007).
28. H. Herzog, K, Darby, Y. J. Hort, J. Shine, Genome Res. 6, 858 (1996),
29. T. E. Webb, M. G. Kaplan, E. A. Barnard, Biochem. Biophys. Res. Commun. 219, 105 (1996).
30. Q. Li, J. B. Schachter, T. K. Harden, R. A. Nicholas, Biochem. Biophys. Res. Commun. 236, 455 (1997).

Materials and Methods

Clinical Details and DNA Extraction.

Informed consent was obtained from all subjects and approval for this study was provided by the Institutional Review Board of Columbia University. The study was conducted in adherence to the Declaration of Helsinki Principles. Peripheral blood samples were collected from members of 6 Pakistani families as well as 50 unrelated healthy control individuals of Pakistani origin. Genomic DNA was isolated from these samples according to standard techniques (S1).

Linkage Analysis.

A whole genome scan on 10 members of ARWH24 was performed using the Affymetrix 10K SNP array. Genespring GT (Agilent Software) was used for quality control measures and to perform a number of analyses. SNPs displaying Mendelian inheritance errors were removed so that the analyzed dataset contained 9887 variations. Haplotypes were inferred from the data by Genespring GT. By using haplotypes, or clusters of snps that tend to be inherited together, the effect of linkage disequilibrium on multipoint linkage analysis is minimized, reducing type II error. Initial analysis included whole-genome autozygosity mapping to identify regions identical by descent that are shared among affected individuals. Methodology employed by the test was carried out according to the manufacturer's protocol (Agilent Technologies, Santa Clara, Calif.).

Parametric linkage analysis was performed twice, once using snp genotypes and once using haplotypes. All tests assumed a recessive mode of inheritance with 100% penetrance and a disease allele frequency of 0.001.

Two-Point/Multipoint Linkage and TDT Analyses.

Two-point and multipoint linkage analyses were performed for markers genotyped in the region where an excess of homozygosity among affected individuals was observed and for which a mutation was subsequently found. A TDT linkage analysis was also performed using the method implemented in the TDTAE program (S2, S3). The purpose of the linkage and TDT analysis is to provide statistical evidence that this gene is the disease gene for pedigrees in this study. These analyses were performed on a total of 4 pedigrees: ARWH2, ARWH5, ARWH15, and ARWH24. Two-point and multipoint linkage analyses were carried out using the MLINK program of the FASTLINK suite of programs (S4-S6). Two-point LOD scores were computed for recombination fraction values of θ=0.0, 0.01, 0.05, 0.10, 0.20, 0.30 and 0.40. The linkage parameters used were a disease frequency of 0.001, and a fully penetrant autosomal recessive mode of inheritance. Marker allele frequencies were estimated from the data obtained using observed and reconstructed genotypes of founders within the pedigrees. To avoid computational errors due to observed allele frequencies of 0.0, the alleles for markers were re-coded using the RECODE program (S7). This re-coding program insured that alleles were numbered sequentially, and that every allelic frequency was non-zero. In addition, the re-coding had no effect on any of the analyses, in terms of power of the methods. Multipoint analyses were performed using the SIMWALK program version 2.6 (S8). Markers for the multipoint analysis were chosen so that the minimum distance between any two markers was 0.5 cM. This constraint was set to avoid inflation in LOD scores due to linkage disequilibrium among markers (S9, S10). The order and distance between the markers used in the multipoint analysis were deduced from the Marshfield genetic map (S11). For TDT analyses, the genotype relative risk parameters were constrained to follow a recessive mode of inheritance. While the TDTAE program was originally designed to perform TOT analyses when observed genotyping errors are present, the method has also be shown in simulations to be robust to missing parental genotype data (S12). Due to the size and complexity of the pedigrees, pedigrees were first broken into nuclear families before performing TOT analyses. Finally, file format manipulation for linkage and TOT analyses was performed using the methods implemented in the MEGA2 software program (S13).

Mutation Analysis.

Exons of P2RY5 were amplified by PCR with gene-specific primers (Table 4). The PCR products were directly sequenced in an ABI Prism 310 Automated Sequencer, using the ABI Prism Big Dye Terminator Cycle Sequencing Ready Reaction Kit (PE Applied Biosystems). Screening assays for the P2RY5 mutations were performed using genomic DNA from 50 control individuals (Table 5).

TABLE 4
Primers for additional microsatellite markers and for mutation analysis of
genes in the candidate region

| | | | SEQ | | SEQ | product |
|---|---|---|---|---|---|---|
| | | | ID | | ID | size |
| Primer name | Position (bp) | Forward (5′ to 3′) | NO: | Reverse (5′ to 3′) | NO: | (bp) |
| LRCHI-MS | 46,039,578- | GTTCCCTTGGAGGTGAAAATGA | 17 | TAACTTGGATTTATCCAGGCTTTC | 58 | 125 |
| | 46,039,702 | | | | | |
| HTR2A-MS | 46,356,893- | CATCTTGATAAGTGTGAAGTCTTTTG | 18 | GTGTGAGCTCATTCTGGTAAACT | 59 | 136 |
| | 46,357,028 | | | | | |
| FNDC3A-MS | 48,484,903- | TTTATTCCTGGGCACTTGGGTT | 19 | AGAGTATAGCAAGGTTGCTGGA | 60 | 156 |
| | 48,485,058 | | | | | |
| P2RY5 ex1 | | CTTTGGTTCCTTAATTAGAGGCCAA | 20 | CCAAGTGCTGGGATTCCATG | 61 | 357 |
| P2RY5 ex 2 | | GTCAGGAGTTCAAGACCAGC | 21 | GTTGTATATGCTGATCTCATCCTAC | 62 | 408 |
| P2RY5 ex3-1 | | CCTAGGTATATTCCCAGCAGAC | 22 | CAACGAGTCCAACCCATAGGATT | 63 | 537 |
| P2RY5 ex3-2 | | GACAAGTGGTTTCATTCTGGTC | 23 | TCCAAATGGCCAATTCCGTG | 64 | 621 |
| P2RY5 ex3-3 | | TCCCAAAGGAGACTGCAGCT | 24 | GTTTTCCATGTGGCTTCTGG | 65 | 598 |
| P2RY5 ex3-4 | | CACGGAATTGGCCATTTGGA | 25 | CAGCAATACAGAGAGTGATTGG | 66 | 606 |
| P2RY5 ex3-5 | | CCAGAAGCCACATGGAAAAC | 26 | GACACTTTTCACAGTTGAAGGAACT | 67 | 581 |
| NUDT15 ex1 | | TAGTGAGCGCGTCACTTCCT | 27 | AAGCTTTGCACGGCGTGAGTT | 68 | 275 |
| NUDT15 ex2 | | AGCCACATGCCCAGCTGATT | 28 | CAGACCACTTGCTCTCCTGA | 69 | 460 |
| NUDT15 ex3 | | GGTTAGCTTACCCAAATAAAGACC | 29 | TTCCCTAACCAGACCTTATTCTTG | 70 | 271 |
| MED4 ex1 | | ACCTGCGTCAGCTCGCTCT | 30 | CGAGAACGAGCACAAACGCA | 71 | 264 |
| MED4 ex2 | | AGTTCACAGTCAATAGCGGACT | 31 | CATTCCTGTTCCCACACAAAGTA | 72 | 273 |
| MED4 ex3 | | ATAGCCTAGCCAATTCAGTTACG | 32 | CTGGCTTTTGGTGAGTGACAAG | 73 | 407 |
| MED4 ex4 | | ATAGTCTGGGTGTCTTCTGCAT | 33 | CCAGAACCTTAACTTTTCAGGTC | 74 | 411 |
| MED4 ex5 | | AAGGAAGGCAGGTGACTGAG | 34 | TATGTGGCAGAGGTTTCTAAGG | 75 | 340 |
| MED4 ex6 | | GAACCAGCGAAAATGAGTATGTC | 35 | ATTCCAACCTTGTCATCTCTGG | 76 | 285 |
| MED4 ex7 | | GCATAAAGGGAAAAGTAAGAACTAG | 36 | CCTGTAGTTTACATTTCCCTGCT | 77 | 329 |
| RCBTB2 ex1 | | GCGAGAACATAACCCTGGAG | 37 | CCAGTGGTACCTGACAGCAT | 78 | 435 |
| RCBTB2 ex2 | | CAAGACAGTATATTCTTTTCCTTCTG | 38 | TACTGACTGGCTGGGAGCAT | 79 | 207 |
| RCBTB2 ex3 | | GTAGGTGATGTGGGCATGGT | 39 | CATTTCAGAGTTGACTTCCTGTG | 80 | 305 |
| RCBTB2 ex4 | | CTGCCTGGTTCCATCGAACA | 40 | CCTATCGGTGACCAATAGGTG | 81 | 206 |
| RCBTB2 ex5 | | CAGAACTGAAGGGGCAGAGAA | 41 | CCCCAAGATCTTCAATTTGGCAA | 82 | 295 |
| RCBTB2 ex6 | | GGAAGATGATTCCTGTTTGGATG | 42 | TTCTCAAGGGCAGCTGCGAA | 83 | 287 |
| RCBTB2 ex7 | | TCATTTGTGGAAGCTGTGCTACA | 43 | CCAGGACTACTATAGAAGTCTAC | 84 | 323 |
| RCBTB2 ex8 | | GTCAGTGTCCAGTTGAGACAG | 44 | GTGGGACATACCATACTGGC | 85 | 301 |
| RCBTB2 ex9 | | CCACATCAGTCTCATGCCAG | 45 | TGATATCTTTGCCAGCGAAGACT | 86 | 264 |
| RCBTB2 ex10 | | CCTACTTTGTGGAAGCAGTGAAA | 46 | ATCACAATCACCAAAGACCAGTG | 87 | 261 |
| RCBTB2 ex11 | | ACTTGTTTCTGCCCCGGTCA | 47 | CCCTGAGCTCTTTCCCAGAT | 88 | 302 |
| RCBTB2 ex12 | | AGTACCAGTAGAGTAGTGCCATA | 48 | CCGCTCCACTGTATGCTTGT | 89 | 353 |
| RCBTB2 ex13 | | TCACAGAGGCCATCTGTCCT | 49 | TAGAAACCACCTTGTCAGTTCCT | 90 | 264 |
| RCBTB2 ex14 | | AGTGGCTGAGTGAGCAACTG | 50 | GCAGACACCTGGGTTCCATT | 91 | 262 |
| RCBTB2 ex15 | | ATGCCATCATTTCCTCCACTTG | 51 | CTCTGGCTTTTGCAGGGGAA | 92 | 325 |
| CYSLTR2 ex1 | | CCTAGAGAGATGTAATCAGTAAGC | 52 | GACTATGAGTGAATGAAGATGGAC | 93 | 1185 |
| LOC647131 ex1 | | CACACACTAGGCCAGTTTTAG | 53 | CAGCAACTACTTTTGTTGAGCCA | 94 | 400 |
| LOC647131 ex2 | | AGTCCCTCTTCTGCTCGCAA | 54 | CCGAACTTGAACGCTTTTCTGAA | 95 | 295 |
| LOC647131 ex3 | | CTGTTGGGGTTGTTTCCTGG | 55 | GTTCGCGTGCTTTCGGAAAG | 96 | 523 |
| FNDC3A ex1 | | TTGATCCTGCCTGGCTGCTT | 56 | AGTCCCTCTTCTGCTCGCAA | 97 | 219 |
| FNDC3A ex2 | | CCTTTGACAAGAATATCTCTTTAGGTT | 57 | CACGAATTAACAGGAACTCACTATTG | 98 | 346 |

TABLE 5
Primers and restriction enzymes for screening assays of P2RY5 mutations

| | | | | | size of the | |
|---|---|---|---|---|---|---|
| | | | | | digested fragments | |
| | | | product | | (bp) | |
| | forward primer | reverse primer | size | | wild type | mutant |
| mutation | (5′ to 3′) | (5′ to 3′) | (bp) | enzyme | allele | allele |
| 69insCATG | TCCCAAAGGAGACTGCAGCT | TCCAAATGGCCAATTC | 317 | NsiI | 317 | 184,133 |
| | (SEQ ID NO: 99) | CGTG | | | | |
| | | (SEQ ID NO: 104) | | | | |
| 172-175delAACT; | CTGCGTCCTCAAAGTCCGAA | GGTAGACAATTGCCAG | 233 | MseI | 151, 42, | 193, 40 |
| 177delG | (SEQ ID NO: 100) | AAATCGA | | | 40 | |
| | | (SEQ ID NO: 105) | | | | |
| 188A > T (D63V) | CAACTTACATGATTAACTTG | GGTAGACAATTGCCAG | 204 | MlyI | 204 | 167, 37 |
| | GCAATGTGAG | AAATCGA | | | | |
| | (C > G change is | (SEQ ID NO: 106) | | | | |
| | introduced) | | | | | |
| | (SEQ ID NO: 101) | | | | | |
| 562A > T (I168F) | CACGGAATTGGCCATTTGGA | CAGCAATACAGAGAGT | 605 | BstBI | 606 | 330, 276 |
| | (SEQ ID NO: 102) | GATTGG | | | | |
| | | (SEQ ID NO: 107) | | | | |
| 565G > A (E189K) | CACGGAATTGGCCATTTGGA | CAGCAATACAGAGAGT | 606 | TaqI | 276, 226, | 502, 104 |
| | (SEQ ID NO: 103) | GATTGG | | | 104 | |
| | | (SEQ ID NO: 108) | | | | |

RT-PCR.

Total RNA was isolated from 10 plucked human scalp hairs of a healthy control individual using the RNeasy® Minikit (Quiagen). 2 μg of total RNA was reverse-transcribed with random primers and SuperScript™ III (Invitrogen). The cDNAs were then amplified by PCR using P2RY5-specific primers (forward 5′-CATCTACAAAGAACCAAGAATTGTGAG-3′ [SEQ ID NO: 11]; reverse 5′-TCCAAATGGCCAATTCCGTG-3′ [SEQ ID NO: 12]). The amplification conditions were 94° C. for 2 min, followed by 40 cycles of 94° C. for 30 sec, 58° C. for 30 sec and 72° C. for 90 sec, with a final extension at 72° C. for 7 min. GAPDH mRNA was amplified as a control. PCR products were run on 1.5% agarose gel.

Indirect Immunofluorescence (IIF), Cell Culture and Western Blots (WB).

IIF on frozen sections of skin and individually dissected hair follicles as well as culture of normal human keratinocytes and WB were performed as described previously (S14), The primary antibodies used were rabbit polyclonal anti-P2Y5 (diluted 1:200 for HF and 1:1,000 for WB; MBL international), guinea pig polyclonal anti-K6irs3 and anti-K6hf (1:2,000; generous gifts from Drs. Lutz Langbein and Jürgen Schweizer, Heidelberg, Germany), rabbit polyclonal anti-cytokeratin 1 (1:5,000; Covance), and rabbit polyclonal anti-β-actin (1:5,000; Sigma).

### REFERENCES

S1. J. Sambrook, E. F. Fritsch, T. Maniatis, Molecular Cloning: A Laboratory Manual. 2nd edn New York, N.Y.: Cold Spring Harbor Laboratory Press (1989).
S2. D. Gordon et al., Eur. J. Hum. Genet. 12, 752 (2004).
S3. D. Gordon, S. C, Heath, X. Liu, J. Ott, Am. J. Hum. Genet. 69, 371 (2001).
S4. G. M. Lathrop, J. M. Lalouel, C. Julier, J. Ott, Proc. Natl. Acad. Sci. U.S.A. 81, 3443 (1984).
S5. R. W. Cottingham, R. M. Idury, A. A. Schaffer, Am. J. Hum. Genet. 53, 252 (1993).
S6. A. A. Schaffer, S. K. Gupta, K. Shriram, R, W. Cottingham, Jr., Hum. Hered. 44, 225 (1994).
S7. D. E. Weeks, RECODE—a program for recoding base-pair sized alleles into integer-numbered alleles. (2000).
S8. E. Sobel, K. Lange, Am. J. Hum. Genet. 58, 1323 (1996).
S9. A. L. Boyles et al., Hum. Hered. 59, 220 (2005).
S10, D. J. Schaid, S. K. McDonnell, L. Wang, J. M. Cunningham, S. N. Thibodeau, Am. J. Hum. Genet. 71, 992 (2002).
S11. K. W. Broman, J. C. Murray, V. C. Sheffield, R. L. White, J. L. Weber, J. Am. J. Hum. Genet. 63, 861 (1998).
S12. S. Barral, C. Haynes, M. A. Levenstien, D. Gordon, BMC Genet. 6, S150 (2005).
S13. N. Mukhopadhyay, L. Almasy, M. Schroeder, W. P. Mulvihill, D. E. Weeks, Bioinformatics 21, 2556 (2005).
S14. H. Bazzi et al., Differentiation 74, 129 (2006).

### Example 3

### Knock-Down of Human P2RY5 mRNA Using RNAi and Real-Time PCR

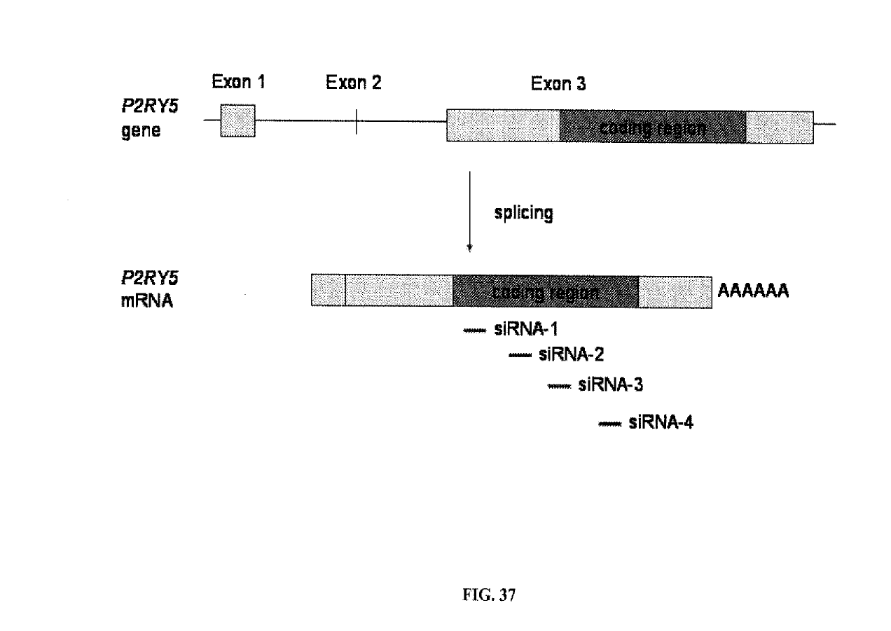

A total of 4 distinct siRNAs were designed using the siRNA design tool in Dharmacon (Chicago, Ill.), and purchased from the company. The target sequences for all four siRNAs are located within the coding region of the P2RY5 (FIG. 37). The sense strand sequence of each siRNA is:

siRNA-1:
[SEQ ID NO: 13]
5′-GGUGUUUGUGCUUGGGUUAtt-3′;
(NM_005767: nt 877-895)
siRNA-2:
[SEQ ID NO: 14]
5′-UGCACUGGCGUGUGGUUAAtt-3′;
(NM_005767: nt 1208-1227)
siRNA-3:
[SEQ ID NO: 15]
5′-GGGUAACAAUGCCUCAGAAtt-3′;
(NM_005767: nt 1279-1297)
and
siRNA-4:
[SEQ ID NO: 16]
5′-GUGGCAGCAGUAAGGACAAtt-3′.
(NM_005767: nt 1595-1613)

These siRNAs were transfected into HaCat cells at 30% confluency using Oligofectamine (Invitrogen). Similarly, non-targeting (NT) siRNA was purchased from Dharrnacon and was used as the calibrator sample in all experiments. 48h after transfection, the cells were harvested and total RNAs were isolated from the cells using RNeasy® Minikit (Qiagen), which were treated with DNase I in order to completely remove DNA. After that, 2 ug of the total RNA was reverse transcribed with oligo-dT primers and the SuperScript™ III reverse transcriptase (Invitrogen). cDNA was assayed for relative levels of P2RY5 and β-actin message via real time PCR. β-actin was used to normalize for slight variations in cDNA concentration in each sample. All samples were run in triplicate using the Relative Quantification Plate Assay in the ABI 7300 Real-Time PCR System and analyzed using the ABI Relative Quantification Study Assay.

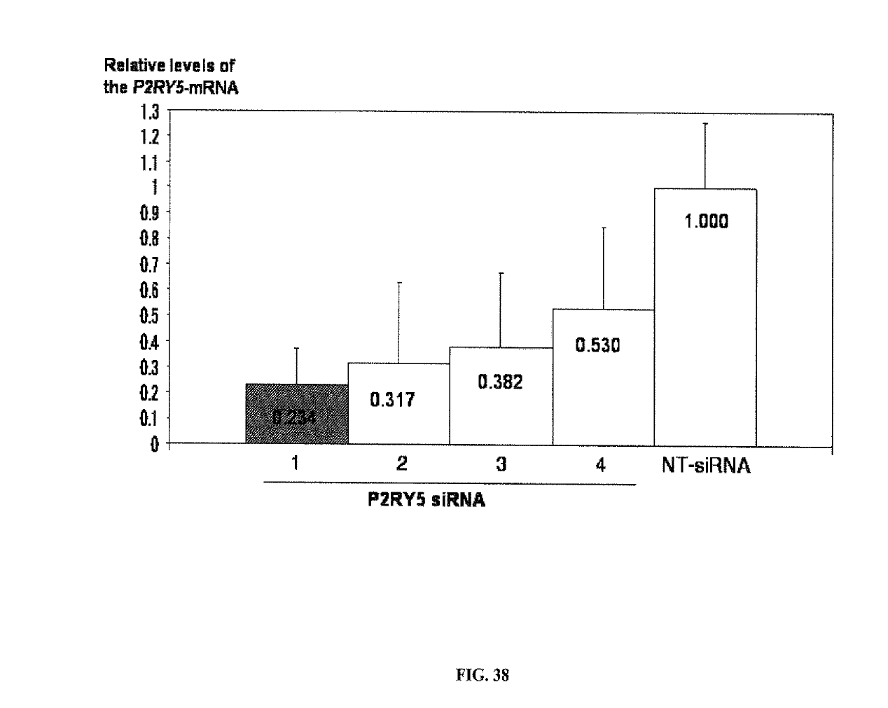

As shown in FIG. 38, all four siRNAs against P2RY5 significantly reduced the expression levels of the P2RY5 mRNA. In particular, siRNA-1 led to approximately 75% reduction of the P2RY5 expression.

### Example 4

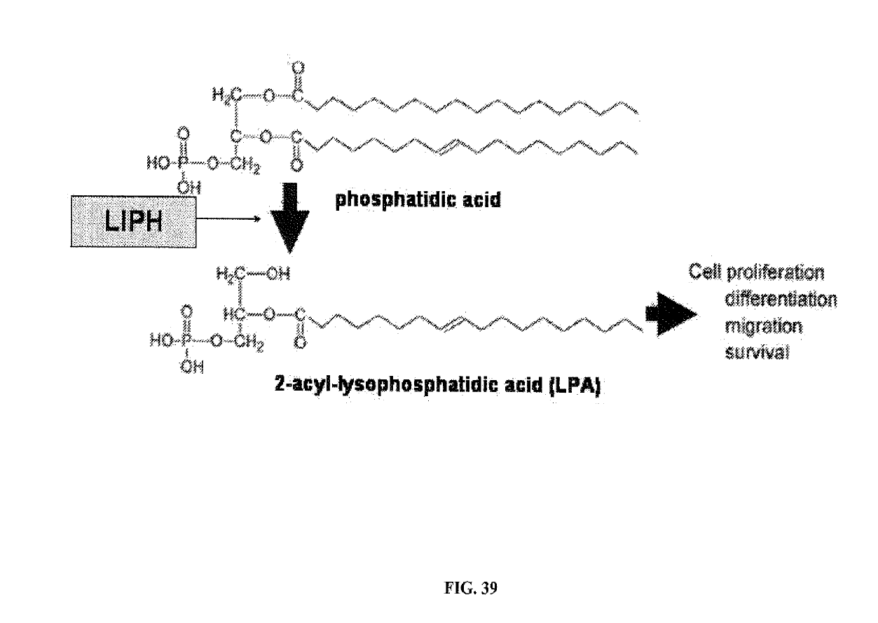

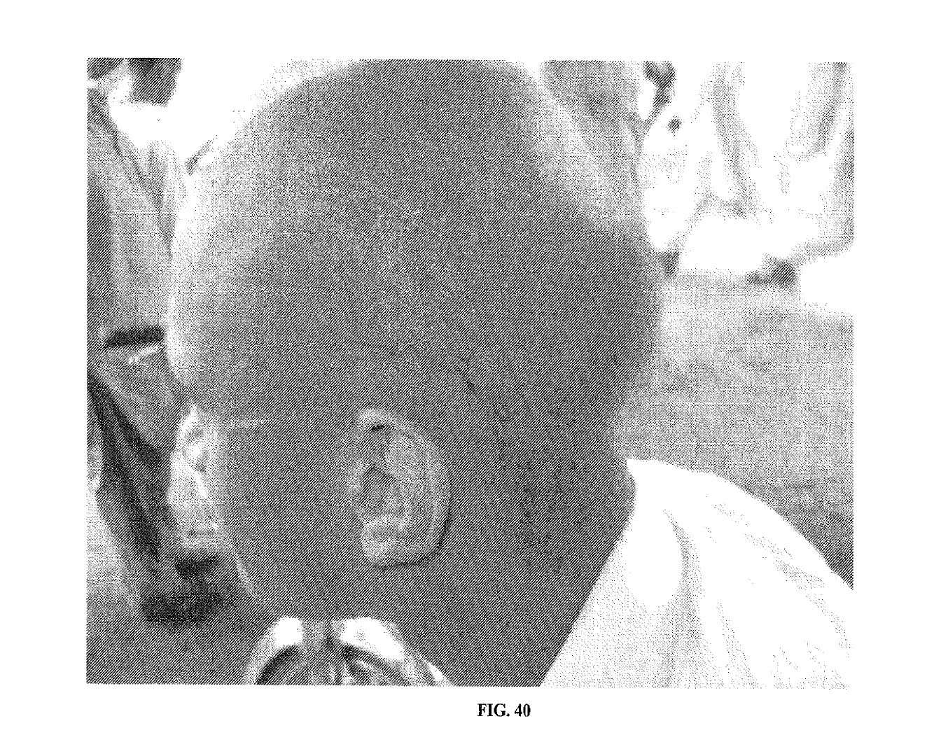

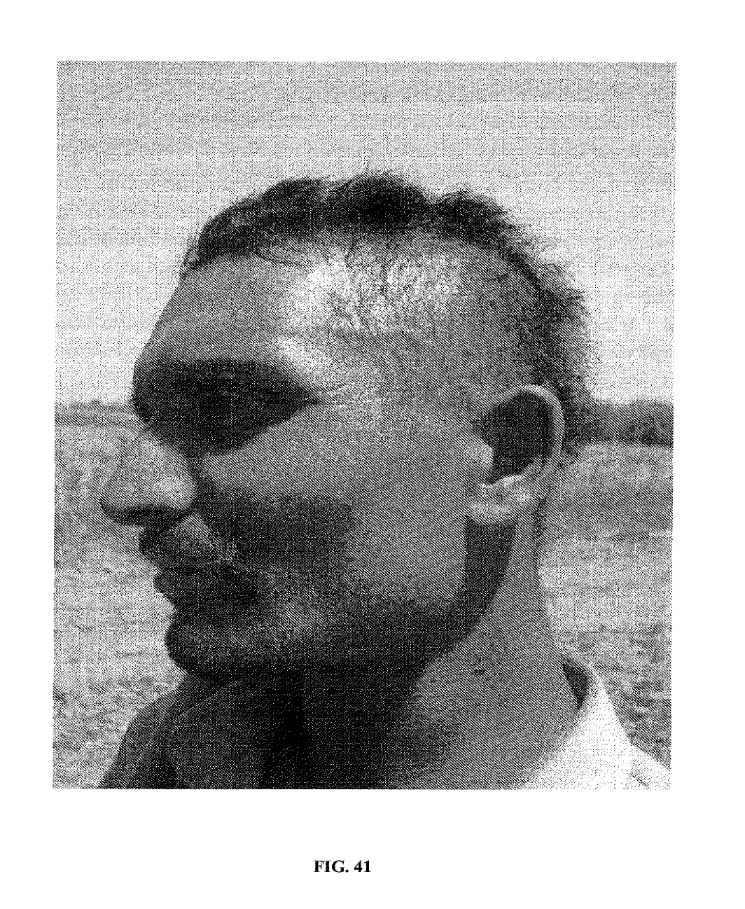

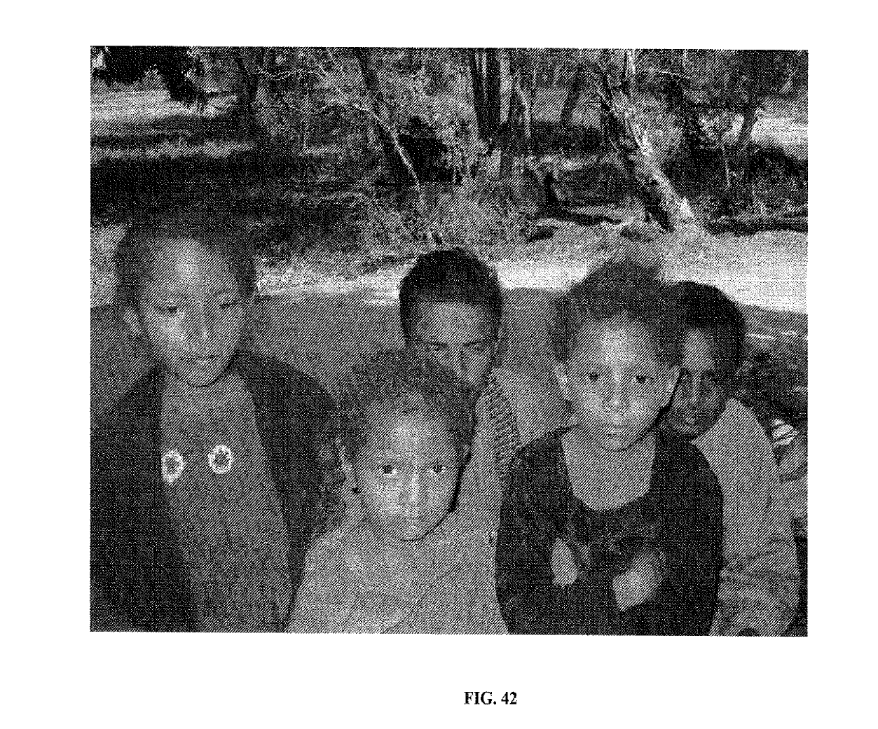

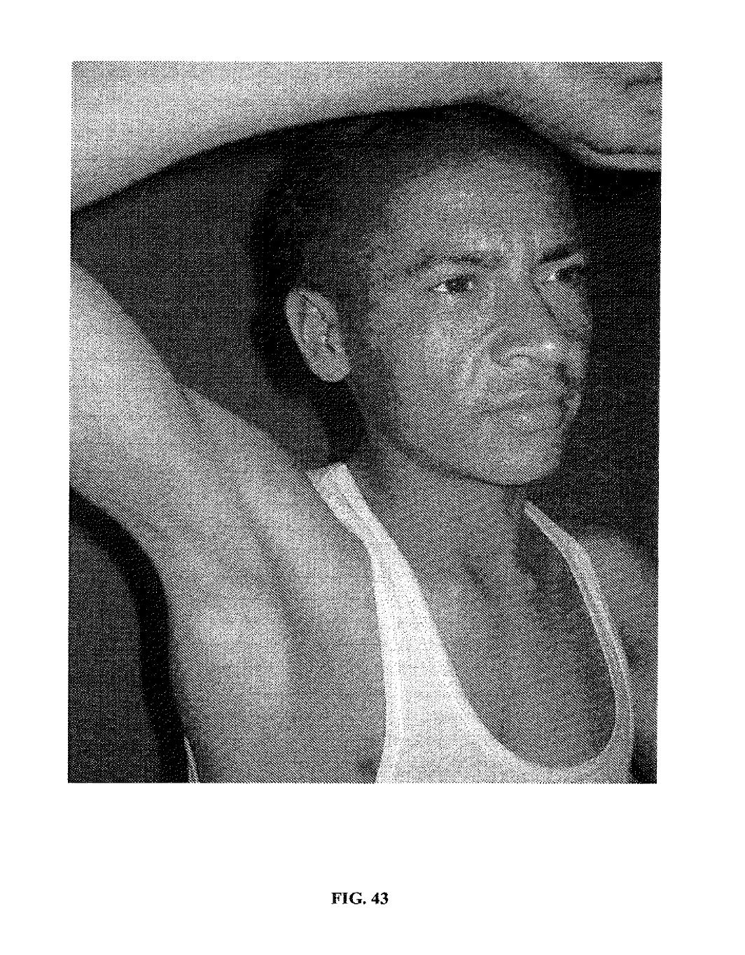

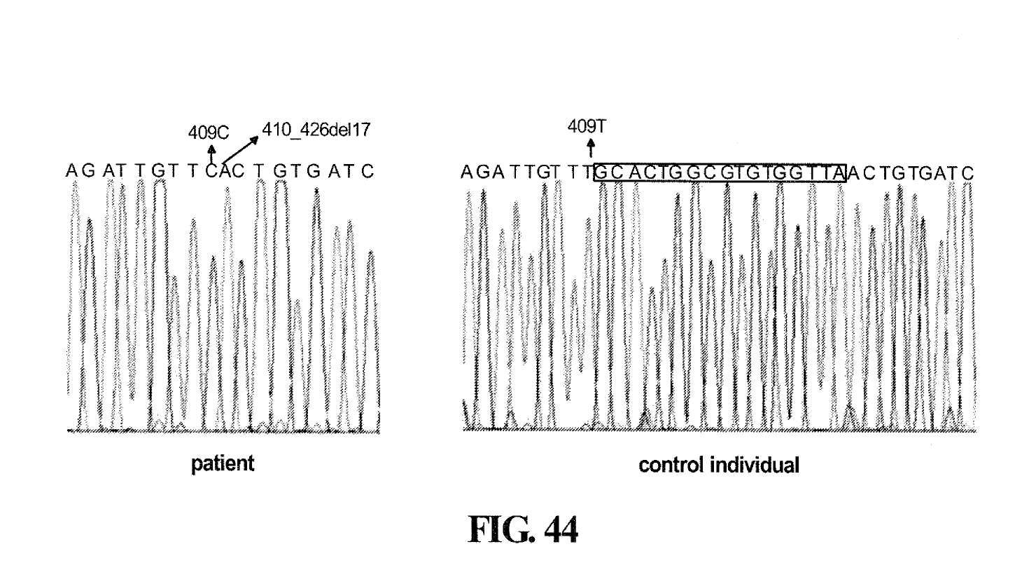

In 2002, two groups cloned the LIPH gene encoding a new member of phospholipase A1 named lipase H (alternatively known as membrane-associated phosphatidic acid-selective phospholipase A1 alpha) on human chromosome 3q27-q28 (Sonoda et al, 2002; Jin et al, 2002). Sonoda et al. have shown that lipase H produces the 2-acyl-lysophosphatidic acid (2-acyl-LPA) from phosphatidic acid (Sonoda et al, 2002) [See FIG. 39].

LPA is an extracellular lipid mediator that shows multiple biological functions such as cell proliferation, antiapotptic activity, and cytoskeletal organization (Sonoda et al, 2002). Recently, a homozygous mutation in the LIPH gene has been shown to cause an autosomal recessive hypotrichosis (Kazantseva et al, 2006). However, the precise expression and the role of lipase H in the hair follicles remain largely unknown.

The human P2RY5 gene has 2 introns and is ˜4.7 kb in size, and is not well studied in the literature. P2RY5 was originally reported as the human homolog of a chicken T-cell-specific receptor gene, known as 6H1. P2RY5 is seven-pass transmembrane protein that is an orphan member of the G-protein coupled receptor (GPCR) superfamily, and by sequence homology bears most resemblance to the P2Y family of purinergic GPCRs (Burnstock G., Introduction: P2 receptors. Curr Top Med Chem. 2004; 4(8):793-803). P2RY5, along with P2RY9 and P2RY10, while bearing homology to the purinergic GPCRs, do not function in this manner. Although P2RY5 was originally reported to exhibit nucleotide-promoted second messenger responses (Webb T E, Kaplan M G, Barnard E A. Identification of 6111 as a P2Y purinoceptor: P2Y5. Biochem Biophys Res Commun. 1996 Feb. 6; 219(1):105-10), a later report refuted these findings (Li Q, Schachter J B, Harden T K, Nicholas R A. The 6H1 orphan receptor, claimed to be the P2RY5 receptor, does not mediate nucleotide-promoted second messenger responses (Biochem Biophys Res Commun. 1997 Jul. 18; 236(2):455-60), thus its true function(s), ligands, and the intracellular signaling cascade that it modulates (if any) remain to be determined. In one instance, the nearest relative of P2RY5, known as P2RY9/GPR23, has been shown to be receptor (e.g., a GPCR coupled to a G-protein, for example, Gq, Gs, Gi, Gz, or G_{12/13}) for lysophosphatidic acid (LPA). Lipase H is a key enzyme in the synthesis of LPA, and mutations in LIPH cause a recessively inherited form of hypotrichosis. Perhaps P2RY5 functions as an LPA receptor in the hair follicle IRS, since mutations in the synthesis of the ligand, LPA, as well as a potential receptor, P2RY5, cause similar phenotypes. To our knowledge, P2RY5 is the first GPCR to be implicated in a human hair disorder, rendering it a druggable target using small molecules. Therapeutic manipulation of P2RY5 could be envisaged for the treatment of excessive or unwanted hair, or as an alternative to current methods of changing the texture of the hair fiber, such as chemical straightening or permanent waving. Our findings indicate that disruption of P2RY5 underlies autosomal recessive woolly hair, and more broadly, uncover a novel gene involved in the determination of hair texture in humans.

### REFERENCES

1. Sonoda H, Aoki J, Hiramatsu T, Ishida M, Bandoh K, Nagai Y, Taguchi R, Inoue K, Arai H (2002) A novel phosphatidic acid-selective phospholipase A1 that produces lysophosphatidic acid. J Biol Chem 277:34254-34263.
2. Jin W, Broedi U C, Monajemi H, Glick J M, Rader D J (2002) Lipase H, a new member of the triglyceride lipase family synthesized by the intestine. Genomics 80:268-273.
3. Kazantseva A, Goltsov A, Zinchenko R, Grigorenko A P, Abrukova A V, Moliaka Y K, Kirillov A G, Guo Z, Lyle S, Ginter E K, Rogaev E I (2006) Human hair growth deficiency is linked to a genetic defect in the phospholipase gene LIPH. Science 314:982-985.

### Example 5

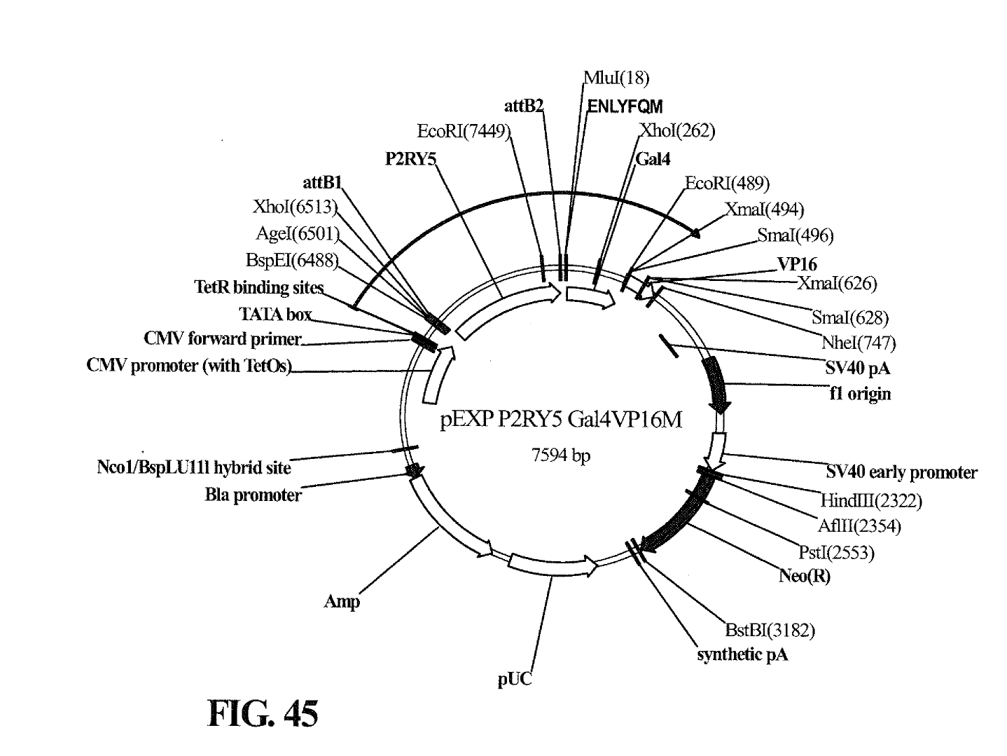

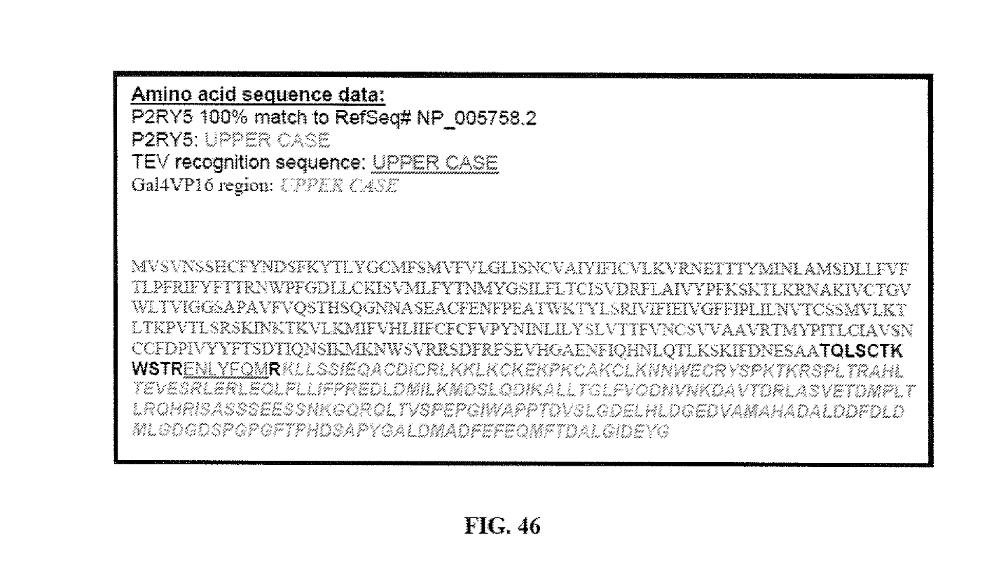

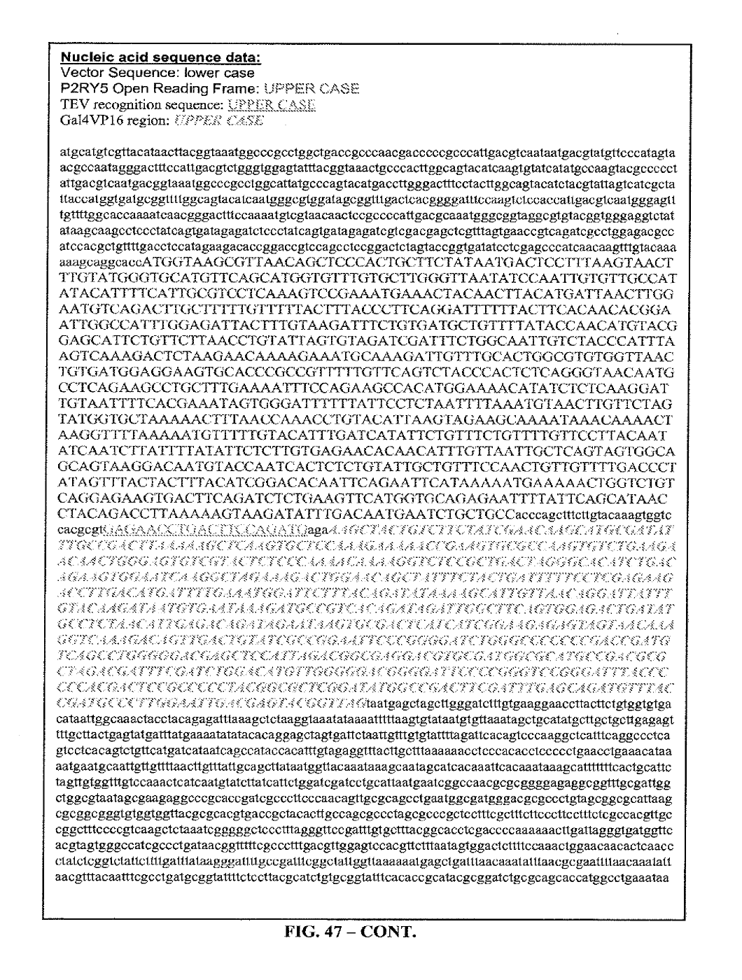

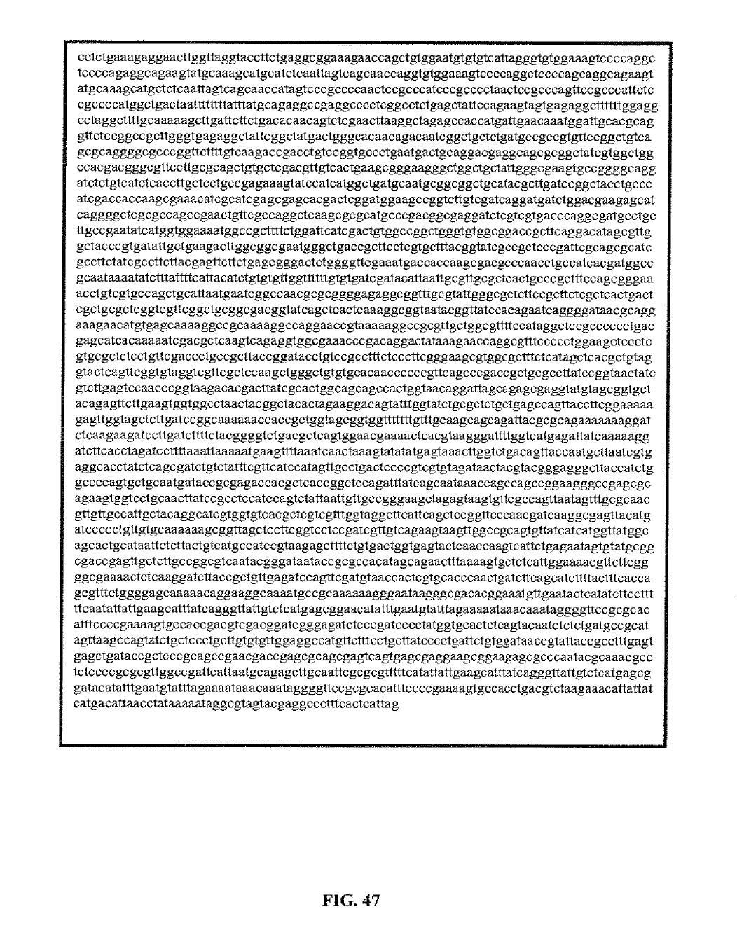

A pENTR221 P2RY5 construct obtained from Invitrogen (matching NM_{—}005767.4) was amplified to remove the stop codon and re-cloned via B×P reaction into a pDONR221 vector backbone. The resulting DNA was subcloned into a Tango™ pDEST Gal4VP16M expression vector containing a geneticin selection marker (FIG. 47). The inserted P2RY5 sequence and junctions were fully sequenced to verify that no mutations had occurred (FIG. 46). A map of the resulting expression construct is shown in FIG. 45.

### Example 6

The vector described in Example 5 was stably integrated into the Tango™ GPCR U2OS parental cell line and to evaluate the receptor cell line for a response to LPA, serum, or PMA. Additionally, single cell clones and a pooled cell line will be isolated by flow cytometry.

Stable Pool Generation.

The Tango™ GPCR U2OS parental cell line (Invitrogen) was transfected with the Tango™ P2RY5 expression plasmid that was described in Example 5. The transfected cells were selected with 200 ug/ml Geneticin® for two weeks until an untransfected control cell culture was dead. The resulting selected pool was then maintained with 100 ug/ml geneticin in culture.

Stable Pool Agonist Testing.

The Tango™ P2RY5 pool of cells was tested for an inducible response to LPA, serum, and PMA.

LPA Activity Testing.

The Tango™ P2RY5 U2OS antibiotic selected pool cells were plated at 10,000 cells/well into a 384 well clear bottom assay plate in Freestyle™ media (Invitrogen). C-LPA (1-oleoyl-sn-glycero-2,3-cyclic-phosphate) or LPA (1-(9Z-octadecenyl)-2-hydroxy-snglycero-3-phosphate) (in Freestyle™ media) was then added to the plate to a final concentration of 10 uM. The plate was placed at 37° C. 5% CO2 for 24 hours for receptor stimulation followed by betalactamase substrate loading for 2 hours.

FBS and PMA Activity Testing

The Tango™ P2RY5 U2OS antibiotic selected pool cells were plated at 10,000 cells/well into a 384 well clear bottom assay plate in Freestyle™ media. FBS (Fetal Bovine 20%) or a PMA concentration dilution series (in Freestyle™ media) was then added to the plate at the indicated concentrations. The plate was placed at 37° C. 5% CO2 for 24 hours for receptor stimulation followed by betalactamase substrate loading for 2 hours. (The P2RY5 cells were also tested with a 48 hour stimulation time with equivalent results.)

Stable Pool Sorting for FBS Response

The antibiotic selected Tango™ P2RY5 U2OS cells were cultured to 80-90% confluence in tissueculture flasks. The growth media was then removed and replaced with Freestyle™ media either with (stimulated) or without (un-stimulated) 20% FBS, The cultures were then placed at 37° C. 5% CO2 for 48 hours for receptor stimulation followed by beta-lactamase substrate loading for 2 hours. Responsive cells were then sorted by FACS into a single pool and three 96 well plates of clones.

Results.

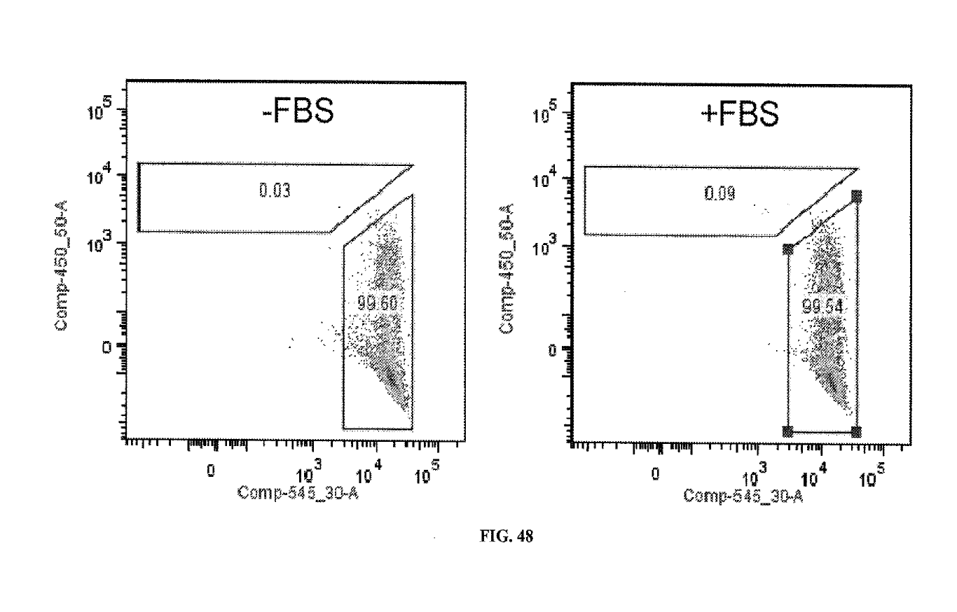

FBS induced a slight beta-lactamase response via the P2RY5 receptor. Responsive cells were sorted into single cell clones and a pooled population (FIG. 48).

Sorted Pool Tested for a Response to FBS

The Tango™ P2RY5 U2OS cells were plated at 10,000 cells/well into a 384 well clear bottom assay plate in Freestyle™ media. FBS was then added to half the wells in the assay to a concentration of 20% (stimulated) while the other half of the assay received an equal volume of Freestyle™ Media (un-stimulated). The plate was placed at 37° C. 5% CO2 for 48 hours for receptor stimulation followed by betalactamase substrate loading for 2 hours.

Results.

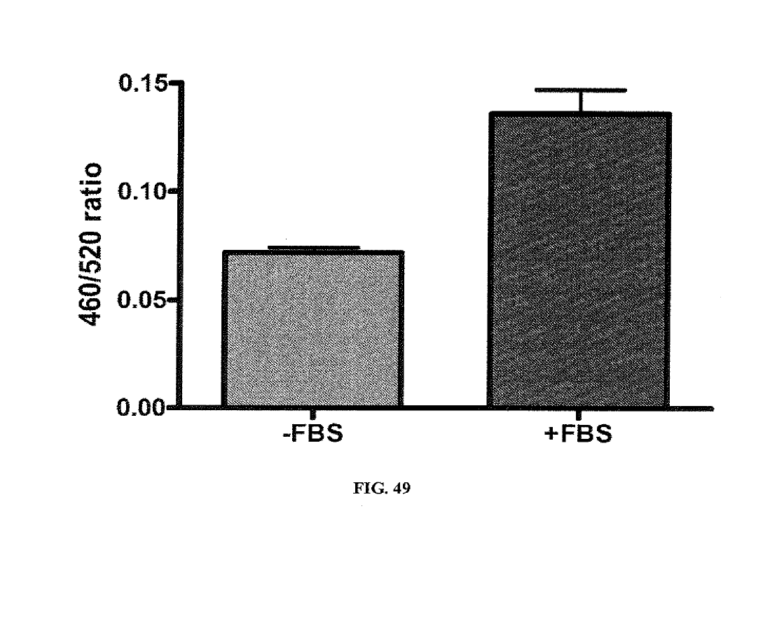

FBS induced a beta-lactamase response in the P2RY5 sorted pool that was greater than the response demonstrated by the original antibiotic selected pool (FIG. 49).

Cell Line Validation—P2RY5 Receptor Expression

The Tango™ P2RY5 antibiotic selected pool and the Tango™ GPCR U2OS parental cell line were cultured for 24 hours in a cover slip tissue culture chamber to allow for cell attachment. The cells were then fixed, permeabilized and immunostained using a VP16 specific primary antibody and Alexa Fluor® 488 conjugated secondary antibody. Fluorescent micrographs were taken using an appropriate fluorescent filter set. Exposure times were identical for both photos.

Results.

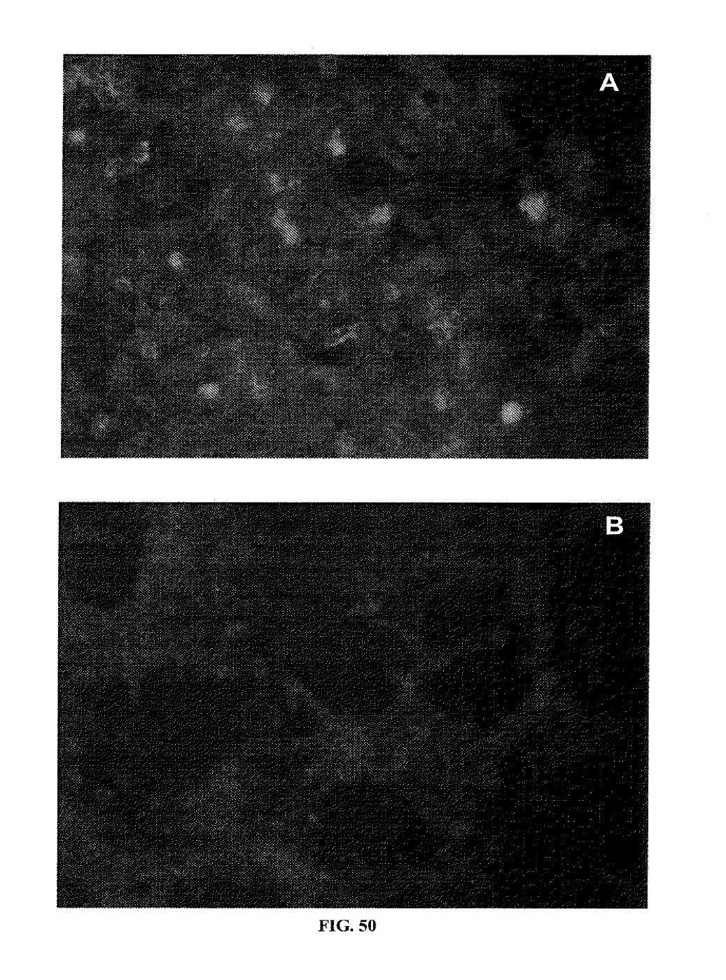

The Tango™ modified P2RY5 is expressed in the antibiotic selected pool (FIG. 50A).

Cell Line Validation—TEV Protease Transfection

The Tango™ P2RY5 cells were plated at 50,000 cells/well into a 96 well clear bottom assay plate in Freestyle™ media. The plate was placed at 37° C. 5% CO2 for 24 hours. The cells were then transfected with a TEV protease expression plasmid. The plate was then placed at 37° C. 5% CO2 for 48 hours followed by beta-lactamase substrate loading for 2 hours.

Results.

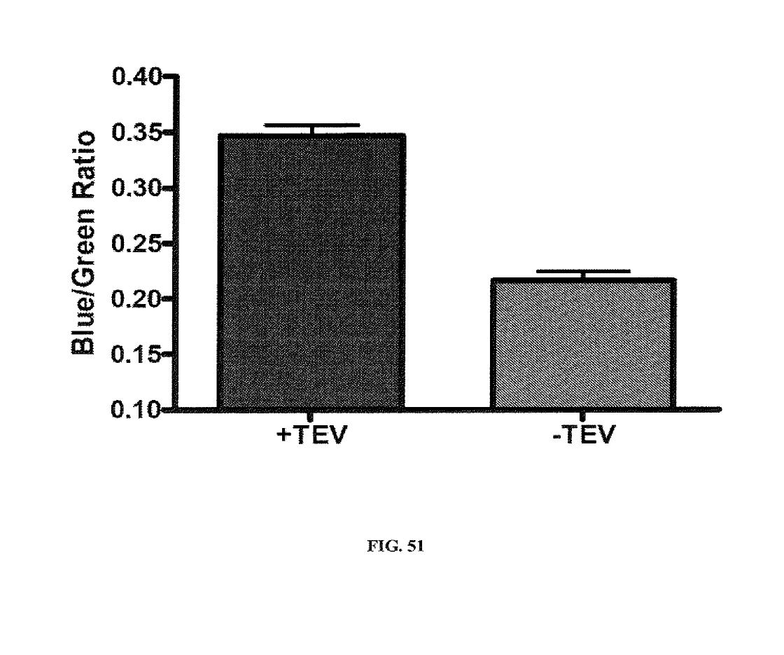

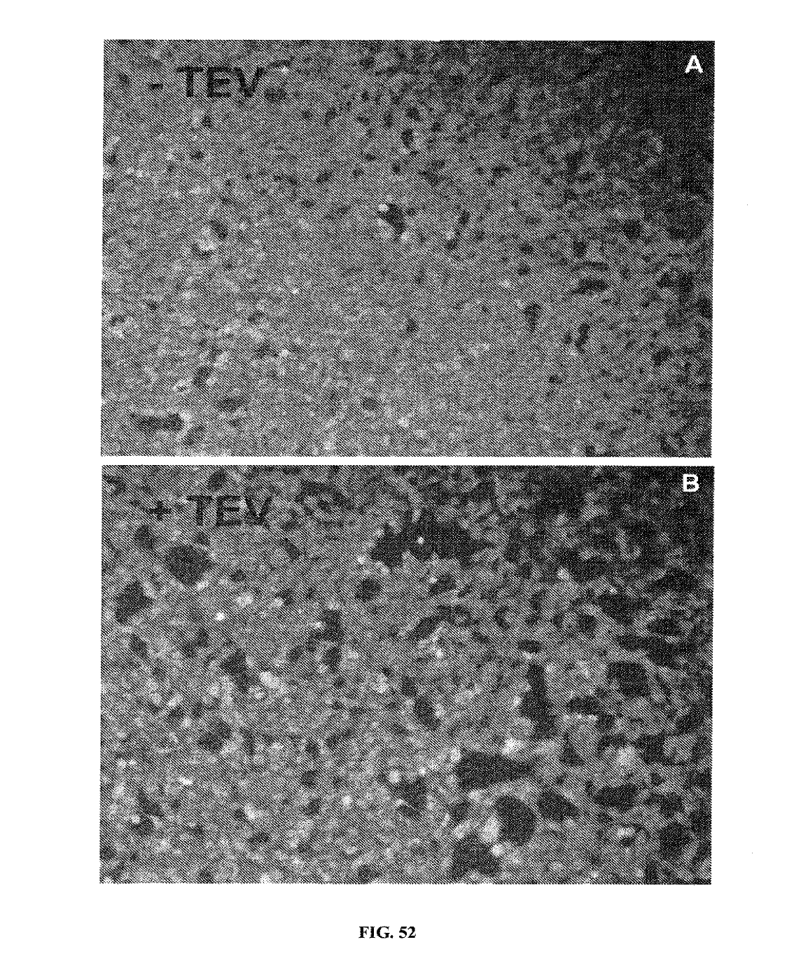

TEV protease transfection can stimulate beta-lactamase reporter activity (FIG. 51 and FIG. 52).

Additional FACS Sorting of Blue Pool

The initial sorted pool demonstrated a slight increase in response to FBS. An additional round of sorting was required to increase the level of ligand induced response of the cell line. The cell pool from the 1^{st} sort was stimulated with dFBS and analyzed for receptor activation via FACS. The cells demonstrated significant activation and were sorted into pooled and cloned cell populations.

The antibiotic selected FACS sorted responsive cell Tango™ P2RY5 U2OS Pool was cultured to 80-90% confluence in two tissue culture flasks, The growth media was then removed and replaced with Freestyle™ media either with (stimulated) or without (un-stimulated) 20% FBS. The cultures were then placed at 37° C. 5% CO₂ for 48 hours for receptor stimulation followed by beta-lactamase substrate loading for 2 hours. Responsive cells were then sorted by FACS into a green cell pool from the un-stimulated cell sample, and into a blue cell pool and three 96 well plates of clones from the FBS stimulated sample.

Results.

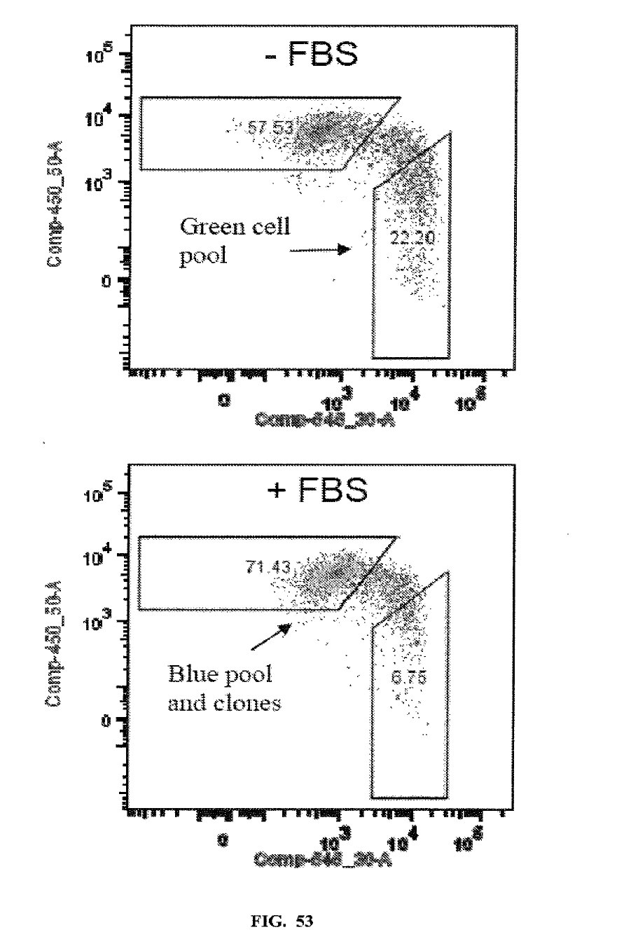

FBS was able to induce a beta-lactamase response through the P2RY5 receptor. Responsive cells were sorted into single cell clones and green un-stimulated and blue stimulated pooled cell populations were collected (FIG. 53).

FES Activity Testing

The Tango™ P2RY5 pools of cells collected were tested for a response to LPA, serum, and PMA.

The Tango™ P2RY5 U2OS sorted pool cells were plated at 10,000 cells/well into a 384 well clear bottom assay plate in FreeStyle™ media. The dFBS was immediately added to plate to final concentration of 20% by volume (stimulated). An equal volume of Free Style™ media was added to the un-stimulated wells. The plate was placed at 37° C. 5% CO₂ for 48 hrs for receptor stimulation followed by beta-lactamase substrate loading for 2 hours.

Results.

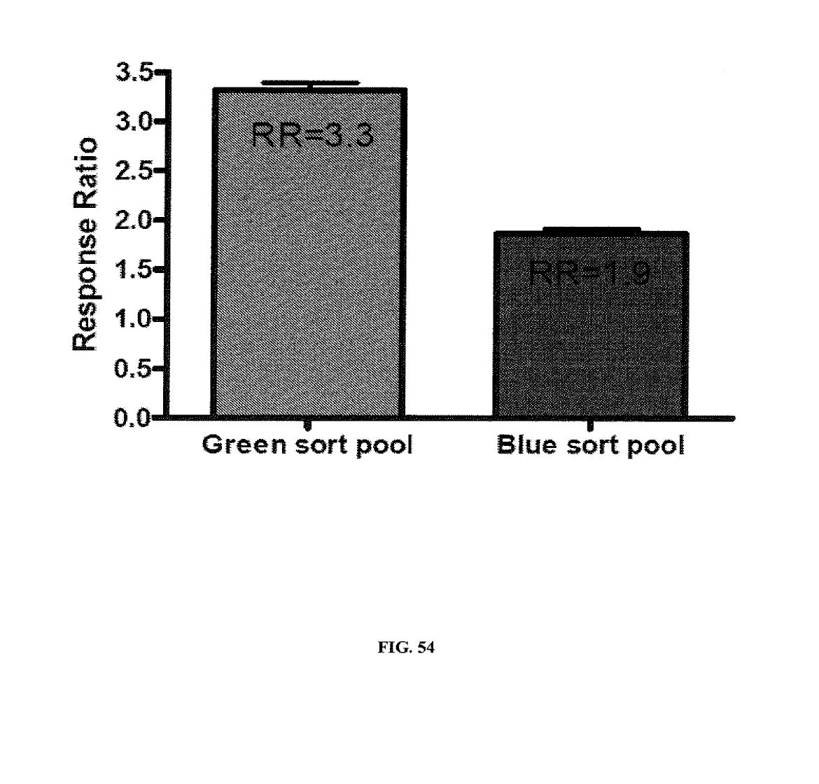

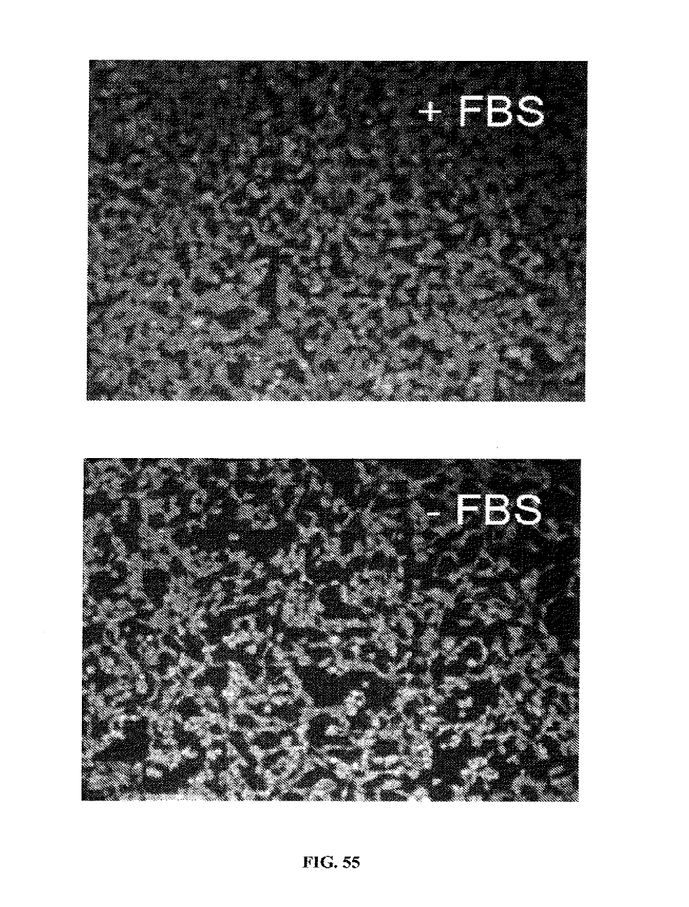

Both the blue and green sorted pools demonstrated an inducible beta-lactamase response to FBS (FIG. 54 and FIG. 55). (The response ratio value is calculated by the division of the 460/530 ratio of the stimulated wells by the 460/530 ratio of the un-stimulated control cells. A response ratio equal to one is indicative of no functional response.)

LPA, cLPA and PMA Activity Testing

The Tango™ P2RY5 U2OS sorted pool cells were plated at 10,000 cells/well into a 384 well clear bottom assay plate in FreeStyle™ media. The plate was placed at 37° C. 5% CO₂ for 20 hours prior to receptor stimulation. cLPA (1-oleoyl-sn-glycero-2,3-cyclic-phosphate), LPA (1-oleoyl-2-hydroxy-sn-glycero-3-phosphate) or PMA (in FreeStyle™ media) was then added to the plate at the indicated concentrations. The plate was placed at 37° C. 5% CO₂ for 5 hours for receptor stimulation followed by beta-lactamase substrate loading for 2 hours.

Results.

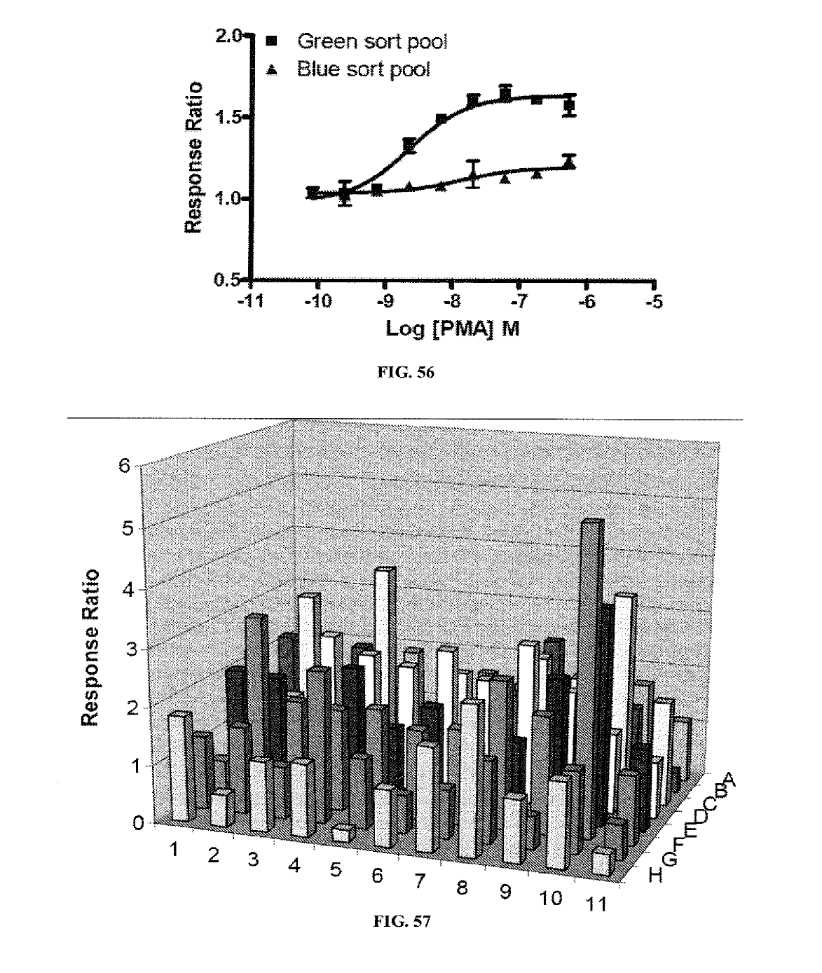

The Green sort pool cell demonstrated a slight inducible beta-lactamase response through the P2RY5 receptor to PMA (FIG. 56).

Conclusions

The Green sort pool from the additional FAGS sorting demonstrated a beta-lactamase response to both FBS and PMA. The PMA response of the Green sort pool can be improved if stimulation time for the assay was increased to 16-20 hours from the 5 hours used in this assay. The clones collected herein will be screened for a response to FBS.

### Example 7

This example is directed to determining the ligand induced response of the FACS isolated clones.

Clone Screening

The Tango P2RY5 U2OS cloned cells were harvested from the 96 well tissue culture plates that the cells were originally sorted into using trypsin/EDTA. The clones were then plated into two wells (for each clone) of a 96 well clear bottom assay plate in growth medium. Each clone was also added to one well of a new 96 well tissue culture plate for culture maintenance. After 24 hours at 37° C./5% CO₂ the growth media was removed from the clones and replaced with Freestyle™ assay medium. One well for each clone also received dialyzed fetal bovine serum (dFBS) to 20% of the final well volume (stimulated). An equal volume of assay Medium was added to the wells that did not receive FBS (un-stimulated). The plate was placed at 37° C./5% CO₂ for 48 hours for receptor stimulation followed by beta-lactamase substrate loading for 2 hours.

Results.

FACS sorted clones demonstrated an inducible beta-lactamase response to dFBS (FIG. 57). Responsive clones were verified visually. (The response ratio value is calculated by the division of the 460/530 ratio of the stimulated wells by the 460/530 ratio of the un-stimulated control cells. A response ratio equal to one is indicative of no functional response.)

Conclusions.

Eighty-eight Tango™ P2RY5 clones were isolated from the sort in Example 6 and were subsequently tested for a receptor response to serum (dFBS). A number of clones demonstrated a beta-lactamase response to serum treatment. Five responding clones were expanded for further testing. Clones expanded: 2C (RR=3.2), 2F (RR=3.2), 4C (RR=3.7), 10E (RR=3.7) and 10F (RR=5.3).

Expanded Clone Testing—FBS Agonist Testing

The Tango™ P2RY5 U2OS sorted clone cells were plated at 10,000 cells/well into a 384 well clear bottom assay plate in Freestyle™ medium. The dFBS was immediately added to the plate to a final concentration of 20% by volume (stimulated). An equal volume of Freestyle™ medium was added to the un-stimulated wells of the plate. The plate was then placed at 37° C./5% CO₂ for 48 hours for receptor stimulation followed by beta-lactamase substrate loading for 2 hours.

Results.

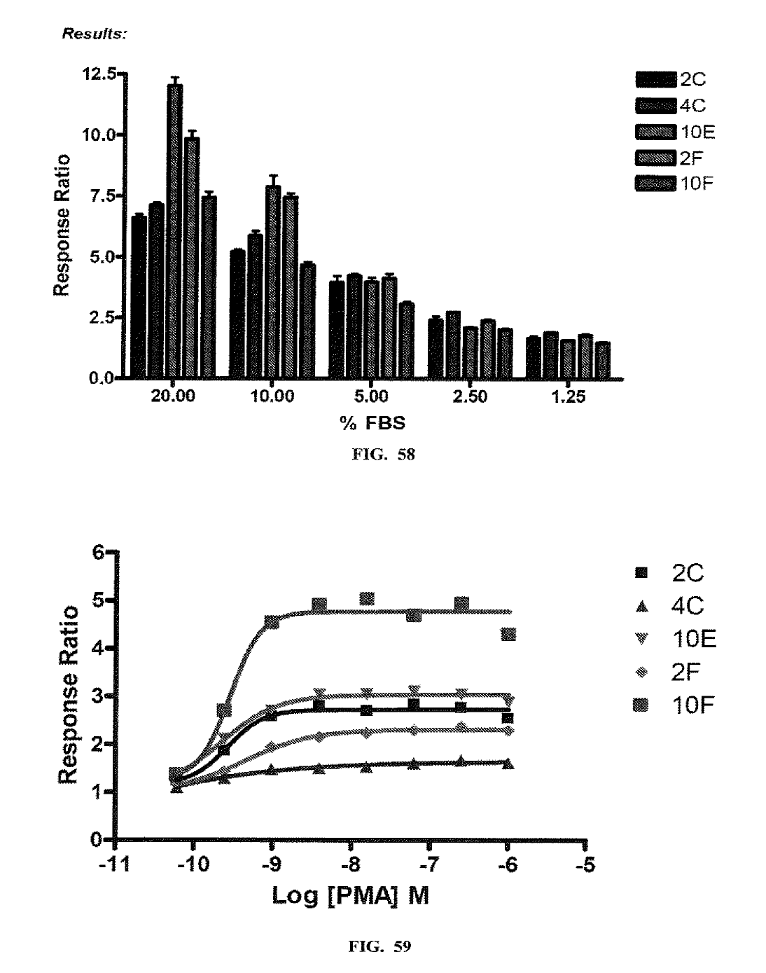

All 5 clones demonstrated a concentration dependant beta-lactamase response to dFBS (FIG. 58). Clone 10E displayed the largest assay window to FBS stimulation. (The response ratio value is calculated by the division of the 460/530 ratio of the stimulated wells by the 460/530 ratio of the un-stimulated control cells. A response ratio equal to one is indicative of no functional response.)

Expanded Clone Testing—PMA Agonist Testing

The Tango™ P2RY5 U2OS sorted clone cells were plated at 10,000 cells/well into a 384 well clear bottom assay plate in Freestyle media. PMA (in Freestyle™ medium) was immediately added to the plate at the indicated concentrations. The plate was placed at 37° C./5% CO₂ for 18 hours for receptor stimulation followed by beta-lactamase substrate loading for 2 hours.

Results.

PMA induced a concentration dependant beta-lactamase response through the P2RY5 receptor in the sorted clones (FIG. 59). Clone 10F displayed the largest assay window to PMA stimulation. (The response ratio value is calculated by the division of the 460/530 ratio of the stimulated wells by the 460/530 ratio of the un-stimulated control cells. A response ratio equal to one is indicative of no functional response.)

Conclusions.

There were 88 Tango™ P2RY5 clones that were isolated from the sort in Example 6. Several clones demonstrated a beta-lactamase reporter response to serum treatment. Five high responding clones were expanded for further testing. Clones expanded: 2C (RR=3.2), 2F (RR=3.2), 4C (RR=3.7), 10E (RR=3.7) and 10F (RR=5.3). The 5 expanded clones all demonstrated a concentration dependant beta-lactamase response to dFBS. Clone 10E displayed the largest assay window to dFBS stimulation. The 5 clones were also determined to be responsive to PMA stimulation. Clone 10F displayed the largest assay window to PMA stimulation. Clone 10F will be subjected to assay validation using PMA as the control compound for the assay. Clone 10F demonstrated the largest assay window in the PMA stimulation assay and also demonstrated a good response to dFBS. PMA stimulation for assay validation will utilize a stimulation time of 16-20 hours since it should be more suitable to concentrated compound exposure than the 48 hours stimulation time for dFBS stimulation. The five clones have been cryopreserved.

### Example 8

This example is directed to the validation of the selected Tango™ P2RY5 U2OS Clone 10F for FITS assay performance in 384 well format.

DMSO Tolerance

The Tango™ P2RY5 U2OS Clone 10F cells were plated at 10,000 cells/well into a 384 well clear bottom assay plate in 32 μl/well Freestyle™ medium. 4 μl/well 10×DMSO in Freestyle™ medium was then added to the wells of the assay plate to bring the final DMSO concentration in the wells to 0% (no DMSO), 0.1%, 0.5% or 1.0% DMSO. 4 μl/well PMA (10× in Freestyle™ medium) was immediately added to the plate to the indicated concentrations. The plate was placed at 37° C./5% CO₂ for 18 hours for receptor stimulation followed by beta-lactamase substrate loading for 2 hours.

Results.

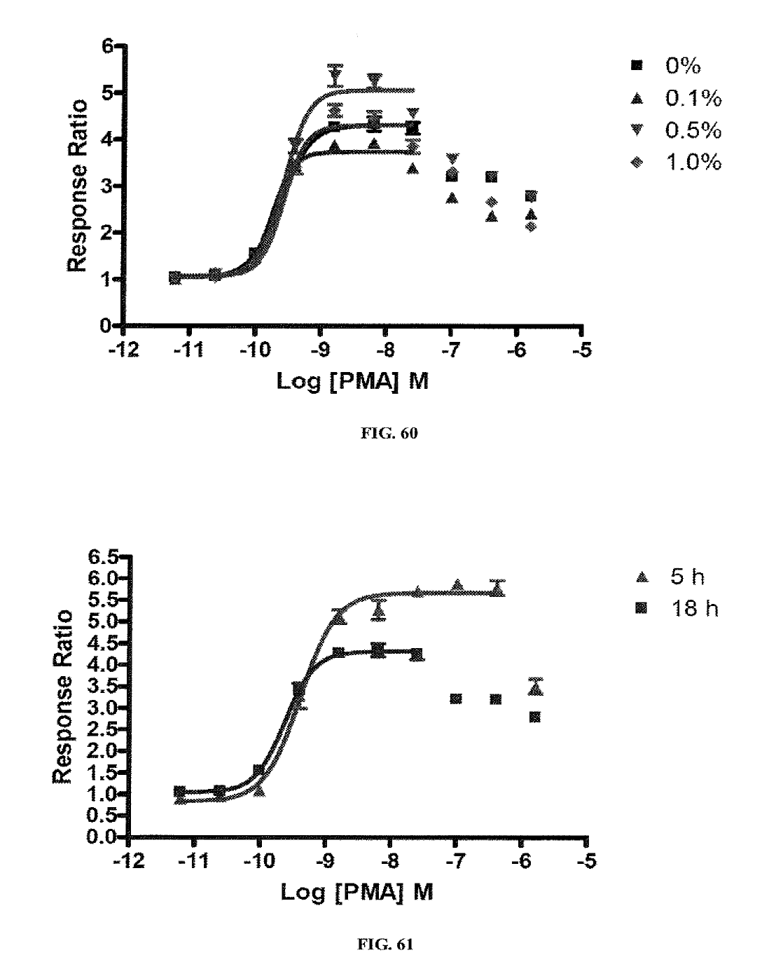

The final concentration of DMSO in the assay does not appear to have an effect on the assay window (FIG. 60). The data points for PMA concentrations that are greater than 100 nM were not included in the curve analysis as there was increasing cell death in those wells. (The response ratio value is calculated by the division of the 460/530 ratio of the stimulated wells by the 460/530 ratio of the un-stimulated control cells. A response ratio equal to one is indicative of no functional response).

Assay Stimulation Time

The Tango™ P2RY5 U2OS Clone 10F cells were plated at 10,000 cells/well into two 384 well clear bottom assay plate in 32 μl/well Freestyle™ medium. One plate was placed at 37° C./5% CO₂ for 18 hours prior to PMA stimulation for 5 hours while PMA was immediately added to the other plate for an 18 hours stimulation. At the time of stimulation 4 μl/well of a 10× stock of DMSO in Freestyle™ medium was added to the wells of the assay plate to bring the final DMSO concentration in the wells to 0.1% DMSO. 4 μl/well PMA (10× in Freestyle™ medium) was then added to the plate to the indicated concentrations. The plates were placed at 37° C./5% CO₂ for 5 or 18 hours for receptor stimulation followed by beta-lactamase substrate loading for 2 h.

Results.

The 5 hour stimulation time demonstrated a larger assay window than the 18 hour stimulation time, and demonstrated less toxic cellular effects at higher PMA concentrations (FIG. 61). The data points for PMA concentrations that are greater than 100 nM (18 hour) or 1 μM (5 hour) were not included in the curve analysis as there was cell death in those wells.

Dividing Vs. Cryopreserved Cells

Tango™ P2RY5 U2OS clone 10F cells were cryopreserved at 2.5×10̂6 cells/ml in Recovery™ freezing medium in liquid nitrogen for several days. The cells were thawed into Freestyle™ assay medium prior to use in the assay. The Tango™ P2RY5 U2OS Clone 10F cells were plated at 10,000 cells/well into two 384 well clear bottom assay plate in 32 μl/well Freestyle™ medium. One plate was placed at 37° C./5% CO₂ for 18 hours prior to PMA stimulation for 5 hours while PMA was immediately added to the other plate for an 18 hour stimulation. At the time of stimulation 4 μl/well of a 10× stock of DMSO in Freestyle™ medium was added to the wells of the assay plate to bring the final DMSO concentration in the wells to 0.1% DMSO. 4 μl/well PMA (10× in Freestyle™ medium) was then added to the plate to the indicated concentrations. The plates were placed at 37° C./5% CO₂ for 5 hours or 18 hours for receptor stimulation followed by beta-lactamase substrate loading for 2 hours.

Results.

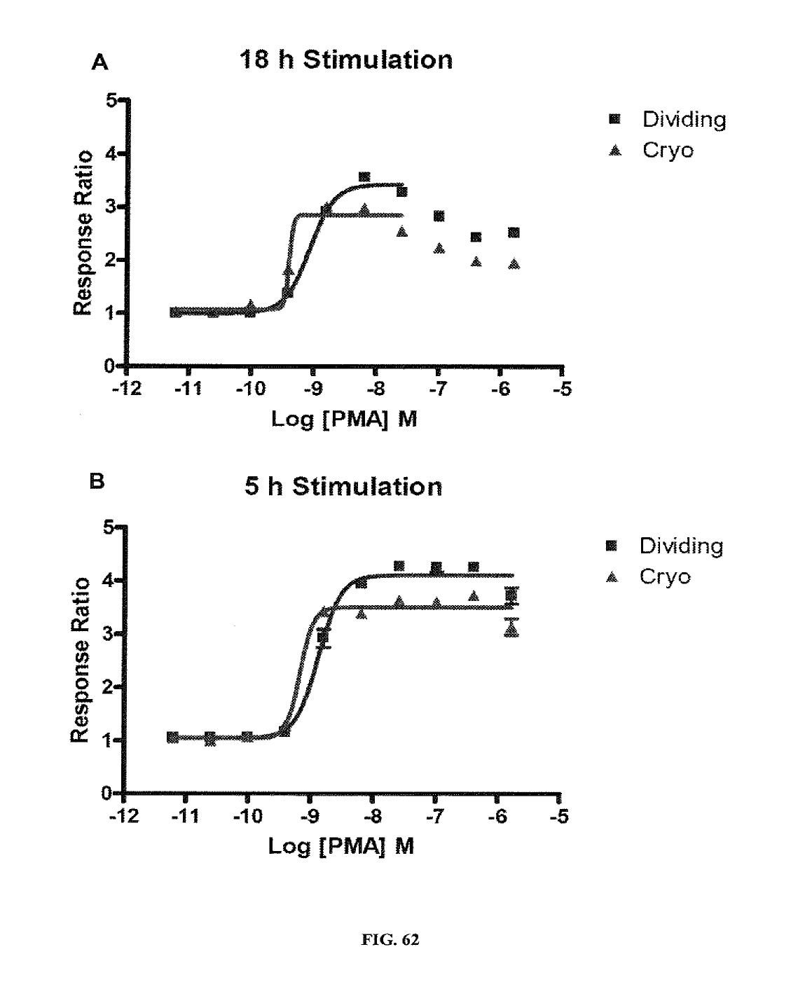

The cryopreserved cells demonstrated a slightly less potent response to PMA than the dividing cells (FIG. 62). The 5 hour stimulation time demonstrated a larger assay window than the 18 hour stimulation time, and demonstrated less toxic cellular effects at higher PMA concentrations. The data points for PMA concentrations that are greater than 100 nM (18 hours) were not included in the curve analysis as there was cell death in those wells.

Assay Reproducibility (Run on 3 Separate Days)

The Tango™ P2RY5 U2OS Clone 10F cells were plated at 10,000 cells/well into a 384 well clear bottom assay plate in 32 μl/well Freestyle™ medium. The plate was placed at 37° C./5% CO₂ for 18 hours prior to receptor stimulation. 4 μl/well 10×DMSO in Freestyle™ medium was then added to the wells of the assay plate to bring the final DMSO concentration in the wells to 0.1%. 4 μl/well PMA (10× in Freestyle™ medium) was immediately added to the plate to the indicated concentrations. The plate was placed at 37° C./5% CO₂ for 5 hours for receptor stimulation followed by beta-lactamase substrate loading forth.

Results.

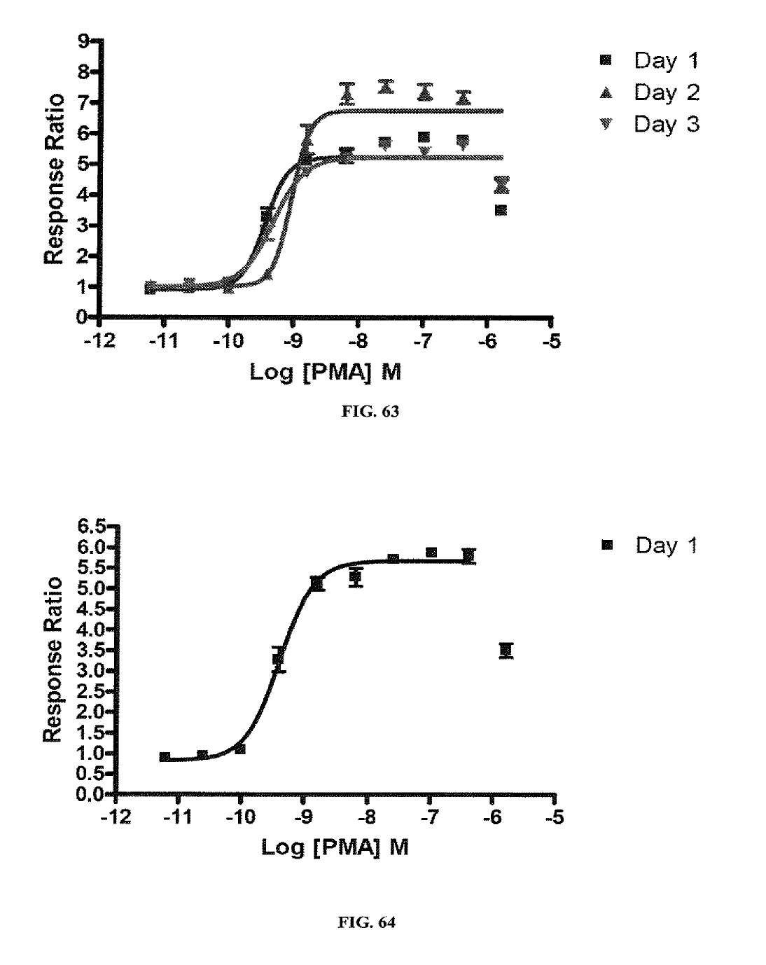

The assay performance is reproducible when run on different days under optimized conditions (FIG. 63).

Conclusions

The optimum stimulation time for the Tango™ P2RY5 assay is 5 hours. The Tango™ P2RY5 assay is tolerant of DMSO concentrations up to 1.0%. The Tango™ P2RY5 cells can be cryopreserved and then thawed directly into the plate for assay. The Tango™ P2RY5 assay demonstrates consistent assay windows and assay quality when assayed on separate days.

### Example 9

Tango™ P2RY5-bla U2OS cells contain the human purinergic receptor P2Y, G-protein coupled, 5 linked to a TEV protease site and a Gal4-VP16 transcription factor stably integrated into the Tango™ GPCR-bla U2OS parental cell line. This parental cell line stably expresses a beta-arrestin/TEV protease fusion protein and the beta-lactamase reporter gene under the control of a UAS response element. The Tango™ P2RY5-bla U2OS cells have been functionally validated for a response to PMA (FIG. 64).

Tango™ P2RY5-bla U2OS Cell-Based Assay Procedure

The following instructions as provided by the manufacturer outline the recommended procedure for determining activity of compounds as modulators of P2RY5 using LiveBLAzer™-FRET BIG Substrate as the readout (Invitrogen product Instructions). If alternative substrates are used (e.g., ToxBLAzer™ DualScreen or LyticBLAzer™ Loading kits), the loading protocol provided with the product should be followed.

Plating Cells.

Cells are harvested and resuspended in Assay Medium (lnvitrogen) to a density of 312,500 cells/ml. 32 μl per well of the Assay Medium is added to the Cell-free Control wells. 32 μl per well of the cell suspension is then added to the Test Compound wells, the Unstimulated Control wells, and Stimulated Control wells. Cells are incubated at 37° C./5% CO₂ for ˜18 hours. An Agonist assay or an Antagonist assay can now be run.

Agonist Assay Plate Setup.

A stock solution of 0.5% DMSO is prepared in Assay Medium (Invitrogen). A 5× stock of Test Compounds in Assay Medium is prepared with 0.5% DMSO as well as a 5× stock of agonist in Assay Medium with 0.5% DMSO. 8 μl of the stock solution of 0.5% DMSO in Assay Medium is then added to the Unstimulated Control and Cell-free Control Wells followed by the addition 8 μl of the 5× stock solution of agonist to the Stimulated Control wells. 8 μl of the 5× stock of Test Compounds is then added to the Test Compound wells followed by subsequent incubation of the Agonist assay plate in a humidified 37° C./5% CO₂ incubator for 5 hours.

Antagonist Assay Plate Setup.

A stock solution of 0.5% DMSO is prepared in Assay Medium. A 10× stock of Test Compounds is prepared in Assay Medium with 0.5% DMSO as well as a 10× stock of agonist in Assay Medium with 0.5% DMSO. A 10× stock of antagonist is also prepared in Assay Medium with 0.5% DMSO. 4 μl of the 10× stock of Test Compounds is added to the Test Compound wells. 4 μl of the stock solution of 0.5% DMSO is then added to the Stimulated Control wells, the Unstimulated Control wells, and the Cell-free Control wells. 4 μl of the 10× stock of antagonist in Assay Medium with 0.5% DMSO is subsequently added to the Antagonist Control wells. The Test Compounds can be incubated with the cells humidified 37° C./5% CO₂ incubator before proceeding, for about a 30-minute incubation. 4 μl of the 10× stock solution of agonist is then added to the Test Compound wells, the Stimulated Control wells, and the Antagonist Control wells, followed by the addition of 4 μl of Assay Medium with 0.5% DMSO to the Unstimulated Control and Cell-free Control wells. The Antagonist assay plate is then incubated in a humidified 37° C./5% CO₂ incubator for 5 hours.

Substrate Preparation, Loading and Incubation.

Substrate preparation, loading and incubation is carried out according to manufacturer's instructions. LiveBLAzer™-FRET B/G Substrate Mixture (CCF4-AM) and the loading of cells should be carried out in the absence of direct strong lighting.

Detection.

Detection is carried out according to the Invitrogen's instructions. Briefly, measurements are made at room temperature from the bottom of the wells, preferably in 384-well, black-wall, clear-bottom assay plates with low fluorescence background. Before reading the plate, dust must be removed from the bottom with compressed air.

A fluorescence plate reader with bottom reading capabilities should be used, and should utilize an Excitation filter at 409/20 nm, an Emission filter at 460/40 nm, as well as an emission filter at 530/30 nm. The following filter selections can be used:

| | Scan 1 | Scan 2 |
|---|---|---|
| Purpose: | Measure fluorescence | Measure FRET signal |
| | in the Blue channel | in the Green channel |
| Excitation filter: | 409/20 nm | 409/20 nm |
| Emission filter: | 460/40 nm | 530/30 nm |

Background subtraction and ratio calculation is carried out according to the Invitrogen's instructions.

Visual observation of intracellular beta-lactamase activity using LiveBLAzer™-FRET B/G Substrate (CCF4-AM) is carried out according to the Invitrogen's instructions.

What is claimed:

1. A method for controlling hair growth in a subject, the method comprising:
(a) administering to the subject an effective amount of a P2RY5 receptor antagonist; thereby controlling hair growth in the subject.

2. The method of claim 1, wherein the subject is a human, a primate, a feline, a canine, or an equine.

3. The method of claim 27, wherein the antagonist is selected from the group consisting of:
(a) an antibody that specifically binds to a P2RY5 protein or fragment thereof;
(b) an antisense RNA or antisense DNA that inhibits expression of P2RY5 polypeptide;
(c) an siRNA that specifically targets a P2RY5 gene;
(d) a peptide comprising at least 10 amino acids f the SEQ ID NO: 1 wherein the peptide competes with endogenous P2RY5 receptor for ligand binding; and
(e) combinations of (a)-(d).

4. The method of claim 1, wherein the location of said hair growth is selected from the group consisting of:
(a) the arms;
(b) the legs;
(c) the bikini area;
(d) the face; and
(e) combinations of (a)-(d).
